(19) United States
(12) Patent Application Publication
(10) Pub. No.: US 2024/0108716 A1
(43) Pub. Date: Apr. 4, 2024

(54) MONOVALENT AND MULTIVALENT VACCINES FOR PREVENTION AND TREATMENT OF DISEASE

(71) Applicant: The Regents of the University of California, Oakland, CA (US)

(72) Inventors: Nicole F. Steinmetz, La Jolla, CA (US); Oscar A. Ortega-Rivera, La Jolla, CA (US); Jonathan K. Pokorski, La Jolla, CA (US)

(21) Appl. No.: 18/271,366

(22) PCT Filed: Jan. 7, 2022
(86) PCT No.: PCT/US2022/011722

§ 371 Date: Jul. 7, 2023

Related U.S. Application Data
(60) Provisional application No. 63/135,492, filed on Jan. 8, 2021.
(60) Provisional application No. 63/237,378, filed on Aug. 26, 2021.

(51) Int. Cl. A61K 39/215; A61K 39/00; A61K 39/385; A61P 31/14

(52) U.S. Cl. CPC A61K 39/215; A61K 39/0005; A61K 39/0012; A61K 39/385; A61P 31/14; A61K 2039/525; A61K 2039/5258; A61K 2039/6093

(57) ABSTRACT

Provided are compositions, methods and uses relating to one or more virus or virus-like particle(s), each of which comprises at least one epitope(s) of a pathogen causing the disease or one or more of the cholesterol checkpoint protein(s).

### CROSS-REFERENCE TO RELATED APPLICATIONS

This application claims the benefit under 35 U.S.C. § 119(e) of U.S. Provisional Application Ser. No. 63/135,492, filed Jan. 8, 2021 and 63/237,378, filed Aug. 26, 2021, the contents of which are hereby incorporated by reference in its entirety.

### STATEMENT OF GOVERNMENT SUPPORT

This invention was made with government support under Grant No. HL137674 awarded by the National Institutes of Health (NIH). The government has certain rights in the invention.

### BACKGROUND

The present disclosure relates generally to the field of treatment and prevention of diseases such as cardiovascular and infectious diseases.

Treatment and Prevention of Cardiovascular Disease

Cardiovascular disease (CVDs) is the number one cause of death globally. An estimated 17.9 million people died from CVD in 2016, representing 31% of all global deaths. Of these deaths, 85% are due to heart attack and stroke and over three quarters of CVD deaths take place in low- and middle-income countries. [World Health Organization (2016) Descriptive note 3 17 May/2017] Atherosclerosis is a chronic inflammatory disease that develops in response to lipid accumulation, immune response, and inflammation. [A. Gisterå et al., Nature Reviews Nephrology 2017; J. G. Robinson et al., Atherosclerosis 2015] The primary events in atherosclerosis are the accumulation and oxidation of lipoproteins such as low-density lipoprotein cholesterol (LDL-C) leading to inflammation of the vascular wall; [A. Gisterå et al., Nature Reviews Nephrology 2017; J. G. Robinson et al. Atherosclerosis 2015] rupture of such plaques can lead to clinical events such as a heart attack and stroke. A primary goal in the treatment of atherosclerosis is to decrease the LDL-C levels in plasma. Statins are the main drugs used to achieve this goal; however, their efficacy is compromised by side effects and the need for lifelong treatments. [J. G. Robinson et al., Atherosclerosis 2015] Immunotherapy holds promise in the management of CVD and the recent US Food and Drug Administration (FDA) approval of monoclonal antibodies (Alirocumab and Evolocumab) targeting the proprotein convertase subtilisin/kexin 9 (PCSK9) pave the way for this development. PCSK9 is a protease that binds and promotes degradation of the LDL receptor. Blocking PCSK9 leads to more LDL receptors on the surface of hepatocyte resulting in lower serum level of LDL-C. [M. S. Sabatine et al., New England Journal of Medicine 2015; J. G. Robinson et al., New England Journal of Medicine 2015; S. E. Nissen et al., JAMA—Journal of the American Medical Association 2016] While this therapy regimen is a promising approach, this passive immunotherapy requires repeated dosing and lifelong treatment; this and the high cost associated with this therapy make it unattainable for a great portion of patients. The costs per dose are approximately $2,300-4,000 per patient for a one-year supply in the United States; these costs are prohibitive for implementation in developing countries. [ICER, Institute for Clinical and Economic Review. Published February 24th, 2019]

Treatment and Prevention of HPV and Associated Diseases

Human papillomavirus (HPV) is a globally-prevalent pathogen and the most common sexually-transmitted infection in the USA. [Centers for Disease Control and Prevention. Genital HPV Infection—CDC Fact Sheet, 2014] There are many strains of HPV, including low-risk types associated with warts, and high-risk types that give rise to various forms of anal, penile, and oropharyngeal cancer, as well as almost all cases of cervical cancer [Zhai, L. et al., Antivir. Res. 2016] Most HPV infections cause no symptoms and are cleared by the immune system, but persistent infections might lead to the more serious health issues listed above. [Centers for Disease Control and Prevention. Genital HPV Infection—CDC Fact Sheet, 2014] Vaccines can protect against the most dangerous strains of HPV, and three were approved. [Petrosky, E. et al., Mmwr. Morb. Mortal. Wkly. Rep. 2015] The latest (Gardasil 9) protects against nine HPV types and prevents 90% of HPV-associated cancers every year. [Zhai, L. et al., Antivir. Res. 2016; Centers for Disease Control and Prevention. Mmwr. Morb. Mortal. Wkly. Rep. 2010; Centers for Disease Control and Prevention. HPV Vaccine Information for Clinicians—Fact Sheet 2012; Kirby, T., Lancet Oncol. 2015]

Despite the protection offered by the HPV vaccine, only 53.7% of girls and 48.7% of boys in the US were vaccinated against HPV in 2019. [Walker, T. Y. et a., Morb. Mortal. Wkly. Rep. 2019] This compares poorly to 66% of girls and 42% of boys who completed the vaccination program in 2011, suggesting that take-up rates are declining. [Stokley, S. et al., Mmwr. Morb. Mortal. Wkly. Rep. 2014] The poor quality of public health services in many developing countries means the vaccination rates are even worse, even though HPV infections are more prevalent. Factors that contribute to the poor acceptance of the HPV vaccine include costs, lack of knowledge about HPV transmission, parental distrust of vaccines, and the prolonged vaccination schedule (which requires three injections to achieve full protection). Based on 2011 data, only 70.7% of girls and 28.1% of boys who receive the first dose go on to complete the course. [Stokley, S. et al., Mmwr. Morb. Mortal. Wkly. Rep. 2014; Reagan-Steiner, S. et al., Mmwr. Morb. Mortal. Wkly. Rep. 2015] Another considerable logistical and fiscal barrier is the cold chain requirement for HPV vaccines, making it untenable to distribute life-saving vaccines in resource-poor areas of the world. Innovating vaccine platforms and delivery devices to break cold chain limitations is therefore an excellent solution to safeguard potent vaccination for both wealthy and lower-income countries.

Treatment and Prevention of COVID and Associated Diseases

The rise of severe acute respiratory syndrome coronavirus 2 (SARS-CoV-2) leading to the coronavirus disease (COVID-19) pandemic highlights the need for platform technologies enabling rapid development of vaccines for emerging viral diseases. An unprecedented collaboration between key players in the vaccine R&D industry and academic labs around the world led to the development, emergency authorization, and administration of millions of doses of nanotechnology-based vaccines around the globe within record time. However, successful vaccination programs are still mostly limited to developed countries and the roll-out of mass vaccinations was burdened by undesirable attributes, such as the requirement of storage at ultra-low temperatures, delivery via injection thus requiring medical staff, the requirement of a prime-boost vaccination schedule, and the methods to scale up the current COVID-19 vaccines were challenging. Thus, a need still exists in the art for the treatment and prevention of COVID-19.

### SUMMARY OF THE DISCLOSURE

Provided herein are active vaccination approaches for the treatment and prevention of cardiovascular and infectious diseases that overcome the shortcomings of statins and monoclonal antibodies Effective vaccinations achieve life-long efficacy after vaccination with lower costs. For example, in one aspect, Applicant's trivalent vaccine for the treatment of cardiovascular disease elicits neutralizing antibodies against its targets and lowers cholesterol. In other embodiments, the trivalent vaccine is given as prime-boost administration or delivered by an implant that comprises, or consists essentially of, or yet further consists of a poly(lactic-co-glycolic acid) (PLGA) polymer implant.

Accordingly, in one aspect, provided herein is a trivalent vaccine targeting cardiovascular checkpoints to lower plasma cholesterol levels and that therefore in one aspect, are useful to prevent stroke/heart attack or other coronary vascular diseases in patients in need thereof, e.g., high risk patients. Without wishing to be bound by the theory, the vaccines elicit neutralizing antibodies to keep cholesterol levels in check. In other embodiments, the vaccines are formulated to comprise peptide epitopes derived from proprotein convertase subtilisin/kexin-9 (PCSK9), apolipoprotein B (ApoB), and cholesteryl ester transfer protein (CETP). In further embodiments, the peptide epitopes of the vaccines are displayed on a virus like particle (VLP) from the phage Qbeta (QP). In some embodiments, the vaccines are delivered by a method comprising, or consisting essentially of, or yet further consisting of, as a prime boost injection or a method using a vaccine delivery device (PLGA-based polymer implant).

In a further aspect, provided is a vaccine (such as trivalent or multivalent) delivered by a method comprising, or consisting essentially of, or yet further consisting of a prime boost injection or by a method comprising, or consisting essentially of, or yet further consisting of a PLGA polymer vaccine implant. Additionally or alternatively, the peptide epitopes are displayed on a virus like particle (VLP) from the bacteriophage Qβ (also referred to herein as phage Qbeta or Qβ).

In some embodiments, provided are COVID vaccines comprising COVID epitopes. In some embodiments, provided are multivalent COVID-19 vaccines, for example, using a cowpea mosaic virus as the display nanoparticle to display COVID epitopes, e.g., SARS-CoV-2 epitopes.

A non-limiting exemplified advantage of the disclosure herein includes slow release and storage not requiring the cold chain. As is to be appreciated by the skilled artisan, the vaccines can display the same or different epitopes that can be specific to the same or different target, e.g., different variants of SARS-CoV-2.

In other aspects, provided herein is a single epitope HPV vaccine or a vaccine for another infectious disease that overcome the challenges of prior art vaccines. Challenges for many vaccinations are the need for multiple doses and cold chain requirements, leading to a lack of compliance and incomplete protection. To address the drawbacks of current HPV and other vaccines, a scalable manufacturing process is used to prepare implantable polymer-protein blends for single-administration sustained delivery. As an example, peptide epitopes from HPV16 capsid protein L2 were conjugated to the virus-like particle derived from bacteriophage Qβ to enhance their immunogenicity. The HPV-Qβ particles were then encapsulated into poly(lactic-co-glycolic acid) (PLGA) (although other polymers can be used) implants using a benchtop melt-processing system. The implants facilitated the slow and sustained release of HPV-Qβ particles without loss of nanoparticle integrity during high temperature melt processing. Mice vaccinated with the implants generated IgG titers comparable to the traditional soluble injections and achieved protection in a pseudovirus neutralization assay. Thus, HPV-Qβ implants are an example of a new vaccine and vaccination platform; because melt-processing is so versatile, the technology offers the opportunity for massive upscale into any geometric form factor. The Qβ technology is highly adaptable allowing the production of vaccines and their delivery devices for multiple strains and/or types of viruses.

In one aspect, provided is a method for one or more of the following in a subject in need thereof: treating or preventing a cardiovascular disease, treating or preventing an atherosclerosis, treating or preventing a hypercholesterolemia, treating or preventing a lipid dyshomeostasis, preventing a heart attack, preventing a stroke, reducing a statin administration dose or frequency or both, reducing a cholesterol level, reducing an oxidized cholesterol level, reducing a low-density lipoprotein cholesterol (LDL-C) level, reducing a level or an activity of one or more cholesterol checkpoint protein(s), producing an antibody recognizing and binding to one or more cholesterol checkpoint protein(s), triggering, enhancing, or prolonging an immune response to one or more cholesterol checkpoint protein(s), or delivering at least one epitope(s) of one or more cholesterol checkpoint protein(s) to the subject. The method comprises, consists essentially of, or consists of administering to the subject, for example an effective amount of, one or more virus or virus-like particle(s), wherein each virus or virus-like particle comprises at least one epitope of the cholesterol checkpoint protein(s), and optionally comprising two or more epitopes that may be the same or different from each other.

In another aspect, provided is a method for one or more of the following in a subject in need thereof: treating or preventing an infectious disease or another disease caused by a pathogen, producing an antibody recognizing and binding to one or more pathogen(s) causing the disease, triggering, enhancing, or prolonging an immune response to one or more pathogen(s) causing the disease, or delivering at least one epitope(s) of the pathogen(s) causing the disease to the subject. The method comprises, consists essentially of, or consists of administering to the subject, for example an effective amount of, one or more virus or virus-like particle(s), each of which comprises at least one epitope of the pathogen and optionally comprising two or more epitopes that may be the same or different from each other.

In yet another aspect, provided is a composition or a vaccine particle comprising, consisting essentially of, or consisting of an optional carrier and one or more virus or virus-like particle(s), each of which comprises at least one epitope of a pathogen causing a disease or each of which comprises at least one epitope of one or more cholesterol checkpoint protein(s) and optionally comprising two or more epitopes that may be the same or different from each other.

The particles also can be delivered by microneedle patches would allow for self-administration in the absence of a health care profession within the developing world. Thus, in one aspect provided herein is a microneedle patch comprising the vaccine particles as disclosed herein and method for vaccination comprising administering to, or placing the patch on the patch on the subject to be treated.

One advantage of the present disclosure is that the active ingredient is not the plant virus or bacteriophage, but rather the complex coupled with the antigenic epitope.

Further provided by this disclosure are the compositions prepared for administration of the disclosed methods, as well as methods for making them. These can be combined with a carrier, such as a pharmaceutically acceptable carrier and or device, e.g., microneedle device or nebulizer for inhalation therapy.

The disclosed methods can be combined with other known methods known to the treating physician or veterinarian.

### BRIEF DESCRIPTION OF THE DRAWINGS

FIG. 1: Qβ VLP vaccine design. Top) Genes encoding unmodified and modified Qβ coat proteins (CP) with the corresponding peptides (QβApoB, QβCETP, and QβPCSK9); genes are not drawn to scale; middle) Vectors used for expression in E. coli BL21 (DE3): unmodified Qβ CP gene was cloned into pCDF_Qβ and served as a control. Modified and unmodified Qβ CP genes were cloned resulting in the following expression vectors: pCDF_Qβ_QβApoB, pCOLA_Qβ_QβCETP, and pRSF_Qβ_QβPCSK9; the peptide-displaying CP was put under control of a second T7 promoter; bottom) hybrid Qβ VLP vaccines; inset shows the number of peptides displayed per VLP as determined by SDS-PAGE analysis. lacI=lactose repressor protein gene; CloDF13 ori=CloDF13 origin of replication; ColA ori=ColA origin of replication; RSF ori=RSF origin of replication; SmR=Streptomycin resistance gene; AmpR=Ampicillin resistance gene promoter; KnR=Kanamycin resistance gene.

FIGS. 2A-2E: Characterization of Qβ VLP vaccines. (FIG. 2A) FPLC chromatogram of unmodified Qβ VLP and Qβ VLP vaccines QβApoB, QβCETP, or QβPCSK9. (FIG. 2B) DLS of unmodified Qβ VLPs and Qβ VLP vaccines. (FIG. 2C) TEM images of negatively-stained unmodified Qβ VLPs and Qβ VLP vaccines (scale bar=100 nm). (FIG. 2D) Electrophoretic mobility of unmodified Qβ VLPs and Qβ VLP vaccines on native 0.8% (w/v) agarose gels stained with Gelred™ Nucleic Acid Gel Stain (VLPs package random host RNA enabling detection by nucleic acid staining). (FIG. 2E) SDS-PAGE analysis of unmodified Qβ VLP and Qβ VLP vaccine candidate under reducing conditions. Unmodified Qβ coat protein (CP, white triangle, ˜14 kDa) and modified Qβ CPs (yellow dot, QβApoB=˜16.4 kDa, red dot, QβCETP=˜15.9 kDa, and blue dot, QβPCSK9=˜16 kDa) were visualized after Coomassie Brilliant Blue staining. All samples were tested in triplicate (n=3) for all the characterization methods, and the pictures and data shown are representative from those triplicates.

FIGS. 3A-3C: Immunization and antibody response. (FIG. 3A) Vaccination schedules for the Qβ VLP vaccines QβApoB, QβCETP or QβPCSK9. Mice in the single antigen group were immunized s.c. using a 100 μg-dose at weeks 0, 2, and 4 (black arrows) of each Qβ, QβApoB, QβCETP or QβPCSK9, respectively. The 3Ag s.c. group received a mixture of the three Qβ VLP vaccines at 100 μg-per VLP dose. Lastly, the 3Ag implant group was immunized using PLGA:VLP implants containing a mixture of the three Qβ VLP vaccines, loaded into the PLGA implant at a 100 μg-per VLP dose (300 μg protein total). (FIG. 3B) Endpoint antibody titers at different weeks after first immunization. Week 0 corresponds to plasma collected prior to the first immunization. Plasma samples from free-peptides (-ApoB, -CETP or -PCSK9) and unmodified Qβ VLP (Qβ) were used as control groups. Plasma samples from five mice (n=5) were tested individually in duplicate. Data is presented as mean±SD. (FIG. 3C) IgG isotype profile (IgG2b/IgG1 ratio), a ratio lower than 1 was considered Th2-biased. Plasma samples from five mice (n=5) from the same group were pooled and tested five times for each Ig isotype. Data is presented as mean±SD.

FIGS. 4A-4K: Plasma levels of ApoB and PCSK9, and CETP in vitro. (FIG. 4A and FIG. 4B) ELISA against LDL and oxidized LDL (oxLDL), respectively. Plasma samples from five mice were pooled and five technical replicas were analyzed (n=5). (FIG. 4C, FIG. 4D and FIG. 4E) Plasma ApoB protein concentration for single antigen, 3Ag s.c., and 3Ag implant groups, respectively. Plasma was evaluated at week 0 (pre-immunization) and week 8 post first immunization. Plasma samples from five mice were pooled and five technical replicas were analyzed twice (n=10). (FIG. 4F and FIG. 4G) CETP activity kinetic curve after incubation with plasma samples and CETP percentage activity after 90 min. Plasma samples from five mice were pooled and three technical replicas were analyzed twice (n=6). (FIG. 411) ELISA of sera from vaccinated groups against mouse PCSK9 protein. Plasma samples from five mice were pooled and three technical replicas were analyzed twice (n=6). (FIG. 41, FIG. 4J and FIG. 4K) PCSK9 protein concentration in plasma from mice receiving the various vaccines at week 0 (pre-immunization) and week 8 post first immunization. Plasma samples from five mice were pooled and five technical replicas were analyzed (n=5). Depleted IgG=plasma after depletion of IgGs to remove IgG-PCSK9 immunocomplexes. Single antigen group=QβApoB, QβCETP or QβPCSK9 VLPs; 3Ag s.c.=mixture of QβApoB, QβCETP, and QβPCSK9 injected s.c.; 3Ag implant=mixture of QβApoB, QβCETP, and QβPCSK9 delivered via PLGA:VLP implants. Plasma samples from free-peptides (-ApoB, -CETP, and -PCSK9) and unmodified Qβ VLP (Qβ) were used as control groups. Data is shown as mean±SD. All comparison between means of vaccinated and control group were done using unpaired t test (two-tailed, 95% confidence value). P values<0.05 were considered as statistically significant.

FIGS. 5A-5C: Total cholesterol concentration in plasma. Total cholesterol was determined at different time points after, pre- and post-immunization (week 0, 8, and 12) for (FIG. 5A) single antigen groups, (FIG. 5B) 3Ag s.c. group, and (FIG. 5C) 3Ag implant group. Single antigen group=QβApoB, QβCETP or QβPCSK9 VLP vaccine; 3Ag s.c.=mixture of QβApoB, QβCETP, and QβPCSK9 injected subcutaneously; 3Ag implant=mixture of QβApoB, QβCETP, and QβPCSK9 prepared as loaded PLGA implants; ns=no significance (p>0.05). Plasma samples from free-peptides (-ApoB, -CETP, and -PCSK9) and unmodified Qβ VLP (Qβ) were used as control groups. Plasma samples from five mice were pooled and four technical replicas were analyzed twice (n=8). Data is shown as mean±SD. All comparison between means of vaccinated and control group were done using unpaired t test (two-tailed, 95% confidence value). P values<0.05 were considered as statistically significant.

FIGS. 6A-6C: Kidney and liver plasma biomarkers. (FIG. 6A) Kidney Injury Molecule-1 (KIM-1); (FIG. 6B) aspartate transaminase (AST); and (FIG. 6C) alanine transaminase (ALT) levels in plasma samples collected from mice receiving the trivalent vaccine candidate (3Ag s.c. group) at week 0 and 12 after first vaccination compared to age-matched mice. 3Ag s.c.=mixture of QβApoB, QβCETP, and QβPCSK9 injected subcutaneously. Dotted lines=threshold for normal values in healthy mice. Plasma samples from five mice were pooled and four technical replicas were analyzed twice (n=4). Data is shown as mean±SD. All comparison between means of vaccinated and control group were done using unpaired t test (two-tailed, 95% confidence value). P values<0.05 were considered as statistically significant.

FIGS. 7A-7B: T-cell activation using ELISpot assay and splenocytes from immunized mice. Splenocytes (5×10⁵ cells per well) from 3Ag s.c. immunized mice were stimulated with medium only (no stimulation control), a mixture of free-peptides (-ApoB, -CETP, and -PCSK9), a mixture of targeted proteins (ApoB, CETP, and PCSK9), unmodified Qβ VLP or PMA/Ionomycin (positive control). (FIG. 7A) Cytokine (IFN-7 and IL-4) producing cells were counted as splenocytes forming colonies (SFC). (FIG. 7B) Representative images of blue (IL-4) and red (IFN-7) spots formed by stimulated splenocytes. Splenocytes from three mice (n=3) were tested individually in triplicate. Data is shown as mean±SD. All comparison between means of vaccinated and control group were done using unpaired t test (two-tailed, 95% confidence value). P values<0.05 were considered as statistically significant. *=p<0.05 compared to medium.

FIGS. 8A-811: The synthesis, characterization, and immunogenicity of the HPV-Qβ conjugates. (FIG. 8A) Schematic representation of the synthesis of four HPV-Qβ conjugates differing in the linker structure and epitope orientation. (FIG. 8B) SDS-PAGE analysis of the Qβ and HPV-Qβ particles: (1) Qβ, (2) GPSL-N-expo-HPV-Qβ, (3) GPSL-C-expo-HPV-Qβ, (4) GGSG-N-expo-HPV-Qβ, and (5) GGSG-C-expo-HPV-Qβ. (FIG. 8C) FPLC chromatogram of GGSG-C-expo-HPV-Qβ. (FIG. 8D) TEM image of GGSG-C-expo-HPV-Qβ (scale bar=100 nm). (FIG. 8E) DLS analysis of GGSG-C-expo-HPV-Qβ. (FIG. 8F) Serum IgG titers against the HPV peptide following immunization with the four HPV-Qβconjugates or Qβ. (FIG. 8G) Serum IgG titers against Qβ following immunization with the four HPV-Qβ conjugates or Qβ. (FIG. 8H) ELISA signals at 405 nm for mice vaccinated with different antigens at different dilutions after the second boost. Mice were subcutaneously injected with 30 μg of each agent on day 0 (prime), 14 (first boost), and 28 (second boost). Blood was collected 7 days after each injection (Data are means±S.D. for n=5 mice per group). Asterisks show significance as determined by unpaired two-tailed student's t-test: ns, not significant p>0.05; *p<0.05; ***p<0.005.

FIGS. 9A-9D: The preparation and characterization of melt-extruded PLGA implants. (FIG. 9A) Equipment for the melt processing of polymer materials. (FIG. 9B) PLGA implants produced by melt extrusion. The long implant bar produced directly from the device is shown at the top, with the short sections ready for implantation shown underneath. The photograph shows a metric ruler. The white bars are plain PLGA+Qβ-HPV, and the green ones are FITC-PLGA+Cy5-Qβ. (FIG. 9C) SEM image showing a cross-section of the PLGA implant loaded with Qβ-HPV. (FIG. 9D) The EDX spectrum sulfur K-series emission signal (SK series) map of the PLGA implant loaded with Qβ-HPV.

FIGS. 10A-10E: Slow release of Qβ-HPV from the PLGA implant. (FIG. 10A) The release curve of the PLGA implants loaded with Qβ-HPV or Qβ, with the particles released into PBS at 37° C. (data are means±standard deviations, n=3). (FIG. 10B) SDS-PAGE analysis of (1) unmodified Qβ standard and Qβ-HPV released from the implant between (2) 0-15 days and (3) 16-30 days. (FIG. 10C) TEM image of the released Qβ-HPV particles on day 30. (FIG. 10D) FPLC analysis of the released Qβ-HPV particles on day 30. (FIG. 10E) DLS analysis of the released Qβ-HPV particles on day 30.

FIGS. 11A-11C: Release of Cy5-Qβ particles from PLGA implants in vivo. (FIG. 11A) Fluorescence images of mice containing FITC-PLGA implants loaded with Cy5-Qβ, showing the Cy5 channel (top) and GFP channel (bottom) at different time-points. Blue square shows implant location. Scale bar shows fluorescence intensity for all images in each channel. (FIG. 11B) Quantitative release profile (Cy5 channel) for the FITC-PLGA implants loaded with Cy5-Qβ (Data are means±s.d. for n=4 mice per group). (FIG. 11C) Quantitative release profile (GFP channel) for the FITC-PLGA implants loaded with Cy5-Qβ(Data are means±s.d. for n=4 mice per group).

FIGS. 12A-12D: Single-dose vaccination using PLGA implants loaded with HPV-Qβ. (FIG. 12A) Vaccination and bleeding schedule for mice with subcutaneous PLGA implants or equivalent HPV-Qβ injections. (FIG. 12B) Serum titers of HPV-specific IgG for mice vaccinated with three subcutaneous injections of 30 μg HPV-Qβ or a single-dose PLGA implant loaded with 100 μg HPV-Qβ, 500 μg HPV-Qβ, a mixture of 100 μg Qβ and 20 μg HPV peptide, or 20 μg HPV peptide alone (Data are means±s.d. for n=6 mice per group). Statistical significance was determined by unpaired two-tailed student's t-test: ns, not significant with p>0.05. (FIG. 12C) HPV-specific IgG subtype ratio for mice vaccinated with a single-dose PLGA implant containing 100 μg HPV-Qβ, or three subcutaneous doses of 35 μg HPV-Qβ. Blood was collected on day 35 (Data are means±s.d. for n=6 mice per group). (FIG. 12D) Serum (day 35) from three mice immunized with various vaccine formulations tested in duplicate for neutralization against HPV16 pseudovirus at ID₆₀ (pseudovirus infectious dose that infects 60-70% of control cells). Infected cells (expressing GFP) were identified by flow cytometry (data are means±standard errors based on the relative percentage of infected cells in wells exposed to serum compared to non-exposed controls).

FIG. 13: DLS of CPMV-based COVID-19 vaccines.

FIGS. 14A-14B: Trivalent Qβ-based COVID-19 vaccine microneedle patch. (FIG. 14A) Digital photograph of a dissolvable active vaccine microneedle patch comprised of an array of 15×15 needles, corresponding scanning electron micrograph (SEM), and energy dispersive X-ray (EDX) elemental analysis of Mg. Scale bars, 5 mm, 400 μm and 250 μm, respectively. (FIG. 14B) Digital photograph of a dissolvable active vaccine microneedle patch comprised of an array of 15×15 needles and corresponding scanning electron micrograph (SEM). Scale bars, 5 mm and 400 μm.

FIGS. 15A-15C: ELISA binding of IgG to peptide. (FIG. 15A) Immunization schedule. The groups were studied as follows: trivalent 3Qβ microneedle (MN) active (500 μg of each Qβ/dose). As control group blank MN were used, n=5 mice per group. (FIG. 15B) Endpoint IgG titers from vaccinated animals at different weeks (0-4) after first immunization. Week 0 corresponds to plasma collected prior to the first immunization. (FIG. 15C) IgG isotype profile (IgG2a/IgG1 ratio) at week 4, a ratio>1 was considered as Th1-biased and <1 as Th2-biased.

FIGS. 16A-16F: Qβ and CPMV-based COVID-19 vaccines' full Ig isotype profile at week 4. ELISA against peptide (FIG. 16A) 570, (FIG. 16B) 636, and (FIG. 16C) 826 using 1:500 pooled plasma from the different groups (n=5) vaccinated with the Qβ vaccines. ELISA against peptide (FIG. 16D) 570, (FIG. 16E) 636, and (FIG. 16F) 826 using 1:1000 pooled plasma from the different groups (n=3) vaccinated with the CPMV570, CPMV636, and CPMV826 vaccines. Data represents the mean±SD.

FIGS. 17A-17B: Trivalent Qβ COVID-19 vaccine delivery strategies. (FIG. 17A) general concept of the active MN patches shows the enhance release of the vaccine after the MN dissolves in the dermis due to the active micro pumping generated by Mg microparticles. (FIG. 17B) Active MN patch and its components.

FIGS. 18A-18E: Synthesis and characterization of CPMV-based COVID-19 vaccines. (FIG. 18A) Conjugation of B cell epitope peptides 106 (1), 153 (2), 386 (3), 420 (4), 454 (5), 460 (6), 469 (7), 564 (8), 820 (9), 1159 (10), 570 (11), 636 (12), 826 (13) to CPMV. Agarose gel (0.8%) stained with (FIG. 18B) GelRed (RNA) and (FIG. 18C) Coomassie blue (protein) The pI value from each peptide conjugated to each particle is shown in the table below the agarose gels. (FIG. 18D) SDS-PAGE: lane M, ladder. S=small capsid protein; S+pep=small capsid protein conjugated with peptide; L=large capsid protein; L+pep=large capsid protein conjugated with peptide. The number of peptides conjugated per particle (S+L conjugated) is reported in the table below the SDS-PAGE gel. Red dotted lines indicate the use of F127-assisted conjugation. (FIG. 18E) TEM images of negatively stained unmodified CPMV (wt) and CPMV-peptide conjugates (COVID-19 vaccines). White bar=100 nm. Particle size was corroborated by DLS, Z-average (d. nm) and polydispersity index (PDI) values were established for each particle.

FIGS. 19A-19C: ELISA against the individual epitopes using plasma from immunized mice. (FIG. 19A) Endpoint IgG titers after prime-boost administration of the various CPMV-based COVID19 vaccines. All CPMV-based COVID19 vaccines yielded epitope-specific IgG at 4 weeks post immunization (p<0.05 vs. pre-bleed). Three biological samples were tested individually (n=3) from each group (mean and SD are shown). (FIG. 19B) IgG titers were determined at weeks 4 and 12 using plasma from mice vaccinated with CPMV570, CPMV636, and CPMV826 as well as native CPMV. Three biological samples were tested individually (n=3) from each group (mean and SD are shown). Unpaired t-test (two-tailed, 95% confidence value) was used to compared between groups. p-values<0.05 were considered as statistically significant. (FIG. 19C) shows the IgG2a/IgG1 ratio which indicates a Th2 bias for CPMV570 and Th1 bias for CPMV636 and CPMV826.

FIGS. 20A-20B: ELISA binding of IgG to SARS-CoV-2 S protein. (FIG. 20A) Absorbance (450 nm) of plasma from animals vaccinated with the different CPMV vaccines and unmodified CPMV as control. All groups were significantly higher than CPMV control (p<0.05). (FIG. 20B) Percentage of inhibition of plasma samples from week 4 against S protein in vitro. The red dotted line represents the cutoff value, any group above that value was considered neutralizing against the recombinant S protein. *=p<0.05 vs unmodified CPMV control. Unpaired t-test (two-tailed, 95% confidence value) was used to compare between groups. p-values<0.05 were considered as statistically significant.

FIGS. 21A-21F: Synthesis and characterization of Qβ-based COVID-19 vaccines. (FIG. 21A) Conjugation of B cell epitope peptides 570 (2), 636 (3), 826 (4) to Qβ. (FIG. 21B) SDS-PAGE: lane M, ladder; CP, capsid protein alone or conjugated (CP+peptide) with a peptide (FIG. 21C) TEM images of negatively stained unmodified Qβ and Qβ-based vaccines. Scale bar=100 nm. (FIG. 21D) Particle size determined by DLS, Z-average (d. nm) and polydispersity index (PDI) value were established for each particle. (FIG. 21E) ELISA against S protein showing absorbance (450 nm) comparison side-by-side of 570, 636, and 826 candidates from CPMV vs Qβ vaccinated mice. Five biological samples were tested in duplicate (n=10) from each group. (FIG. 21F) ELISA against corresponding peptides showing endpoint IgG titers compared side-by-side of 570, 636, and 826 candidates from CPMV vs Qβ vaccinated mice. Five biological samples were tested individually (n=5) from each group. Unpaired t-test (two-tailed, 95% confidence value) was used to compared between groups. p-values<0.05 were considered as statistically significant.

FIGS. 22A-22C: Trivalent Qβ COVID-19 vaccine candidate delivery strategies. (FIG. 22A) Concept of implant application s.c. highlighting the sustained and slow release; and passive MN patches showing the release of the vaccine after the MN dissolves in the dermis. The positions of the devices in the arm are for illustration only. Photographs of an implant and a MN patch are shown. Detailed description of the PLGA-based implant (FIG. 22B) and MN patch (FIG. 22C) with the trivalent Qβ COVID-19 vaccine (Qβ570, Qβ636, and Qβ826).

FIGS. 23A-23D: ELISA of sera against peptide. (FIG. 23A) Immunization schedule. The groups were studied as follows: each Qβ vaccine alone (100 μg of single Qβ/dose), trivalent s.c. (3Qβ s.c., 100 μg of each Qβ/dose), trivalent implant (3Qβ Implant, a single dose of 200 μg of each Qβ), trivalent MN (passive, 50 μg of each Qβ/dose). As control groups free peptide, blank implant and blank MN were used, n=5 mice per group. (FIG. 23B) ELISA against the corresponding peptide (top=570; middle=636; bottom=826) showing endpoint IgG titers from vaccinated animals at different weeks (0-12) after the first immunization. Week 0 corresponds to plasma collected prior to the first immunization. (FIG. 23C) ELISA against the corresponding peptide (blue=570; red=636; green=826) showing IgG isotype profile (IgG2a/IgG1 ratio) at week 4, a ratio>1 was considered as Th1-biased and <1 as Th2-biased. (FIG. 23D) T cell activation by ELISpot assay. Splenocytes (1×10⁶ cells per well) from immunized mice with Qβ570; Qβ636; Qβ826; 3Qβ s.c. were stimulated with medium only (no stimulation control), single free-peptide (570, 636, 826) or a mixture (peptides 570+636+826), recombinant SARS-CoV-2 S protein, unmodified Qβ or PMA/Ionomycin (positive control). Cytokine (IFN-γ and IL-4) producing cells were counted as splenocytes forming colonies (SFC). Splenocytes from three mice were tested in duplicate for each stimulation (n=6). Data is represented as mean±SD. Mann Whitney test (two-tailed, 95% confidence value) was used to compared against control (medium only). p-values<0.05 were considered as statistically significant.

FIGS. 24A-24B: Sera from Qβ vaccine groups analyzed by ELISA against SARS-CoV-2 S protein and sVNT assay. (FIG. 24A) Absorbance (450 nm) of sera from animals vaccinated with the different Qβ vaccine strategies and their corresponding controls. All groups were significantly higher at week 2 and 4 as compared to their controls (p<0.05). Five biological samples were tested in duplicate (n=10) from each group. Red dotted line represents the background signal from control groups. (FIG. 24B) Percentage of inhibition of plasma samples from week 4 against HRP-RBD in vitro (sVNT assay). The red dotted line represents the cutoff value, any group above that value was considered neutralizing against the recombinant S protein. *=p<0.05 vs control groups (free peptide, blank implant, and blank MN). Five biological samples were tested individually (n=5) from each group. Control − and control + are internal controls for the technique and are PBS and ACE2 inhibitor, respectively.

FIGS. 25A-25C: SARS-CoV-2 S protein mutations from variants of concern (VOCs). (FIG. 25A) Punctual mutations that are unique or shared among VOCs are represented for each variant and then consolidated. S protein length is not at scale. (FIG. 25B) B cell epitopes alignment and S protein model. Amino acid alignment of the validated epitopes (570, 636, and 826) and surrounding regions for reference SARS-CoV-2 (USA_WA1/2020), SARS, and the variants of interest. (FIG. 25C) 3D model of the trimeric prefusion SARS-CoV-2 S protein. Highlighted colors indicate the actual position of the corresponding epitopes: Blue=553-570, Red=625-636, and Green=809-826.

FIG. 26: SARS-CoV-2 S protein domain map. Subunits and domains are not shown to scale. The positions of B-cell epitopes are shown, and the color code matches Table 1. SP=signal peptide; NTD=N-terminal domain; RBD=receptor-binding domain; FP=fusion peptide; HR1=heptad repeat 1; HR2=heptad repeat 2; TM=transmembrane domain; CP=cytoplasmic domain.

FIGS. 27A-27E: Synthesis and characterization of CPMV vaccines. (FIG. 27A) The two-step conjugation of B-cell epitope peptides 317, 362, 988, 1173, and 1209 to wild-type CPMV. (FIG. 27B) CPMV vaccines and the number of peptides displayed per VNP. (FIG. 27C) SDS-PAGE: lane M=size markers; lanes 1 and 6=wild-type CPMV; lane 2=CPMV-1173; lane 3=CPMV-1209; lane 4=CPMV-317; lane 5=CPMV-988; lane 7=CPMV-362. S=small capsid protein; S-pep=small capsid protein conjugated with peptide; L=large capsid protein; L-pep=large capsid protein conjugated with peptide. (FIG. 27D) TEM images of negatively stained wild-type CPMV and the vaccines. For CPMV, CPMV-1173 and CPMV-1209, the white bar=50 nm; for CPMV-317, CPMV-988 and CPMV-362, the white bar=100 nm. (FIG. 27E) Particle size determined by DLS, showing the Z-average diameter (d. nm) and polydispersity index (PDI) for each candidate.

FIGS. 28A-28D: Analysis of vaccine candidate immunogenicity by ELISA. (FIG. 28A) Immunization schedule using 100 μg of each vaccine candidate or 5 μg free peptide per injection (n=5 mice per group). (FIG. 28B) Endpoint IgG titers from animals vaccinated with red=CPMV-317, green=CPMV-988, pink=CPMV-1173, yellow=CPMV-1209, or blue=CPMV-362, at various times in weeks (W0-W10) after the first immunization (W0 corresponds to plasma collected prior to the first immunization). (FIG. 28C) Endpoint IgG titers from animals vaccinated with free peptides (color code identical to panel B). (FIG. 28D) IgG isotype profile (IgG1/IgG2a ratio) 0-10 weeks after the first immunization (values<1 defined as Th1-biased and values>1 defined as Th2-biased). Statistical significance: *p<0.0001 vs W0.

FIGS. 29A-29B: Binding of IgG to the SARS-CoV-2 S protein as determined by ELISA (n=5 mice per group). (FIG. 29A) Endpoint IgG titers from animals vaccinated with red=CPMV-317, green=CPMV-988, pink=CPMV-1173, yellow=CPMV-1209, or blue=CPMV-362, at various times in weeks (W0-W10) after the first immunization (W0 corresponds to plasma collected prior to the first immunization). (FIG. 29B) Endpoint IgG titers from animals vaccinated with free peptides (color code identical to panel A). Statistical significance: *p<0.0001 vs W0.

FIGS. 30A-30C: ELISpot assay with splenocytes from vaccinated mice. Isolated splenocytes (5×10⁵ cells per well) from immunized mice (n=3) were stimulated with medium only (negative control), the matching free peptides (20 μg/ml), unmodified CPMV (10 μg/ml) or PMA (50 ng/ml) plus ionomycin (1 μg/ml) as a positive control. Cytokine-producing cells (IFN-γ or IL-4) were counted as spot-forming colonies (SFCs) from (FIG. 30A) week 2 (W2) or (FIG. 30B) W10 after the first immunization. (FIG. 30C) Representative images of blue (IL-4) and red (IFN-7) spots formed by stimulated splenocytes from W2 or W10 after the first immunization. Statistical significance: *p<0.001 vs normal medium.

FIGS. 31A-31J: In vivo release of CPMV-Cy5 and the immunogenicity of pentavalent CPMV implants. (FIG. 31A) Administration schedule of CPMV-Cy5 implants and lymph node (LN) collection on different days (n=12; n=2 animals per day were euthanized for LN collection). (FIG. 31B) Administration schedule of pentavalent CPMV implants (CPMV-317, CPMV-362, CPMV-988, CPMV-1173, and CPMV-1209), n=5. 300 g of each CPMV vaccine per implant was used, this pentavalent implant dose is equivalent to a prime-boost-boost of 100 μg per dose for individual soluble vaccines. (FIG. 31C) Ventral view of mouse, showing localization of the different LNs collected (two LNs collected per site). (FIG. 31D) In vivo fluorescence images of mice implanted (blue square) with CPMV-Cy5, showing the Cy5 channel at different time points. (FIGS. 31E-31I) ELISA showing endpoint IgG titers (W0-W6) against peptides 317, 362, 988, 1173, and 1209, respectively. (FIG. 31J) ELISA showing endpoint IgG titers (W0 and W6) against the SARS-CoV-2 S protein. Statistical significance: *p<0.05; **p<0.01; ***p<0.0001 vs week W0.

FIG. 32: Immunofluorescence staining of T cells (CD4, blue), B cells (CD45R, red), dendritic cells (CD11c, green), CPMV (CPMV-Cy5, gray), and merged images of lymph nodes (cervical, axillary and inguinal) at different time points after the administration of implants. Scale bar=100 μm. Yellow arrows indicate the accumulation of CPMV-Cy5.

FIGS. 33A-33E: CPMV-based vaccines' full Ig isotype profile at week 4. The values are presented as mean±SD (n=5).

FIGS. 34A-34C: Characterization of CPMV-Cy5 conjugated particles. (FIG. 34A) Non-reducing 0.8% agarose gel of CPMV and CPMV-Cy5 conjugated particles visualized with Gel Red nucleic acid stain, Coomassie blue for protein staining, and Cy5 channel to identify CPMV-Cy5 conjugated particles. Cy5 channel shows effective conjugation of Cy5 dyes to CPMV coat proteins. (FIG. 34B) CPMV-Cy5 and CPMV-pentavalent implants imaged under Cy5 channel and visible light. (FIG. 34C) CPMV and lyophilized CPMV were loaded side-by-side under non-reducing 0.8% agarose gel and the integrity of viral particles evaluated by nucleic acid staining (top) and protein Coomassie blue staining (bottom). Ejected RNA from viral particles can be observed on lyophilized CPMV since RNA and protein position does not match as intact CPMV.

FIG. 35: DLS and TEM of wild-type CPMV and CPMV released from PLGA polymer implants.

FIG. 36: Anti-CPMV IgG titers from mice receiving a single dose of the CPMV-Cy5 implants or pentavalent CPMV (mixed CPMV-317, CPMV-362, CPMV-988, CPMV-1173, and CPMV-1209) or three doses (2 weeks apart each) of single soluble CPMV-317, CPMV-362, CPMV-988, CPMV-1173, or CPMV-1209. Naïve group represent plasma from non-vaccinated mice. The IgG titers were determined by ELISA against wild type CPMV nanoparticles. Plasma tested corresponds to week 6 post implant administration or soluble injection. The values are presented as mean±SD of pooled plasma per group tested in duplicate (n=2).

### DETAILED DESCRIPTION

### Definitions

As it would be understood, the section or subsection headings as used herein is for organizational purposes only and are not to be construed as limiting or separating or both limiting and separating the subject matter described.

Throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular forms “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

The term “protein”, “peptide” and “polypeptide” are used interchangeably and in their broadest sense to refer to a compound of two or more subunit amino acids, amino acid analogs or peptidomimetics. The subunits (which are also referred to as residues) may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

In some embodiments, a fragment of a protein is at least about 3 amino acids (aa) long, or at least about 4 aa long, or at least about 5 aa long, or at least about 6 aa long, or at least about 7 aa long, or at least about 8 aa long, or at least about 9 aa long, or at least about 10, aa long, or at least about 15, aa long, or at least about 20 aa long, or at least about 25 aa long, or at least about 30 aa long, or at least about 35 aa long, or at least about 40 aa long, or at least about 50 aa long, or at least about 60 aa long, or at least about 70 aa long, or at least about 80 aa long, or at least about 90 aa long, or at least about 100 aa long, or at least about 120 aa long, or at least about 150 aa long, or at least about 200, or longer.

As used herein, an amino acid (aa) or nucleotide (nt) residue position in a sequence of interest “corresponding to” an identified position in a reference sequence refers to that the residue position is aligned to the identified position in a sequence alignment between the sequence of interest and the reference sequence. Various programs are available for performing such sequence alignments, such as Clustal Omega and BLAST. In one aspect, equivalent polynucleotides, proteins and corresponding sequences can be determined using BLAST (accessible at blast.ncbi.nlm.nih.gov/Blast.cgi, last accessed on Aug. 1, 2021).

It is to be inferred without explicit recitation and unless otherwise intended, that when the present disclosure relates to a polypeptide, amino acid sequence, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity, or at least about 85% homology or identity, or alternatively at least about 90% homology or identity, or alternatively at least about 95% homology or identity, or alternatively at least about 96% homology or identity, or alternatively at least about 97% homology or identity, or alternatively at least about 98% homology or identity, or alternatively at least about 99% homology or identity (in one aspect, as determined using the Clustal Omega alignment program) and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complementary sequence.

In some embodiments, a first sequence (nucleic acid sequence or amino acid) is compared to a second sequence, and the identity percentage between the two sequences can be calculated. In further embodiments, the first sequence can be referred to herein as an equivalent and the second sequence can be referred to herein as a reference sequence. In yet further embodiments, the identity percentage is calculated based on the full-length sequence of the first sequence. In other embodiments, the identity percentage is calculated based on the full-length sequence of the second sequence.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

In some embodiments, the terms “first” “second” “third” “fourth” or similar in a component name are used to distinguish and identify more than one components sharing certain identity in their names. For example, “first virus or virus-like particle” and “second virus or virus-like particle” are used across the specification to distinguishing two virus or virus-like particles. However, this is not limited to the order in which these are present.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.

A “composition” as used herein, refers to an active agent, such as a compound as disclosed herein and a carrier, inert or active. The carrier can be, without limitation, solid such as a bead or resin, or liquid, such as phosphate buffered saline.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

Administration or treatment in “combination” refers to administering two agents such that their pharmacological effects are manifest at the same time. Combination does not require administration at the same time or substantially the same time, although combination can include such administrations. In one aspect, administration of the VLP conjugated particles is provided as the sole active agent, including in one aspect, the exclusion of an adjuvant.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Provided herein in the experimental examples are effective amounts determined in a murine or rat animal model, which can be converted to an effective dose by converting the reported μg of particle per g of the mouse. A typical adult mouse is from about 15 to about 35 g for a female mouse and about 20 g to about 30 g for a male mouse. In general, one will desire to administer an amount of the compound that is effective to achieve a serum level commensurate with the concentrations found to be effective in vivo. These considerations, as well as effective formulations and administration procedures are well known in the art and are described in standard textbooks.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor, or delay, slowing, or prevent of relapse. In one aspect, treatment excludes prophylaxis.

In one embodiment, the term “disease” or “disorder” as used herein refers alternatively to cardiovascular disease, high cholesterol and associated disease, HPV infection and associate diseases, cancer or a tumor (which are used interchangeably herein), a status of being diagnosed with such disease, a status of being suspect of having such disease, or a status of at high risk of having such disease. In one aspect, the disease or disorder is cardiovascular disease and associated disorders. In one aspect, the disease or disorder is HPV infection and associated disorders such as related cancers. In one embodiments, the term “disease” or “disorder” as used herein refers to a coronavirus infection, a status of being diagnosed with such infection, a status of being suspect of having such infection, a status of having being exposed to a coronavirus, or a status of at high risk of being exposed to a coronavirus. In one embodiment, the coronavirus is a respiratory virus. In a further embodiment, the disease is Coronavirus disease 2019 (COVID-19) caused by SARS-CoV-2. In yet a further embodiment, the disease is Severe acute respiratory syndrome (SARS) caused by SARS-CoV-1.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. In some embodiments, the term “cancer” is used interchangeably with the term “tumor”.

As used herein, an ablative therapy is a treatment destroying or ablating cancer tumors. In one embodiment, the ablative therapy does not require invasive surgery. In other embodiments, the ablative therapy refers to removal of a tumor comprising, or consisting essentially of, or yet further consisting of surgery. In some embodiments, the step ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g. IL-2, interferon's, cytokine inducers).

Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives.

In some embodiments, the coronavirus as used herein refers to a severe acute respiratory syndrome (SARS) associated coronavirus (SARS-CoV). In some embodiments, the coronavirus is either or both of SARS-CoV-1 and SARS-CoV-2. In some embodiments, the coronavirus comprises a virus selected from the group consisting of an Alphacoronavirus; a Colacovirus such as Bat coronavirus CDPHE15; a Decacovirus such as Bat coronavirus HKU10 or Rhinolophus ferrumequinum alphacoronavirus HuB-2013; a Duvinacovirus such as Human coronavirus 229E; a Luchacovirus such as Lucheng Rn rat coronavirus; a Minacovirus such as a Ferret coronavirus or Mink coronavirus 1; a Minunacovirus such as Miniopterus bat coronavirus 1 or Miniopterus bat coronavirus HKU8; a Myotacovirus such as Myotis ricketti alphacoronavirus Sax-2011; a nyctacovirus such as Nyctalus velutinus alphacoronavirus SC-2013; a Pedacovirus such as Porcine epidemic diarrhea virus or Scotophilus bat coronavirus 512; a Rhinacovirus such as Rhinolophus bat coronavirus HKU2; a Setracovirus such as Human coronavirus NL63 or NL63-related bat coronavirus strain BtKYNL63-9b; a Tegacovirus such as Alphacoronavirus 1; a Betacoronavirus; a Embecovirus such as Betacoronavirus 1, Human coronavirus OC43, China Rattus coronavirus HKU24, Human coronavirus HKU1 or Murine coronavirus; a Hibecovirus such as Bat Hp-betacoronavirus Zhejiang2013; a Merbecovirus such as Hedgehog coronavirus 1, Middle East respiratory syndrome-related coronavirus (MERS-CoV), Pipistrellus bat coronavirus HKU5 or Tylonycteris bat coronavirus HKU4; a Nobecovirus such as Rousettus bat coronavirus GCCDC1 or Rousettus bat coronavirus HKU9, a Sarbecovirus such as a Severe acute respiratory syndrome-related coronavirus, Severe acute respiratory syndrome coronavirus (SARS-CoV) or Severe acute respiratory syndrome coronavirus 2 (SARS-CoV-2, COVID-19); a Deltacoronavirus; an Andecovirus such as Wigeon coronavirus HKU20; a Buldecovirus such as Bulbul coronavirus HKU11, Porcine coronavirus HKU15, Munia coronavirus HKU13 or White-eye coronavirus HKU16; a Herdecovirus such as Night Heron coronavirus HKU19; a Moordecovirus such as Common moorhen coronavirus HKU21; a Gammacoronavirus; a Cegacovirus such as Beluga Whale coronavirus SW1; and an Igacovirus such as Avian coronavirus.

Symptoms of a coronavirus infection include, but are not limited to, mild symptoms, such as fatigues, tingling, tingling or numbness in the hands and feet, dizziness, confusion, brain fog, body ache, chills, loss of appetite, nausea, vomiting, abdominal pain or discomfort, loss of smell, inability to taste, muscle weakness, photophobia, adenopathy, headaches, cough, dry cough, shortness of breath, sore throat, lower extremity weakness/numbness, diarrhea, low blood O₂, sneezing, runny nose or post-nasal drip; severe symptoms, such as ventilatory use, high fever, severe cough, delirium, seizures, stroke, systematic inflammation, cytokine storm; and other symptoms, such as fever, swollen adenoids, pneumonia, bronchitis, and Dyspnea.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence comprising, or consisting essentially of, or yet further consisting of hydrogen bonding and other chemical interactions. Such molecules are known in the art and include, for example, those in which peptide linkages substitute for phosphate linkages in the backbone of the molecule.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

In various embodiments, an epitope peptide refers to a peptide comprising, consisting essentially of, or consisting of an epitope.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 103 M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7th Ed., W.H. Freeman & Co., 2013; Murphy, Janeway's Immunobiology, 8th Ed., Garland Science, 2014; Male et al., Immunology (Roitt), 8th Ed., Saunders, 2012; Parham, The Immune System, 4th Ed., Garland Science, 2014.

In terms of antibody structure, an immunoglobulin has heavy (H) chains and light (L) chains interconnected by disulfide bonds. There are two types of light chain, lambda (k) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located (heavy chain regions labeled CDHR and light chain regions labeled CDLR). Thus, a CDHR3 is the CDR3 from the variable domain of the heavy chain of the antibody in which it is found, whereas a CDLR1 is the CDR1 from the variable domain of the light chain of the antibody in which it is found. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, the term “epitope”, also known as antigenic determinant, is the part of an antigen that is recognized by the immune system, specifically by antibodies, B cells, or T cells. For example, the epitope is the specific piece of the antigen to which an antibody binds. The part of an antibody that binds to the epitope is called a paratope. Although epitopes are usually non-self proteins, sequences derived from the host that can be recognized (as in the case of autoimmune diseases) are also epitopes. In some embodiments, an epitope of a protein antigen is a conformational epitope or a linear epitope, based on its structure and interaction with the paratope

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, the term “virus-like particle” (VLP) refers to a structure that in at least one attribute resembles a virus, optionally which had not been demonstrated to be infections. In some embodiments, a VLP refers to a viral capsid conjugated to an antigen or epitope. In further embodiments, the VLP is non-replicating. In yet further embodiments, the VLP lacks all or part of the viral genome; for example, the replicative and infectious components of the viral genome. In another aspect, the VLP refers to the viral coat protein. Additionally or alternatively, the VLP does not infect a subject as disclosed herein, such as an animal. In some embodiments, the VLP only infects plants. In one aspect, the term VLP also refers to the virus or coat protein of the virus of VLP.

The VLP conjugated to the polypeptide or epitope with an optional linker can have a variety of diameters and number of attached polypeptides or epitopes and can be linked with either the C-terminus or the N-terminus of the polypeptide or epitope exposed, with an optional linker linking the polypeptide or epitope to the VLP. In some aspects, the VLP conjugated to the polypeptide or epitope has a diameter selected from the group of from about 1 nm to about 100 nm; from about 1 nm to about 75 nm; from about 1 nm to about 50 nm; from about 1 nm to about 25 nm; from about 1 nm to about 25 nm; from about 5 nm to about 100 nm; from about 5 nm to about 50 nm; or from about 5 nm to about 25 nm, or from about 15 nm to about 25 nm, or about 20 nm. In some embodiments, the VLP conjugated to the polypeptide or epitope has a diameter of from about 25 nm to about 60 nm, or from about 25 nm to about 50 nm, or from about 20 nm to about 40 nm, or from about 15 nm to about 50 nm, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm.

In some aspects, the number of polypeptides or epitopes per VLP (or virus or coat protein) is from about 5:1 to about 100:1, or from about 20:1 to about 100:1, or alternatively from about 10:1 to about 50:1, or alternatively from about 10:1 to about 40:1, or alternatively from about 10:1 to about 35:1, or alternatively from about 10:1 to about 100:1, or alternatively from about 10:1 to about 75:1, or alternatively from about 10:1 to about 50:1, or alternatively from about 10:1 to about 45:1, or alternatively from about 10:1 to 40:1, or alternatively from about 10:1 to about 35:1, or alternatively from about 10:1 to about 30:1, or alternatively from about 10:1 to about 25:1, or alternatively from about 10:1 to about 20:1, or alternatively from about 5:1 to about 75:1, or alternatively from about 5:1 to about 50:1, or alternatively from about 5:1 to about 45:1, or alternatively from about 5:1 to 40:1, or alternatively from about 5:1 to about 35:1, or alternatively from about 5:1 to about 30:1, or alternatively from about 5:1 to about 25:1, or alternatively from about 5:1 to about 20:1, or alternatively from about 5:1 to about 50:1, or alternatively from about 25:1 to about 60:1; alternatively from about 30:1 to about 50:1, or alternatively from about 35:1 to about 45:1, or alternatively about 40:1, or alternatively about 50; or alternatively about 40, or alternatively about 30, or alternatively about 25, or alternatively about 20, or alternatively about 15, or alternatively about 10, or alternatively about 5, per VLP.

In some aspects, the conjugated VLP (or virus or coat protein) has a defined valency per surface area of the VLP, also referred to herein as “density.” In these aspects, the polypeptide or epitope density per VLP is from about 0.005 of the polypeptide or epitope/100 nm² to about 100 of the polypeptide or epitope/100 nm² of the surface area of the VLP, or alternatively from about 0.406 of the polypeptide or epitope/100 nm² to about 50 of the polypeptide or epitope/100 nm²; or alternatively from about 0.05 of the polypeptide or epitope/100 nm² to about of 25 the polypeptide or epitope/100 nm². In certain aspects, the polypeptide or epitope density per VLP is from about 0.4 of the polypeptide or epitope/100 nm² to about 25 of the polypeptide or epitope/100 nm², or from about 0.4 of the polypeptide or epitope/100 nm² to about 20 the polypeptide or epitope/100 nm², or from about 0.4 of the polypeptide or epitope/100 nm² to about 15 of the polypeptide or epitope/100 nm², or from about 0.4 of the polypeptide or epitope/100 nm² to about 14 of the polypeptide or epitope/100 nm², or from about 0.4 of the polypeptide or epitope/100 nm² to about 13 of the polypeptide or epitope/100 nm², or from about 0.4 of the polypeptide or epitope/100 nm² to about 12 of the polypeptide or epitope/100 nm², or from about 0.4 of the polypeptide or epitope/100 nm² to about 11.6 of the polypeptide or epitope/100 nm², or from about 0.4 of the polypeptide or epitope/100 nm² to about 11.5 of the polypeptide or epitope/100 nm², or from about 0.4 of the polypeptide or epitope/100 nm² to about 11 of the polypeptide or epitope/100 nm², or from about 0.4 of the polypeptide or epitope/100 nm² to about 10 of the polypeptide or epitope/100 nm², or from about 0.4 of the polypeptide or epitope/100 nm² to about 9 of the polypeptide or epitope/100 nm², or from about 0.4 of the polypeptide or epitope/100 nm² to about 8 of the polypeptide or epitope/100 nm², or from about 0.4 of the polypeptide or epitope/100 nm² to about 7 of the polypeptide or epitope/100 nm², or from about 0.4 of the polypeptide or epitope/100 nm² to about 6 of the polypeptide or epitope/100 nm², or from about 0.4 of the polypeptide or epitope/100 nm² to about 5 of the polypeptide or epitope/100 nm², or from about 0.4 of the polypeptide or epitope/100 nm² to about 4 of the polypeptide or epitope/100 nm², or from about 0.4 of the polypeptide or epitope/100 nm² to about 3 of the polypeptide or epitope/100 nm², or from about 0.4 of the polypeptide or epitope/100 nm² to about 2.5 of the polypeptide or epitope/100 nm², or from about 0.4 of the polypeptide or epitope/100 nm² to about 2 of the polypeptide or epitope/100 nm², or from about 0.4 of the polypeptide or epitope/100 nm² to about 1.5 of the polypeptide or epitope/100 nm².

The terms “coat protein” and “viral coat protein” are used interchangeably, and refer to a protein, at least a portion of which is present on the surface of a viral particle, such as a bacteriophage Qβ or a cowpea chlorotic mottle virus. In some embodiments, a coat protein refers to a protein that creates the tightly assembled structure of the protective shell (also referred to as a capsid) for a virus and prevents degradation of the viral genome, such as by environmental factors.

As used herein, “capsid” is a generic term used to indicate any type of viral shell, particle or coat, including a protein capsid, a lipid enveloped structure, a protein-nucleic acid capsid, or a combination thereof (e.g., a lipid-protein envelope surrounding a protein-nucleic acid capsid).

Bacteriophage Qβ (Qbeta or alternatively QB) is a member of the levivirida family. It is a small virus that is about 25 nm thick and is a coliphage with an RNA that is 4217 nucleotides long. As described in biology.kenyon.edu/BMB/jsmol2019/EAIJ/NewVersion81.html#:˜:text=Bacteriophage%20 QB%20is%20a%20member,%2C%20%26%20Finn%2C%202009).&text=Members%20of%20the%20leviviridae%20family,et%20at.%2C%202018) last accessed on Aug. 17, 2021, QB has 20 faces each composed of six subunits and 12 vertices each composed of 5 subunits. Members of the leviviridae family form icosahedral capsids from 180 coat protein subunits around a 4.2 kb sense-strand RNA genome. Each of these coat proteins (capsomers) has about 132 residues of amino acids. Bacteriophage QB is a positive strand RNA virus. Positive strand RNA viruses have genomes that are functional mRNAs. For instance, QB's genome codes for 4 proteins: A1, A2, CP and QB replicase. QB has other proteins like the B-subunit of a replicase, the maturation protein A2 and a minor protein A1. The penetration of the virus into a host cell is quickly followed by translation to produce RdRps and other viral proteins that are required for the production of more viral RNAs. QB ssRNA adsorb to bacterial sex pili proteins and infect. Like other RNA viruses, QB replicates its genome by utilizing virally encoded RNA polymerase (RdRp). The genome is used as the template for the synthesis of other RNA strands. Upon infection, the B-subunit interacts with host proteins to form a complex. The complex contains RNA-helicases to unwind DNA and NTPases that are useful for polymerization. Once the complex forms, the transcription of the genome, a copy of the genome, and mRNAs begin. Phage MS2 has the same genome as QB.

Bacteriophage Qβ coat protein self-assembles to form an icosahedral capsid with a T=3 symmetry, about 26 nm in diameter, and consisting of 89 capsid proteins dimers (178 capsid proteins). It is also involved in viral genome encapsidation through the interaction between a capsid protein dimer and the multiple packaging signals present in the RNA genome. Binding of the capsid proteins to the viral RNA induces a conformational change required for efficient T=3 shell formation. Additionally, it acts as a translational repressor of viral replicase synthesis late in infection. This latter function is the result of capsid protein interaction with an RNA hairpin which contains the replicase ribosome-binding site. See, for example, Gorzelnik et al., Proc Natl Acad Sci USA. 2016 Oct. 11; 113(41):11519-11524; Basnak et al., J Mol Biol. 2010 Feb. 5; 395(5):924-36; and Lim et al., J Biol Chem. 1996 Dec. 13; 271(50):31839-45.

In some embodiments, a VLP derived from bacteriophage Qβ comprise, or consists essentially of, or yet further consists of, a plurality of coat proteins. In some embodiments, the coat protein is a wild-type bacteriophage Qβ coat protein. In further embodiments, the coat protein is modified, e.g., comprising, or consisting essentially of, or yet further consisting of, one or more substitutions, insertions, and/or deletions. In some embodiments, a bacteriophage Qβ coat protein comprises, or alternatively consists essentially of, or yet further consists of the sequence as set forth in the UniProtKB ID P03615: MAKLETVTLGNIGKDGKQTLVLNPRGVNPTNGVASLSQAGAVPALEKRVTVSVSQP SRNRKNYKVQVKIQNPTACTANGSCDPSVTRQAYADVTFSFTQYSTDEERAFVRTEL AALLASPLLIDAIDQLNPAY (SEQ ID NO: 24) or an equivalent thereof.

A bacteriophage Qβ hairpin loop refers to a portion of a Qβ RNA where a Qβ coat protein can bind to. In some embodiments, the hairpin loop serves as a packaging signal directing an RNA comprising the hairpin loop to be encapsidated in a capsid comprising, or consisting essentially of, or yet further consisting of a Qβ coat protein.

Cowpea chlorotic mottle virus (CCMV) is a spherical plant virus that belongs to the Bromovirus genus. Several strains have been identified and include, but not limited to, Car1 (Ali, et al., 2007. J. Virological Methods 141:84-86), Car2 (Ali, et al., 2007. J Virological Methods 141:84-86, 2007), type T (Kuhn, 1964. Phytopathology 54:1441-1442), soybean (S) (Kuhn, 1968. Phytopathology 58:1441-1442), mild (M) (Kuhn, 1979. Phytopathology 69:621-624), Arkansas (A) (Fulton, et al., 1975. Phytopathology 65: 741-742), bean yellow stipple (BYS) (Fulton, et al., 1975. Phytopathology 65: 741-742), R (Sinclair, ed. 1982. Compendium of Soybean Diseases. 2^{nd} ed. The American Phytopathological Society, St. Paul. 104 pp.), and PSM (Paguio, et al., 1988. Plant Diseases 72(9): 768-770). Also, see, for example, WO2021/108202.

In some embodiments, a VLP derived from CCMV comprise, or consists essentially of, or yet further consists of, a plurality of coat proteins. In some embodiments, the coat protein is a wild-type CCMV coat protein, optionally expressed by Car1, Car2, type T, soybean (S), mild (M), Arkansas (A), bean yellow stipple (BYS), R, or PSM strain. In further embodiments, the coat protein is modified, e.g., comprising, or consisting essentially of, or yet further consisting of, one or more substitutions, insertions, and/or deletions. In some cases, the CCMV coat protein comprise, or consists essentially of, or yet further consists of, the sequence as set forth in the UniProtKB ID P03601: MSTVGTGKLTRAQRRAAARKNKRNTRVVQPVIVEPIASGQGKAIKAWTGYSVSKW TASCAAAEAKVTSAITISLPNELSSERNKQLKVGRVLLWLGLLPSVSGTVKSCVTETQ TTAAASFQVALAVADNSKDVVAAMYPEAFKGITLEQLTADLTIYLYSSAALTEGDVI VHLEVEHVRPTFDDSFTPVY (SEQ ID NO: 25), or an equivalent thereof.

In some cases, the engineered VLP from CCMV is prepared by the method as described in Ali et al., “Rapid and efficient purification of Cowpea chlorotic mottle virus by sucrose cushion ultracentrifugation,” Journal of Virological Methods 141: 84-86 (2007).

Cowpea mosaic virus (CPMV) is a plant-infecting member of the order Picornavirales, with a relatively simple, non-enveloped capsid that has been extensively studied and a positive-sense, single-stranded RNA genome. For CPMV, the genome is bipartite, with RNA-1 (6 kb) and RNA-2 (3.5 kb) being separately encapsidated. CPMV has an icosahedral capsid structure, which is ˜30 nm in diameter and is formed from 60 copies each of a Large (L) and Small (S) coat protein. These two coat proteins are processed from a single RNA-2-encoded precursor polyprotein (VP60) by the action of the 24 K viral proteinase which is encoded by RNA-1. Thus capsid assembly, as well as viral infection, is dependent on the presence of both genomic segments in an infected plant cell.

The terms “CPMV” “CPMV virus” or “CPMV particles” are used interchangeably, referring to a CPMV comprising, or alternatively consisting essentially of, or yet consisting of a capsid and an RNA genome (which is also referred to herein as a viral genome) encapsidated in the capsid. In some embodiments, the CPMV particles have been treated, prepared and/or inactivated by a method as disclosed herein. In some embodiments, the CPMV particle further comprises a heterologous RNA, which is heterologous to (i.e., not naturally presented in) a native CPMV free of any human intervention.

In some embodiments, the CPMV can be substitute by another plant virus, for example another plant retrovirus. However, a bacteriophage or mammalian virus can be used in some embodiments of the invention. When a plant virus is used, in some embodiments the plant virus is a plant picornavirus. A plant picornavirus is a virus belonging to the family Secoaviridae, which together with mammalian picomaviruses belong to the order of the Picornavirales. Plant picomaviruses are relatively small, nonenveloped, positive-stranded RNA viruses with an icosahedral capsid. Plant picomaviruses have a number of additional properties that distinguish them from other picomaviruses, and are categorized as the subfamily secoviridae. In some embodiments, the virus particles are selected from the Comovirinae virus subfamily. Examples of viruses from the Comovirinae subfamily include Cowpea mosaic virus, Broad bean wilt virus 1, and Tobacco ringspot virus. In a further embodiment, the virus particles are from the Genus comovirus. A preferred example of a comovirus is the cowpea mosaic virus particles. Other suitable plant virus includes, but is not limited to bean pod mottle virus (BPMV) or rice tungro spherical virus.

The virus can be obtained according to various methods known to those skilled in the art. In embodiments where plant virus particles are used, the virus particles can be obtained from the extract of a plant infected by the plant virus. For example, cowpea mosaic virus can be grown in black eyed pea plants, which can be infected within 10 days of sowing seeds. Plants can be infected by, for example, coating the leaves with a liquid containing the virus, and then rubbing the leaves, preferably in the presence of an abrasive powder which wounds the leaf surface to allow penetration of the leaf and infection of the plant. Within a week or two after infection, leaves are harvested and viral nanoparticles are extracted. In the case of cowpea mosaic virus, 100 mg of virus can be obtained from as few as 50 plants. Procedures for obtaining plant picornavirus particles using extraction of an infected plant are known to those skilled in the art. See Wellink J., Meth Mol Biol, 8, 205-209 (1998). Procedures are also available for obtaining virus-like particles. Saunders et al., Virology, 393(2):329-37 (2009). The disclosures of both of these references are incorporated herein by reference.

As used herein, a cholesterol checkpoint protein refers to a protein, changes of which (such as its level or activity in a subject) affect the cholesterol level in a subject. In some embodiments, higher level, or higher activity, or both of a cholesterol checkpoint protein is positively associated with an increased cholesterol level in a subject.

As used herein, the term “sample” and “biological sample” and “agricultural sample” are used interchangeably, referring to sample material derived from a subject. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples may include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Agricultural samples include soil, foliage or any plant tissue or surface or other sample suspected of harboring virus. In addition, the sample can include industrial samples, such as those isolated from surfaces and the environment.

In some embodiments, the sample may be an upper respiratory specimen, such as a nasopharyngeal (NP) specimen, an oropharyngeal (OP) specimen, a nasal mid-turbinate swab, an anterior nares (nasal swab) specimen, or nasopharyngeal wash/aspirate or nasal wash/aspirate (NW) specimen.

In some embodiments, the samples include fluid from a subject, including, without limitation, blood or a blood product (e.g., serum, plasma, or the like), umbilical cord blood, amniotic fluid, cerebrospinal fluid, spinal fluid, lavage fluid (e.g., bronchoalveolar, gastric, peritoneal, ductal, ear, arthroscopic), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a liquid biological sample is a blood plasma or serum sample. The term “blood” as used herein refers to a blood sample or preparation from a subject. The term encompasses whole blood, blood product or any fraction of blood, such as serum, plasma, buffy coat, or the like as conventionally defined. In some embodiments, the term “blood” refers to peripheral blood. Blood plasma refers to the fraction of whole blood resulting from centrifugation of blood treated with anticoagulants. Blood serum refers to the watery portion of fluid remaining after a blood sample has coagulated. Fluid samples often are collected in accordance with standard protocols hospitals or clinics generally follow. For blood, an appropriate amount of peripheral blood (e.g., between 3-40 milliliters) often is collected and can be stored according to standard procedures prior to or after preparation.

The polynucleotides used in the present disclosure, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a polynucleotide of almost any length may be employed, with the total length preferably being limited by the ease of preparation and use in the intended recombinant polynucleotide. In some cases, a polynucleotide can encode a polypeptide sequence with additional heterologous coding sequences, for example to allow for detection or purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

Recombinant DNA technology can be employed wherein a polynucleotide which encodes a polypeptide of the disclosure is inserted into an expression vector, transformed or transfected into an appropriate host cell and cultivated under conditions suitable for expression.

In some embodiments, the cell as disclosed herein is a eukaryotic cell or a prokaryotic cell.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The host cell can be a prokaryotic or a eukaryotic cell.

“Eukaryotic cells” comprise all of the life kingdoms except Monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called an episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

Vaccines for the Treatment of Cardiovascular Disease

This disclosure provides a vaccine comprising, or consisting essentially of, or consisting of a virus or a virus-like particle(s) (VLP) comprising a cholesterol checkpoint protein(s) for the lowering of cholesterol and treating or preventing cardiovascular diseases. In one embodiment the epitope(s) does not activate a T cell or a cytotoxic T cell when administered to a subject.

In one aspect, the cholesterol checkpoint protein is selected from any one or any two or all three of: proprotein convertase subtilisin/kexin-9 (PCSK9), apolipoprotein B (ApoB), or cholesteryl ester transfer protein (CETP). In one aspect, each of the virus or virus-like particle(s) comprises at least one epitope peptide comprising, or consisting essentially of, or yet further consisting of an amino acid sequence selected from (a) a fragment of ApoB (KTTKQSFDLSVKAQYKKNKH (SEQ ID NO: 1)), (b) a fragment of CETP (FGFPEHLLVDFLQSLS (SEQ ID NO: 2)), or (c) which is a fragment of PSCK9 (NVPEEDGTRFHRQASKC (SEQ ID NO: 3)), or an equivalent of each thereof. In one aspect, the fragments further comprise 3 amino acids (aa), or 4, or 5, or 6, or 7, or 8, or 9, or 10, or 11, or 12, or 13, or 14, or 15, or 16, or 17, or 18, or 19, or 20, or 25 aa on the N- and/or c-terminus of the fragment, wherein the additional amino acids of the wild-type reference protein. As used herein, an equivalent comprises an amino acid sequence having at least 70%, or alternatively at least 75%, or alternatively at least 80%, or alternatively at least 85%, or alternatively at least 90%, or alternatively at least 95%, or alternatively at least 97%, or alternatively at least 99%, or 100% sequence identity to the reference amino acid SEQ ID NOs: 1-3, and similar biological activity as the reference amino acid. Further provided is a vaccine composition that comprises a plurality of a virus or virus-like particle(s) comprising one or more cholesterol checkpoint protein(s) that may be the same or different from each other.

In one aspect, the virus or virus-like particle is a bacteriophage virus or virus like particle, or a plant virus or virus like particle. In another aspect, the virus or virus-like particle is a bacteriophage Qβ virus or virus like particle. In a further aspect, the virus or virus-like particle is a plant picornavirus virus or virus like particle, or a filamentous plant virus or virus-like particle, e.g., wherein the plant virus or virus-like particle is of the or Alphafexiviridae family. Alternatively, the plant virus or virus-like particle is a cowpea mosaic virus-like particle or a potato virus X virus-like particle. Yet further, the plant virus particle or virus-like particle is a rod-shaped virus or virus-like particle. Yet further, the rod-shaped virus or virus-like particle is a tobacco mosaic virus or virus-like particle. The diameters and number of cholesterol checkpoint proteins or fragments thereof can vary as disclosed herein. In one aspect, the VLP comprises from about 40 to about 75 epitopes per virus or VLP. The epitope can be linked to the virus or VLP at the N-terminus or the C-terminus.

For example, the at least one of the epitope(s), or a protein fragment comprising at least one checkpoint protein or a protein fragment is conjugated directly or indirectly to the virus or the virus-like particle, or a coat protein of the virus or the virus-like particle. Alternatively, the at least one check point protein or a protein fragment comprising at least one of the epitope(s) is conjugated indirectly comprising, or consisting essentially of, or yet further comprising of a linker to the virus or virus-like particle, or a coat protein of the virus or the virus-like particle at the C-terminus or the N-terminus. In one aspect, the linker comprises an amino acid sequence of (GSG)n, (GGSG)n, (GPSL)n, or (GGSGGGSG)n, wherein n is an integer from 1 to 5, or 1 to 4, or 1 to 3, or 2, or 3, or 4 or 5, or wherein the linker is an SM(PEG)₈ bifunctional linker comprising an NHS group and a maleimide group, or wherein the linker comprises an N-terminal cysteine residue conjugated to triple glycine (GGG) and an N-hydroxysuccinimide-PEG4-maleimide linker SM-PEG4. In a further aspect, the C-terminus or the N-terminus of the epitope(s), a peptide comprising at least one of the epitope(s), or a protein fragment comprising at least one of the epitope(s) is conjugated directly or indirectly to the N-terminus or the C-terminus of a coat protein of the virus or the virus-like particle.

Vaccine and Pharmaceutical Compositions

Also provided are pharmaceutical or vaccine formulations that can comprise one, two or all there of the cholesterol checkpoint protein fragments as described above conjugated to the virus or virus-like particle (e.g. Qβ) and a carrier, such as a pharmaceutically acceptable carrier. In one embodiment, the formulation comprises all three of the cholesterol checkpoint protein fragments. As understood by one of skill in the art, the formulation can comprise a plurality of virus-like particle, e.g., Qβ, in a variety of ratios of components: 1:1:1; 1:2:1; 1:3:2; 2:1:2; 3:1:2: 2:3:4, as determined by the treating physician or veterinarian. The VLPs comprising the cholesterol checkpoint protein fragments can be the same or different from each other. In one aspect, the formulation comprises from about 0.05 to 0.5 mg of total checkpoint protein fragments per μl of carrier.

In one aspect, the formulation vaccine comprises, or consists essentially of, or yet further consists of three virus or virus-like particles, wherein a first virus or virus-like particle comprises an epitope peptide comprising an amino acid sequence of KTTKQ SFDLS VKAQYKKNKH (SEQ ID NO: 1), a second virus or virus-like particle comprises an epitope peptide comprising an amino acid sequence of FGFPE HLLVD FLQSL S (SEQ ID NO: 2), and a third virus or virus-like particle comprises an epitope peptide comprising an amino acid sequence of NVPEE DGTRF HRQAS KC (SEQ ID NO: 3), or an equivalent of each thereof. In another aspect, the VLP comprises SEQ ID NO: 1 and SEQ ID NO: 2, or alternatively, SEQ ID NO: 2 and SEQ ID NO: 3, or alternatively SEQ ID NO: 1 and SEQ ID NO; 3, or alternatively SEQ ID NOS: 1, 2 and 3. In one aspect, the VLP comprises from about 40 to about 75 epitopes per virus or VLP. The epitope can be linked to the virus or VLP at the N-terminus or the C-terminus.

In a further embodiment, the formulation comprises, or consists essentially of, or consist of, two virus or virus-like particles, wherein a first virus or virus-like particle comprises an epitope peptide comprising an amino acid sequence selected from one of KTTKQSFDLSVKAQYKKNKH (SEQ ID NO: 1), FGFPEHLLVDFLQSLS (SEQ ID NO: 2), or NVPEEDGTRFHRQASKC (SEQ ID NO: 3) or an equivalent of each thereof, and a second virus or virus-like particle comprises the remaining two amino acid sequencers in the one or two epitope peptide(s). In an alternative aspect, the epitope(s) or a protein fragment comprising at least one of the epitope(s) is present on the outer surface of the virus or the virus-like particle. In one aspect, the VLP comprises from about 40 to about 75 epitopes per virus or VLP. The epitope can be linked to the virus or VLP at the N-terminus or the C-terminus.

For example, the at least one of the epitope(s), or a protein fragment comprising at least one checkpoint protein or a protein fragment is conjugated directly or indirectly to the virus or the virus-like particle, or a coat protein of the virus or the virus-like particle. Alternatively, the at least one check point protein or a protein fragment comprising at least one of the epitope(s) is conjugated indirectly comprising, or consisting essentially of, or yet further comprising of a linker to the virus or virus-like particle, or a coat protein of the virus or the virus-like particle at the C-terminus or the N-terminus. In one aspect, the linker comprises an amino acid sequence of (GSG)n, (GGSG)n, (GPSL)n, or (GGSGGGSG)n, wherein n is an integer from 1 to 5, or 1 to 4, or 1 to 3, or 2, or 3, or 4 or 5, or wherein the linker is an SM(PEG)₈ bifunctional linker comprising an NHS group and a maleimide group, or wherein the linker comprises an N-terminal cysteine residue conjugated to triple glycine (GGG) and an N-hydroxysuccinimide-PEG4-maleimide linker SM-PEG4. In a further aspect, the C-terminus or the N-terminus of the epitope(s), a peptide comprising at least one of the epitope(s), or a protein fragment comprising at least one of the epitope(s) is conjugated directly or indirectly to the N-terminus or the C-terminus of a coat protein of the virus or the virus-like particle.

In a further aspect, the formulation further comprises a stabilizer or cryopreservative and the formulation is frozen or lyophilized for ease of use and storage.

In one embodiment, the formulation comprises a plurality of virus or virus-like particles which each particle comprises an epitope or fragment as described herein, to provide a formulation comprising the 3 peptide fragments in one formulation. The virus or virus-like particles of the plurality can be the same or different from each other.

The vaccine can be combined with one or more appropriate drugs or compounds to treat or prevent cardiovascular diseases as is apparent to one of skill in the art.

Lastly, to address delivery requirements of the vaccines Applicant created slow-release PLGA:VLP implants using hot melt-extrusion. [P. W. Lee et al., ACS Nano 2017; D. M. Wirth et al., Polymer 2019; S. Shao et al., Vaccines 2021] VLPs withstand the rigors of the high-temperature process; VLPs released from hot-melt extruded PLGA:VLP implants maintain their structural and immunogenic properties. [P. W. Lee et al., ACSNano 2017; D. M. Wirth et al., Polymer 2019; S. Shao et al., Vaccines 2021] This manufacturing process is continuous, solvent free, and can lead to the high-throughput production of vaccine delivery devices.

In a further aspect, the carrier comprises a slow-release implant, e.g., encapsulated within a degradable polymer matrix. In one aspect, the degradable polymer matrix comprises a melt-processable degradable polymer material that is biocompatible and, upon degradation, produces substantially non-toxic products, wherein the melt-processable degradable polymer material is a melt-processable biodegradable polymer, and wherein the degradable polymer material has a melt temperature below the degradation temperature of the virus or virus-like particle(s). A non-limiting example of such includes poly(lactic-co-glycolic acid) (PLGA) or a copolymer thereof or wherein the slow-release implant comprises one or more of: about 50% to about 99% PLGA, about 1% to about 50% virus or virus-like particle(s), or PEG8000, or wherein the slow-release implant comprises one or more of: about 80% PLGA, about 10% virus or virus-like particle(s), or about 10% PEG8000 optionally by weight, or wherein the slow-release implant comprises one or more of: about 75% PLGA, about 10% VNPs, or about 15% PEG8000, optionally wherein the % is indicated a weight percentage.

In another aspect, provided herein is a composition comprising, consisting essentially of, or consisting of a component or a combination provided herein, and at least one pharmaceutically acceptable excipient. In one aspect, the carrier excludes an adjuvant.

Compositions, including pharmaceutical compositions comprising, consisting essentially of, or consisting of a component or a combination as described herein, can be manufactured by means of conventional mixing, dissolving, granulating, dragee-making levigating, emulsifying, encapsulating, entrapping, or lyophilization processes. The component or combination can be formulated in conventional manner using one or more physiologically acceptable carriers, diluents, excipients, or auxiliaries which facilitate processing of the component or combination provided herein into preparations which can be used pharmaceutically.

The component or combination of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

In one embodiment, this technology relates to a composition comprising a component or a combination as described herein and a carrier.

In another embodiment, this technology relates to a pharmaceutical composition comprising a component or a combination as described herein and a pharmaceutically acceptable carrier.

In another embodiment, this technology relates to a pharmaceutical composition comprising a therapeutically effective amount of a component or a combination as described herein and a pharmaceutically acceptable carrier.

The pharmaceutical compositions for the administration of a component or a combination as disclosed herein can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each component provided herein is included in an amount sufficient to produce the desired effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the component or the combination can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the component or the combination provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

Methods for Making Vaccines and Formulations for Treating Cardiovascular Disease

Applicant also provides methods to make the vaccines and formulations. In one aspect, the VLP is a bacteriophage Qβ or CPMV or other VLP and the epitope is conjugated to the virus, VLP (e.g., Qβ) or CPMV via a SM(PEG)₈ bifunctional linker containing an NH group and a maleimide group. The virus, VLP or CPMV is mixed in excess of the SM(PEG)₈ and unreacted SM(PEG)₈ is removed. The particles are recovered and reacted with an excess of epitopes or peptides at room temperature. Unreacted peptides are removed.

Alternatively, a polynucleotide encoding the VLP, CMPV or bacteriophage Qβ coat protein with an optional N-terminal cysteine and linker such as GGG is conjugated to the polynucleotide encoding the epitope and inserted into a plasmid for expression in a eukaryotic or prokaryotic system. A non-limiting example of E. coli expression vector is the pDUET-1 expression vector for expression in E. coli, e.g., E. coli B121 (DE3). Thus, in one aspect, also provided herein are polynucleotides encoding the VLP, CMPV or bacteriophage Qβ coat protein with an optional N-terminal cysteine and linker such as GGG and the epitope(s) of interest. The polynucleotides can further comprise a regulatory element for expression in an appropriate system, e.g., a promoter or enhancer. Also provided is a vector comprising the polynucleotide, as well as host cells comprising the polynucleotide and/or vectors. The vectors can be a plasmid or viral vector and the host cells can be eukaryotic or prokaryotic cells. The polynucleotides are expressed by growing the host cells under conditions for expression of the polynucleotides and the vaccine product is isolated from the cell culture.

Also provided are methods for preparing the compositions by admixing the vaccine with a carrier such as a pharmaceutically acceptable carrier. The compositions and/or formulations can further comprise a cryopreservative or stabilizer. The compositions can be frozen or lyophilized.

PGLA implants can be prepared using a melt-processing system. Particles can be lyophilized before melt extrusion. Components are mixed and then loaded into the melt-processing system and heated for an effective amount of time and pressure for extrusion. Implants are dried and cut as desired.

Microneedle fabrication methods are known in the art and described herein.

Methods of Treating Cardiovascular Disease

Also provided herein is a method for one or more of the following in a subject in need thereof: treating or preventing a cardiovascular disease, treating or preventing an atherosclerosis, treating or preventing a hypercholesterolemia, treating or preventing a lipid dyshomeostasis, preventing a heart attack, preventing a stroke, reducing a statin administration dose or frequency or both, reducing a cholesterol level, reducing an oxidized cholesterol level, reducing a low-density lipoprotein cholesterol (LDL-C) level, reducing a level or an activity of one or more cholesterol checkpoint protein(s), producing an antibody recognizing and binding to one or more cholesterol checkpoint protein(s), triggering, enhancing, or prolonging an immune response to one or more cholesterol checkpoint protein(s), or delivering at least one epitope(s) of one or more cholesterol checkpoint protein(s) to the subject, comprising administering to the subject one or more virus or virus-like particle(s), wherein each virus or virus-like particle comprises at least one epitope of the cholesterol checkpoint protein(s), and optionally comprising two or more epitopes that may be the same or different from each other. In one aspect, the virus or virus-like particle comprises at least one epitope of the cholesterol checkpoint protein(s), and optionally comprising two or more epitopes that may be the same or different from each other are as described herein. Non-limiting examples of treatment include one or more of delivering at least one epitope of the cholesterol checkpoint protein(s) to the subject, producing an antibody recognizing and binding to the one or more cholesterol checkpoint protein(s) in the subject, triggering, enhancing, or prolonging an immune response to the one or more cholesterol checkpoint protein(s), reducing the level or the activity of the one or more cholesterol checkpoint protein(s) in the subject, reducing the total cholesterol level in the subject, reducing the oxidized cholesterol level in the subject, reducing the LDL-C level in the subject, reducing a statin administration dose or frequency or both, treating or preventing a cardiovascular disease, treating or preventing an atherosclerosis, treating or preventing a hypercholesterolemia, treating or preventing a lipid dyshomeostasis, preventing a heart attack, or preventing a stroke.

In a particular aspect of this disclosure, the method comprises administering to the subject a plurality of virus or the virus-like particles (e.g., Qβ) as described herein, which in one aspect comprises the following three amino acid sequences KTTKQSFDLSVKAQYKKNKH (SEQ ID NO: 1), FGFPEHLLVDFLQSLS (SEQ ID NO: 2), and NVPEEDGTRFHRQASKC (SEQ ID NO: 3) or an equivalent of each thereof.

The virus or virus-like particle is administered to a mammal, e.g. a human patient in need of such treatment. In one embodiment, the level is in a biological sample of the subject that is optionally selected from: plasma, peripheral blood, or serum.

Methods of administration are known in the art and can comprise one or more doses or using a single formulation as described herein, e.g., wherein the method does not comprises repeating the administering step. For example, the slow-release implant is administered by a microneedle patch or by injection. Alternatively, administration comprising repeating the administering step for about once, about twice, about three times, about four times, about five times, or more, and wherein two subsequent administrations are about 1 day to about 1 year apart, or about 1 week apart, or about 2 weeks apart, or about 3 weeks apart, or about 4 weeks apart, or about 1 month apart, or about 2 months apart, or about 3 months apart, or about 6 months apart. Yet further and alternatively, the administering step is not repeated for more than 6 times, or more than 7 times, or more than 8 times, or more than 9 times, or more than 10 times, or more than 15 times, or more than 20 times.

As disclosed herein, Applicant evaluated the delivery of trivalent VLP vaccines targeting ApoB, CETP, and PCSK9 “cholesterol checkpoint” proteins using the PLGA:VLP implant delivery strategy. VLPs were designed and expressed in E. coli. Vaccination was carried out in Balb/C mice using soluble mixtures vs. slow-release PLGA:VLP implants; monovalent and trivalent vaccines were evaluated and efficacy was determined based on antibody titers against the target proteins, reduction of total cholesterol levels in plasma, lowered abundance of ApoB and PCSK9 proteins, and inhibition of CETP (the latter was tested in vitro using sera from immunized mice). Finally, the immunological and physiological safety of this multitarget method was validated.

In one aspect of the method, the epitope(s) does not activate a T cell or a cytotoxic T cell in the subject.

In a further aspect, the method does not comprise administering to the subject an additional adjuvant.

In one embodiment, the virus or virus-like particle(s) comprises at least one epitope from each of PCSK9, ApoB, and CETP, and whereby achieving one or more of the following effects in synergy: reducing the total cholesterol level in the subject, reducing the oxidized cholesterol level in the subject, reducing the LDL-C level in the subject, reducing a statin administration dose or frequency or both, treating or preventing a cardiovascular disease, treating or preventing an atherosclerosis, treating or preventing a hypercholesterolemia, treating or preventing a lipid dyshomeostasis, preventing a heart attack, or preventing a stroke.

Dosages and Dosing Regimens for Treating or Preventing Cardiovascular Disease

The appropriate amount and dosing regimen of the vaccine, component or the combination, when present to be administered to the subject according to any of the methods disclosed herein, may be determined by one of ordinary skill in the art.

In some embodiments, the vaccine, component or the combination as disclosed herein, may be administered to a subject in need thereof, either alone or as part of a pharmaceutically acceptable formulation, once a week, once a day, twice a day, three times a day, or four times a day, or even more frequently.

Administration of the vaccine, component or the combination as disclosed herein may be effected by any method that enables delivery of the component or the combination to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the vaccine, composition or formulation are well-known in the relevant art and would be understood to be encompassed by the skilled artisan once provided the teachings disclosed herein.

Vaccines for the Prevention or Treatment HPV or COVID, and Associated Diseases

HPV Vaccines

The HPV capsid is composed of two proteins the major capsid protein L1, which forms the virus structure, and the minor capsid protein L2, which is required for genome encapsidation and infection [Trus, B. et al. Microsc. Microanal. 2005; Buck, C. B. et al, J. Virol. 2008; Chen, X. S. et al, Mol. Cell 2000]. All three approved HPV vaccines comprise virus-like particles (VLPs) that spontaneously self-assemble from protein L1, which is immunogenic and can elicit high titers of HPV-specific antibodies. However, one limitation of L1 is that the vaccines are type-specific unless they contain multiple VLPs representing each target strain. Accordingly, Gardasil 9 achieves protection against nine types of HPV because it contains nine different VLPs. [European Medicines Agency. Gardasil 9 Public Assessment Report, 2015]. In contrast to L1, the immunogenicity of protein L2 is low, probably because it is only transiently displayed on the virion surface during infection [Tyler, M. et al., Vaccine 2014]. However, some epitopes of L2 are highly conserved in diverse HPV types, and HPV vaccines based on L2 epitopes can therefore elicit broadly-neutralizing antibodies that protect against a wide range of HPV types [Tumban, E. et al., PLoS ONE 2011; Zhai, L. et al., Antivir. Res. 2017]. L2 does not assemble into VLPs, therefore vaccines based on this protein must incorporate it into a carrier. Various nanoparticles were developed for this purpose because it is easy to control their physicochemical properties (e.g., size, shape, and hardness) and they can be modified to display multiple copies of the epitope, enhancing their immunogenicity [Shao, S. et al., Nat. Chem. 2015; Shukla, S. et al., Biomaterials 2017; Xia, Y. et al. Nat. Mater. 2018].

Challenges remain to produce vaccines that are stable without the cold chain, to be distributed worldwide. Further, the ideal candidate would be delivered using a vaccine delivery device, such as a microneedle, enabling self-administration in the absence of a healthcare professional. Toward this goal, Applicant developed a process to produce implants for sustained release of HPV vaccines. Such delivery devices hold the potential to overcome the need for repeat dosing. Biodegradable polymers are ideal slow-release carriers for antigens, as sustained exposure to antigens over time leads to an improved immunological response. [Yue, H. et al., Vaccine 2015; Demento, S. L. et al., Biomaterials 2012; Desai, K. G. H. et al., J Control. Release Off. J. Control. Release Soc. 2013; Irvine, D. J. et al., Adv. Drug Deliv. Rev. 2020] Applicant also discloses a melt-processing method to encapsulate VLPs into biodegradable polymers and when implanted intact VLPs were released slowly over time. The advantage of melt-processing to develop slow-release implants is its massive scalability, reproducibility, and solvent-free nature. One reason that proteins can undergo the high temperatures necessary for melt-encapsulation, is the reduced hydration state, which enhances thermal stability. Furthermore, VLPs are able to undergo the rigors of melt-processing due to their exceptional stability, with negligible degradation or aggregation at temperatures of ˜100° C. When the implants were loaded with immunogenic VLPs and implanted subcutaneously, the resulting antibody titers were similar to the traditional schedule with three doses, providing confidence that the implants could be used as a single-dose vaccine platform.

In one aspect, Applicant discloses herein a single-dose HPV vaccine in which L2 peptide antigens from the high-risk HPV16 strain were displayed on the surface of VLPs derived from bacteriophage Qβ. In one aspect, the vaccines use a bioconjugate chemistry protocol to achieve multivalent presentation of L2 antigens on the Qβ carrier. A combination of size exclusion chromatography (SEC), transmission electron microscopy (TEM), and dynamic light scattering (DLS), and gel electrophoresis was performed to validate the structural integrity and epitope display of the HPV-Qβ. Immunogenicity was validated in healthy mice and the antibody titers and subtypes were determined using the ELISA-based protocols. The HPV-Qβ particles were then encapsulated in poly(lactic-co-glycolic acid) (PLGA) implants, using a benchtop melt-processing system, and implanted into mice. The release rate of the VLPs were monitored in vitro and in vivo and confirmed that the HPV-Qβ particles released remained intact. Finally, immunizations were carried out by comparing the implant vs. soluble injections. Data showed that the antibody titers generated by the single-dose HPV-Qβ/PLGA implant were equivalent to traditional injection schedules and played a neutralizing role against the HPV pseudovirus.

Also provided herein is one or more virus or virus-like particle(s) (VLP), each of which comprises at least one epitope of a pathogen and optionally comprising two or more epitopes of the pathogen that may be the same or different from each other. In one embodiment the epitope(s) does not activate a T cell or a cytotoxic T cell when administered to a subject.

In one aspect, the virus or virus-like particle is a bacteriophage virus or virus like particle, or a plant virus or virus like particle. In another aspect, the virus or virus-like particle is a bacteriophage Qβ virus or virus like particle. In a further aspect, the virus or virus-like particle is a plant picornavirus virus or virus like particle, or a filamentous plant virus or virus-like particle, e.g., wherein the plant virus or virus-like particle is of the or Alphafexiviridae family. Alternatively, the plant virus or virus-like particle is a cowpea mosaic virus-like particle or a potato virus X virus-like particle. Yet further, the plant virus particle or virus-like particle is a rod-shaped virus or virus-like particle. Yet further, the rod-shaped virus or virus-like particle is a tobacco mosaic virus or virus-like particle.

In one aspect, the pathogen is Human papillomavirus (HPV) and accordingly, the epitopes are from HPV. As discussed in more detail below, the one or more virus or VLP are useful in the treatment or prevention of an HPV infection, or an-HPV related cancer, or both. Non-limiting examples of HPV-related cancers include a cervical cancer, an oropharyngeal cancer, an anal cancer, a penile cancer, a vaginal cancer, or a vulva cancer.

In one embodiment the HPV is selected from one or more of HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, or HPV82.

In one another embodiment, the epitope(s) is of an HPV capsid protein, such as for example, an HPV capsid protein L1 or an HPV capsid protein L2, or both.

Alternatively, the epitope(s) is present in the HPV16 capsid protein L2 or a fragment thereof, e.g., amino acid 17 to 31 of HPV16 L2.

Yet further, the epitope(s) is present in a peptide comprising, or consisting essentially or, or yet further consisting of an amino acid sequence of QLYKTCKQAGTCPPD (SEQ ID NO: 4, which is amino acid 17 to amino acid 31 of HPV16 L2 protein), or a fragment of an HPV capsid protein aligned with SEQ ID NO: 4, or an equivalent of each thereof. In one aspect, the epitopes further comprise 3 amino acids (aa), or 4, or 5, or 6, or 7, or 8, or 9, or 10, or 11, or 12, or 13, or 14, or 15, or 16, or 17, or 18, or 19, or 20, or 25 aa on the N- and/or c-terminus of the fragment, wherein the additional amino acids of the wild-type reference protein. As used herein, an equivalent comprises an amino acid sequence having at least 70%, or alternatively at least 75%, or alternatively at least 80%, or alternatively at least 85%, or alternatively at least 90%, or alternatively at least 95%, or alternatively at least 97%, or alternatively at least 99%, or 100% sequence identity to the reference amino acid SEQ ID NO: 4, and similar biological activity as the reference amino acid. Further provided is a plurality of a virus or virus-like particle(s) comprising one or more HPV epitopes that may be the same or different from each other.

For example, the at least one of the HPV epitope(s), or a protein fragment is conjugated directly or indirectly to the virus or the virus-like particle, or a coat protein of the virus or the virus-like particle. Alternatively, the HPV epitope(s) or a protein fragment comprising at least one of the epitope(s) is conjugated indirectly comprising, or consisting essentially of, or yet further comprising of a linker to the virus or virus-like particle, or a coat protein of the virus or the virus-like particle at the C-terminus or the N-terminus. In one aspect, the linker comprises an amino acid sequence of (GSG)n, (GGSG)n, (GPSL)n, or (GGSGGGSG)n, wherein n is an integer from 1 to 5, or 1 to 4, or 1 to 3, or 2, or 3, or 4 or 5, or wherein the linker is an SM(PEG)₈ bifunctional linker comprising an NHS group and a maleimide group, or wherein the linker comprises an N-terminal cysteine residue conjugated to triple glycine (GGG) and an N-hydroxysuccinimide-PEG4-maleimide linker SM-PEG4. In a further aspect, the C-terminus or the N-terminus of the epitope(s), a peptide comprising at least one of the epitope(s), or a protein fragment comprising at least one of the epitope(s) is conjugated directly or indirectly to the N-terminus or the C-terminus of a coat protein of the virus or the virus-like particle.

Vaccines for the Prevention of COVID

The difficulties faced during the COVID-19 pandemic offer learning opportunities to improve upon for future vaccine efforts against any emerging pathogen. Therefore, it is critical to develop novel platform technologies and delivery devices to produce vaccines that are highly stable, do not require cold-chain distribution, are effective after a single dose, are massively scalable with ease of fabrication, and can be self-administered.

Toward these goals, Applicant developed plant virus and bacteriophage-based COVID-19 vaccines displaying peptides epitopes from the SARS-CoV-2 S protein. The virus nanoparticle, here cowpea mosaic virus (CPMV) or a virus-like particle derived from Qβ bacteriophage serves as an adjuvant and delivery technology enabling efficient uptake by draining lymph nodes and processing by professional antigen-presenting cells.

CPMV is a plant virus that self-assembles into 30 nm-sized icosahedral capsids that consists of 60 copies each of a small and large coat protein unit; the particles have pseudo-T3 symmetry. CPMV can be produced through molecular farming in plants and has been validated as a vaccine platform in preclinical models, and more recently its application as cancer immunotherapy has been reported. Qβ is expressed as a VLP that consists of 180 identical copies of a coat protein and the 30 nm-sized particles have T3 symmetry. The Qβ-based nanoparticles can be mass-produced through bacterial fermentation with several Qβ-based vaccines undergoing clinical testing. CPMV and Qβ have high thermal stability and therefore can undergo hot-melt extrusion to formulate slow-release polymer blends as well as fast-soluble microneedle delivery devices. Applicant adapted these methods to produce COVID-19 vaccines and their delivery devices. Molecular farming of CPMV, fermentation of Qβ, as well as hot melt extrusion of polymer blends and MN polymer micromolding are vastly scalable techniques and therefore suitable approaches for the development of novel vaccine platform technologies.

Any vaccine requires an antigen, adjuvant, and delivery technology; for the target antigen, Applicant focused on B cell epitopes from the SARS-CoV-2 S protein. Current COVID-19 vaccines and most candidates undergoing development target full-length spike (S) glycoprotein or the receptor binding domain (RBD), which play a key role in viral entry. While vaccines based on full-length S protein have shown tremendous efficacy, concerns have been raised because of reduced efficacy against emerging new variants of SARS-CoV-2, variants of concern (VOCs). Applicant report on our efforts using B cell epitopes that are highly conserved amongst SARS-CoV-2 variants. Another rationale to target B cell epitopes is that antibody responses are more targeted as compared to the broad spectrum of antibody and cellular responses when immunization is carried out with full length protein. Earlier studies on SARS and MERS vaccine candidates have pointed to risks of antibody-dependent enhancement (ADE) of infection as well as cellular response mediated Th2 immunopathology. Therefore, developing peptide epitope-based SARS-CoV-2 vaccine strategies may yield a safer vaccine with antibody responses consistently targeting and neutralizing across VOCs.

In one aspect, Applicant reports herein the development of trivalent CPMV- and Qβ-based COVID-19 vaccines and demonstrate efficacy when administered using a prime-boost schedule and injection or microneedle patches; the latter offering opportunity for self-administration. Applicant also demonstrates the efficacy of a slow-release injectable implant after a single administration. Antibody titers and immune responses were studied, and neutralization potency was assayed using a surrogate receptor binding assay as well as a SARS-CoV-2 neutralization assay using human cells.

99| In another aspect, Applicant provides herein one or more virus or VLP that comprises one or more epitopes of a coronavirus for the treatment or prevention of a severe acute respiratory syndrome (SARS) associated coronavirus (SARS-CoV). Non-limiting examples of such comprise SARS-CoV-1 (also referred to herein as SARS), or SARS-CoV-2, or both SARS-CoV-1 and SARS-CoV-2.

In one embodiment, the epitope(s) of the SARS-CoV-2 is of a spike protein (S protein) of a coronavirus. In another aspect, the epitope(s) are B-cell epitopes identified in the convalescent sera from recovered SARs patients (e.g., SEQ ID NOS: 5-9). Non-limiting examples of such include an epitope(s) that is present in one or more peptide(s) that comprise, or consist essentially of, or yet further consists of at one or more amino acid sequence(s) selected from KGIYQTSN (SEQ ID NO: 5, amino acid (aa) 310 to aa 317 of SARS-CoV-2 S protein), AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), EIDRLNEVAKNLNESLIDLQEL (SEQ ID NO: 8, amino acid (aa) 1182 to aa 1209 of SARS-CoV-2 S protein), ATRFASVYAWNRKRISN (SEQ ID NO: 9, amino acid (aa) 346 to aa 362 of SARS-CoV-2 S protein), YNSASFSTFKCYGVSPTK (SEQ ID NO: 10, aa 369 to aa 386 of SARS-CoV-2 S protein), LPDPSKPSKRSFIED (SEQ ID NO: 11, aa 806 to aa 820 of SARS-CoV-2 S protein), FRKSN (SEQ ID NO: 12, aa 456 to aa 460 of SARS-CoV-2 S protein), PSKPSKRSFIEDLLFNKV (SEQ ID NO: 13, aa 809 to aa 826 of SARS-CoV-2 S protein), TESNKKFLPFQQFGRDIA (SEQ ID NO: 14, aa 553 to aa 570 of SARS-CoV-2 S protein), TESNKKFLPFQQ (SEQ ID NO: 15, aa 553 to aa 564 of SARS-CoV-2 S protein), HADQLTPTWRVY (SEQ ID NO: 16, aa 625 to aa 636 of SARS-CoV-2 S protein), FKEELDKYFKNH (SEQ ID NO: 17, aa 1148 to aa 1159 of SARS-CoV-2 S protein), FASTEKSNIIRGWIF (SEQ ID NO: 18, aa 92 to aa 106 of SARS-CoV-2 S protein), PFLGVYYHKNNKSWM (SEQ ID NO: 19, aa 135 to aa 153 of SARS-CoV-2 S protein), EVRQIAPGQTGKIAD (SEQ ID NO: 20, aa 406 to aa 420 of SARS-CoV-2 S protein), NNLDSKVGGNYNYLYR (SEQ ID NO: 22, aa 439 to aa 454 of SARS-CoV-2 S protein), LFRKSNLKPFERDIS (SEQ ID NO: 23, aa 455 to aa 469 of SARS-CoV-2 S protein), or a fragment of a coronavirus S protein aligned with each thereof, or an equivalent of each thereof, the epitopes being the same or different from each other. Bolded amino acids indicate B cell epitopes used that contained a mutation from variants of concern, with the bolded amino acids indicating a deletion or mutation. In one aspect, the virus or VLP comprises from about 30 to about 120 peptide epitopes per virus or VLP, and ranges in between as disclosed herein. They can further comprise a linker and be linked with the C-terminus or the N-terminus exposed.

In one aspect, the virus or VLP comprises SEQ ID NOS: 6 to 8. In another aspect, the virus or VLP comprises SEQ ID NOS: 13, 14 and 16.

In one aspect, the epitopes further comprise 3 amino acids (aa), or 4, or 5, or 6, or 7, or 8, or 9, or 10, or 11, or 12, or 13, or 14, or 15, or 16, or 17, or 18, or 19, or 20, or 25 aa on the N- and/or C-terminus of the epitope, wherein the additional amino acids of the wild-type reference protein. As used herein, an equivalent comprises an amino acid sequence having at least 70%, or alternatively at least 75%, or alternatively at least 80%, or alternatively at least 85%, or alternatively at least 90%, or alternatively at least 95%, or alternatively at least 97%, or alternatively at least 99%, or 100% sequence identity to the reference amino acid SEQ ID NOs: 5-23, and similar biological activity as the reference amino acid. Further provided is a plurality of a virus or virus-like particle(s) comprising one or more coronavirus epitopes that may be the same or different from each other.

Alternatively, the epitope(s) is present in one or more peptide(s) comprising, or consisting essentially of, or yet further consisting of at least one or more amino acid sequence(s) selected from AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), EIDRLNEVAKNLNESLIDLQEL (SEQ ID NO: 8, amino acid (aa) 1182 to aa 1209 of SARS-CoV-2 S protein), YNSASFSTFKCYGVSPTK (SEQ ID NO: 9, aa 369 to aa 386 of SARS-CoV-2 S protein), PSKPSKRSFIEDLLFNKV (SEQ ID NO: 10, aa 809 to aa 826 of SARS-CoV-2 S protein), TESNKKFLPFQQFGRDIA (SEQ ID NO: 11, aa 553 to aa 570 of SARS-CoV-2 S protein), HADQLTPTWRVY (SEQ ID NO: 12, aa 625 to aa 636 of SARS-CoV-2 S protein), PFLGVYYHKNNKSWM (SEQ ID NO: 13, aa 135 to aa 153 of SARS-CoV-2 S protein), or a fragment of a coronavirus S protein aligned with each thereof, or an equivalent of each thereof or wherein the epitope(s) is present in one or more peptide(s) comprising at least one amino acid sequence(s) selected from AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), or EIDRLNEVAKNLNESLIDLQEL (SEQ ID NO: 8, amino acid (aa) 1182 to aa 1209 of SARS-CoV-2 S protein), or a fragment of a coronavirus S protein aligned with each thereof, or wherein the epitope(s) is present in one or more peptide(s) comprising at least one amino acid sequence(s) selected from YNSASFSTFKCYGVSPTK (aa 369 to aa 386 of SARS-CoV-2 S protein), PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), PFLGVYYHKNNKSWM (aa 135 to aa 153 of SARS-CoV-2 S protein), or a fragment of a coronavirus S protein aligned with each thereof, or wherein the epitope(s) is present in one or more peptide(s) comprising at least one amino acid sequence(s) selected from PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), or an equivalent or fragment of a coronavirus S protein aligned with each thereof.

In one aspect, the epitopes further comprise 3 amino acids (aa), or 4, or 5, or 6, or 7, or 8, or 9, or 10, or 11, or 12, or 13, or 14, or 15, or 16, or 17, or 18, or 19, or 20, or 25 aa on the N- and/or c-terminus of the fragment, wherein the additional amino acids of the wild-type reference protein. As used herein, an equivalent comprises an amino acid sequence having at least 70%, or alternatively at least 75%, or alternatively at least 80%, or alternatively at least 85%, or alternatively at least 90%, or alternatively at least 95%, or alternatively at least 97%, or alternatively at least 99%, or 100% sequence identity to the reference amino acid SEQ ID NOs: 6-13, and similar biological activity as the reference amino acid. Further provided is a vaccine composition that comprises a plurality of a virus or virus-like particle(s) comprising one or more coronavirus epitopes that may be the same or different from each other.

In a further aspect, provided herein is a plurality of the one or more virus or virus-like particles, each of which comprises at least one epitope(s), or one or more peptide(s) each of which comprises at least one of the epitope(s), or one or more protein fragment(s) each of which comprises at least one of the epitope(s), e.g., SEQ ID NO: 4, or alternatively, SEQ ID NOS: 5-23 or alternatively SEQ ID NO: 6-13, and equivalents of each thereof. In another aspect, the virus or VLP comprises SEQ ID NOS: 6 to 8. In another aspect, the virus or VLP comprises SEQ ID NOS: 13, 14 and 16.

In a further aspect, the epitope(s), a peptide comprising thereof, or a protein fragment comprising thereof is present on the outer surface of the virus or virus-like particle. In another aspect, the epitope(s), a peptide comprising at least one of the epitope(s), or a protein fragment comprising at least one of the epitope(s) is conjugated directly or indirectly to the virus or virus-like particle, or a coat protein of the virus or virus-like particle. Yet further, the epitope(s), a peptide comprising at least one of the epitope(s), or a protein fragment comprising at least one of the epitope(s) is conjugated indirectly comprising, or consisting essentially of, or yet further consisting of a linker to the virus or virus-like particle or a coat protein of the virus or virus-like particle.

In one aspect, the linker comprises an amino acid sequence of (GSG)n, (GGSG)n, (GPSL)n, or (GGSGGGSG)n, wherein n is an integer from 1 to 5, or 1 to 4, or 1 to 3, or 2, or 3, or 4 or 5, or wherein the linker is an SM(PEG)₈ bifunctional linker comprising an NHS group and a maleimide group, or wherein the linker comprises an N-terminal cysteine residue conjugated to triple glycine (GGG) and an N-hydroxysuccinimide-PEG4-maleimide linker SM-PEG4. In a further aspect, the C-terminus or the N-terminus of the epitope(s), a peptide comprising at least one of the epitope(s), or a protein fragment comprising at least one of the epitope(s) is conjugated directly or indirectly to the N-terminus or the C-terminus of a coat protein of the virus or the virus-like particle. Non-limiting examples of such are provided above and include for example, an amino acid sequence comprising, or consisting essentially thereof, or yet further consisting of GSG, GPSL, or GGSGGGSG, or wherein the linker is an SM(PEG)₈ bifunctional linker comprising an NHS group and a maleimide group, or wherein the linker comprises an N-terminal cysteine residue conjugated to triple glycine (GGG) and an N-hydroxysuccinimide-PEG4-maleimide linker SM-PEG4. In one embodiment, the C-terminus or the N-terminus of the epitope(s), a peptide comprising at least one of the epitope(s), or a protein fragment comprising at least one of the epitope(s) is conjugated directly or indirectly to the N-terminus or the C-terminus of a coat protein of the virus or virus-like particle.

In a further aspect, the virus or VLP of this disclosure is detectably labeled.

Pharmaceutical Compositions

Also provided are pharmaceutical compositions and formulations that can comprise one, two or all there of the epitopes described above conjugated to the virus or virus-like particle (e.g. Qβ) and a carrier, such as a pharmaceutically acceptable carrier. In a further aspect, provided herein is composition comprising a plurality of the one or more virus or virus-like particles, each of which comprises at least one epitope(s), or one or more peptide(s) each of which comprises at least one of the epitope(s), or one or more protein fragment(s) each of which comprises at least one of the epitope(s), e.g., SEQ ID NO: 4, or alternatively, SEQ ID NOS: 5-23 or alternatively SEQ ID NO: 6-13, and equivalents of each thereof.

As understood by one of skill in the art, the composition or formulation can comprise a plurality of virus-like particles, e.g., Qβ, in a variety of ratios of components non-limiting examples of such that include: 1:1:1; 1:2:1; 1:3:2; 2:1:2; 3:1:2: 2:3:4, of any one of SEQ ID NOS: 5-23 or alternatively 6-13, or equivalents of each thereof. The virus or VLP can be the same or different from each other. The epitopes can be the same or different from each other.

In one aspect, epitope(s), or a protein fragment comprising at least one of the epitope(s) is conjugated indirectly comprising, or consisting essentially of, or yet further comprising of a linker to the virus or virus-like particle, or a coat protein of the virus or the virus-like particle. In one aspect, the linker comprises an amino acid sequence of GSG, GPSL, or GGSGGGSG, or wherein the linker is an SM (PEG)₈ bifunctional linker comprising an NHS group and a maleimide group, or wherein the linker comprises an N-terminal cysteine residue conjugated to triple glycine (GGG) and an N-hydroxysuccinimide-PEG4-maleimide linker SM-PEG4. In a further aspect, the C-terminus or the N-terminus of the epitope(s), a peptide comprising at least one of the epitope(s), or a protein fragment comprising at least one of the epitope(s) is conjugated directly or indirectly to the N-terminus or the C-terminus of a coat protein of the virus or the virus-like particle.

In another aspect, the vaccine composition further comprises an additional active agent to treat or prevent HPV and associated diseases or alternatively, COVID and associated diseases.

In a further aspect, the formulation further comprises a stabilizer or cryopreservative and the formulation is frozen or lyophilized for ease of use and storage.

In a further aspect, the carrier comprises a slow-release implant, e.g., encapsulated within a degradable polymer matrix. In one aspect, the degradable polymer matrix comprises a melt processable degradable polymer material that is biocompatible and, upon degradation, produces substantially non-toxic products, wherein the melt processable degradable polymer material is a melt processable biodegradable polymer, and wherein the degradable polymer material has a melt temperature below the degradation temperature of the virus or virus-like particle(s). A non-limiting example of such includes poly(lactic-co-glycolic acid) (PLGA) or a copolymer thereof or wherein the slow-release implant comprises one or more of: about 50% to about 99% PLGA, about 1% to about 50% virus or virus-like particle(s), or PEG8000, or wherein the slow-release implant comprises one or more of: about 80% PLGA, about 10% virus or virus-like particle(s), or about 10% PEG8000 optionally by weight, or wherein the slow-release implant comprises one or more of: about 75% PLGA, about 10% VNPs, or about 15% PEG8000, optionally wherein the % is indicated a weight percentage.

In another aspect, provided herein is a composition comprising, consisting essentially of, or consisting of a component or a combination provided herein, and at least one pharmaceutically acceptable excipient.

Compositions, including pharmaceutical compositions comprising, consisting essentially of, or consisting of a component or a combination as described herein, can be manufactured by means of conventional mixing, dissolving, granulating, dragee-making levigating, emulsifying, encapsulating, entrapping, or lyophilization processes. The component or combination can be formulated in conventional manner using one or more physiologically acceptable carriers, diluents, excipients, or auxiliaries which facilitate processing of the component or combination provided herein into preparations which can be used pharmaceutically.

The vaccine component or combination of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

In one embodiment, this technology relates to a vaccine composition comprising a component or a combination as described herein and a carrier.

In another embodiment, this technology relates to a pharmaceutical composition comprising a vaccine component or a combination as described herein and a pharmaceutically acceptable carrier.

In another embodiment, this technology relates to a pharmaceutical composition comprising a therapeutically effective amount of a vaccine component or a combination as described herein and a pharmaceutically acceptable carrier.

The pharmaceutical compositions for the administration of a component or a combination as disclosed herein can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each component provided herein is included in an amount sufficient to produce the desired effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the vaccine component or the combination can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the component or the combination provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

Methods for Treating and Preventing HPV Infection and Associated Disease

This disclosure also provides a method for one or more of the following in a subject in need thereof: treating or preventing an HPV infection or related disease or another disease caused by HPV, producing an antibody recognizing and binding to HPV, triggering, enhancing, or prolonging an immune response to HPV causing the disease, or delivering at least one epitope(s) of HPV to the subject comprising, or consisting essentially of, or consisting of administering to the subject one or more virus or virus-like particle(s), each of which comprises at least one epitope of HPV. In one aspect, the subject is mammal or a human in need of such treatment. As is apparent, the virus or VLP is administered in an effective amount for the treatment desired and subject being treated. Various embodiments of the HPV vaccine are disclosed above and incorporated herein to more fully describe the disclosed methods.

In one aspect, the disease is an HPV infection, or an HPV-related cancer, or both. Non-limiting examples of such cancers are selected from: a cervical cancer, an oropharyngeal cancer, an anal cancer, a penile cancer, a vaginal cancer, or a vulva cancer.

The virus or VPL containing the HPV epitope(s) are described above, compositions and formulations comprising such are described herein. In one particular aspect, the epitope(s) is of an HPV capsid protein, e.g., the HPV is selected from one or more of HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, or HPV82.

In a further aspect, the method comprises administering an HPV capsid protein that comprises, or consists essentially of, or further consists of an HPV capsid protein L1 or an HPV capsid protein L2, or both. In a particular aspect, the epitope(s) is of a HPV16 capsid protein L2 is administered to the subject.

In a particular aspect, the HPV epitope(s) is present in a peptide comprising, or consisting essentially of, or consisting of an amino acid sequence of QLYKTCKQAGTCPPD (SEQ ID NO: 4, which is amino acid 17 to amino acid 31 of HPV16 L2 protein), or a fragment or an equivalent of an HPV capsid protein aligned with SEQ ID NO: 4.

Methods of administration are known in the art and can comprise one or more doses or using a single formulation as described herein, e.g., wherein the method does not comprises repeating the administering step. For example, the slow-release implant is administered by a microneedle patch or by injection. Alternatively, administration comprising repeating the administering step for about once, about twice, about three times, about four times, about five times, or more, and wherein two subsequent administrations are about 1 day to about 1 year apart, or about 1 week apart, or about 2 weeks apart, or about 3 weeks apart, or about 4 weeks apart, or about 1 month apart, or about 2 months apart, or about 3 months apart, or about 6 months apart. Yet further and alternatively, the administering step is not repeated for more than 6 times, or more than 7 times, or more than 8 times, or more than 9 times, or more than 10 times, or more than 15 times, or more than 20 times.

In one aspect of the method, the epitope(s) does not activate a T cell or a cytotoxic T cell in the subject.

In a further aspect, the method does not comprise administering to the subject an additional adjuvant.

HPV Dosages and Dosing Regimens

The appropriate amount and dosing regimen of the vaccine component or the combination, when present to be administered to the subject according to any of the methods disclosed herein, may be determined by one of ordinary skill in the art.

In some embodiments, the vaccine component or the combination as disclosed herein, may be administered to a subject in need thereof, either alone or as part of a pharmaceutically acceptable formulation, once a week, once a day, twice a day, three times a day, or four times a day, or even more frequently.

Administration of the vaccine component or the combination as disclosed herein may be effected by any method that enables delivery of the component or the combination to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the vaccine, composition or formulation are well-known in the relevant art and would be understood to be encompassed by the skilled artisan once provided the teachings disclosed herein.

Methods of Making the Virus or VLP for the Treatment or Prevention of HPV or HPV-Related Disease

Applicant also provides methods to make the vaccines and formulations. In one aspect, the VLP is a bacteriophage Qβ or CPMV or other VLP and the epitope is conjugated to the virus, VLP (e.g., Qβ) or CPMV via a SM(PEG)₈ bifunctional linker containing an NH group and a maleimide group. The virus, VLP or CPMV is mixed in excess of the SM(PEG)₈ and unreacted SM(PEG)₈ is removed. The particles are recovered and reacted with an excess of epitopes or peptides at room temperature. Unreacted peptides are removed.

Alternatively, a polynucleotide encoding the VLP, CMPV or bacteriophage Qβ coat protein with an optional N-terminal cysteine and linker such as GGG is conjugated to the polynucleotide encoding the epitope and inserted into a plasmid for expression in a eukaryotic or prokaryotic system. A non-limiting example of E. coli expression vector is the pDUET-1 expression vector for expression in E. coli, e.g., E. coli B121 (DE3). Thus, in one aspect, also provided herein are polynucleotides encoding the VLP, CMPV or bacteriophage Qβ coat protein with an optional N-terminal cysteine and linker such as GGG and the epitope(s) of interest. The polynucleotides can further comprise a regulatory element for expression in an appropriate system, e.g., a promoter or enhancer. Also provided is a vector comprising the polynucleotide, as well as host cells comprising the polynucleotide and/or vectors. The vectors can be a plasmid or viral vector and the host cells can be eukaryotic or prokaryotic cells. The polynucleotides are expressed by growing the host cells under conditions for expression of the polynucleotides and the vaccine product is isolated from the cell culture.

Also provided are methods for preparing the compositions by admixing the vaccine with a carrier such as a pharmaceutically acceptable carrier. The compositions and/or formulations can further comprise a cryopreservative or stabilizer. The compositions can be frozen or lyophilized.

PGLA implants can be prepared using a melt-processing system. Particles can be lyophilized before melt extrusion. Components are mixed and then loaded into the melt-processing system and heated for an effective amount of time and pressure for extrusion. Implants are dried and cut as desired.

Microneedle fabrication methods are known in the art and described herein.

Method for Treating or Preventing COVID Infections and Associated Diseases

Also provided herein is a method for treating or preventing infection or disease resulting from a coronavirus comprising or consisting essentially thereof or consisting of administering to the subject an effective amount of a virus or VLP comprising an epitope from a coronavirus, as described herein. Various embodiments of the vaccines are described above and incorporated herein to more fully describe the methods as disclosed herein.

In one aspect, the coronavirus is a severe acute respiratory syndrome (SARS) associated coronavirus (SARS-CoV), e.g., the SARS-CoV comprises, or consists essentially of, or further consists of SARS-CoV-1 (also referred to herein as SARS), or SARS-CoV-2, or both SARS-CoV-1 and SARS-CoV-2.

In one embodiment, the epitope(s) that is administered to the subject is of a spike protein (S protein) of a coronavirus. Non-limiting examples of such include an epitope(s) is present in one or more peptide(s) comprising, or consisting essentially of, or consisting of at least one amino acid sequence(s) selected from KGIYQTSN (SEQ ID NO: 5, amino acid (aa) 310 to aa 317 of SARS-CoV-2 S protein), AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), EIDRLNEVAKNLNESLIDLQEL (SEQ ID NO: 8, amino acid (aa) 1182 to aa 1209 of SARS-CoV-2 S protein), ATRFASVYAWNRKRISN (SEQ ID NO: 9, amino acid (aa) 346 to aa 362 of SARS-CoV-2 S protein), YNSASFSTFKCYGVSPTK (SEQ ID NO: 10, aa 369 to aa 386 of SARS-CoV-2 S protein), LPDPSKPSKRSFIED (SEQ ID NO: 11, aa 806 to aa 820 of SARS-CoV-2 S protein), FRKSN (SEQ ID NO: 12, aa 456 to aa 460 of SARS-CoV-2 S protein), PSKPSKRSFIEDLLFNKV (SEQ ID NO: 13, aa 809 to aa 826 of SARS-CoV-2 S protein), TESNKKFLPFQQFGRDIA (SEQ ID NO: 14, aa 553 to aa 570 of SARS-CoV-2 S protein), TESNKKFLPFQQ (SEQ ID NO: 15, aa 553 to aa 564 of SARS-CoV-2 S protein), HADQLTPTWRVY (SEQ ID NO: 16, aa 625 to aa 636 of SARS-CoV-2 S protein), FKEELDKYFKNH (SEQ ID NO: 17, aa 1148 to aa 1159 of SARS-CoV-2 S protein), FASTEKSNIIRGWIF (SEQ ID NO: 18, aa 92 to aa 106 of SARS-CoV-2 S protein), PFLGVYYHKNNKSWM (SEQ ID NO: 19, aa 135 to aa 153 of SARS-CoV-2 S protein), EVRQIAPGQTGKIAD (SEQ ID NO: 20, aa 406 to aa 420 of SARS-CoV-2 S protein), NNLDSKVGGNYNYLYR (SEQ ID NO: 22, aa 439 to aa 454 of SARS-CoV-2 S protein), LFRKSNLKPFERDIS (SEQ ID NO: 23, aa 455 to aa 469 of SARS-CoV-2 S protein), or a fragment or equivalent thereof of a coronavirus S protein aligned with each thereof, the epitopes being the same or different from each other. In a further aspect, the method comprises or consists essentially of, or further consists of an epitope(s) is present in one or more peptide(s) comprising, or consisting essentially of, or consisting of at least one amino acid sequence(s) selected from AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), EIDRLNEVAKNLNESLIDLQEL (SEQ ID NO: 8, amino acid (aa) 1182 to aa 1209 of SARS-CoV-2 S protein), YNSASFSTFKCYGVSPTK (aa 369 to aa 386 of SARS-CoV-2 S protein), PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), PFLGVYYHKNNKSWM (aa 135 to aa 153 of SARS-CoV-2 S protein), or a fragment or an equivalent of a coronavirus S protein aligned with each thereof, or wherein the epitope(s) is present in one or more peptide(s) comprising, or consisting essentially of, or consisting of at least one amino acid sequence(s) selected from AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), or EIDRLNEVAKNLNESLIDLQEL (SEQ ID NO: 8, amino acid (aa) 1182 to aa 1209 of SARS-CoV-2 S protein), or a fragment or an equivalent thereof of a coronavirus S protein aligned with each thereof, or wherein the epitope(s) is present in one or more peptide(s) comprising, or consisting essentially of, or consisting of at least one amino acid sequence(s) selected from YNSASFSTFKCYGVSPTK (aa 369 to aa 386 of SARS-CoV-2 S protein), PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), PFLGVYYHKNNKSWM (aa 135 to aa 153 of SARS-CoV-2 S protein), or a fragment of a coronavirus S protein aligned with each thereof, or wherein the epitope(s) is present in one or more peptide(s) comprising, or consisting essentially of, or consisting of at least one amino acid sequence(s) selected from PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), or a fragment of a coronavirus S protein aligned with each thereof.

In one aspect, the virus or VLP comprises SEQ ID NOS: 6 to 8. In another aspect, the virus or VLP comprises SEQ ID NOS: 13, 14 and 16.

In another aspect, the method comprises, or consists essentially of, or consists of administering to the subject one or more virus or virus-like particles, each of which comprises at least one coronavirus or spike protein epitope(s), or one or more peptide(s) each of which comprises, or consists essentially of, or further consists of at least one of the epitope(s), or one or more protein fragment(s) each of which comprises, or consists essentially of, or further consists of at least one of the epitope(s). In another aspect, a plurality of virus or VPL are administered to the subject that may be the same or different from each other. In one aspect, the VPL is CPMV and/or Qβ. In a further aspect, the epitope(s), a peptide comprising thereof, or a protein fragment comprising thereof is present on the outer surface of the virus or virus-like particle. The coronavirus or spike protein epitope(s), a peptide comprising, or consisting essentially of, or consisting of at least one of the epitope(s), or a protein fragment comprising, or consisting essentially of, or consisting of at least one of the epitope(s) is conjugated directly or indirectly to the virus or virus-like particle, or a coat protein of the virus or virus-like particle.

Any appropriate method of administration can be used. In one aspect, the method does not comprises repeating the administering step. Alternatively, the virus or virus-like particle(s) is administrated to the subject in a slow-release implant or is encapsulated within a degradable polymer matrix. In one aspect, the degradable polymer matrix comprises, or consists essentially of, or further consists of a melt processable degradable polymer material that is biocompatible and, upon degradation, produces substantially non-toxic products, wherein the melt processable degradable polymer material is a melt processable biodegradable polymer, and wherein the degradable polymer material has a melt temperature below the degradation temperature of the virus or virus-like particle(s). For example, the degradable polymer material comprises, or consists essentially of, or further consists of poly(lactic-co-glycolic acid) (PLGA) or a copolymer thereof. In one aspect, the slow-release implant comprises, or consists essentially of, or further consists of one or more of: about 50% to about 99% PLGA, about 1% to about 50% virus or virus-like particle(s), or PEG8000, or wherein the slow-release implant comprises, or consists essentially of, or further consists of one or more of: about 80% PLGA, about 10% virus or virus-like particle(s), or about 10% PEG8000, or wherein the slow-release implant comprises, or consists essentially of, or further consists of one or more of: about 75% PLGA, about 10% VNPs, or about 15% PEG8000, optionally wherein the % indicates a weight percentage. In a further aspect, the slow-release implant is loaded into a microneedle patch.

In one aspect, the administration is repeated for about once, about twice, about three times, about four times, about five times, or more, and wherein two subsequent administrations are about 1 day to about 1 year apart, or about 1 week apart, or about 2 weeks apart, or about 3 weeks apart, or about 4 weeks apart, or about 1 month apart, or about 2 months apart, or about 3 months apart, or about 6 months apart. Alternatively, the administering step is not repeated for more than 6 times, or more than 7 times, or more than 8 times, or more than 9 times, or more than 10 times, or more than 15 times, or more than 20 times.

In a further aspect, the epitope(s) does not activate a T cell or a cytotoxic T cell in the subject. In a yet further aspect, the method does not comprise administering to the subject an additional adjuvant.

In a yet further aspect, than one epitopes are delivered by the virus or virus-like particle(s), whereby showing a synergistic effect.

COVID Vaccine Dosages and Dosing Regimens

The appropriate amount and dosing regimen of the vaccine component or the combination, when present to be administered to the subject according to any of the methods disclosed herein, may be determined by one of ordinary skill in the art.

In some embodiments, the vaccine component or the combination as disclosed herein, may be administered to a subject in need thereof, either alone or as part of a pharmaceutically acceptable formulation, once a week, once a day, twice a day, three times a day, or four times a day, or even more frequently.

Administration of the vaccine component or the combination as disclosed herein may be effected by any method that enables delivery of the component or the combination to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the vaccine, composition or formulation are well-known in the relevant art and would be understood to be encompassed by the skilled artisan once provided the teachings disclosed herein.

Methods of Making the Virus or VLP for the Treatment or Prevention of COVID Infections and Associated Diseases

Applicant also provides methods to make the vaccines and formulations. In one aspect, the VLP is a bacteriophage Qβ or CPMV or other VLP and the epitope is conjugated to the virus, VLP (e.g., Qβ) or CPMV via a SM(PEG)₈ bifunctional linker containing an NH group and a maleimide group. The virus, VLP or CPMV is mixed in excess of the SM(PEG)₈ and unreacted SM(PEG)₈ is removed. The particles are recovered and reacted with an excess of epitopes or peptides at room temperature. Unreacted peptides are removed.

Alternatively, a polynucleotide encoding the VLP, CMPV or bacteriophage Qβ coat protein with an optional N-terminal cysteine and linker such as GGG is conjugated to the polynucleotide encoding the epitope and inserted into a plasmid for expression in a eukaryotic or prokaryotic system. A non-limiting example of E. coli expression vector is the pDUET-1 expression vector for expression in E. coli, e.g., E. coli B121 (DE3). Thus, in one aspect, also provided herein are polynucleotides encoding the VLP, CMPV or bacteriophage Qβ coat protein with an optional N-terminal cysteine and linker such as GGG and the epitope(s) of interest. The polynucleotides can further comprise a regulatory element for expression in an appropriate system, e.g., a promoter or enhancer. Also provided is a vector comprising the polynucleotide, as well as host cells comprising the polynucleotide and/or vectors. The vectors can be a plasmid or viral vector and the host cells can be eukaryotic or prokaryotic cells. The polynucleotides are expressed by growing the host cells under conditions for expression of the polynucleotides and the vaccine product is isolated from the cell culture.

Also provided are methods for preparing the compositions by admixing the vaccine with a carrier such as a pharmaceutically acceptable carrier. The compositions and/or formulations can further comprise a cryopreservative or stabilizer. The compositions can be frozen or lyophilized.

PGLA implants can be prepared using a melt-processing system. Particles can be lyophilized before melt extrusion. Components are mixed and then loaded into the melt-processing system and heated for an effective amount of time and pressure for extrusion. Implants are dried and cut as desired.

Microneedle fabrication methods are known in the art and described herein.

Kits

Also provided are kits for use in a method of any one of embodiments as disclosed herein comprising, or consisting essentially of, or consisting of an optional instruction for use and at least one of: the one or more virus or virus-like particle(s) optionally comprising the at least one epitope(s), one or more of the epitope(s), one or more peptide(s) or protein fragment(s) each of which comprises, or consists essentially of, or further consists of at least one of the epitope(s), or one or more of the peptides as disclosed herein.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the invention.

### Examples

Various aspects and embodiments of this disclosure are provided in the below Experimental Examples.

Experiment No. 1—Cardiovascular Vaccines

Materials and Methods

Qβ virus-like particle vaccines production. Bacteriophage Qβ virus-like particles (VLP) were expressed as previously reported.[30] Genes encoding for Qβ coat protein (CP) (NCBI accession: P03615) and Qβ CP-modified with target peptides (ApoB:[12]KTTKQSFDLSVKAQYKKNKH, CETP:[16] FGFPEHLLVDFLQSLS, and PCSK9:[20]NVPEEDGTRFHRQASKC) were codon optimized for E. coli expression and synthesized and cloned by GenScript Biotech Co. into pDUET-1 expression vectors. A linker of GSG was introduced between the C-terminus of Qβ CP and the target peptide. Four vectors were obtained and named corresponding to the carrying genes, pCDF_Qβ (unmodified Qβ CP), pCDF_Qβ_QβApoB (unmodified Qβ CP and QβApoB), pCOLA_Qβ_QβCETP (unmodified Qβ CP and QβCETP), and pRSF_Qβ_QβPCSK9 (unmodified Qβ CP and QβPCSK9). Three different plasmids were used to test whether the trivalent vaccine could be obtained through co-expression in the same cell. This was tested; however, yields of VLP-CETP were low; therefore, Applicant decided to express the vaccines side-by-side through transformation of E. coli B121 (DE3) [New England BioLabs] using one plasmid at a time. Positive bacteria colonies were selected and grown for 16 h at 37° C. and 250 rpm in LB medium [Thermo Fisher Scientific] with corresponding antibiotics (pCDF_Qβ and pCDF_Qβ_QβApoB, 25 μg/ml streptomycin [Sigma-Aldrich]; pCOLA_Qβ_QβCETP and pRSF_Qβ_QβPCSK9, 50 μg/ml kanamycin [Sigma-Aldrich]); freezer stocks were prepared using 20% (v/v) sterile glycerol and kept at −80° C. until use. The freezer stock of each transformed bacteria was grown first for 16 h at 37° C. and 250 rpm in 10 ml of MagicMedia™ [Invitrogen] with corresponding antibiotics added; then the culture was scaled up to 200 ml in the same medium and cultured for 20-24 h at 37° C. and 300 rpm. The cell pellet was harvested by centrifugation at 5,000×g for 20 min at 4° C. and frozen at −80° C. overnight. After that, the pellet was resuspended in 10 ml of lysis buffer [GoldBio] per gram of wet cell mass, a lysis cocktail (1 mg/ml lysozyme [GoldBio], 2 μg/ml of DNase [Promega] and 2 mM MgCl₂ [Fisher Scientific]) was added and the reaction mix was incubated at 37° C. for 1 h. To complete the lysis, sonication was performed at Amp 30% and 5-5 sec cycles for 10 min on ice. The lysate was centrifuge at 5,000×g, 4° C. for 30 min and the clear supernatant was collected. To purify Qβ VLPs and the three vaccines QβApoB, QβCETP, and QβPCSK9, the collected supernatant was precipitated by adding 10% (w/v) PEG8000 (Thermo Fisher Scientific) at 4° C. for 12 h on a rotisserie. The precipitated fraction was pelleted by centrifugation at 5,000×g and dissolved in 40 ml PBS (137 mM NaCl, 2.7 mM KCl, 10 mM Na₂HPO₄, and 1.8 mM KH₂PO₄, pH 7.4) before extraction with a 1:1 (v/v) butanol/chloroform. The aqueous fraction was collected by centrifugation as above (at 5,000×g) and finally the VLPs were purified on a 10-40% sucrose velocity gradient by ultracentrifugation at 9,6281×g for 4.5 h. The light-scattering VLP band was collected and pelleted by ultracentrifugation at 16,0326×g for 3 h. The purified VLPs were resuspended in PBS and stored at 4° C. until further use.

Antigen in silico characterization. Target peptide antigens ApoB=KTTKQSFDLSVKAQYKKNKH,[12] CETP=FGFPEHLLVDFLQSLS,[16] and PCSK9=NVPEEDGTRFHRQASKC[20] were analyzed using an online peptide calculator (https://pepcalc.com/) to determine their isoelectric point. The sequence identity of the human-specific peptide antigens to corresponding mouse proteins (ApoB=NP_033823.2 and PCSK9=AAP31672.1; mice lack CETP) was determined using protein BLAST software (https://blast.ncbi.nlm.nih.gov/).

VLP characterization. The Qβ-based VLPs (Qβ, QβApoB, QβCETP, and QβPCSK9) were characterized as previously described[29] using fast protein liquid chromatography (FPLC), transmission electron microscopy (TEM), dynamic light scattering (DLS), sodium dodecylsulfate polyacrylamide gel electrophoresis (SDS-PAGE), and agarose gel electrophoresis. The Qβ VLPs concentration was determined by measuring the total protein using a Pierce BCA assay kit (Thermo Fisher Scientific). FPLC was performed using an AKTA-FPLC 900 system fitted with Superose 6 Increase 10/300 GL columns (GE Healthcare) using PBS as the mobile phase at flow rate 0.5 mL/min. TEM images were acquired on a FEI Tecnai Spirit G2 Bio TWIN transmission electron microscope. Samples were mounted on 400-mesh hexagonal copper grids and stained with 2% (v/v) uranyl acetate. DLS was carried out on a Malvern Instruments Zetasizer Nano at 25° C. in plastic disposal cuvettes. SDS-PAGE was performed under reducing conditions on NuPAGE 12% Bis-Tris protein gels [Thermo Fisher Scientific] at 120 mV for 35 min. and stained with Coomassie Brilliant Blue. Agarose gels (0.8% (w/v) in TAE buffer) were pre-stained with Gelred™ Nucleic Acid Gel Stain [GoldBio] and samples ran under non-reducing conditions at 100 mV for 30 min. The gel images were acquired using the ProteinSimple FluorChem® imaging system.

Preparation of PLGA-loaded VLP implants by hot melt-extrusion. The Poly(lactic-co-glycolic acid) PLGA implants were prepared using our previously reported desktop melt-processing system.[27-29] PLGA [Akina Inc. 50:50 LG Ratio, MW 10-15 kDa] was ground in a mechanical blender [Magic Bullet™] for 5 minutes. The resultant powder was progressively passed through fine mesh screens (25, 35, 45, 60, 80, 120, 170, 230 porosity (standard mesh)) [Sigma-Aldrich] and large clumps re-blended. Powder which passed through a −45 mesh was used for implant formulation. The VLP vaccines were individually lyophilized. To obtain the trivalent vaccines the QβApoB, QβCETP, and QβPCSK9 VLPs were mixed at equal ratio before hot melt-extrusion. Implant formulation was as follows: 80% PLGA, 10% VLPs and 10% PEG8000 (by weight). The components were mixed in a 2-mL centrifuge tube by vortexing for 20 min, then loaded into the hot melt-processing system (maximum loading capacity=60 mg) and heated to 70° C. for 90 s. The air pressure was gradually increased up to 10 psi to extrude the implant bars cylinders, which were dried for 1 h and kept in vacuumed bags with desiccants until use.

Animal immunization. All animal experiments were approved by IACUC of UC San Diego (the Assurance number is D16-00020 and the protocol number is S18021). Six-to-eight weeks old female C57BL/6J mice were purchased from Jackson Laboratory and kept under controlled conditions with chow and water ad libitum. Five animals were assigned per group. For s.c. injection with liquid formulations, the concentration of each VLP vaccines, QβApoB, QβCETP, and QβPCSK9, was adjusted to 1 mg/ml in PBS and 100 μl were injected s.c. (100 μg/dose) every two weeks for a total of three times (prime+2 boosts). For the mixture of QβApoB, QβCETP, and QβPCSK9 (3 antigens or 3Ag s.c. group) each VLP vaccines was concentrated at 3 mg/ml in PBS, mixed 1:1:1 just before injecting 100 μl s.c. (100 μg/dose/Qβ VLP vaccine) using the same prime-boost administration schedule. For the PLGA:VLP implant (3Ag implant group), the implants were cut into 0.3-0.5 cm lengths according to their weight to yield 300 μg of each VLP vaccines (to match the 300 μg total dose per three injections with liquid format). Implants were administered using an 18G needle [BD Co.] s.c. behind the neck. As control groups, 10 μg/dose of the free peptides [synthesized by GenScript Biotech Co.] were used for prime-boost administration; further a 300 μg/implant of unmodified Qβ VLP served as a control.

Blood collection was done just before injections or implant administration (week 0), and then at weeks 2, 4, 8, and 12. Mice fasted for 4 h prior to retroorbital blood draw and blood was collected in lithium-heparin treated tubes [Thomas Scientific]. Plasma was collected by centrifugation at 2,000×g for 10 min at room temperature (RT); plasma was kept at −80° C. until use.

ELISA against peptides. Endpoint total IgG titers were determined by enzyme-linked immunosorbent assay (ELISA) against the corresponding peptide displayed in the VLP vaccines (QβApoB, QβCETP, and QβPCSK9). 96-well, maleimide-activated plates [Thermo Fisher Scientific] were prepared following manufacturer directions. In brief, the plates were coated with 100 μl/well of each peptide (containing an N-terminal cysteine) at 25 μg/ml in coating buffer (0.1 M sodium phosphate, 0.15 M sodium chloride, 10 mM EDTA, pH 7.2) overnight at 4° C. Three washes with 200 μl/well of PBST (PBS+0.5% (v/v) Tween-20 [Thermo Scientific]) were done between every step. Then, plates were blocked for 1 h at RT using 200 μl/well of 10 μg/ml L-cysteine [Sigma-Aldrich]. After washing, 2-fold serial dilutions of plasma samples from immunized animals in coating buffer were added and incubated for 1 h at RT. After washing, an HRP-labeled goat anti-mouse IgG secondary antibody [Thermo Fisher Scientific] diluted 1:5,000 in PBST was added (100 μl/well) and incubated for 1 h at RT. After a final washing step, 1-Step Ultra TMB substrate [Thermo Fisher Scientific] was added (100 μl/well) and developed for 10 min; the reaction was then stopped using 2N H₂SO₄ (100 μl/well) [Thermo Scientific]. The absorbance was read at 450 nm on a Tecan microplate reader. The endpoint antibody titers were defined as the reciprocal serum dilution at which the absorbance exceeded two times the background value (blank wells without plasma sample).

Immunoglobulin (Ig) isotyping. The ELISA was adapted from the protocol described above; instead of serial plasma dilutions, here samples a 1:1,000 dilutions were tested using sera collected 8 post-immunization. The secondary antibodies used were an HRP-labeled goat anti-mouse IgG1 (1:5,000) or IgG2b (1:5000) secondary antibody [Abcam]. The IgG2b/IgG1 ratio was reported for each group and a ratio lower than 1 was considered as Th2 response.

ELISA against target protein. To determine whether the antibodies raised were indeed specific against the target protein, ELISA against intact, full-length target proteins (PCKS9 or LDL) was performed. First, 96-well PolySorp plates [Thermo Fisher Scientific] were coated using 100 μl of recombinant mouse PCKS9 [Abcam] (10 μg/ml in PBS) or 100 μl of human LDL [Thermo Fisher Scientific] (10 μg/ml in PBS) or 100 μl human oxidized LDL [Thermo Fisher Scientific] (10 μg/ml in PBS) and incubated at 4° C. overnight. After washing with PBST, the plates were blocked with 200 μL PBST+2% (w/v) BSA for 1 h at RT. The ELISA protocols described above were used for detection and quantification of PCKS9 or (oxidized) LDL-specific antibody titers. Plasma samples collected 8 weeks post-immunization were analyzed. The plasma samples were evaluated per animal or pooled by group and tested in triplicate.

ApoB and PCSK9 plasma levels. The ApoB and PCSK9 plasma levels were determined using blood draws from week 0 and week 8 post first immunization. The ApoB and PCSK9 protein plasma concentration was determined using commercial ELISA kits [Abcam] following the manufacturer's protocols. The plasma samples were evaluated per animal or pooled by group and tested in triplicate.

CETP inhibition assay in vitro. To determine whether antibodies produced would bind and block the activity of CETP, a commercial CETP Activity Assay Kit II [Abcam] was utilized. Plasma samples collected 4 weeks post-immunization were tested following the manufacturer's protocols. Results were reported as a kinetic curve of relative fluorescent units (Ex480/Em511) over the 90 min time course and as CETP percentage activity at 90 min. The plasma samples were evaluated per animal or pooled by group and tested in triplicate.

Measurement of total cholesterol levels. Total cholesterol from plasma samples was determined using a commercial Amplex™ Red Cholesterol Assay [Thermo Fisher Scientific] following the manufacturer's protocols. In brief, plasma samples collected at week 0, 8 and 12 post t immunization were diluted 1:200 and analyzed. The plasma samples were evaluated per animal or pooled by group and tested in triplicate.

Measurement of liver and kidney biomarkers. To determine the safety of the VLP vaccines and their implant formulations, especially focusing on the trivalent formulation, toxicity plasma biomarkers were assessed. For liver damage the concentrations of the enzymes aspartate transaminase (AST) and alanine transaminase (ALT) were established by Aspartate Aminotransferase Activity kit [Abcam] and Alanine Transaminase Activity Assay Kit [Abcam], respectively. For kidney damage the concentration of kidney injury molecule-1 (KIM-1) by Mouse KIM-1 ELISA Kit [Abcam] was determined. Here, plasma samples collected at week 0 and 12 weeks post-immunization were tested. The plasma samples were evaluated per animal or pooled by group and tested in triplicate.

ELISPOT assay. Briefly, 96-well ELISPOT plates [Cellular Technology Ltd] were coated with a 1166 anti-mouse interferon-gamma (IFN-γ) capture antibody and anti-mouse interleukin-4 (IL-4) capture antibody overnight at 4° C. Splenocyte suspensions collected 2 weeks post immunization using the VLP 3Ag s.c. group were analyzed and added to the plates (5×10⁵ cells/well) following stimulation with medium alone (negative control), a mixture of free-peptides (20 g/ml) of each epitope, a mixture of the target proteins (5 g/ml each; recombinant mouse PCSK9 protein [Abcam], human LDL [Thermo Fisher Scientific], and recombinant human CETP [MyBioSource]), unmodified Qβ VLP (10 g/ml), or 50 ng/ml phorbol 12-myristate 13-acetate (PMA)/1 μg/ml Ionomycin [Sigma-Aldrich] (positive control) at 37° C. and 5% CO₂ for 24 h. The plates were washed with PBST and the incubated with 1:1000 anti-murine IFN-γ (FITC-labeled) and 1:666 anti-murine IL-4 (Biotin-labeled) antibodies for 2 h at RT. Plates were washed, and 1:1000 streptavidin-alkaline phosphatase (AP) and anti-FITC-HRP secondary antibodies were added to each well and incubated for 1 h at RT. Plates were washed with PBST and distilled water, then incubated with AP substrate for 15 min at RT, washed with distilled water and incubated with HRP substrate for 10 min at RT. Plates were rinsed with water and air-dried at RT overnight. Colored spots were quantified using an Immunospot S6 ENTRY Analyzer. The splenocytes were evaluated per animal and tested in triplicate for each stimulant.

Results and Discussions

Characterization of VLP vaccines. Applicant selected the following peptide antigens; these antigens have been validated as B cell epitopes: ApoB=KTTKQSFDLSVKAQYKKNKH,[12]; CETP=FGFPEHLLVDFLQSLS,[16] and PCSK9=NVPEEDGTRFHRQASKC;[20] (Table 1).

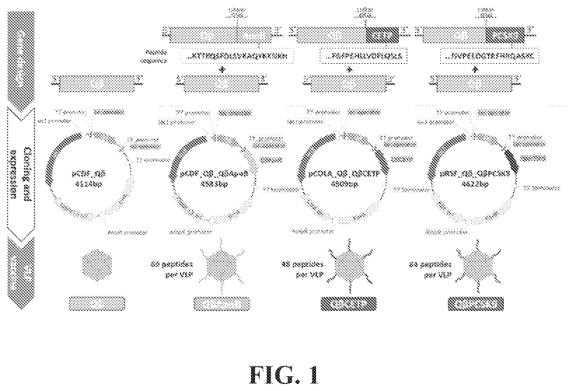

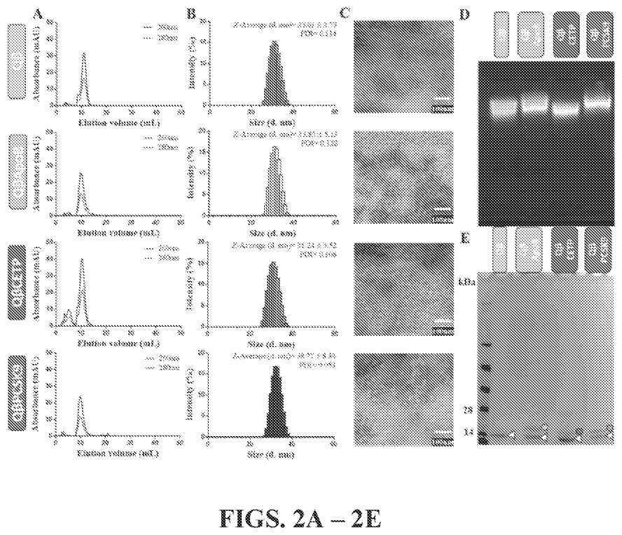

The peptide epitopes were cloned into VLP expression vectors for display at the C-terminus of the Qβ coat protein (CP) via a GSG linker. Unmodified Qβ CP and the epitope-displaying Qβ CPs (QβApoB, QβCETP, and QβPCSK9, respectively) were cloned in the same vector to produce hybrid Qβ VLP containing both, unmodified and modified Qβ CP (FIG. 1). Applicant chose the hybrid assembly by expressing free and modified CP, because it has been previously established that hybrid assembly is required for the genetic display strategy; free coat protein is required for assembly yielding intact VLPs.[30] The VLP vaccines were expressed in E. coli and purified over sucrose gradients via ultracentrifugation. Purified VLPs were characterized using a combination of size exclusion chromatography (fast protein liquid chromatography, FPLC), dynamic light scattering (DLS), transmission electron microscopy (TEM), and gel electrophoresis (native agarose gels and denaturing SDS-PAGE) to confirm their structural integrity and degree of antigen incorporation (FIG. 2).

FPLC analysis revealed a single peak for each of the Qβ VLP vaccines, indicating that the VLPs were intact (no free CPs were detected) and free of aggregation (FIG. 2A). DLS measurement revealed the hydrodynamic diameter of the VLPs being close to 30 nm for QβApoB and QβCETP VLPs; QβPCSK9 VLPs showed in increase in size with a diameter of 38 nm (FIG. 2B); this increase in size may be attributed to interparticle interactions promoted by formation of disulfide bonds between the C-terminal Cys side chain on the PCSK9 antigen (Table 1, FIG. 1). Extensive particle aggregation, however, was not apparent by any method (FPLC, DLS, or TEM). TEM imaging of negatively-stained VLPs further indicated that particle preparation yielded monodisperse and intact VLPs (FIG. 2C).

Agarose gels further confirmed the presence of intact VLPs (FIG. 2D); the electrophoretic mobility of ApoB- and PCSK9-epitope displaying VLPs was slightly reduced compared to native Qβ or QβCETP VLPs, which can be explained by the higher IP of these antigens (Table 1). Lastly, SDS-PAGE analysis confirmed the presence of unmodified Qβ CP (˜14 kDa) and modified Qβ CPs (QβApoB=˜16.4 k Da, QβCETP=˜15.9 kDa, and QβPCSK9=˜16.0 kDa) for each of the preparation (FIG. 2E), and according to the relative intensity of the bands the percentage of modified Qβ CPs (QβApoB=˜38%, QβCETP=26%, and QβPCSK9=35%). Therefore, the number of peptide epitopes per VLP lies between 50-70 peptides per VLP (each VLP is comprised of 180 copies of the CPs). Overall, these results indicate that intact Qβ VLPs displaying the antigens ApoB, CETP, and PCSK9 were successfully produced.

TABLE 1
Peptide antigen properties.

| | | | Identity | |
|---|---|---|---|---|
| | Sequence | | to | Source |
| Antigen | (length) | IP | mouse | (reference) |
| ApoB | KTTKQ SFDLS | 10.6 | 91% | Human[12] |
| | VKAQY KKNKH | | | |
| | (20 aa) | | | |
| CETP | FGFPE HLLVD | 4.1 | N/A | Human[16] |
| | FLQSL S | | | |
| | (16 aa) | | | |
| PCSK9 | NVPEE DGTRF | 7.1 | 94% | Human[20] |
| | HRQAS KC | | | |
| | (17 aa) | | | |

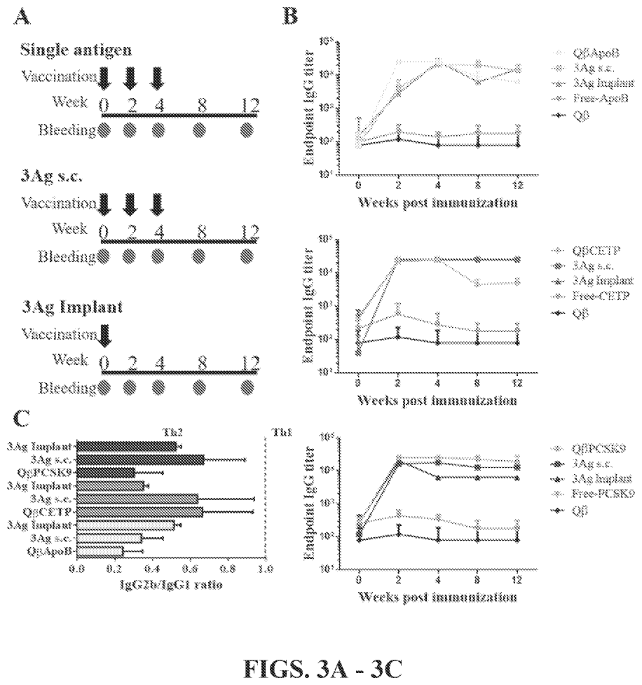

Immunogenicity of Qβ VLP vaccines. After characterization of the Qβ VLP vaccines, Applicant determined whether s.c. immunization using single antigen VLPs (QβApoB, QβCETP, or QβPCSK9) versus a mixture of the three Qβ VLP vaccine candidates (the trivalent vaccine candidate), either injected or delivered via a slow-release implant, could produce antibody titers against the target proteins (FIG. 3A). The implants of the following composition 80% PLGA, 10% VLP, and 10% PEG8000, were produced as previously reported; the VLPs are released from the implants over a time course over 30 days.[27-29] Applicant found the monovalent vs. trivalent vaccinations resulted in comparable antibody endpoint titers; VLP display of the peptide antigens was effective to yield high antibody titers without the need of additional adjuvants. Furthermore, immunization using the slow-release implants versus prime-boost administration schedules using soluble VLPs was equally effective. Antibody titers were detectable as early as 2-weeks post immunization with maximum titers achieved at week 4 post immunization. The soluble peptides were, as anticipated, not effective to raise target-specific antibodies (FIG. 3B).

For the ApoB peptide groups, the titers where slightly higher at the week two comparing the monovalent versus trivalent vaccines; however, at later time points the 3Ag groups resulted in slightly higher titers (FIG. 3B). For CETP peptide, titers remained the same between groups and slightly decreased only for the monovalent vaccine candidate during weeks 8 and 12 (FIG. 3B). For PCSK9 peptide, antibody titers overall were consistent between groups; with the 3Ag implant resulting in slightly reduced antibody titers over longer time periods (FIG. 3B). Overall though, the various vaccination groups, monovalent versus trivalent administered as soluble vaccine versus slow-release implant, were consistent. The subtle variations may be explained by differences and bias of antigen processing by antigen processing cells (APC).

The next goal was to establish what IgG isotypes (IgG1 and IgG2b) were elicited and whether the ratio would be altered when Qβ VLPs was injected as a single antigen or in a mixture (3Ag) either s.c. (prime-boost) or implant (slow-release). Data indicate that IgG2b/IgG1 ratios remained essentially the same between groups; and IgG2b/IgG1 ratios were lower than 1. IgG1 antibodies are primarily induced via Th2-type cytokines (e.g. IL-4), and the production of IgG2b antibodies reflects the involvement of Th1-type cytokines (e.g. IFN-γ);[31] therefore, the IgG2b/IgG1 ratio of <1 can be interpreted as a T helper cells 2 (Th2) biased response (FIG. 3C). This is consistent with previous reports on cardiovascular vaccine formulations incorporating these peptide epitopes.[12],[16],[20]

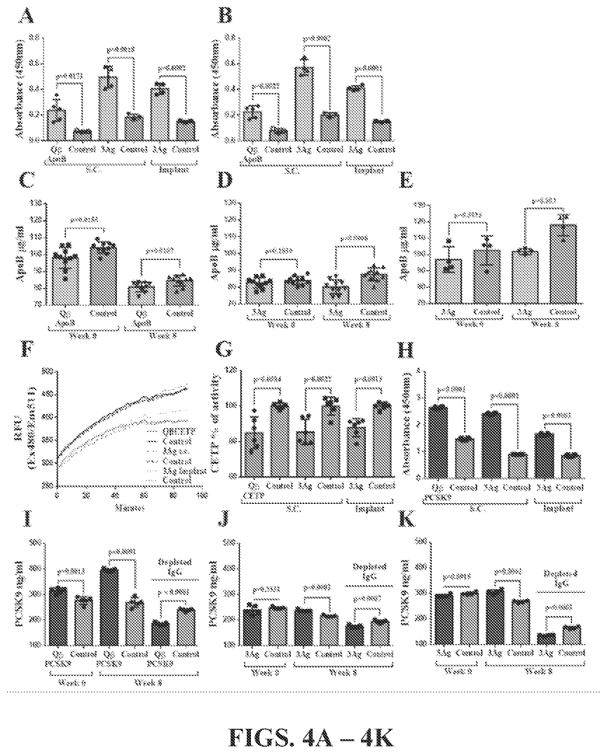

Interaction and specificity of antibodies raised from the VLP vaccinated mice against the target proteins, ApoEB, CETP, and PCSK9. Once Applicant confirmed that antibodies against the peptide epitopes were raised, Applicant also confirmed that these antibodies indeed recognize the naïve target protein. First, plasma was isolated from animals receiving the monovalent QβApoB vaccines versus the trivalent 3Ag vaccines and sera were tested against human LDL and oxidized LDL (oxLDL). ELISA confirmed recognition of LDL and oxLDL (FIG. 4A and FIG. 4B). ApoB is the primary apolipoprotein of cholesterol and the anti-atherosclerotic effect of anti-ApoB antibodies is related to preferential inhibition of inflammation and reversion of cholesterol transportation by altering the pathway by which macrophages phagocytize ox-LDL.[32] Applicant also confirmed that vaccination using the monovalent QβApoB or trivalent 3Ag vaccines lowered plasma levels of ApoB protein (FIGS. 4C-4E). It is important to note that the difference was more profound when analyzing sera obtained from the trivalent vaccine groups (3Ag s.c. and 3Ag implant) versus sera obtained from mice immunized with the monovalent ApoB vaccine candidate. This effect could be attributed to the synergy of simultaneous PCSK9 neutralization in the trivalent vaccine candidate group (FIG. 4J and FIG. 4K). Binding of the sera to PCSK9 protein was validated by direct ELISA; sera from all vaccinated groups but not control groups were found to recognize PCSK9 (FIG. 411). Then, the PCSK9 plasma concentration was examined at week 8 post first vaccination; data indicate increased levels of PCSK9 and this phenomenon was observed across all vaccine groups; others have made similar observations and the apparent increase can be explained by detection of IgG-PCSK9 immunocomplexes.[17-21] Therefore, total IgG was depleted prior to assessing the plasma levels of PCSK9 again. Now, vaccinated groups (using any formulation or administration schedule) showed a significant reduction in PCSK9 levels, indicting effectiveness of the vaccination approach (FIGS. 41-4K). Finally, Applicant evaluated the neutralizing effect of the various vaccines on CETP activity; because mice do not express CETP, an in vitro was established. Results showed that plasma from the three groups were effective to inhibit the CETP activity by up to 15% (FIG. 4F and FIG. 4G). Together, these findings demonstrate that antibodies are generated from vaccination using the monovalent or trivalent vaccines administered as prime-boost or slow-release implant are effective in that they recognize and neutralize their target proteins and reduce target protein concentration in plasma.

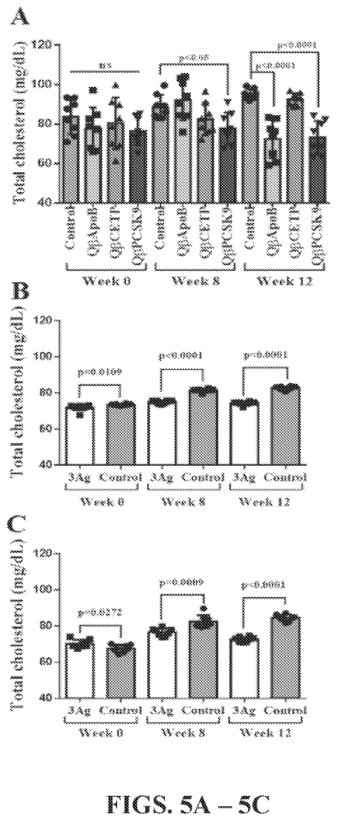

Total cholesterol plasma levels in naïve versus vaccinated mice. After corroborated that antibodies generated from the VLP vaccines bind, neutralize, and lower their target proteins (FIG. 4), Applicant set out to determine plasma cholesterol levels comparing the various vaccines. Applicant first measured the effect of monovalent vaccines and determined that immunization with QβApoB and QβPCSK9 led to a significant reduction on total cholesterol at week 12 post first vaccination, and only QβPCSK9 group showed a lower concentration at week 8 as well (FIG. 5A). QβCETP did not indicate a significant difference compared to control (FIG. 5A), although plasma samples from this group effectively neutralized recombinant CETP in vitro (FIG. 4F and FIG. 4G). This data is as expected, because mice lack CETP,[33] therefore further experiments with transgenic mice expressing CETP[19] or other animal model such as rabbit[16] could help to elucidate how important could be the effect on CETP inhibition using our QβCETP or trivalent vaccine candidate. Data also indicate that the trivalent vaccines, whether administered as prime-boost or slow-release implant, were effective in decreasing cholesterol levels in mice (FIG. 5B and FIG. 5C). These results correlate with antibody titers (FIG. 3) and reduction of PCSK9 and ApoB proteins levels in plasma (FIG. 4). Data are also consistent with previous reports indicating a key role of these proteins on regulation of plasma cholesterol levels.[12-14,17-21]

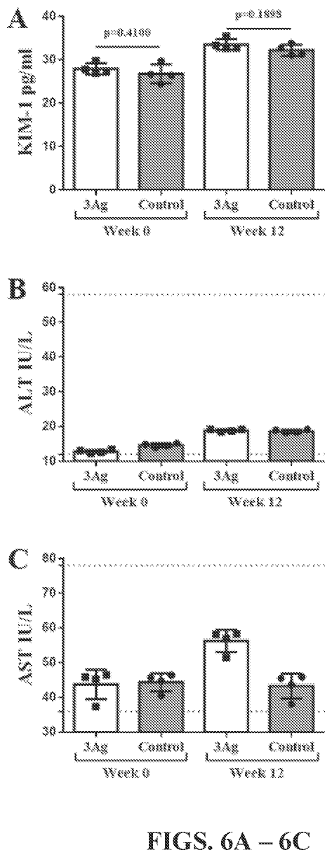

Qβ VLP vaccine candidate safety in healthy mice. Evaluation of safety is critical, because self-antigens are the therapeutic target. Safety parameters such as the absence of a target-specific T-cell response (Th and cytotoxic T-cells) and blood biomarkers were measured. While safety of the monovalent vaccines has been demonstrated,[12-21] a trivalent vaccination strategy has not yet been reported. First, Applicant examined the concentration of Kidney Injury Molecule-1 (KIM-1), a transmembrane glycoprotein and blood biomarker of kidney injury,[34] pre- and post-immunization (week 0 and 12). There was not difference in KIM-1 levels comparing mice receiving the trivalent vaccine candidate versus the control group, i.e. age-matched mice (FIG. 6A), demonstrating that blocking multiple self-proteins (i.e. PCSK9 and ApoB; CETP is not expressed in mice) did not alter kidney physiology in healthy animals. Second, Applicant measured the activity of the enzymes aspartate transaminase (AST) and alanine transaminase (ALT) in plasma as a biomarker for liver injury.[35] ALT levels were comparable in the vaccinated versus the age-matched mice control group; some elevation of the AST levels were observed in mice receiving the trivalent vaccine candidate (FIG. 6B and FIG. 6C)—however, levels were in the normal range.[36] The data indicate safety of the trivalent vaccine candidate with no apparent kidney or liver damage.

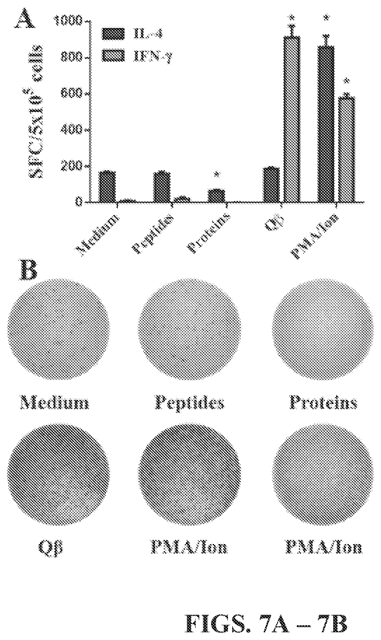

Finally, Applicant assessed whether T cell activation was primed upon vaccination using the trivalent vaccine candidate; this was assayed using splenocytes and ELISpot assay. Applicant monitored the production of IFN-γ and IL-4 cytokines, which are linked to a Th1 versus Th2 profile, respectively, and compared splenocytes isolated from mice vaccinated using the mixture of QβApoB, QβCETP, and QβPCSK9 (3Ag s.c. group). Stimulation of splenocytes with Qβ VLPs yielded comparable levels of IFN-γ and IL-4, respectively, when comparing the Qβ versus trivalent vaccine candidate groups; PMA/Ionomycin served as a positive control (FIG. 7A). T cell responses against the vaccination platform, the Qβ VLPs, are apparent and expected, because the VLPs contain a mixture of epitopes. Importantly, stimulation with free-peptides or a mixture of the target proteins, ApoB, CETP, and PCSK9, did not elicit a significant IFN-γ production compared to the medium only-control group (FIG. 7A). Splenocytes stimulated with a mixture of whole targeted proteins resulted in a reduction of IL-4 levels (FIG. 7A). These data indicate that T cell activation against the target proteins ApoB, CETP, and PCSK9 is absent, thus laying a foundation that the trivalent vaccine candidate is safe and does not elicit T cell mediated immunotoxicity directed against the self-proteins; this is consistent with the safety parameters established with others works on self-antigen vaccines.[17,18,21] Data are also in agreement with the IgG isotype, which Applicant also found to have a Th2 bias (see FIG. 3B).

Experiment No. 2—HPV Vaccines

Materials and Methods

Expression of Qβ. Bacteriophage Qβ VLPs were expressed and purified, as previously described [28]. Chemically competent BL21(DE3) Escherichia coli cells (New England Biolabs) transformed with pET28CP containing the Qβ coat protein sequence were plated onto lysogeny broth (LB) agar (Thermo Fisher Scientific) containing 50 μg/mL kanamycin (Gold Biotechnology). The isolated colonies were picked into 100 mL of autoclaved LB medium plus kanamycin and cultured for 12 h at 37° C. to saturation. Applicant added 50 mL of the culture to 1 L MagicMedia (Thermo Fisher Scientific, Carlsbad, CA, USA) and incubated at 37° C. for another 24 h, shaking at 300 rpm. The cell pellets were collected by centrifugation at 1500×g and frozen at −80° C. overnight. The pelleted cells were resuspended in 100 mL phosphate-buffered saline (PBS) on ice and lysed using a probe sonicator for 10 min. The lysate was centrifuged at 10,080×g, the supernatant was collected, and the Qβ particles were precipitated by adding 10% w/v PEG8000 (Thermo Fisher Scientific) at 4° C. for 12 h on a rotisserie. The precipitated fraction was pelleted by centrifugation at 10,080×g and dissolved in 40 mL PBS before extraction with a 1:1 v/v butanol/chloroform. The aqueous fraction was collected by centrifugation as above (at 10,080×g) and Qβ particles were purified on a 10-40% sucrose velocity gradient by ultracentrifugation at 96,281×g for 4.5 h. The light-scattering Qβ layer was collected and pelleted by ultracentrifugation at 160,326×g for 3 h. The purified Qβ particles were dissolved in PBS as a stock solution for later experiments.

Synthesis of HPV-Qβ. HPV peptides were conjugated to Qβ via an SM(PEG)₈ bifunctional linker containing an NHS group and a maleimide group (Thermo Fisher Scientific). In brief, 20 mg of Qβ was mixed with a 500-fold molar excess of SM(PEG)₈ to Qβ in 0.5 mL PBS (pH 7.4), at room temperature for 1 h. The unreacted SM(PEG)₈ was removed using a 100 kDa cut-off centrifuge filter at 3000×g for 15 min, and the recovered VLPs were washed with 0.5 mL PBS. The washed VLPs were resuspended in 0.5 mL PBS and reacted with a 500-fold molar excess of HPV L2 peptides at room temperature for 2 h. The unreacted peptides were removed by centrifugal filtration as above; the recovered HPV-Qβ vaccines were washed twice in 0.5 mL deionized water, and then dialyzed against deionized water for 24 h.

Synthesis of Qβ-Cy5. Bacteriophage Qβ (20 mg) was labeled with Cy5 through conjugation via a 500-fold molar excess of sulfo-Cy5 NHS ester (Lumiprobe) in 0.5 mL 0.1 M potassium phosphate buffer (referred to as KP buffer; K₂HPO₄ and KH₂PO₄, pH 8.3), for 4 h at room temperature. The unreacted components were removed by centrifugal filtration as above, and the Cy5-Qβ particles were dialyzed against and then stored in deionized water.

Preparation of Qβ-Loaded PLGA Implants through Melt-Extrusion. The loaded PLGA implants were prepared using a new desktop melt-processing system[29]. EXPANSORB PLGA (PCAS, 50:50, MW˜20,000 Da) and FITC-PLGA (Akina) were ground to a fine powder. The Qβ, HPV-Qβ, or Cy5-Qβ particles were lyophilized before melt extrusion. The formulation of all implants was 80% PLGA, 10% VLP, and 10% PEG8000. The powdered components were mixed in a 2-mL centrifuge tube by vortexing for 20 min, then loaded into the melt-processing system (maximum load=60 mg) and heated to 70° C. for 90 s. The air pressure was gradually increased up to 10 psi to extrude the implant cylinders, which were dried for 1 h and cut into 0.3-0.5 cm lengths, according to the weight.

Implant Analysis by SEM-EDX. Cross-sections of the implants were coated with carbon and observed by scanning electron microscopy with energy dispersive X-ray spectroscopy (SEM-EDX), using an FEI Quanta 600 scanning electron microscope coupled to a Bruker XFlash 6/60 EDX spectroscope. The EDX maps were prepared using the FEI AZtec software.

Characterization of Particles. The VLPs were characterized by fast protein liquid chromatography (FPLC), transmission electron microscopy (TEM), dynamic light scattering (DLS), and sodium dodecylsulfate polyacrylamide gel electrophoresis (SDS-PAGE). FPLC was performed using an AKTA-FPLC 900 system fitted with Superose 6 Increase 10/300 GL columns (GE Healthcare), using PBS (pH 7.4) as the mobile phase at a flow rate of 0.5 mL/min. TEM images were acquired on an FEI Tecnai Spirit G2 Bio TWIN transmission electron microscope. Samples were mounted on 400-mesh hexagonal copper grids and stained with 2% uranyl acetate. DLS was carried out on a Malvern Instruments Zetasizer Nano at 25° C., in plastic disposal cuvettes. SDS-PAGE was performed on NuPAGE 12% Bis-Tris protein gels (Thermo Fisher Scientific) at 150 mV for 75 min. The gels were stained with Coomassie Brilliant Blue and images were acquired using the ProteinSimple FluorChem R imaging system.

Analysis of the Loaded PLGA Implants In Vitro. Qβ-loaded PLGA implants (˜3 mg implant containing 300 μg VLPs) were placed in a 1.5-mL centrifuge tube and immersed in 500 μL PBS at 37° C. At each time-point, 200-μL samples were removed and replaced with 200 μL fresh PBS. The quantity of released VLPs was determined by measuring the total protein concentration using a Pierce BCA assay kit (Thermo Fisher Scientific).

Analysis of Cy5-Qβ Loaded FITC-PLGA Implants In Vivo. All animal experiments were carried out in accordance with recommendations from the UCSD's Institutional Animal Care and Use Committee (ethical approval: UCSD IACUC protocol S18021). Female BALB/c mice aged 8 weeks (Jackson Laboratory, Bar Harbor, ME, USA) were anesthetized for the implantation process before shaving hair from the back and wiping the skin with Alcohol Prep pads. The FITC-PLGA implants loaded with Cy5-Qβ VLPs (˜1 mg implant containing 100 μg VLPs) were introduced subcutaneously via an 18-gauge needle by pushing the implant out of the needle with a sterilized stainless-steel wire (0.51 mm diameter). The needle was withdrawn and the skin wiped with Alcohol Prep pads. Fluorescence images were acquired on an IVIS 200 imaging system at different time-points and were analyzed using Living Image v3.0.

Immunization. For vaccines in solution, female BALB/c mice aged 8 weeks were vaccinated subcutaneously by injection on days 0, 14, and 28, with blood collected 7 days after each vaccination. For the PLGA implants, mice were vaccinated by subcutaneous implantation, as described above, and blood was collected on days 7, 14, 21, 35, and 49. To compare the different HPV-Qβ conjugates, n=5 mice were immunized three times with 30 μg of the specific HPV-Qβ conjugate (or the Qβ as a control). For the conjugates vs. mixture vs. peptide experiment, n=6 mice were vaccinated with 30 μg of the specific HPV-Qβ conjugate, a mixture of 30 μg Qβ and 5 μg of the HPV peptide, or 5 μg of the HPV peptide alone. For the evaluation of single-dose vaccination using the loaded PLGA implants, the PLGA implants were subcutaneously introduced into n=6 mice. The implants contained 100 μg HPV-Qβ, 500 μg HPV-Qβ, a mixture of 100 μg Qβ, and 20 μg HPV peptide, or 20 μg HPV peptide alone. To make a direct comparison among the three different vaccination techniques, three different groups of mice each comprising n=6 mice were vaccinated subcutaneously with a single dose of 100 μg of the HPV-Qβ conjugate, three doses of 30 μg of the HPV-Qβ conjugate, and a single dose HPV-Qβ-loaded PLGA implants, loaded at 100 μg. Sera were separated from the collected blood through centrifugation at 2000×g for 20 min and were stored at 4° C. (short term) or −80° C. (long term).

Serum Analysis and Determination of Antibody Titers. The abundance of antibodies against HPV peptides and Qβ particles in serum samples was determined by the enzyme-linked immunosorbent assay (ELISA). For the detection of α-HPV antibodies, 96-well Pierce maleimide-activated plates (Thermo Fisher Scientific) were coated with 100 μL HPV peptides (10 μg/mL) per well in coating buffer (0.1 M sodium phosphate, 0.15 M sodium chloride, 10 mM EDTA, pH 7.2). After overnight incubation at 4° C., the plates were washed three times with PBS containing 0.1% Tween-20 (PBST) and then blocked with 150 μL cysteine (10 μg/mL) per well for 1 h, at room temperature. After three washes in PBST, serial dilutions of serum in PBST containing 1% bovine serum albumin (BSA; Roche Diagnostics, Mannheim, Germany) were added and incubated at 37° C. for 1 h. After three washes in PBST, Applicant added 100 μL of an alkaline phosphatase-labeled goat anti-mouse IgG secondary antibody (Thermo Fisher Scientific) diluted 1:2000 in PBST+1% BSA for 30 min, at 37° C. Finally, the plates were washed five times with PBST and developed with 100 μL of the 1-step PNP substrate (Thermo Fisher Scientific) for 30 min at 37° C. The reaction was stopped by adding 100 μL of 1 M NaOH and the absorbance was read at 405 nm on a Tecan microplate reader. For the detection of α-Qβ antibodies, 96-well PolySorp plates (Thermo Fisher Scientific) were coated with 100 μL Qβ (10 μg/mL in PBS) and incubated at 4° C. overnight. After three washes in PBST, the plates were blocked with 150 μL PBST+2% BSA at 37° C. for 1 h. The subsequent steps were as described for the detection of HPV peptides.

The IgG subtype was also determined by ELISA. The procedure was similar to that described above for Qβ and HPV peptides, but the secondary antibody was replaced with goat anti-mouse antibodies specific for IgG2a, IgG2b, and IgG1 (diluted 1:2000), as part of the IgG subtype antibody kit (Sigma-Aldrich). After three washes in PBST, Applicant added an alkaline phosphatase-labeled rabbit anti-goat antibody (Sigma-Aldrich) diluted 1:3000, then washed five times in PBST. Finally, the plates were developed by adding the 1-step PNP substrate, as described above. For all ELISAs, the antibody titers were defined as the reciprocal serum dilution at which the absorbance exceeded the background value by >0.2.

Pseudovirus Production and Purification. HPV16 pseudovirus encapsidating the reporter plasmid pfwB (Addgene #37329) encoding green fluorescent protein (GFP) was produced in 293 TT cells, as previously described [30]. In brief, 293 TT cells were transfected with a mixture of two plasmids—pfwB and p16Llw (Addgene #37320) containing the sequence of each HPV shell. After 48 h, the cells were lysed and mature pseudovirus was purified on an Optiprep gradient (27:33:39) through ultracentrifugation at 125,755×g for 6 h. Gradient fractions corresponding to the L1 band were collected and characterized by SDS-PAGE, agarose gel electrophoresis, and flow cytometry, as recommended[30]. HPV16 pseudovirus stocks were titrated in pgsa-745 cells to yield 50-60% GFP-positive cells (percentage of infectivity) for the L2-based neutralization assay.

L2-Based Neutralization Assay. MCF10A cells were cultured in 96-well plates to produce the extracellular matrix (ECM), followed by the addition of the HPV pseudovirus (PsHPV), as previously described [26]. The ECM-PsHPV were incubated overnight, then removed and replaced with two-fold serial-dilutions of serum collected from immunized mice or the growth medium, as a control. The plates were incubated for 6 h before adding pgsa-745 cells. After 48 h, HPV16 pseudovirus infectivity and neutralization were assessed by flow cytometry (Accuri) based on the expression levels of GFP in infected cells. The reciprocal of the highest serum dilution that inhibited 50% of the infection relative to the control serum was considered the neutralization titer.

Statistical Analysis. Comparisons of the differences between two different groups were performed using unpaired two-tailed student's t-test (GraphPad Prism software); *p<0.05; **p<0.01; ***p<0.005; ns, not significant p>0.05. Values were expressed as means±standard deviations. Sample sizes are stated in the figure legends.

Results and Discussion

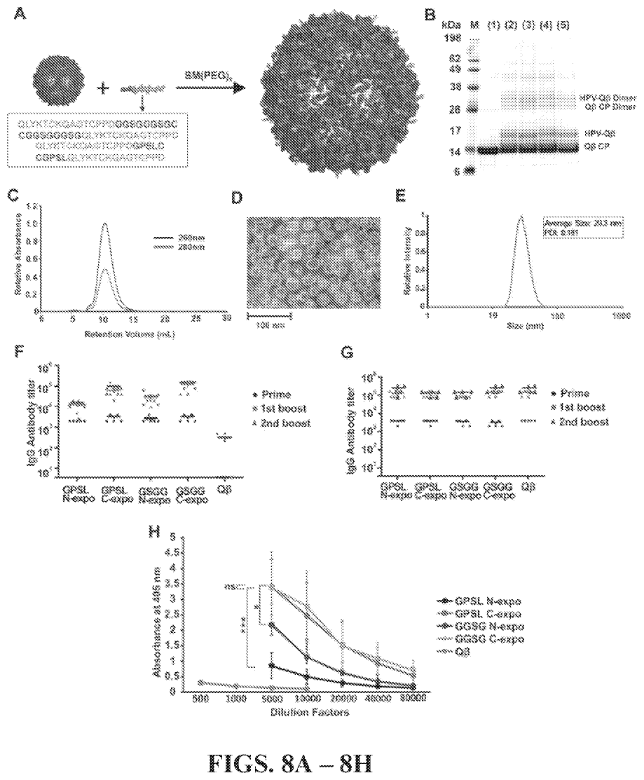

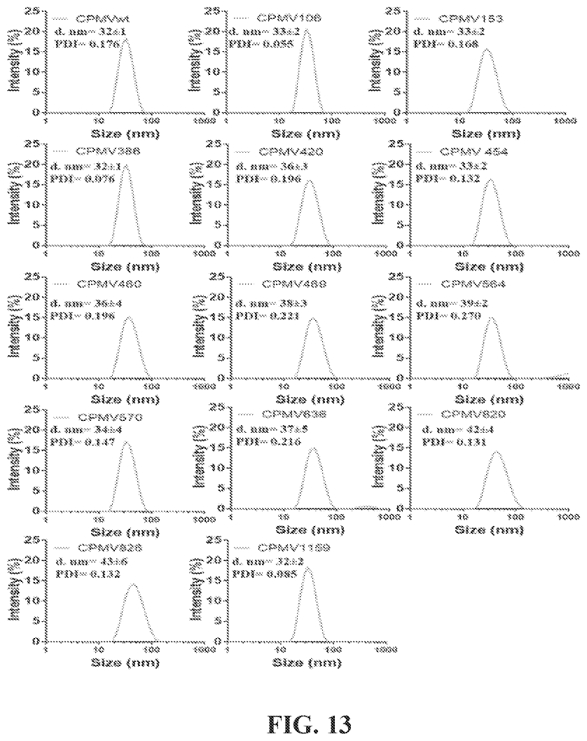

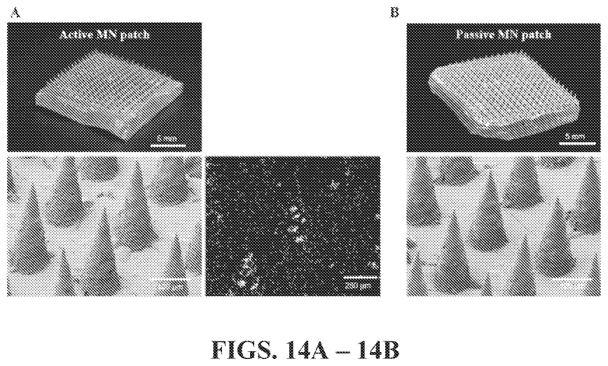

The HPV16 epitope L217-31 is ideal for the development of an effective HPV vaccine because it is highly conserved among diverse HPV isolates [15-17]. Different linkers and epitope orientations can have a significant effect on the immunogenicity of peptides [31,32], thus, Applicant designed four peptides containing the L217-31 epitope, featuring alternative linkers (GPSL or GGSGGGSG) and orientations (epitope exposed at the N-terminus or C-terminus), as shown in Table 2. The peptides were conjugated to the surface of the VLPs derived from bacteriophage Qβ in a two-step procedure, where a bifunctional PEG was first conjugated via the NHS-chemistry to surface amines of Qβ. Next, terminal cysteines from the HPV epitopes were conjugated via a maleimide linker to the PEG-Qβ conjugate (FIG. 8A). SDS-PAGE analysis of the products confirmed the presence of unmodified Qβ coat protein with a molecular weight of 14.2 kDa and an additional band at ˜17 kDa, corresponding to the coat protein conjugated to the HPV peptide (FIG. 8B). Unmodified and conjugated VLPs retained the dimeric assemblies under the electrophoresis conditions, as expected due to the relative strength of the dimeric interaction. The relative intensity of the bands indicated that ˜30% of the Qβ coat proteins were conjugated to HPV peptides (GPSL-N-expo 32.2%, GPSL-C-expo 31.1%, GGSG-N-expo 33.8%, GGSG-C-expo 31.8%). FPLC analysis revealed a single peak, indicating that the VLPs were monodisperse and did not form aggregates (FIG. 8C and FIG. 13). DLS revealed that the diameter of the VLPs was 28+3 nm, which was consistent with the TEM images (FIG. 8D, FIG. 8E and FIG. 13). These results indicated that the conjugation of HPV peptides did not alter the overall size or shape of the VLP as anticipated, given the low molecular weight of the peptides.

TABLE 2
Number of SARS-CoV-2 epitopes displayed
per CPMV or Qβ particle.

| Vaccine candidate | Peptides displayed/per particle |
|---|---|
| CPMV106 | 44 |
| CPMV153 | 28 |
| CPMV386 | 40 |
| CPMV420 | 64 |
| CPMV454 | 32 |
| CPMV460 | 63 |
| CPMV469 | 64 |
| CPMV564 | 63 |
| CPMV570 | 59 |
| CPMV636 | 60 |
| CPMV820 | 70 |
| CPMV826 | 67 |
| CPMV1154 | 58 |
| Qβ570 | 98 |
| Qβ636 | 107 |
| Qβ826 | 105 |

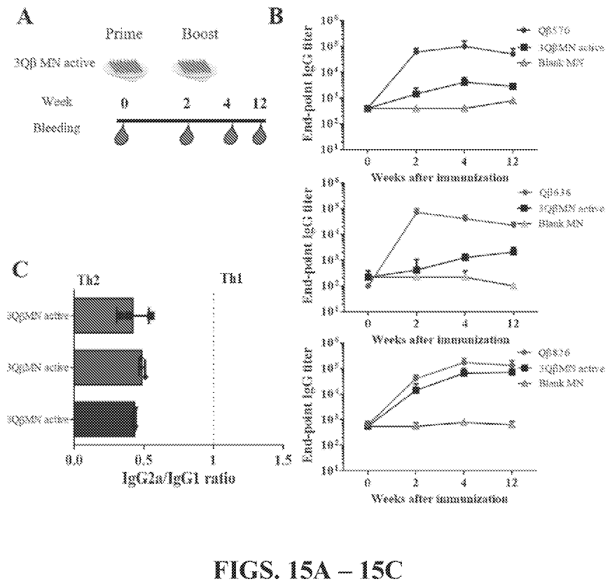

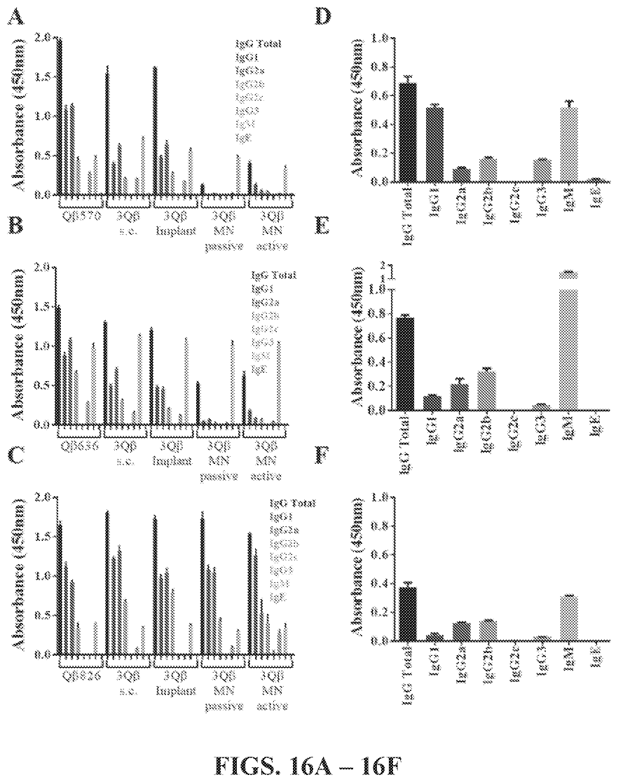

Applicant found that all four conjugated VLPs (but not the unmodified Qβ particles) elicited the production of anti-HPV IgG in mice, but that the higher titers were achieved when the epitope was exposed at the C-terminus (FIG. 8F). All four conjugated VLPs and unmodified Qβ particles generated similar IgG titers against the Qβ carrier, as would be expected for a VLP (FIG. 8G). There was no significant difference in titer when comparing the two constructs with the HPV peptide exposed at the C-terminus but at different linkers, even when using different dilution factors for the second boost (FIG. 811 and FIG. 14). Ultimately, Applicant selected the construct with the C-terminal epitope and the GGSGGGSG linker for the subsequent experiments and refers to this construct hereafter as HPV-Qβ. The conjugation of HPV16 epitope L217-31 to Qβ was necessary to enhance its immunogenicity, because the injection of a mixture of unmodified Qβ and the free HPV peptide elicited antibodies against Qβ but not against HPV, and no antibodies against HPV were generated by the injection of the free HPV peptide alone (FIG. 15).

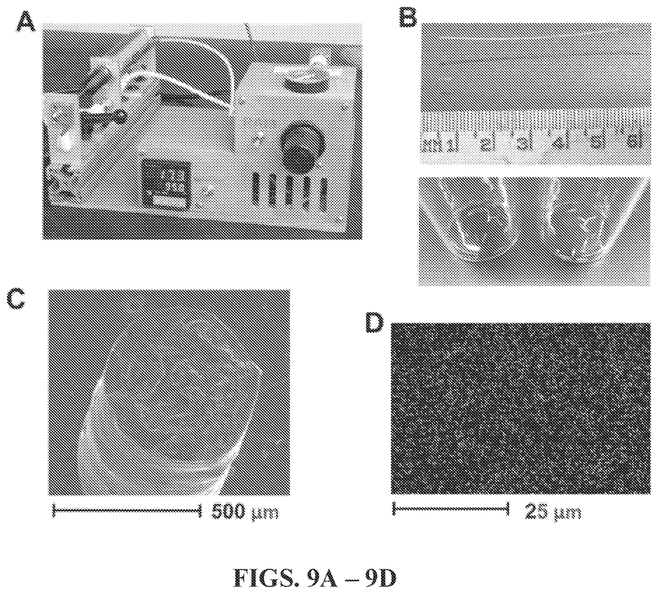

HPV-Qβ was encapsulated into the PLGA implants through melt-extrusion, as previously reported [25]. Parameters that were previously optimized included temperature, such that the temperature was well above the T_{g} of PLGA, to allow for flow at a reasonably low pressure and residence time in the extruder, to ensure complete melting of the polymer. This method can be easily adapted to other vaccines but requires optimization for each candidate to determine temperature stability, candidate release rate, etc. To optimize the control of the implant parameters, Applicant used a benchtop melt-processing system (FIG. 9A) [29]. Taking advantage of the stable extrusion pressure and adjustable nozzle size, the PLGA implants were produced with an accurate diameter of less than 0.8 mm, so that they fit inside 18-gauge needles, for convenient administration. The loaded PLGA implants were extruded as implant cylinders, 5-6 mm in length, which were cut into segments of 0.3-1 mm for implantation (FIG. 9B). SEM imaging of implant cross-sections revealed a uniform cylinder of ˜0.5 mm diameter (FIG. 9C). Homogeneous dispersion of HPV-Qβ particles was verified by the EDX analysis, specifically the sulfur K-series emission signal map (FIG. 9D). Applicant's prior experience indicates that homogeneous dispersion within the implant improves the linearity of release and negates the aggregation of VLPs from the released fraction.

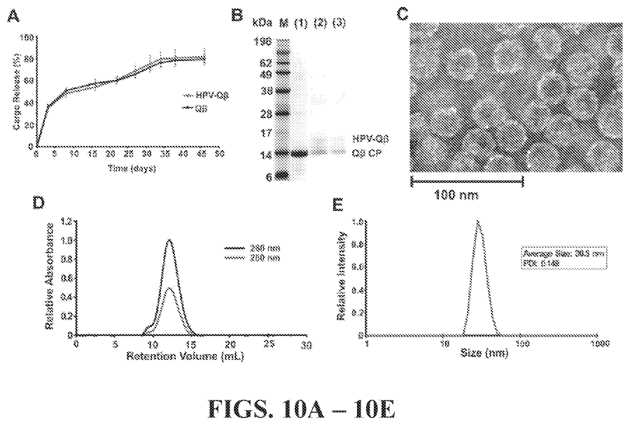

The loaded PLGA implants were shown to slowly release the VLPs into PBS, with continuous release occurring over a period of up to 35 days at 37° C. (FIG. 10A). Released fractions indicated that the HPV peptides remained conjugated following the melt-extrusion process, as confirmed by the presence of ˜17 kDa bands when the samples taken on days 15 and 30 were analyzed by SDS-PAGE (FIG. 10B). TEM and DLS confirmed the presence of intact HPV-Qβ particles with a diameter of ˜30 nm (FIG. 10C, FIG. 10D). FPLC analysis of the released HPV-Qβ particles showed that the melt-extrusion process did not promote aggregation either during encapsulation or upon release (FIG. 10E).

The in vivo subcutaneous environment is more complex than PBS and might influence the release of the cargo from the PLGA implants. Applicant sought to evaluate the release kinetics of the implanted Qβ using fluorescence molecular tomography in vivo. Unmodified Qβ was used for this set of studies, rather than HPV-Qβ due to the competing chemical reactions for surface conjugation of fluorophores (i.e., HPV-Qβ exhausted the most reactive amines for conjugation). To track the release of the particles in vivo, Qβ was labeled with Cy5 and the incorporation of the fluorophore was confirmed by FPLC and SDS-PAGE (FIG. 16). FPLC analysis revealed a peak in the 647 nm channel (Cy5 absorbance), at a retention volume of ˜11 mL, corresponding to the Qβ particles (FIG. 16A). SDS-PAGE analysis showed a band corresponding to the anticipated molecular weight of Cy5-Qβ, with overlapping signals in the blue fluorescence and Coomassie Brilliant Blue channels (FIG. 16B).

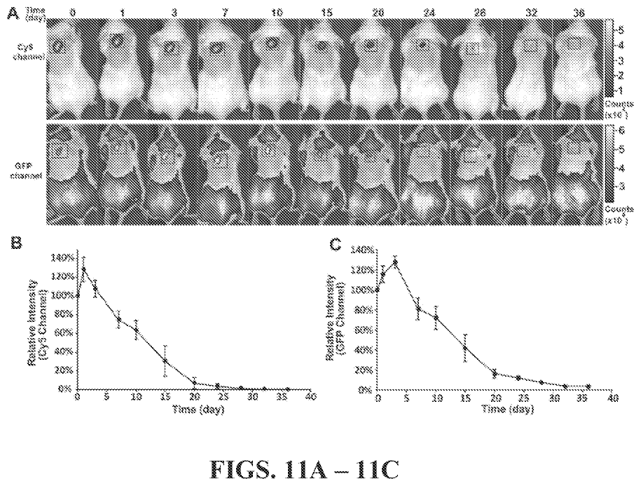

To track the in vivo fate of the encapsulated VLPs and the polymeric implant, an FITC-PLGA matrix loaded with Cy5-Qβ particles was implanted subcutaneously into mice and fluorescence images were captured at different time-points (FIG. 11A). This allowed for separate imaging of polymer degradation and cargo release. The profiles of the Cy5 and GFP channels are shown in FIG. 11B and FIG. 11C, respectively. A slight increase in fluorescence intensity was observed in both channels, immediately after implantation. The fluorescence intensity in both channels then declined continually from day 3 to day 32, indicating the degradation of the PLGA and the slow release of the VLPs. The degradation of PLGA was closely correlated to the decline of the VLP signal. This experiment confirmed that the PLGA implants produced by melt-extrusion retained their ability to release cargo slowly over time in vivo and that degradation kinetics of the polymer play the main role in cargo release.

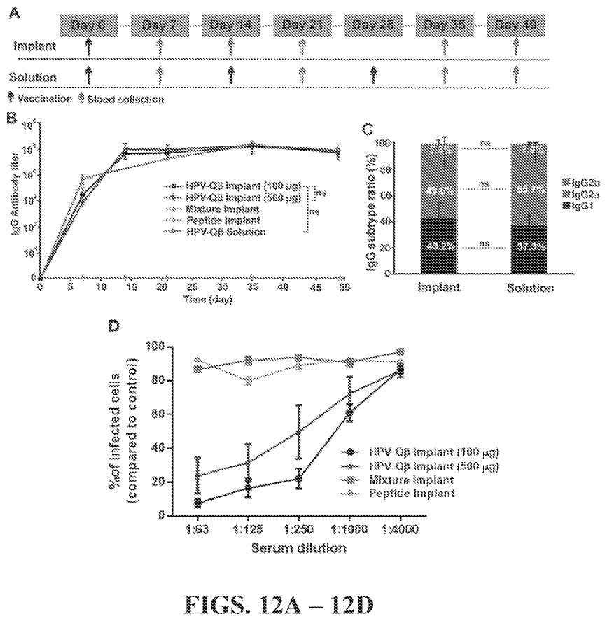

To assess the efficacy of the vaccine candidate delivered using the vaccine delivery device, Applicant subcutaneously vaccinated the mice with PLGA implants loaded with HPV-Qβ at two different doses (100 μg and 500 μg). As controls, Applicant vaccinated mice with PLGA implants loaded with a mixture of the HPV peptide and unmodified Qβ, or with the HPV peptide alone. Finally, Applicant also subcutaneously immunized mice with three doses of free HPV-Qβ (30 μg per dose), as a conventional vaccination control. The immunization and bleeding schedule is summarized in FIG. 12A.

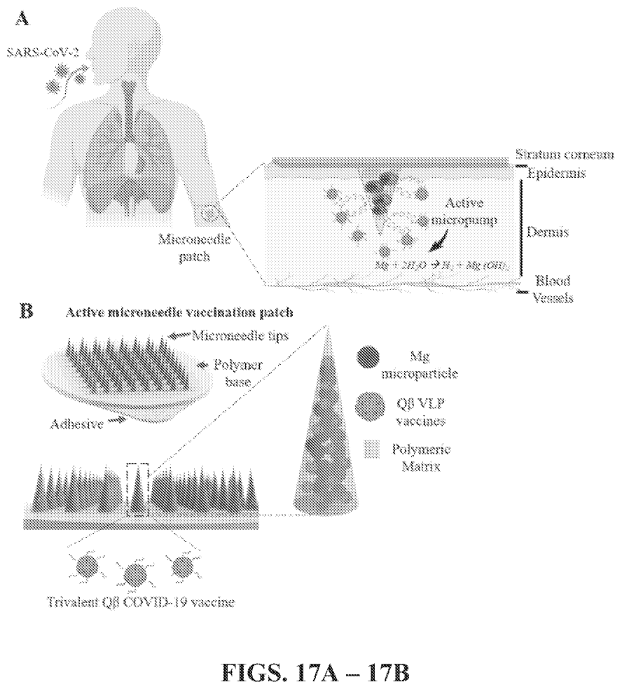

Applicant compared the anti-HPV IgG titers in serum samples taken from mice in each of the experimental and control groups (FIG. 12B). The PLGA implants loaded with HPV-Qβ elicited an intense immune response, with the high HPV-specific IgG titers starting from day 14 and lasting to day 49. There was no significant difference in the IgG titers between the groups receiving 100 μg and 500 μg HPV-Qβ in the implant, indicating that 100 μg HPV-Qβ is an adequate quantity of the antigen for immunization. The high titers elicited by the single-dose HPV vaccine implant were maintained over 49 days and were similar to the titers elicited by the prime, first boost, and second boost schedule, with free VLPs. The PLGA implants loaded with the HPV peptide or a mixture of the HPV peptide and unmodified Qβ particles did not elicit a significant immune response against HPV, demonstrating that the PLGA matrix had no impact on the immunogenicity of the antigens but only ensured their slow release (FIG. 12B). The anti-Qβ IgG titer was consistent with that against the HPV peptide. High IgG titers against Qβ were detected from day 14 to day 49 in mice vaccinated with PLGA implants containing HPV-Qβ or the mixture of unmodified Qβ and the HPV peptide (FIG. 17A).

The IgG subtype ratio following vaccination indicates the mechanism of the immune response. Mice vaccinated with the single-dose HPV-Qβ vaccine based on the PLGA implant, showed a similar HPV-specific IgG subtype ratio to mice in the conventional vaccination control group (FIG. 12C). In both cases, IgG2a was the predominant subtype (˜50%), followed by IgG1 (˜40%) and IgG2b (˜7%). Interestingly, the Qβ-specific IgG subtype ratio was distinct, with IgG2a accounting for up to 70% and IgG1 only 15% of the total IgG titer in both single-dose vaccine group and the conventional vaccination control group (FIG. 17B). This indicates that the cleavage of the bond between the HPV epitope and Qβ carrier is a specific step during immune response, with the separate components then following different immune response pathways.

Additionally, Applicant aimed to evaluate to compare a single-dose soluble injection versus a single-dose biodegradable implant, in an effort to evaluate the necessity for a prime-boost regime with this vaccine candidate. Three groups were studied—(1) a single dose of biodegradable 100 μg HPV-Qβ loaded PLGA implant, (2) a single dose of subcutaneous injection of 100 μg of HPV-Qβ solution, and (3) three subcutaneous injections of 30 μg HPV-Qβ solution. The vaccination and bleeding schedule is shown in FIG. 12A. Results indicate comparable levels of HPV-specific IgG antibody titers independent of the schedule (prime boost vs. single administration) or delivery strategy (injection vs. implant), throughout the entire time of observation for up to 90 days. This indicates that this particular HPV-Qβ vaccine candidate might be effective after single dosing.

Applicant find that in the case of slow release from the implant, the IgG antibody titer was slightly lower after 14 days and reached the same level as obtained by the other methods after 28 days. In all cases, the antibodies remained similar even after three months. Since the lifetime of HPV antibodies in mouse is found to be much longer (>90 days) than the release time of the vaccine cargo from the implant (about 20-25 days), a simple model of exponential loss of antibody in the mouse and an approximately linear release of vaccine cargo from the implant supports Applicant's observations. The model demonstrates that decay kinetics would dominate the concentration of antibodies in blood in the long run and would be essentially same for all methods of administration for this vaccine. Hence, Applicant conclude that the mice vaccinated with the slow-release implants obtain, after a reasonable time, IgG titers comparable to that obtained by the traditional one single full dose or three-dose strategy. While this result might be disappointing, Applicant would like to point out that subcutaneous injections are very expensive and require trained healthcare professionals, thus limiting its availability in developing and poor countries. Furthermore, Applicant speculates that at a lower dose, the implant group might show some advantages of the single dose injection; and this will be tested in future work. If this holds true; this would reduce production costs, which would also be of benefit. Nevertheless, the implications of melt-manufacturing of implantable devices have the potential to be immense. Microneedle patches can be manufactured through injection molding, which has the advantage of massive scalability. These devices show similar efficacy when self-administered, and given the known stability of Qβ, might eliminate cold-chain requirements. Applicant anticipates that this technique would be of great value for vaccines that require a prime-boost dosing regimen. Additionally, several recent studies also point out the benefits of sustained release of antigen over bolus injection in terms of better antigen recognition features and enhancement of germinal center responses.[33-34]

In order to demonstrate the efficacy of the single-dose HPV-Qβ vaccine, serum antibodies from the vaccinated mice were tested for their ability to prevent the infection of pgsa-745 cells by HPV pseudovirus in an in vitro neutralization assay, as previously reported for other vaccines [26]. Data indicate that antibodies raised were indeed neutralizing. The relative number of infected GFP-positive cells was significantly lower when the serum from animals immunized with HPV-Qβ (100 or 500 μg) was added during the assay, compared to serum from animals vaccinated with a mixture of unmodified Qβ and the HPV peptide, or the HPV peptide alone (FIG. 12D). The neutralization titers for each treatment were 1:750 for HPV-Qβ (100 μg), 1:250 for HPV-Qβ (500 μg), and effectively zero neutralizing activity for the free peptide, with or without unmodified Qβ.

Experiment No. 3—SARS-COVID-2

Materials and Methods

CPMV propagation. Cowpea mosaic virus (CPMV) viral nanoparticles were propagated and purified as established elsewhere.[22] Purified CPMV was stored in potassium phosphate (KP) buffer (0.1 M, pH 7.0) at 4° C. Concentrations of CPMV were obtained by UV spectroscopy at 260 nm using the molar extinction coefficients ε_{CPMV}=8.1 ml mg⁻¹ cm⁻¹.

Qβ Virus-like particle production. Bacteriophage Qβ VLPs were expressed as previously reported.[12-13] The gene encoding for Qβ coat protein (CP) (NCBI accession: P03615) was codon optimized for BL21 E. coli expression and synthesized/cloned by GenScript Biotech Co. into pCDF_Qβ. Pure Qβ was resuspended in PBS pH 7.4 and quantified using total protein Pierce BCA assay kit (Thermo Fisher Scientific).

Antigen in silico characterization. Peptide antigens (B cell epitopes) from SARS-CoV-2 spike protein (accession ID: YP_009724390.1) were selected from previous reports[23-27] and examined using an online peptide calculator (https://pepcalc.com/) to determine molecular weight and isoelectric point (pI) (Table 3).

CPMV and Qβ vaccine candidate synthesis. B cell epitopes from SARS-CoV-2 spike protein (accession ID: YP_009724390.1) with an N-terminal cysteine residue and triple glycine linker (C-GGG-peptide) were obtained from GenScript Biotech Co. (Table 3). Peptides were conjugated to CPMV or Qβ (CPMV/Qβ) using a two-step bioconjugation method.[10] First, solvent exposed lysine residues on CPMV/Qβ, were chemically modified with the (NHS)-activated ester moiety of a heterobifunctional linker SM(PEG)₄ to form a CPMV/Qβ-PEG-maleimide intermediate. Specifically, 4 mg of CPMV/Qβ were reacted with SM(PEG)₄ linker (3000 mol eq) in a 1 mL reaction volume of 10 mM KP buffer (pH 7.4) for 2 hours at room temperature. CPMV/Qβ intermediates were purified using Amicon Ultra-0.5 mL centrifugal filters at 10,000×g for 5 minutes and washed 3× using 10 mM KP buffer. Second, the N-terminal cysteine of the peptide epitopes was reacted with the maleimide groups displayed on the CPMV/Qβ intermediates to form the multivalent VNP-peptide conjugates. Specifically, 4 mg of CPMV/Qβ intermediates were reacted with peptide (6000 mol eq for CPMV and 700 mol eq for Qβ) for 12 hours at room temperature on a rotary inverter. The resulting CPMV/Qβ-peptide conjugates were purified and pelleted by ultracentrifugation at 4° C. and 52,000×g over a 30% (w/v) sucrose cushion. Pellets were washed 3× and resuspended using 10 mM KP buffer. Finally, CPMV/Qβ-peptide conjugates were then dialyzed overnight using a 30 kDa dialysis membrane at 4° C. to ensure complete removal of excess reagents. Of note, conjugation of the following peptides (106, 153, 454, and 826) required modifications to standard bioconjugation protocol (briefly discussed below).

TABLE 3
B cell epitopes from SARS-CoV-2 S protein.

| | | | Original | | | | |
|---|---|---|---|---|---|---|---|
| | | | paper | | | | |
| S | Location | | peptide | Length | | Water | |
| domain | (name^{#}) | Sequence | ID | (aa) | pI | solubility | Reference |
| S1 | 369-386 | YNSASFSTFKCYGVSPTK | n/a | 18 | 9.35 | Poor | 23 |
| S2 | 806-820 | LPDPSKPSKRSFIED | n/a | 15 | 6.95 | Good | 24 |
| S1 | 456-460 | FRKSN | n/a | 5 | 11.39 | Good | |
| S2 | 809-826 | PSKPSKRSFIEDLLFNKV*⁺ | S21P2 | 18 | 10.61 | Good | 25 |
| S1 | 553-570 | TESNKKFLPFQQFGRDIA | S14P5 | 18 | 9.64 | Good | |
| S1 | 553-564 | TESNKKFLPFQQ | S1-93 | 12 | 9.64 | Good | 26 |
| S1 | 625-636 | HADQLTPTWRVY | S1-105 | 12 | 7.54 | Poor | |
| S2 | 1148-1159 | FKEELDKYFKNH | S2-78 | 12 | 7.53 | Good | |
| S1 | 92-106 | FASTEKSNIIRGWIF* | S92-106 | 15 | 9.87 | Poor | 27 |
| S1 | 139-153 | PFLGVYYHKNNKSWM* | S139-153 | 15 | 10.21 | Poor | |
| S1 | 406-420 | EVRQIAPGQTGKIAD | S406-420 | 15 | 6.92 | Good | |
| S1 | 439-454 | NNLDSKVGGNYNYLYR* | S439-454 | 16 | 9.10 | Good | |
| S1 | 455-469 | LFRKSNLKPFERDIS | S455-469 | 15 | 10.67 | Good | |

^{#}Nomenclature: in the following the peptides are referred to the 3-4 number code; underlined in bold.
*required F127 for stable conjugation to CPMV, or + to Qβ

Due to insolubility in aqueous media, peptides 106, 153, and 454 required formulation by Pluronic F127 (poloxamer 407, Sigma-Aldrich). Briefly, each respective peptide was dissolved at 10 mg/ml concentration along with 10% (w/w) F127 in DMSO and the solution was transferred into a 1 kDa molecular weight cutoff dialysis membrane (Spectra-Por, Spectrum Labs). Peptides were dialyzed against 10 mM KP buffer (pH 7.4) for 2 hours in a 1 L volume to promote F127 micellization and encapsulation of the hydrophobic peptide (buffer was exchanged at 30 mins and 1 hour). Formulated peptides were then used in bioconjugation reaction with F127-coated VNP intermediates (coating process described below). Of note, peptide 826 did not require formulation, however, it still required coated VNP intermediates. 4 mg of CPMV/Qβ-PEG-maleimide intermediate was first mixed with 4% (w/w) F127 in 10 mM KP buffer and incubated on ice for 1 minute followed by 10 seconds of vortexing. Next, the sample was equilibrated to room temperature for 5 minutes followed by 10 seconds of vortexing to induce CPMV/Qβ coating with F127. Finally, coated CPMV/Qβ intermediates were subject to reaction with peptides 106, 153, 454, or 826 (3000 mol eq for CPMV and 500 mol eq for Qβ) for 12 hours at room temperature on a rotary inverter. Purification was as described above.

CPMV and Qβ vaccine candidate characterization. To verify peptide conjugation, 10 μg of CPMV/Qβ and purified CPMV/Qβ-vaccines were analyzed by sodium dodecylsulfate polyacrylamide gel electrophoresis (SDS-PAGE) performed under reducing conditions on NuPAGE 4-12% Bis-Tris protein gels (Thermo Fisher Scientific) and stained with GelCode Blue Safe protein stain (ThermoFisher Scientific). The gel images were acquired using the ProteinSimple FluorChem® imaging system and densitometry analysis (ImageJ 1.44o software, http://imagej.nih.gov/ij) was used to quantify the number of peptides conjugated per CPMV/Qβ. CPMV/Qβ integrity was confirmed by transmission electron microscopy (TEM) using FEI Tecnai Spirit G2 Bio TWIN instrument following uranyl acetate staining. Particle were concentrated at 0.5 mg/ml in KP buffer and the size was corroborated by dynamic light scattering (DLS) using a Malvern Instruments Zetasizer Nano at 25° C. in plastic disposable cuvettes.

Hot-melt extrusion to formulate trivalent Qβ/PLGA slow-release implants. The Poly(lactic-co-glycolic acid) PLGA implants were prepared using our previously reported desktop melt-processing system.[12-13],[28-29] In brief, a fine PLGA powder (Akina Inc. 50:50 LG Ratio, MW 10-15 kDa) was prepared by passing through a −45 mesh (Sigma-Aldrich). The Qβi vaccines were individually lyophilized. To obtain a trivalent vaccine candidate, Qβ570, Qβ636 and Qβ826 were mixed at equal ratio before hot melt-extrusion. Implant formulation was as follows: 80% PLGA, 10% Qβ, and 10% PEG8000 (by weight). The dry components were mixed by vortexing and loaded into the hot melt-processing system. The barrel was heated to 70° C. for 90 s and the piston was set to 10 psi (69 kPa) for extrusion. Implants were dried and stored at room temperature with desiccants until further use.

Microneedle (MN) formulation of trivalent Qβ vaccines.

PDMS microneedle mold fabrication. The procedure of fabrication of polydimethylsiloxane (PDMS) negative MN silicone molds was developed by casting a PDMS (86:14, base curing agent) solution (SYLGARD 184) onto a master MN acrylate mold array. Prior to casting, PDMS was degassed at 2500 rpm for a period of 5 minutes within a closed desiccator connected to a vacuum pump running at 23 in Hg (78 kPa). Silicone molds were left at room temperature overnight prior demolding. Following the curing process, negative molds were demolded, and custom resized by a blade cut. Prior the MN fabrication, silicone molds were cleaned and thrice washed with soap, ultrasonicated, sterilized at 80° C. and stored in a sealed container.

Dissolvable microneedle fabrication process. Active MN (3Qβ MN active, trivalent candidate) patches were fabricated by following a micromolding procedure by employing negative MN silicon molds. Briefly, a volume of 50 μL of an Mg microparticle (TangShanWeiHao Magnesium Powder Co., Ltd. China) 2-propanol solution (50 mg/mL) was poured onto to negative MN molds to pack the cavities homogeneously. Subsequently, a volume of 250 μL of a 10% (w/v) polyvinylpyrrolidone (PVP, MW=360 K, Sigma Aldrich) aqueous solution (pH 11.5 and pH 7) was cast over silicone molds within a sealed desiccator at 23 in Hg (78 kPa) for a total time of 10 min. Bubbles at the mold/interface were removed, and repetitive additions of polymer solution were casted until reaching a final volume of 750 L. The corresponding payload (500 μg of each Qβ570, Qβ636 and Qβ826) was incorporated onto the mold and allowed to dry for 48 h at room temperature within a sealed container. A transfer base adhesive (3M double sided tape) was applied as the backing of the MN vaccine patch and further demolded from silicone templates. Passive MN (3Qβ MN passive) patches were formulated by following the same protocol but without the incorporation of Mg microparticles. The corresponding active and passive MN patches were stored within a closed container at room temperature prior to use.

MN Patch Characterization. SEM images were obtained with the use of an FEI Quanta 250 ESEM instrument (Hillsboro, Oregon, USA). Prior to imaging, both active and passive MN patch samples were sputtered with iridium (Emitech K575X Sputter Coater), providing a fine grain metal deposition, and imaged with acceleration voltages of 2-5 keV. Digital images of the vaccine MN patch (active and passive) were taken with a Sony a6000 digital camera coupled with a 55 mm panagor 2.8 macro lens.

Immunization of mice. All animal experiments were carried out in accordance with the Institutional Animal Care and Use Committee (IACUC) Office of the University of California, San Diego. Eight weeks old male Balb/c mice were purchased from Jackson Laboratory and kept under controlled conditions with chow and water ad libitum. For subcutaneous (s.c.) injection with liquid format, each CPMV or Qβ vaccine candidate was concentrated at 1 mg/ml in PBS buffer (137 mM NaCl, 2.7 mM KCl, 10 mM Na₂HPO₄, and 1.8 mM KH₂PO₄, pH 7.4) and 100 μl were injected s.c. (100 μg/dose) using a prime-boost protocol and injections carried out two weeks apart. For the trivalent in liquid format (s.c.) Qβ570, Qβ636, and Qβ826 (3Qβ s.c. group) each Qβ vaccine candidate was concentrated at 3 mg/ml in PBS, mixed 1:1:1 just prior administration (100 μL s.c. using 100 μg of each vaccine candidate or 100 μg in total) using the same prime-boost administration schedule. For immunization using the Qβ/PLGA slow-release implants, a single-dose implant with 200 μg of each Qβ vaccine candidate (to equate the 100 μg/dose prime-boost s.c. injection) was administered using an 18G needle (BD Co.) s.c. behind the neck. Trivalent Qβ MN patches (either active or passive) were administered by pressuring for 5 min against the naked skin and then the patches were wrapped tight with tape overnight. MN patches were administered using the same prime-boost administration schedule. As control groups, 5 μg/dose of the three free peptides (570, 636, and 826; the dose was matched to the peptide dose delivered by the CPMV and Qβ vaccines), a blank implant (PLGA:PEG only), and blank MN (PVP only) were used. Five mice were assigned for each group. Blood was collected (in lithium-heparin treated tubes (Thomas Scientific) by retroorbital bleeding before immunization (week 0), and then at weeks 2, 4, and 12 post-immunization. Plasma was collected by centrifugation at 2,000×g for 10 min at room temperature and kept at −80° C. until use.

IgG titers against peptide and S protein. Enzyme-linked immunosorbent assay (ELISA) was used to determine endpoint total IgG titers against the corresponding peptide epitope displayed in the CPMV or Qβ vaccines. 96-well, maleimide-activated plates (Thermo Fisher Scientific) were prepared following manufacturer's directions. In brief, the plates were coated with 100 μl/well of each peptide (same as used for conjugation) at 25 μg/ml in coating buffer (0.1 M sodium phosphate, 0.15 M sodium chloride, 10 mM EDTA, pH 7.2) overnight at 4° C. Plates were washed three times using 200 μl/well of PBST (PBS+0.5% (v/v) Tween-20 (Thermo Scientific)) between every step. Plates were blocked for 1 h at room temperature using 200 μl/well of 10 μg/ml L-cysteine (Sigma-Aldrich). After washing, 2-fold serial dilutions of plasma samples from immunized animals in coating buffer were added and incubated for 1 h at room temperature. After washing, an HRP-labeled goat anti-mouse IgG secondary antibody (Thermo Fisher Scientific) diluted 1:5,000 in PBST was added (100 μl/well) and incubated for 1 h at room temperature. After a final washing step, 1-Step Ultra TMB substrate (3,3′,5,5′-Tetramethylbenzidine, Thermo Fisher Scientific) was added (100 μl/well) and developed for 10 min; the reaction was then stopped using 2N H₂SO₄ (100 μl/well) (Thermo Scientific). The IgG titer against SARS-CoV-2 S protein was determined as described above for peptide but using 96-well nickel activated plates (Thermo Fisher Scientific) coated with 200 ng His-tagged S protein per well (GenScript Biotech Co.). Plasma samples were diluted 1:1000 in PBS. Same secondary antibody dilution and substrate as above was used to develop the assay. The absorbance was read at 450 nm on a Tecan microplate reader. The endpoint antibody titers were defined as the reciprocal serum dilution at which the absorbance exceeded two times the background value (blank wells without plasma sample).

Antibody isotyping. The ELISA was adapted from the protocol against peptide described above; instead of serial plasma dilutions, samples from week 4 (diluted 1:1,000 in coating buffer) were tested. The secondary antibodies used were an HRP-labeled goat anti-mouse IgG1 (Invitrogen PA174421, 1:5,000), IgG2a (Thermo Scientific A-10685, 1:1,000), IgG2b (Abcam ab97250, 1:5,000), IgG2c (Abcam ab9168, 1:5,000), IgG3 (Abcam ab98708, 1:5,000), IgE (Invitrogen PA184764, 1:1,000), IgM (Abcam ab97230, 1:5,000). The IgG2a/IgG1 ratio was reported for each group and a ratio higher than 1 was considered as Th1 response.

ELISpot assay. A mouse IFN-γ/IL-4 Double-Color ELISPOT kit (Cellular Technology Ltd) was used. Briefly, 96-well ELISPOT plates were coated using an anti-mouse interferon-gamma (IFN-γ) capture antibody and anti-mouse interleukin-4 (IL-4) capture antibody overnight at 4° C. (both were used at a 1:166 dilution). Splenocyte suspensions collected from three mice after 2 weeks or 10 weeks post immunization with each Qβ vaccine candidate were analyzed and added to the plates (1×10⁶ cells/well) following stimulation with 100 μl of medium alone (negative control), free-peptides (20 μg/ml) of each epitope, unmodified Qβ (10 μg/ml), or 50 ng/ml phorbol 12-myristate 13-acetate (PMA)/1 μg/ml Ionomycin (Sigma-Aldrich) (positive control) at 37° C. and 5% C02 for 24 h. The plates were washed with PBST and the incubated with anti-murine IFN-γ (FITC-labeled, 1:1000 dilution) and anti-murine IL-4 (biotin-labeled, 1:666 dilution) antibodies for 2 h at RT. Plates were washed, and streptavidin-alkaline phosphatase (AP, 1:1000 dilution) and anti-FITC-horseradish peroxidase secondary antibodies (HRP, 1:1000 dilution) were added to each well and incubated for 1 h at room temperature. Plates were washed with PBST and distilled water, then incubated with AP substrate for 15 min at room temperature, washed with distilled water and incubated with HRP substrate for 10 min at room temperature. Plates were rinsed with water and air-dried at room temperature overnight. Colored spots were quantified using an Immunospot S6 ENTRY Analyzer. The splenocytes were evaluated per animal and tested in triplicate for each stimulant.

SARS-CoV-2 Surrogate Virus Neutralization Test. The SARS-CoV-2 Surrogate Virus Neutralization Test (sVNT) kit (GenScript Co.) is a blocking ELISA that mimics the virus neutralization process. The kit contains two key components: a HRP-conjugated recombinant SARS-CoV-2 RBD fragment (HRP-RBD) and a human ACE2 receptor protein (hACE2). The protein-protein interaction between HRP-RBD and hACE2 can be blocked by neutralizing antibodies against SARS-CoV-2 RBD. The assay was performed following manufacturer's directions. In brief, plasma samples were diluted 1:10 and then mixed 1:1 with HRP-RBD solution and incubated at 37° C. for 30 min. Mixed solution was then added to pre-coated hACE2 plates and incubated at 37° C. for 15 min. Then the plate was washed 4 times and a colorimetric signal was developed incubating the plate with TMB (HRP substrate) for 15 min at room temperature and then absorbance was read at 450 nm. A positive and negative control was tested in the same way as plasma samples. The results were presented as percentage of inhibition rate and were calculated as follows:

Inhibition=(1−(OD value of sample/OD value of negative control))*100

A cutoff value was set up according to % inhibition of control plasma (CPMV, free peptide, blank implant, or blank MN) and plasma samples with inhibition values >than cutoff values were considered as neutralizing.

Neutralization assay with SARS-CoV-2. Reduction of virus-induced cytopathic effect (Primary CPE assay) [30] was performed through the Biopharmaceutical Product Development Services offered by The National Institute of Allergy and Infectious Diseases (NIAID). SARS-CoV-2 strain USA_WA1/2020 was used. Briefly, confluent or near-confluent cell culture monolayers of Vero 76 cells were prepared in 96-well disposable microplates the day before testing. Cells were maintained in MEM (Sigma-Aldrich) supplemented with 5% (v/v) FBS. For antiviral assays the same medium was used but with FBS reduced to 2% (v/v) and supplemented with 50-μg/ml gentamicin. The pooled plasma samples (week 4 post immunization) from each CPMV vaccine candidate and unmodified CPMV (negative control) were tested in 10-fold serial dilutions. Five microwells were used per dilution: three for infected cultures and two for uninfected toxicity cultures. Controls consisted of six wells that were infected and not treated (virus controls) and six that were untreated and uninfected (cell controls) on every plate. Plasma samples and virus were mixed (1:1 ratio) and incubated 1 h at 37° C. before being added to cells. After incubation, growth media was removed from the cells and the plasma sample/SARS-CoV-2 was applied in 0.1 ml volume to wells. SARS-CoV-2, normally at ˜60 CCID₅₀ (50% cell culture infectious dose) in 0.1 ml volume was added to the wells designated for virus infection. Medium devoid of SARS-CoV-2 was placed in toxicity control wells and cell control wells. Plates were incubated at 37° C. with 5% CO₂ until marked CPE (>80%) was observed in virus control wells. The plates were then stained with 0.011% (w/v) neutral red for approximately two hours at 37° C. in a 5% CO₂ incubator. The neutral red medium was removed, and the cells rinsed with PBS to remove residual dye. The PBS was completely removed, and the incorporated neutral red was eluted with 50% Sorensen's citrate buffer/50% (v/v) ethanol for at least 30 minutes. Neutral red dye penetrates living cells, thus, the more intense the red color, the larger the number of viable cells present in the wells. The dye content in each well was quantified at 540 nm wavelength. The dye content in each set of wells is converted to a percentage of dye present in untreated control wells and normalized based on the virus control. The 50% effective (EC₅₀, virus-inhibitory) concentrations and 50% cytotoxic (CC₅₀, cell-inhibitory) concentrations are then calculated by regression analysis. The quotient of CC₅₀ divided by EC₅₀ gives the selectivity index (SI) value. Plasma samples showing SI values≥10 were considered neutralizing.

Statistical analysis. Data is presented as the mean±SD. Single comparisons were made with unpaired, two-tailed t-tests using SPSS Statistics software or GraphPad Prism 6. P values lower than 0.5 were considered as statistically significant with 95% confidence. Replicates per experiment are detailed in each method section or figure.

Results and Discussion

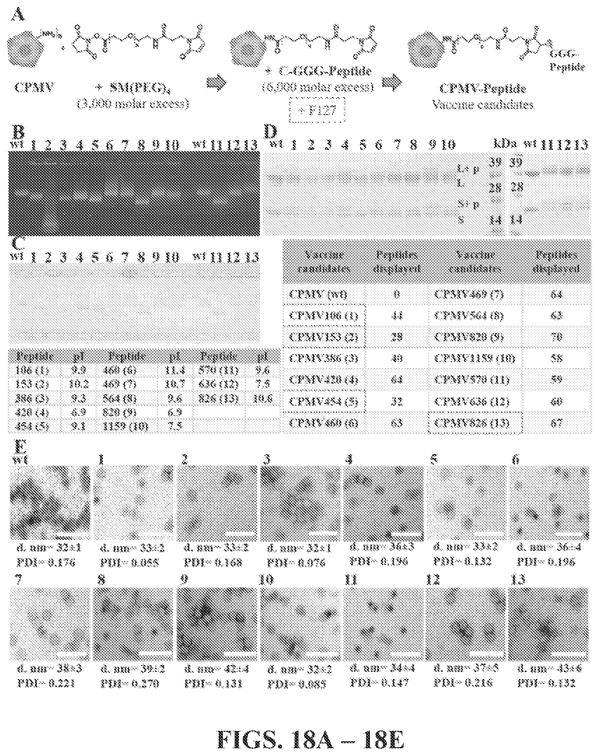

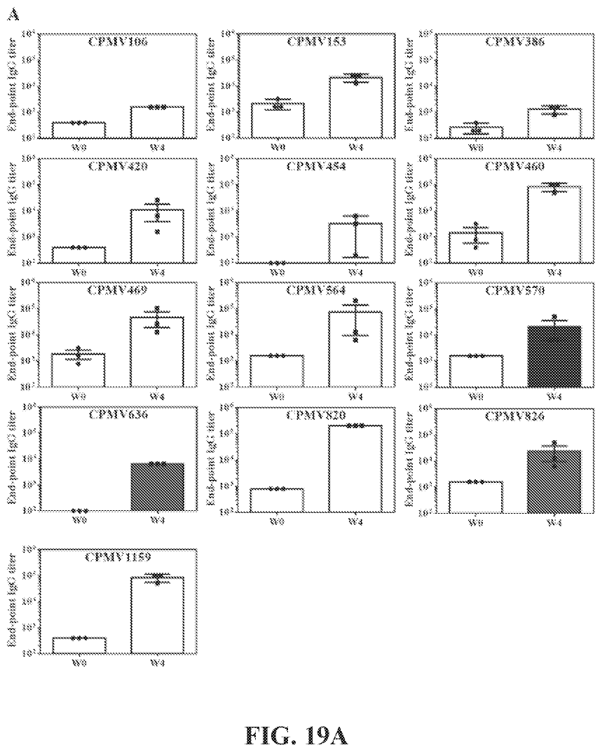

Characterization of CPMV-based vaccines. Applicant selected 13 B cell epitopes (Table 4) which were identified from sera of patients who recovered from COVID-19[23-26] or that were shown by others to be neutralizing when displayed on VLP technologies.[27] Applicant first considered the CPMV platform technology. CPMV can be engineered to display epitopes through genetic fusion[31,32] or chemical conjugation; Applicant opted for the latter because it provides a plug-and-play strategy particularly suitable for pandemic or epidemic responses. The adjuvant and display platform, CPMV, could be stockpiled and target epitopes could be conjugated as needed. The long-term stability of the platform and ease of vaccine manufacture allows for a rapid response to mutants (VOCs) or new emerging infectious diseases. Applicant adapted previously published procedures[10] and achieved conjugation of each of the 13 candidate B cell epitopes using a two-step conjugation scheme (FIG. 18A): first CPMV was conjugated with an SM(PEG)₄ linker. The NHS handle of the SM(PEG)₄ linker reacts to solvent-exposed Lys side chains on CPMV[33] and the introduced maleimide group is then coupled to the C-terminal Cys residue on the C-GGG-peptide sequence. Several peptides (106, 153, 454, and 826) resulted in extensive aggregation, therefore a formulation approach in which a surfactant (Pluronic F127) was added to the reaction mix to stabilize the reagents was used. Post-conjugation the surfactant was removed and yielded stable conjugates (the detailed methods will be reported elsewhere [bioXriv]). Resulting CPMV-based SARS-CoV-2 vaccines were purified by ultracentrifugation and characterized using a combination of dynamic light scattering (DLS), transmission electron microscopy (TEM), and gel electrophoresis (native agarose gels and denaturing sodium dodecyl sulfate polyacrylamide gel electrophoresis (SDS-PAGE)) to confirm their structural integrity and degree of antigen incorporation (FIG. 18, Table 4, FIG. 13).

TABLE 4
Number of SARS-CoV-2 epitopes displayed
per CPMV or Qβ particle.

| Vaccine candidate | Peptides displayed/per particle |
|---|---|
| CPMV106 | 44 |
| CPMV153 | 28 |
| CPMV386 | 40 |
| CPMV420 | 64 |
| CPMV454 | 32 |
| CPMV460 | 63 |
| CPMV469 | 64 |
| CPMV564 | 63 |
| CPMV570 | 59 |
| CPMV636 | 60 |
| CPMV820 | 70 |
| CPMV826 | 67 |
| CPMV1154 | 58 |
| Qβ570 | 98 |
| Qβ636 | 107 |
| Qβ826 | 105 |

DLS exposed the hydrodynamic diameter of the CPMV vaccines with diameters ranging between 32 and 42 nm (FIG. 18E and FIG. 13). Overall, the size is in good agreement with the size of CPMV, 31 nm as reported based on its crystal structure.[7] The increase in hydrodynamic diameter observed for several CPMV vaccines may be a property of the peptide conjugated; particle aggregation was not detected by native agarose gel electrophoresis, DLS or TEM (see FIG. 18). TEM imaging of negatively stained CPMV corroborated that particle preparation yielded monodisperse and intact CPMV nanoparticles measuring ˜31 nm in size (FIG. 18E). Native agarose gels further validated the presence of intact CPMV (FIG. 18B and FIG. 18C) with RNA and protein co-localizing after nucleic acid and protein staining. Changes in electrophoretic mobility can be attributed to peptide conjugation; first conjugation of the peptides is targeting the Lys side chains, which reduces the overall positive charge on CPMV leading to increased mobility toward the anode. Differences in mobility comparing the various COVID-19 vaccines is explained by the varying charges of the peptide epitopes conjugated (FIG. 18B and FIG. 18C; Table 4); the lower the IP the higher the electrophoretic mobility. Lastly, SDS-PAGE analysis confirmed the presence of modified and unmodified small and large coat protein (CP) (˜24 kDa and ˜42 kDa) (FIG. 18D); band analysis tool and Image J software indicates that peptide conjugation yielded 23 to 58% modification of the CPs; or in other words 27 to 70 peptides were displayed per CPMV (Table 4). Data indicate that intact CPMV-based COVID-19 vaccines were successfully produced.

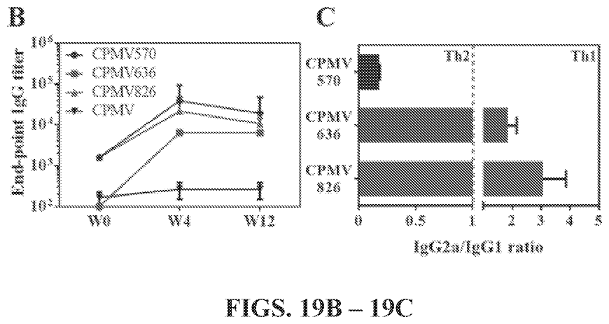

Immunogenicity and neutralizing activity of the CPMV-based COVID-19 vaccines. Applicant first assessed the efficacy of the CPMV-based COVID-19 vaccines using a prime-boost administration schedule (FIG. 19A) with two doses (100 μg each) two weeks apart applied s.c. All 13 candidates raised antibodies against their respective epitopes as observed using ELISA against the peptide epitopes (FIG. 19). The endpoint antibody titers tested at week 4, 2 weeks after the prime-boost immunization, were more prominent in some candidates than others, with the highest levels observed for CPMV1159, CPMV820, and CPMV460 (endpoint IgG titers of 1:85,333, 1:204,800, and 1:85,333 respectively; with increments of two or more magnitudes vs. controls [1:400, 1:800, and 1:1,466, respectively]); remaining candidates CPMV420, CPMV469, CPMV564, CPMV570, CPMV636, CPMV826 had moderate titers (endpoint IgG titers of 1:11,200, 1:46,933, 1:74,666, 1:38,400, 1:6,400, and 1:23,466, respectively; with increments of at least one order of magnitude vs. controls [1:400, 1:1,866, 1:1,600, 1:1,600, 1:100, and 1:1,600, respectively]), except for CPMV106, CPMV153, CPMV386, and CPMV454 which showed only modest level of response (endpoint IgG titers of 1:1,600, 1:21,333, 1:1,333, and 1:3,266, respectively; with increments less than one order of magnitude vs. controls [1:400, 1:2,133, 1:266, and 1:100, respectively]) (FIG. 19). The poor immunogenicity observed for the CPMV106, CPMV153, CPMV386 and CPMV454 may be explained by the number of the peptides conjugated, which were the lowest among all vaccines (FIG. 18D; <50 peptides displayed per particle). Then, Applicant also selected 3 candidates (those that were found to be neutralizing, see below) for longitudinal study and found that the antibody titers were maintained over 12 weeks (FIG. 19B). Lastly, the plasmas from the same 3 candidates (CPMV570, CPMV636, and CPMV826) were analyzed for antibody isotyping. FIG. 19C shows the IgG2a/IgG1 ratio which indicates a Th2 bias for CPMV570 and Th1 bias for CPMV636 and CPMV826. CPMV-based vaccines have been previously reported to induce Th1-biased responses (with IgG2a>IgG1 ratio>1).[4],[10],[38] Applicant's data indicates that the epitope itself can influence the resulting immune response with only candidate 570 shifting toward a Th2 bias.

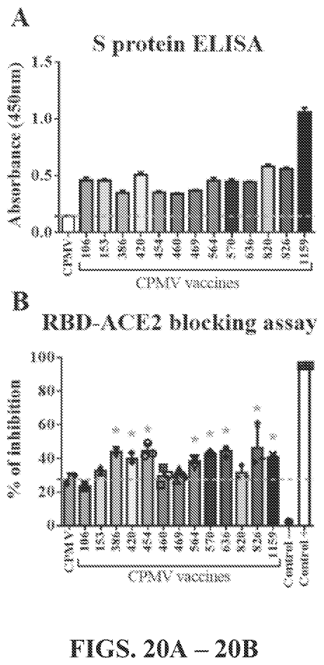

Next, Applicant tested mouse plasma against the S protein via ELISA and confirmed that IgG-specific toward SARS-CoV-2 S protein were indeed raised (FIG. 20A); each candidate tested positive, however, CPMV1159 gave particularly high signals (consistent with the peptide-based ELISA; see FIG. 19). On the other hand, CPMV820 which gave high titers when probed against the peptide epitope, resulted in signals comparable to the other CPMV-based COVID19 vaccines. Therefore, Applicant found no direct correlation between the titers against the epitope vs. S protein, and this can be explained by the relative location and surface availability of the epitopes under the assay conditions.

To assay for SARS-CoV-2 neuralization, Applicant used two assays: a SARS-CoV-2 Surrogate Virus Neutralization Test (sVNT) and a SARS-CoV-2 neutralization assay using Vero 76 cells. The sVNT is based on an RBD to ACE2 receptor binding assay and was used as a pre-screening tool to select candidates with neutralizing or inhibitory effect on the RBD-ACE2 interaction. Sera from eight of the 13 COVID-19 vaccines demonstrated an inhibitory effect (FIG. 20B) with subtle differences noted between the various constructs (negative controls, i.e. plasma from mice immunized with CPMV had no inhibitory effect on the RBD-to-ACE2 binding). Of note, while binding data indicate CPMV 1154 to have increased interaction with S protein compared to other candidates (FIG. 20A), this did not correlate with increased inhibitory effect; CPMV 1154 inhibitory effect was comparable to other candidates tested.

All 13 candidates were subjected to a SARS-CoV-2 neutralization assay using SARS-CoV-2 strain USA_WA1/2020 and Vero 76 cells. Data indicate three candidates (CPMV570, CPMV636, and CPMV826; Table 5) to be neutralizing for SARS-CoV-2; these candidates were also inhibitory using the sVNT. Most striking are the high neutralizing titers achieved, especially for the CPMV826 candidate which reached a neutralization titer (neu titer) of 960. This is comparable to titers reported in neutralization assays using Moderna's mRNA-1273 vaccine.[34] Of note, the CPMV-based B cell epitope vaccines outperformed neu titers reported using other VLP display strategies. Applicant found that three candidates neutralized SARS-CoV-2 with neu titers of 480 for candidates CPMV570 and CPMV636, and neu titer of 960 for candidate CPMV826, and this was achieved without addition of any adjuvants. In contrast, B cell epitopes of SARS-CoV-2 S presented on hepatitis B core protein (HBc) particles adjuvanted with Alum yielded neu titers of only 80.[27] Interestingly though, the B cell epitopes 106, 153, 420, 454, and 469 that produced neutralizing antibodies when presented on HBc particles albeit at low titers (inhibition of SARS-CoV-2 pseudovirus was reported at rates of 40-50% at 1:20 dilution),[27] did not yield neutralizing responses when displayed using CPMV. Nevertheless, higher dosing or adding of the Alum adjuvant may also yield comparable responses.

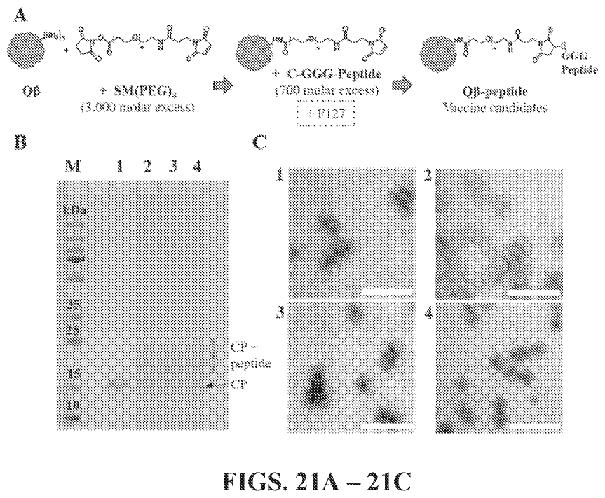

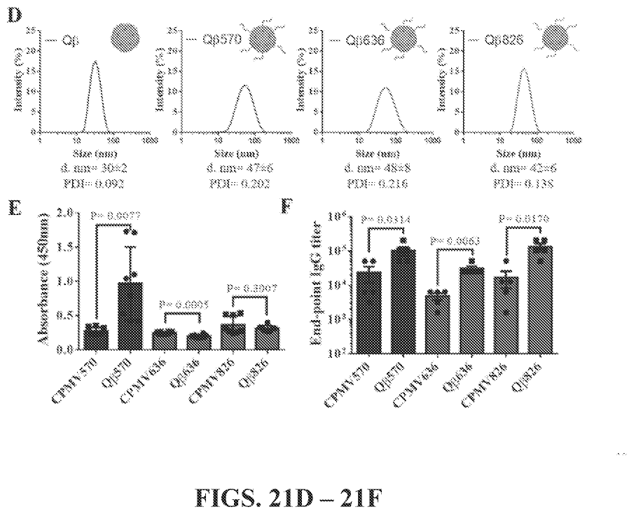

Validation of the three candidate epitopes (570, 636, 826) using the Qβ VLP platform technology. Only some but not all of the 13 epitope candidates produced neutralizing SARS-CoV-2 titers. Peptide epitopes that yielded neutralizing responses were epitopes 570, 636, and 826, and these were identified from convalescent patients' sera.[25-26] To further validate these epitopes Applicant tested whether the epitopes could be transferred to another platform, specifically using the VLPs from bacteriophage Qβ. Qβ VLPs have been widely used as a vaccine platform. Because the Qβ VLPs can be produced using bacterial expression (an industry standard for production of biologics)—as opposed to molecular farming of CPMV—the Qβ-based vaccines may offer a platform more readily translated into cGMP manufacturing for clinical testing. In fact, several Qβ-based vaccines targeting chronic diseases have undergone or are undergoing clinical testing.[5-6],[11] Qβ VLPs were produced through expression in E. coli and then chemically modified with the selected peptide epitopes 570, 636, and 826 using chemical conjugation protocols as established for CPMV (FIG. 21A). Also, for the characterization, Applicant followed the procedures described for CPMV. SDS-PAGE analysis confirmed the presence of unmodified Qβ CP (˜14 kDa) and peptide-modified Qβ CPs. The band pattern indicates that up to three peptides were conjugated per CP ((FIG. 21B), and this is in agreement with up to 4 available amines per Qβ CP (3 from solvent exposed Lys side chains and the N-terminus).[35-37] Band density analysis was performed to estimate the degree of modification and Applicant determined 54%, 59%, and 58% of the CPs of Qβ570, Qβ636, Qβ826, respectively, were modified with their respective peptide epitope. This equates to 98 to 107 peptides per Qβ particle (each Qβ VLP is comprised of 180 identical copies of a CP unit, Table 4). TEM and DLS data confirmed the presence of monodisperse nanoparticles, broken particles or aggregation was not apparent (FIG. 21C+FIG. 21D). DLS measurements indicated a significant increase in the hydrodynamic diameter from 30 nm for unmodified Qβ to 47, 48, and 42 nm for the Qβ570, Qβ636, and Qβ826 formulations. A profound increase in hydrodynamic diameter was also apparent for some of the CPMV-based vaccines (see FIG. 18) and this has also been document for HBc particles displaying epitopes from SARS-CoV-2 S protein.[27] This “swelling effect” may be even more profound for Qβ vs. CPMV, because the density of peptides displayed for Qβ was higher compared to CPMV (Table 4). Overall data indicated that intact Qβ-based COVID-19 vaccines Qβ570, Qβ636, and Qβ826 were produced. Applicant used a prime-boost schedule to immunize mice and confirmed that the peptide epitopes maintained their immunogenicity when presented on Qβ yielding high epitope- and S-protein specific antibodies (FIG. 21E and FIG. 21F). For the ELISA against S protein, the Qβ570 vaccine candidate gave rise to higher antibody titers (3 times higher absorbance) compared to CPMV570; for the 636 formulation there was a trend with Qβ636 producing increased antibody titers vs. CPMV636; for Qβ826 and CPMV826 there was no difference (FIG. 21E). When testing the sera against the immobilized peptide, Qβ-based vaccines candidates (Qβ570, Qβ636, and Qβ826) were higher (up to one log order of magnitude) vs. CPMV570, CPMV636, and CPMV826, respectively (FIG. 21F). This may be explained with the higher payload delivery of Qβ which displayed up to twice the number of peptides compared to CPMV (Table 4); this is consistent with reports demonstrating that highly ordered repetitive arrays of epitopes are effective for the induction of immune responses and breaking B cell tolerance.[5-6] Nevertheless, data indicate CPMV and Qβ to be suitable platforms for peptide display; here Applicant chose to move forward with the Qβ-based platform to produce mono- and trivalent COVID-19 formulations that were delivered as soluble injections as well as slow-release implants and via microneedle patches.

TABLE 5
Neutralization assay against SARS-CoV-2 in vitro.

| | | SARS-CoV-2 | | | |
|---|---|---|---|---|---|
| Plasma sample | Drug assay name | EC₅₀ | CC₅₀ | SI₅₀ | Neu titer |
| CPMV106 | Visual | 59 | 167 | 2.8 | 120 |
| | Neutral Red | 59 | 167 | 2.8 | |
| CPMV153 | Visual | 33 | 167 | 5.1 | 240 |
| | Neutral Red | 17 | 167 | 9.8 | |
| CPMV386 | Visual | 17 | 167 | 9.8 | 480 |
| | Neutral Red | 17 | 167 | 9.8 | |
| CPMV420 | Visual | 59 | 167 | 2.8 | 120 |
| | Neutral Red | 58 | 167 | 2.9 | |
| CPMV454 | Visual | 59 | 167 | 2.8 | 120 |
| | Neutral Red | 56 | 167 | 3 | |
| CPMV460 | Visual | 30 | 167 | 5.6 | 240 |
| | Neutral Red | 26 | 167 | 6.4 | |
| CPMV469 | Visual | 59 | 167 | 2.8 | 120 |
| | Neutral Red | 56 | 167 | 3 | |
| CPMV564 | Visual | 59 | 167 | 2.8 | 120 |
| | Neutral Red | 51 | 167 | 3.3 | |
| CPMV570 | Visual | 15 | 167 | 11 | 480 |
| | Neutral Red | 12 | 167 | 14 | |
| CPMV636 | Visual | 15 | 167 | 11 | 480 |
| | Neutral Red | 13 | 167 | 13 | |
| CPMV820 | Visual | 59 | 167 | 2.8 | 120 |
| | Neutral Red | 55 | 167 | 3 | |
| CPMV826 | Visual | 7.4 | 167 | 23 | 960 |
| | Neutral Red | 6.3 | 167 | 27 | |
| CPMV1159 | Visual | 59 | 167 | 2.8 | 120 |
| | Neutral Red | 51 | 167 | 3.3 | |
| CPMV | Visual | 167 | 167 | 0 | <60 |
| | Neutral Red | 167 | 167 | 0 | |

EC50 = compound concentration that reduces viral replication by 50%
CC50 = compound concentration that reduces cell viability by 50%
SI50 = CC50/EC50
SI50 values > 10 are considered as neutralizing.
Neu titer = Neutralization titer at EC50

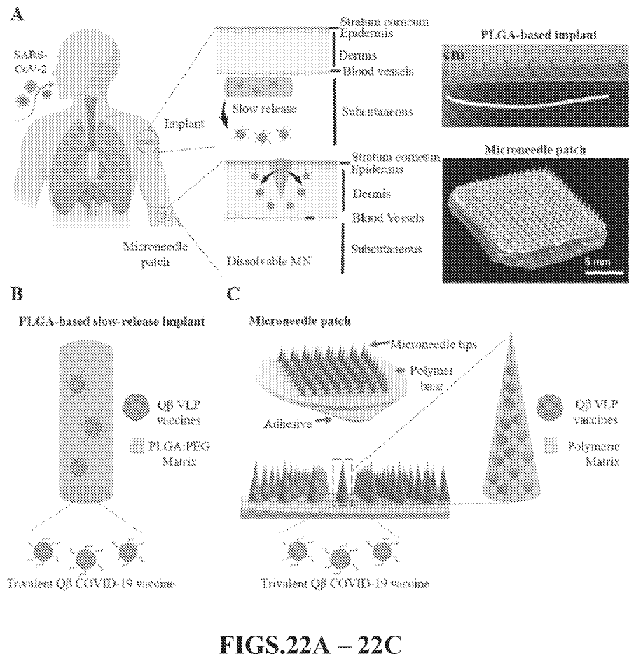

Trivalent Qβ-based COVID-19 formulations and their delivery devices. Applicant established protocols for hot-melt extrusion protocols yielding degradable PLGA:Qβ implants that released the candidate vaccines over a ˜1 month time frame after s.c. administration in mice. A combination of in vitro, ex vivo, and in vivo assays demonstrated that the released Qβ-based vaccines remained structurally sound and biologically active. Building on this data, in this work Applicant formulated a trivalent Qβ-based COVID-19 vaccine candidate to be delivered as a single dose using a degradable PLGA:Qβ implant formulated with 800% PLGA (50:50 LG ratio), 10% PEG8000, and 10% trivalent Qβ vaccine (Qβ570, Qβ636, and Qβ826) (by weight). The trivalent vaccine candidate was obtained by mixing Qβ570, Qβ636, and Qβ826 at equivalent weight percentages. After mixing all the components the melt-extrusion was performed and a solid and uniform rod-shape (0.5 mm×70 mm) implant was generated (FIG. 22A+FIG. 22B). The slow-release formulation and soluble version of the trivalent Qβ570, Qβ636, and Qβ826 vaccines were compared to monovalent formulations. In addition, Applicant formulated the trivalent Qβ-based COVID-19 (Qβ570, Qβ636, and Qβ826) vaccines as a MN patch by casting mixtures of Qβ570, Qβ636, and Qβ826 and polyvinylpyrrolidone (PVP) into silicon molds. Two designs were employed, passive and active, where the latter contains Mg microparticles to enable micro-mixing to provide a propulsive force to improve the distribution of the payload from the device. While significant improvement in efficacy comparing the active vs. passive active MN delivery technology was previously demonstrated for cancer immunotherapy delivered directly into dermal melanomas,[14],[40] active dermal delivery of the vaccines conferred no enhanced efficacy over the traditional (passive) MN patch. Therefore, in the following Applicant focus on the comparison of the following delivery strategies: ‘passive’ or traditional MN vs. implant vs. hypodermic injection using the mono- or trivalent vaccines (Qβ570, Qβ636, and Qβ826). The patch dimensions were designed to be 1.2 cm×1.2 cm, and the array comprised 225 conical-shaped MNs (850 μm in height and diameter of 400 μm; FIG. 14). Characterization of the MN arrays by scanning electron microscopy (SEM) and corresponding energy dispersive X-ray elemental analysis (EDX) verified the structural integrity of MN structures in the array, both displaying tip sharpness and size reproducibility (FIG. 22A, FIG. 22C and FIG. 14). Previously made MN arrays made from PVP from our group have demonstrated the necessary mechanical stability and strength requirements for efficient patch application.[14],[40] The MN patches were designed to dissolve upon contact with the skin; as MNs breach dermal barriers and remain embedded within the application area, the polymer rapidly dissolves,[14],[40] therefore releasing Qβ570, Qβ636, and Qβ826 simultaneously. The MN fabrication (materials, process, and design aspect) was carefully optimized to retain antigenicity without the need of use of high temperatures or harsh organic solvents, thus transiently delivering the vaccines without leaving any sharp-based waste after application. After fabrication, both MN patch modalities demonstrated to be stable at room temperature and dry conditions for up to 1 month.

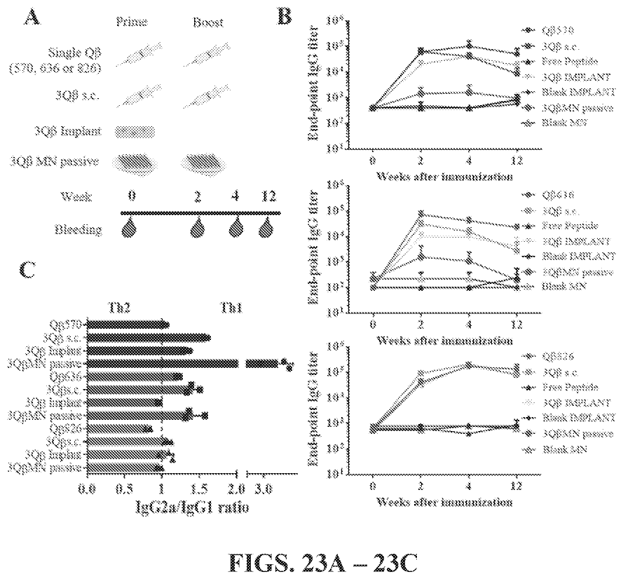

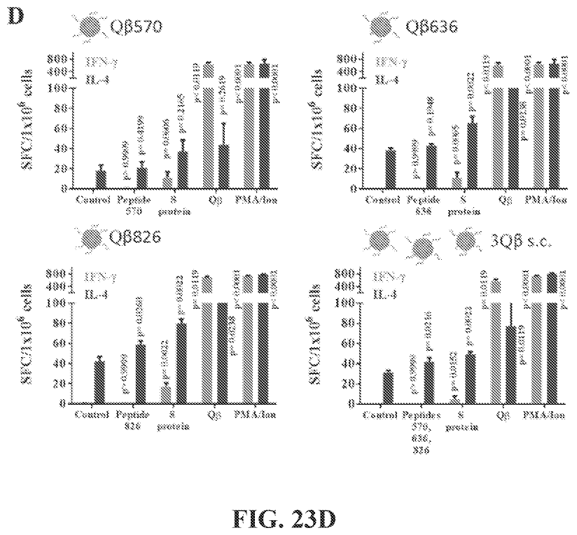

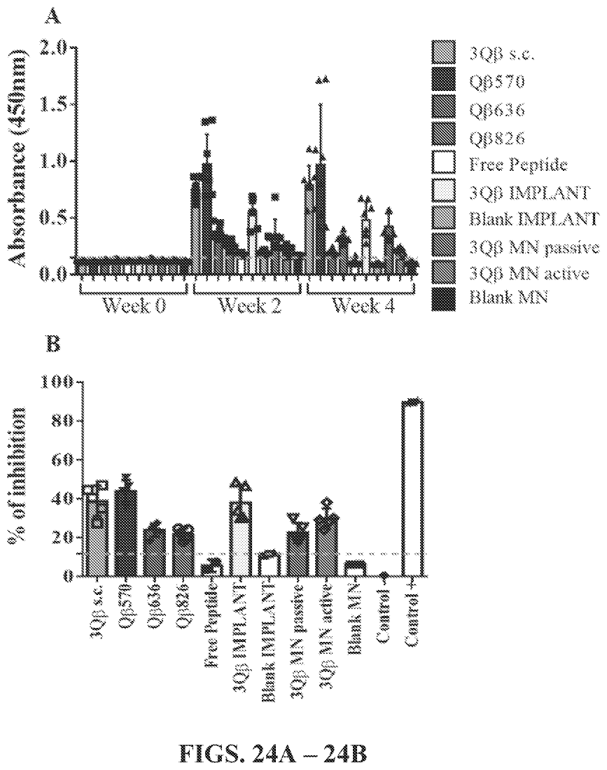

Immunogenicity of trivalent Qβ vaccine. Next, Applicant established whether immunization using single antigen formulations (Qβ570, Qβ636, or Qβ826) vs. the trivalent Qβ vaccine candidate would yield desired antibody responses against peptide epitopes (FIG. 23 and SARS-CoV-2 S protein (see FIG. 24). The vaccines were delivered by (i) injection s.c., (ii) injectable slow-release implant or (iii) MN patches. The injections and MN delivery followed a prime-boost schedule while the PLGA/Qβ blends were administered as single dose; the implants are injectable and are placed s.c. (FIG. 23A). In previous work, Applicant demonstrated that this particular formulation: 80% PLGA, 10% Qβ, and 10% PEG8000 (by weight) releases Qβ from the implants over a time course over 30 days.[12-13],[28-29] The sustained release alleviates the need for repeated dosing. Overall, Applicant found that monovalent and trivalent vaccines yielded comparable antibody titers against their target antigen (FIG. 23B); this is consistent with our previous work on multivalent vaccine mixtures.[12] Prime-boost administration of soluble vaccines (single antigen vs. trivalent 3Qβ groups) and single administration of the slow-release implant yielded comparable antibody titers against each antigen tested (FIG. 23B; FIG. 15A+FIG. 15B), demonstrating that single dose administration of the PLGA/3Qβ is sufficient. With the 3Qβ MN group reduced titers against the target epitope 570 and 636 were noted; however, the antibody titers against peptide epitope 826 were comparable to any other group tested (FIG. 23B; FIG. 15B).

While soluble and implant-based vaccines were delivered s.c., the MN patches are delivered dermally (FIG. 22). Resident APCs are more abundant and diverse in the dermis compared to the s.c. tissue;[41-43] differences in charge and hydrophobicity of the peptide epitopes may alter release from the MN patches and impact in vivo trafficking and subsequent processing by antigen presenting cells (APC). Data may indicate more effective processing of the peptide epitope 826 which is highly positively charged (FIG. 23B). The reduced antibody levels may be explained also by delivery of a reduced dose using the MN approach: here the active ingredient, i.e. 3Qβ vaccine candidate, is released only from the needles making the base of the MN patch a void volume. Applicant performed BSA release studies and determined that ˜10% of the protein is released from the needles. Thus, to produce a 100 μg dose per Qβ candidate, a total of 1 mg Qβ per candidate or total of 3 mg of protein would need to be loaded per MN patch. The maximum loading dose however was 1.5 mg protein which equates to 500 μg per Qβ candidate, thus providing an effective dose of ˜50 μg vs. 100 μg (used for the soluble injection). The limitation is the volume of the device and protein concentrations that could not be increased above 60 mg/ml. From a translational point of view this is unlikely a barrier as the MN patch size could be increased.

Antibody titers were monitored over 12 weeks; overall antibody titers were maintained with a slight decrease for observed for the 636 and 570 peptide formulations (FIG. 23B and FIG. 21B). Again, the data demonstrates the potency of the 826 formulations which maintained high titers of antibodies over the 3-months time course (later time points were not considered). Our next goal was to establish what immunoglobulin isotypes (IgG subclasses: IgG1, IgG2a, IgG2b, IgG2b, IgG2c, IgG3; IgM, IgE) were provoked. IgG1 is a subclass of IgG that are known to be primarily induced via Th2-type cytokines (e.g., interleukin-4 (IL-4)), and the generation of IgG2a antibodies is involved with the presence of Th1-type cytokines (e.g., interferon-gamma (IFN-γ)).[44] Data indicate that IgG2a/IgG1 ratios remained essentially the same between groups; and IgG2a/IgG1 ratios were ≥1 (FIG. 23C). Interestingly, the 3Qβ MN active group showed a ratio<1 for any epitope tested (FIG. 15C). Therefore, vaccines produced a Th1/Th2 balanced immune response, except for the 3Qβ MN active group that exhibited a Th2 biased profile (FIG. 15C); the production of hydrogen as a result of dissolving Mg particles used for the micro-mixing may influence the cytokine/chemokine profiles and therefore bias the immune response. Applicant also tested for additional IgG isotypes and detected IgG2b at similar levels compared to IgG2a, indicating a slight bias toward a Th1 profile; IgG2c is not expressed in Balb/c and hence was not detected; IgM was perceived in all the groups and titers were dependent of the epitope used; lastly IgE, an isotype related to allergic diseases was not detected, indicating safety (FIG. 15). Comparing the profiles obtained for CPMV and Qβ, data indicate that both, the epitope and carrier impact whether Th1 or Th2 bias is established.

Applicant also assessed whether T cell activation was primed upon a single dose of monovalent or trivalent vaccine candidate s.c.; splenocytes were analyzed by ELISpot 14 days post administration s.c. Stimulation of splenocytes with unmodified Qβ yielded comparable levels of IFN-γ and IL-4 when compared to positive control phorbol 12-myristate 13-acetate (PMA)/Ionomycin (FIG. 23D). T cell responses against the vaccination platform, the Qβ VLP, was expected due to the VLPs containing a mixture of epitopes.[12] When stimulated with S protein, IFN-γ and IL-4 secretion was observed for any vaccine candidate and which is in agreement with the Th1/Th2 balanced immune response (see FIG. 23C+FIG. 23D).

Immunogenicity against recombinant S protein. ELISA assays confirmed that Qβ-based COVID-19 vaccines, mono- or trivalent, and delivered as soluble vaccine or via vaccine delivery devices yielded antibody responses recognizing intact SARS-CoV-2 S protein (FIG. 24A). Furthermore, the sVNT assay confirmed that antibodies from all vaccination groups were effective at inhibiting inhibit RBD-ACE2 interactions (FIG. 24B). Overall, data indicated that the epitopes 570, 636, and 826 produce robust immune responses against SARS-CoV-2 S protein and epitopes can be transferred from one to another vaccination platform. Lastly, while Applicant did not observe a synergistic effect when using the trivalent vaccine formulations in the RBD-based neutralization assay, it is possible that the trivalent vaccine candidate would produce synergistic or enhanced neutralization effects in vivo.

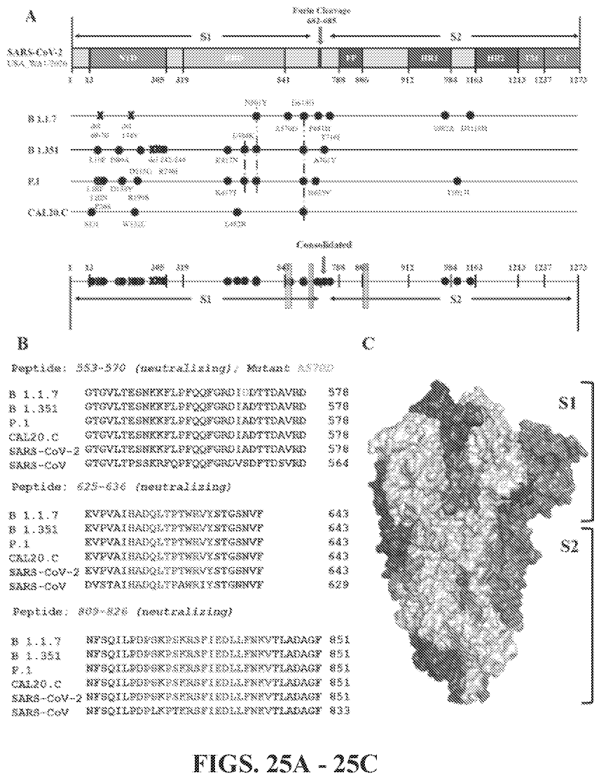

SARS-CoV-2 variants and the trivalent vaccines. Since the beginning of the pandemic variants of SARS-CoV-2 have been reported with mutations occurring in the S protein.[45-47] The SARS-CoV-2 S protein is a class I fusion protein produced as a large 1273 amino acid inactive precursor (S0). Proteolytic cleavage of the S protein generates the highly variable S1 subunit and S2 subunit that is conserved across human coronaviruses. [48-49] The N-terminal domain (NTD) and the receptor-binding domain (RBD) are located in the S1 subunit. The fusion peptide (FP), two heptad repeats (HR1 and HR2), central helix (CH), transmembrane (TM) domain, and cytoplasmic tail (CT) are located in the S2 subunit. Three S1/S2 protomers non-covalently associate to form the functional S-trimer.[50] Mutations and emergence of variants is a natural response of the virus to selective pressure either to chronic COVID-19 development, to vaccination or to antiviral treatments. 400 distinct mutation sites were reported in the S1 and S2 regions with the highest frequency of mutations occurring in the S1/RBD region.[48] Not surprisingly, VOCs harbor numerous RBD mutations with troubling characteristics such as greater binding affinity (up to 10-fold) with hACE2 receptor as one of the main contributing factors toward increased infectivity (B 1.1.7/N501Y)[51] and complete evasion from current monoclonal antibody therapy (Bamlanivimab) of B1.351/501Y.V2 and P1/501Y.V3.[48] Among the 13 B-cell epitopes in our peptide library, five peptide sequences (106, 153, 420, 454, 570) contain mutations present in one or more VOCs (Table 6). Notably, all these mutations are located in the S1 subunit, and two are located in the RBD (FIG. 25A). Among the validated epitopes (570, 636, 826) only epitope 570 has a single substitution present in B 1.1.7 variant (A570D), located in the N-terminal domain. Peptide epitopes 636 and 826 are not affected by the mutations reported to date, therefore showing promise as broadly neutralizing vaccines (FIG. 25B). While a mutation within the 636 epitope sequence has not yet occurred, mutations have been reported in the surroundings of the furin cleavage sequence (D614G, P681H, H655Y, A701V, T716I) suggesting that this area is susceptible to mutations. Of remarkable attention is the epitope 826, which is situated in the S2 domain adjacent to the fusion protein region and outside of the HR1 and HR2 regions. The low degree of mutation rate in this region[48] and the high degree of homology between SARS-CoV-2 and SARS within the 18AA peptide consensus sequence (FIG. 25) makes this an attractive target for vaccine development. The CPMV826 vaccine candidate yielded high neutralizing antibody titers, comparable to Moderna's vaccine,[34],[52] highlighting the functional relevance of this epitope. The underlying mechanism for the high neutralizing titer against the 826 epitope warrants further investigation.

TABLE 6
B cell epitopes used that contained a
mutation from variants of concern.
BOLD letters indicate a
deletion/mutation.

| S | Loca- | Se- | | | Mutation |
|---|---|---|---|---|---|
| Domain | tion | quence | Length | Variant | site |
| S1 | 92-106 | FASTEKSN | 15 | B 1.526 | T95I |
| | | IIRGWIF | | | |
| S1 | 139-153 | PFLGVYYH | 15 | B 1.1.7 | del |
| | | KNNKSWM | | | 144Y |
| S1 | 406-420 | EVRQIAPG | 15 | B 1.3551 | K417N |
| | | QTGKIAD | | + P1 | |
| S1 | 439-454 | NNLDSKVG | 16 | CAL20.C | L452R |
| | | GNYNYLYR | | | |
| S1 | 553-570 | TESNKKFL | 18 | B 1.1.7 | A570D |
| | | PFQQFGRD | | | |
| | | IA | | | |

Experiment No. 4—Cowpea Mosaic Vaccine

Here, Applicant report how plant VNPs based on CPMV conjugated with B-cell epitopes can be developed as a vaccine candidate for beta-coronavirus neutralization. Soluble CPMV vaccines were evaluated using prime-boost immunization. The ongoing COVID-19 pandemic highlights the need for vaccines and delivery devices that overcome cold chain requirements and are effective after a single administration. Therefore, Applicant also formulated the CPMV vaccines as a slow-release polymer implant prepared by hot melt-extrusion, a highly scalable process technology suited for epidemic or pandemic response. Our previous research showed that VNPs can withstand hot melt-extrusion, yielding slow-release polymer melts that release structurally intact and biologically active VNPs; in previous work virus-like particles (VLPs) derived from a bacteriophage were considered[42-46]—here Applicant apply these technologies to the plant virus CPMV.

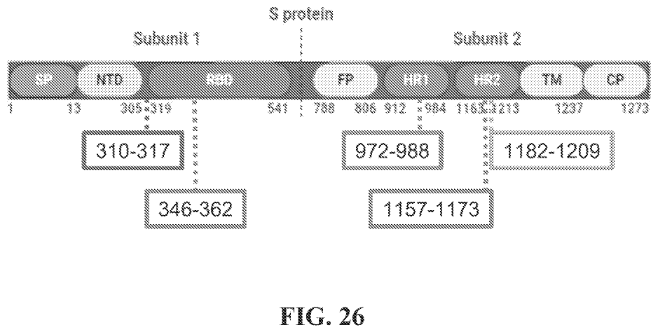

Selection of B-cell epitopes. Applicant selected five B-cell epitopes that were previously identified as targets of antibodies from sera of convalescent patients with SARS[37-38] or that were shown to neutralize SARS-CoV when used as a source of antigen in a vaccine candidate.[39] Four of the epitopes shared 100% sequence identity between the SARS-CoV and SARS-CoV-2 S proteins, whereas epitope 362 showed 76% sequence identity between the viruses (Table 7).[24] Two of the epitopes (317 and 362) are located within or immediately adjacent to the RBD, which binds the receptor ACE2, whereas the other three are located in the HR1/HR2 stalk region of the S2 subunit, which is required for cell fusion (FIG. 26).[47-48]

TABLE 7
B-cell epitopes from the SARS-CoV-2/
SARS-COV S proteins.

| Loca- | | Solu- | | | Iden- | |
|---|---|---|---|---|---|---|
| tion | Se- | bil- | | | tity | |
| (name)* | quence | ity** | pI | Length | %*** | Source |
| 310-317 | KGIYQ | Good | 9.5 | 8 | 100 | 24, 37 |
| | TSN | | | | | |
| 346-362 | ATRFA | Good | 11.8 | 17 | 76 | This |
| | SVYAW | | | | | work |
| | NRKRI | | | | | |
| | SN | | | | | |
| 972-988 | AISSV | Good | 4.2 | 17 | 100 | 24, 38 |
| | LNDIL | | | | | |
| | SRLDK | | | | | |
| | VE | | | | | |
| 1157-1173 | KNHTS | Good | 4.0 | 17 | 100 | 24, 38 |
| | PDVDL | | | | | |
| | GDISG | | | | | |
| | IN | | | | | |
| 1182-1209 | EIDRL | Good | 3.7 | 22 | 100 | 24, 39 |
| | NEVAK | | | | | |
| | NLNES | | | | | |
| | LIDLQ | | | | | |
| | EL | | | | | |

*Each peptide is named according to the C-terminal residue position and is color-coded.
**Theoretical solubility in water.
***Compared to SARS-CoV.

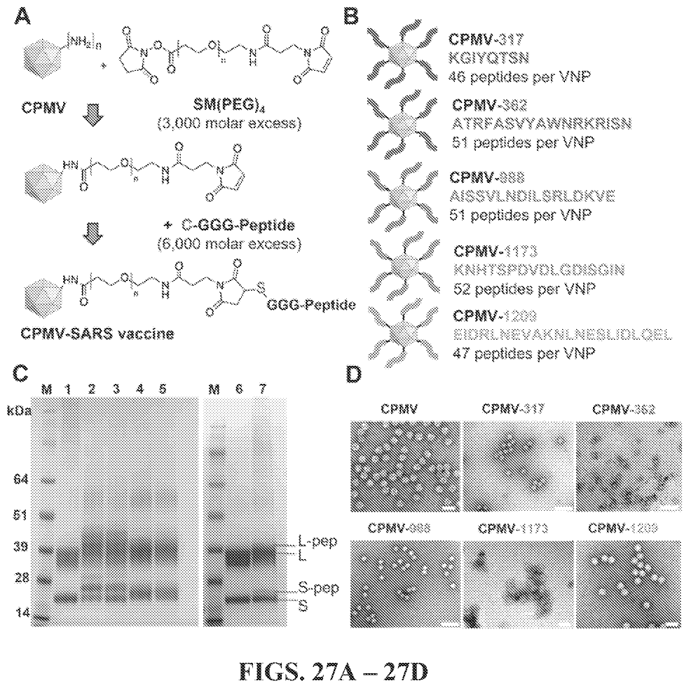

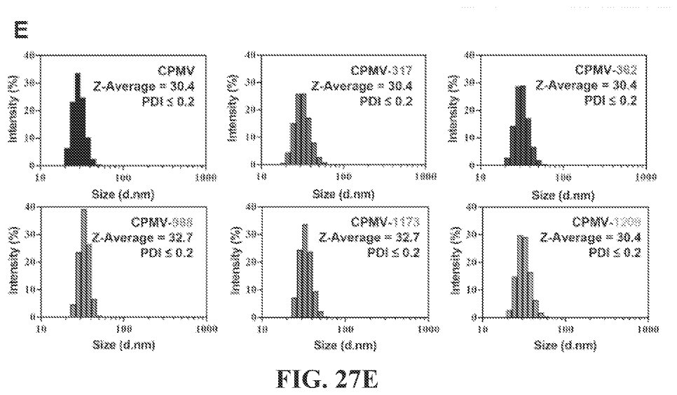

Preparation of CPMV-based vaccines. Each of the five B-cell epitopes was synthesized as a synthetic peptide with N-terminal cysteine followed by a GGG linker for conjugation to CPMV particles using a two-step procedure (FIG. 27A, FIG. 27B). The vaccines were purified by ultracentrifugation and characterized by DLS, TEM and SDS-PAGE under reducing conditions to confirm structural integrity and determine the degree of antigen incorporation (FIG. 27C-27E). SDS-PAGE analysis indicated that each VNP presented 46-52 peptides (FIG. 27C). Efficiency of the conjugations varied by peptide—it was interesting to note the band laddering effect for the CPMV-1173 and CPMV-1209 (FIG. 27C, lane 2+3) which indicates that more than one peptide was conjugated per coat protein; this is possible because the S and L protein each present with multiple lysine side chains (2 per S and 3 per L.[49] Nevertheless, the overall peptide loading per CPMV was comparable amongst the five vaccines. DLS and TEM confirmed that the VNPs were monodisperse before and after conjugation (FIG. 27D+FIG. 27E) and that their hydrodynamic diameter increased only marginally from 30.4 nm for the wild-type particles to 32.7 nm for each vaccine candidate (FIG. 27D, FIG. 27E). Applicant also observed no clouding of the suspension, therefore data indicate stable formulations free of protein aggregation.

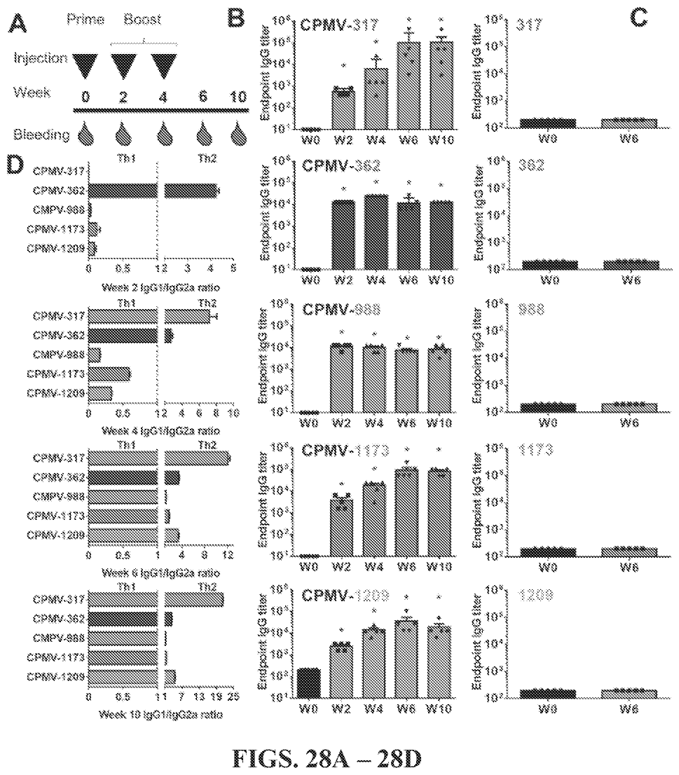

Immunogenicity of the CPMV-based vaccines. All five vaccines were injected into mice with a prime-boost-boost schedule and antibodies against all five peptides were recovered (FIG. 28A, FIG. 28B). Endpoint IgG titers against CPMV-317 increased steadily, from 1:560 (week 2) to 1:6160 (week 4), 1:100,480 (week 6) and 1:105,600 (week 10). For CPMV-988, IgG titers were already high after 2 weeks (1:11,520) and then gradually declined to 1:10,240 (week 4), 1:7680 (week 6) and 1:8320 (week 10). For CPMV-1173, IgG titers initially increased from 1:3840 (week 2) to 1:18,560 (week 4) and 1:92,160 (week 6) but then declined to 1:81,920 (week 10). Similarly, the IgG titers against CPMV-1209 increased from 1:2560 (week 2) to 1:14,080 (week 4) and 1:35,840 (week 6) before falling to 1:19,200 (week 10). Finally, endpoint IgG titers against CPMV-362 remained relatively constant at 1:12,800 (week 2), 1:25,600 (week 4), 1:11,520 (week 6) and 1:12,800 (week 10), albeit with a transient increase at week 4. As expected, free peptides were not immunogenic, and no IgG titers were detected after priming and two boosts (FIG. 28C).

Ig isotypes (FIG. 33) and IgG subclasses (FIG. 28D) were investigated to determine whether or not the candidates induced a Th cell response based on the IgG1/IgG2a ratio (values<1 defined as Th1-biased and values>1 defined as Th2-biased). After priming (week 2) all candidates except CPMV-362 showed a Th1-biased profile, but this switched to a Th2-biased profile after the second boost (FIG. 28D).

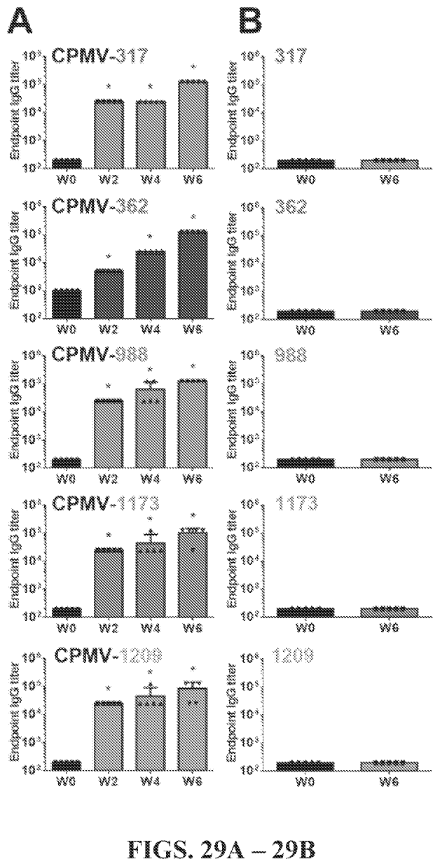

Immunoreactivity of serum against the SARS-CoV-2 S protein. The plasma samples used to determine IgG titers and isotype profiles were also used to provide a preliminary indication of immunoreactivity against the SARS-CoV-2 S protein. ELISA experiments revealed that IgG from the plasma samples taken on weeks 2, 4 and 6 from all mice injected with the vaccines was able recognize and the SARS-CoV-2 S protein (FIG. 29A). Four of the candidates showed week-2 titers of 1:25,000, the exception being CPMV-362 with a titer of only 1:5000. By week 6, three of the candidates achieved titers of 1:125,000, with CPMV-1173 slightly lower at 1:105,000 and CPMV-1209 lowest at 1:85,000. Plasma from mice injected with the free peptides did not bind to the S protein (FIG. 29B).

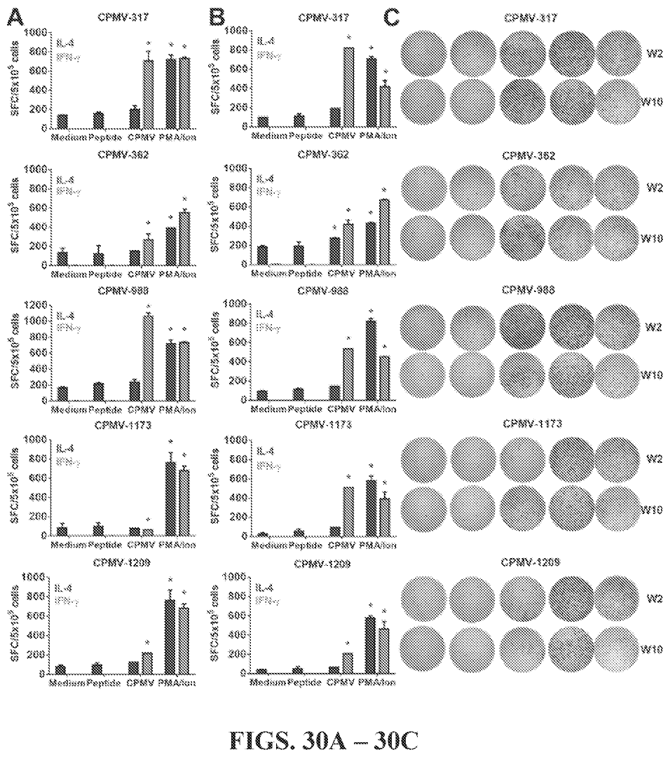

Analysis of T-cell responses to the vaccines. The T-cell responses to the vaccines were evaluated using an ELISpot assay following the vaccination of mice with a single dose (prime) or the complete schedule (prime and two boosters). Splenocytes were collected from immunized animals and stimulated with each peptide (317, 988, 1173, 1209 or 362), with normal cell culture medium as a negative control, or with CPMV plus PMA and ionomycin as a positive control. ELISpot analysis of splenocytes from animals receiving the vaccines (CPMV-317, CPMV-988, CPMV-1173, CPMV-1209 or CPMV-362) did not show the presence of IFN-γ spot-forming colonies (SFCs) when stimulated with the matching peptide regardless of whether the VNPs were delivered as a single dose (FIG. 30A) or three doses (FIG. 30B), which supports the evidence of a Th2-biased profile provided by the IgG subclass ratios (FIG. 30D). However, Applicant also observed no significant increase in the abundance of IL-4 SFCs following stimulation with the matching peptides (FIG. 30A, FIG. 30B), indicating that basal levels of IL-4 are sufficient for a Th2-biased response. Splenocytes stimulated with CPMV resulted in the appearance of more IFN-γ SFCs (FIG. 30A, FIG. 30B), demonstrating that CPMV itself triggers a Th1 response as previously reported. [50] As expected, stimulation with PMA and ionomycin triggered an increase in both IFN-γ and IL-4 SFCs. Representative images from each vaccine group and the various stimulants are shown in FIG. 30C.

Neutralization of SARS-CoV and SARS-CoV-2. Having confirmed the presence of IgG recognizing the peptide epitopes and S protein following immunization with all five vaccines, Applicant tested the plasma for the presence of neutralizing antibodies against SARS-CoV and SARS-CoV-2. Pooled plasma from each vaccinated group was pre-incubated separately with each virus and then added to Vero 76 cells to evaluate the impact on virus cytopathicity. The EC₅₀ and CC₅₀ were determined visually and from neutral red absorbance readings, and were used to calculate the SI₅₀ with values≥10 indicating neutralizing activity. The neutralization titers are reported in Table 8. Only the plasma from mice injected with CPMV-988, CPMV-1173 and CPMV-1209 was able to neutralize SARS-CoV, with similar neutralization titers but a slightly higher SI₅₀ value in the case of CPMV-1209 (Table 8). None of the samples were able to neutralize SARS-CoV-2 in the same assay (Table 8).

TABLE 8
Neutralization assay data for plasma samples taken
from mice injected with the five CPMV vaccines.

| | | SARS-CoV^{Ψ} | | | | | | |
|---|---|---|---|---|---|---|---|---|
| Plasma | Assay | | | | Neu. | SARS-CoV-2^{£} | | |
| sample | name^{#} | EC₅₀ | CC₅₀ | SI₅₀ | titer | EC₅₀ | CC₅₀ | SI₅₀ |
| CPMV- | Visual | 44 | >250 | 5.7 | 160 | >63 | >63 | 0 |
| 317 | Neutral red | 41 | >250 | 6.1 | 160 | >63 | >63 | 0 |
| CPMV- | Visual | 60 | >250 | 4.2 | 80 | >63 | >63 | 0 |
| 362 | Neutral red | 51 | >250 | 4.9 | 80 | >63 | >63 | 0 |
| CPMV- | Visual | 22 | >250 | 11 | 320 | >63 | >63 | 0 |
| 988 | Neutral red | 22 | >250 | 11 | 320 | >63 | >63 | 0 |
| CPMV- | Visual | 22 | >250 | 11 | 320 | >63 | >63 | 0 |
| 1173 | Neutral red | 23 | >250 | 11 | 320 | >63 | >63 | 0 |
| CPMV- | Visual | 14 | >250 | 18 | 320 | >63 | >63 | 0 |
| 1209 | Neutral red | 16 | >250 | 16 | 320 | >63 | >63 | 0 |
| CPMV | Visual | >250 | >250 | 0 | 40 | >63 | >63 | 0 |
| | Neutral red | >250 | >250 | 0 | 40 | >63 | >63 | 0 |

EC₅₀ = concentration (μg/ml) that reduces viral replication by 50%; CC₅₀ = concentration (μg/ml) that reduces cell viability by 50%; SI₅₀ = CC₅₀/EC₅₀; SI₅₀ values > 10 are considered as evidence of neutralizing activity. Neu. titer = viral neutralization titer at EC₅₀. Bold values represent the vaccines considered as neutralizing.
^{#}Each assay was performed to test the cytopathic effect.
^{Ψ}We used a range concentration of eight two-fold serial dilutions (250 to 1.9 μg/ml or dilution 1:40-1:5120).
^{£}We used a range concentration of eight two-fold serial dilutions (63 to 0.5 μg/ml or dilution 1:16-1:2048).

EC₅₀=concentration (g/ml) that reduces viral replication by 50%; CC₅₀=concentration (g/ml) that reduces cell viability by 50%; SI₅₀=CC₅₀/EC₅₀; S150 values>10 are considered as evidence of neutralizing activity. Neu. titer=viral neutralization titer at EC₅₀. Bold values represent the vaccines considered as neutralizing. ^{#}Each assay was performed to test the cytopathic effect. ^{Ψ}Applicant used a range concentration of eight two-fold serial dilutions (250 to 1.9 μg/ml or dilution 1:40-1:5120). ^{£}Applicant used a range concentration of eight two-fold serial dilutions (63 to 0.5 μg/ml or dilution 1:16-1:2048).

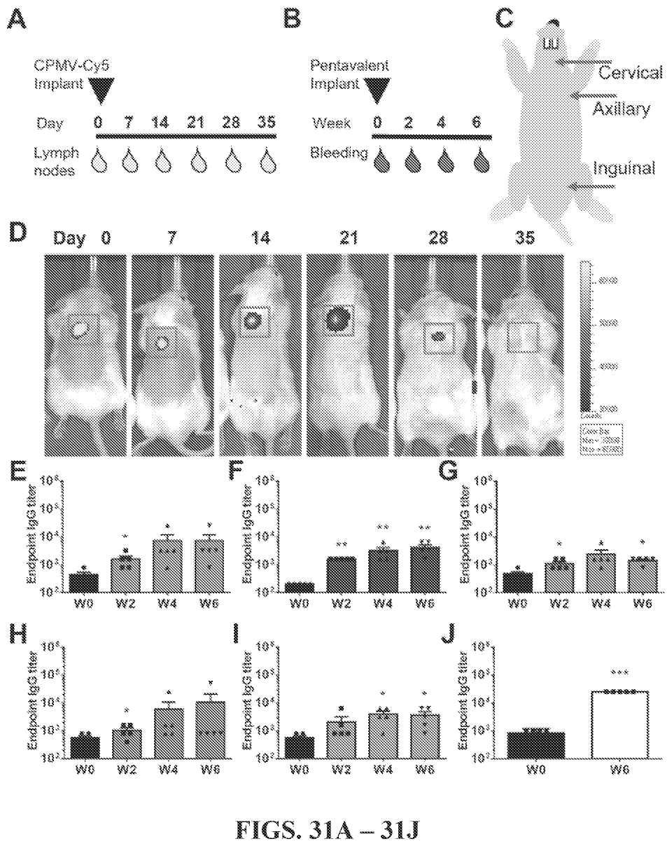

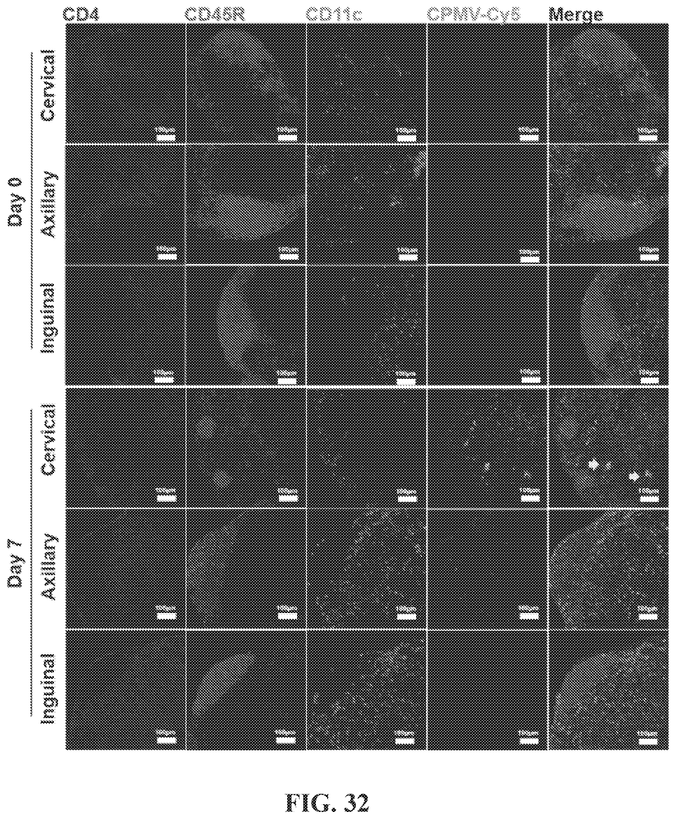

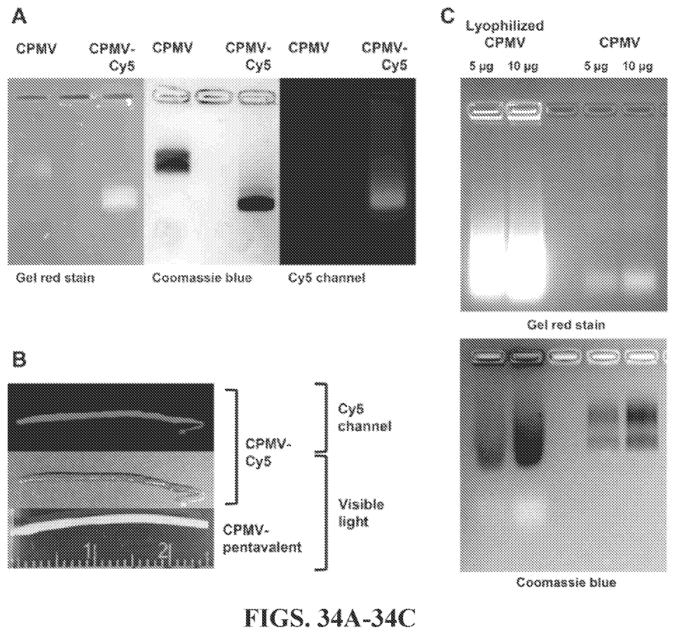

Analysis of pentavalent vaccine implants. The five CPMV-based vaccines were formulated as a cylindrical implant by hot-melt extrusion using a mixture of the pentavalent VNPs, PLGA and PEG8000 (10:75:15, w/w/w). The dose loaded for each pentavalent implant (300 μg of each CPMV vaccine) was equivalent to the three doses (100 μg each dose; prime-boost-boost) soluble injection schedule. The same approach was used to formulate CPMV-Cy5 VNPs (the characterization of CPMV-Cy5 is reported in the Supporting Information, FIG. 34) to trace their slow-release profiles post-implantation and their fate during lymph node drainage. Lymph nodes were collected every week for 5 weeks from animals carrying the CPMV-Cy5 implant (FIG. 31A) and blood was taken every 2 weeks for 6 weeks from animals carrying the pentavalent implant (FIG. 31B). Applicant collected cervical, axillary and inguinal lymph nodes, as shown in FIG. 31C. The gradual release of CPMV-Cy5 from the implant over 5 weeks was confirmed by the progressive loss of fluorescence from the implantation zone (FIG. 31D).

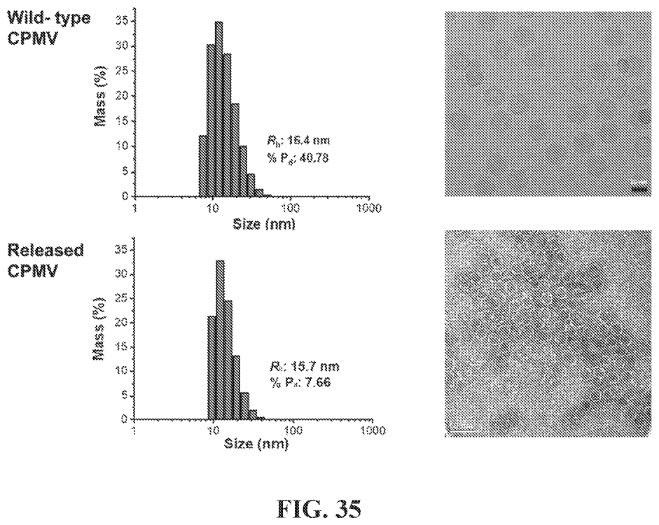

In addition to the fluorescence imaging studies of CPMV-Cy5/PLGA implants, Applicant also determined whether CPMV was intact when released from the implants in vitro. CPMV recovered from implants were subjected to DLS sizing experiments and TEM imaging. DLS and TEM are consistent with intact and monodispersed nanoparticles being released from the implants with sizes measuring ˜30 nm and indistinguishable features compared to CPMV (FIG. 35).

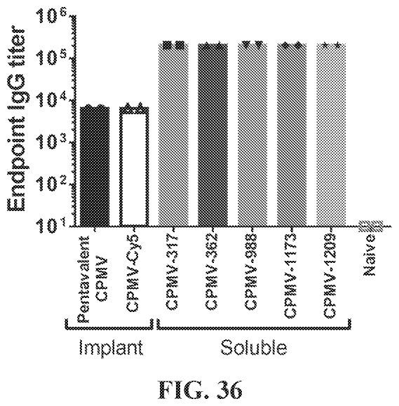

In vivo imaging experiments show that the loss of fluorescence from the CPMV-Cy5 implant matched the gradual increase in antibody titers against all five peptides (FIG. 31E-31I) and against the SARS-CoV-2 S protein (FIG. 31J) from the pentavalent implant, confirming that the released VNPs are immunogenic and stimulate the humoral immune response. The antibody titers against the CPMV carrier itself were 1:6400 for the pentavalent CPMV implant and CPMV-Cy5 implant, but 1:204,800 for all individually soluble injected CPMV vaccines (CPMV-317, CPMV-362, CPMV-988, CPMV-1173, and CPMV-1209) (FIG. 36). Furthermore, the IgG titers achieved by the pentavalent implant (FIG. 31E-31I) were reported for each individual vaccine (CPMV-317, CPMV-362, CPMV-988, CPMV-1173, and CPMV-1209) over the different time points (week 0 to week 6). For CPMV-317 titers were 1:1600, 1:7200, and 1:7200 (week 2, 4, and 6, respectively); for CPMV-362 titers were 1:1600, 1:3200, and 1:4160 (week 2, 4, and 6, respectively); for CPMV-988 titers were 1:1120, 1:2400, and 1:1440 (week 2, 4, and 6, respectively); for CPMV-1173 titers were 1:1040, 1:6080, and 1:10880 (week 2, 4, and 6, respectively); and for CPMV-1209 titers were 1:2080, 1:4000, and 1:3680 (week 2, 4, and 6, respectively). The antibody titer at week 6 against the SARS-CoV-2 S protein was 1:25000, which corresponds to all the IgG generated against the five epitopes used in the CPMV vaccines that can bind the S protein.

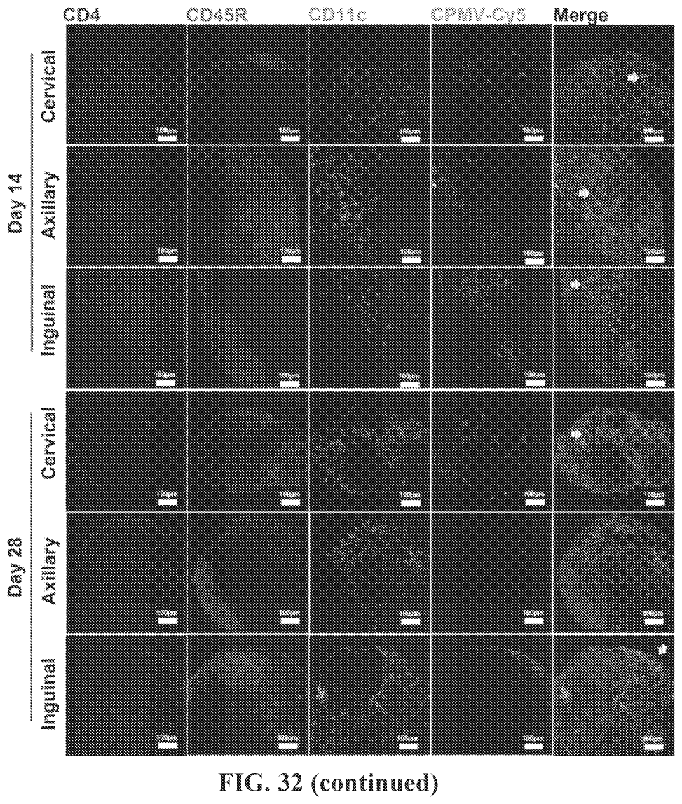

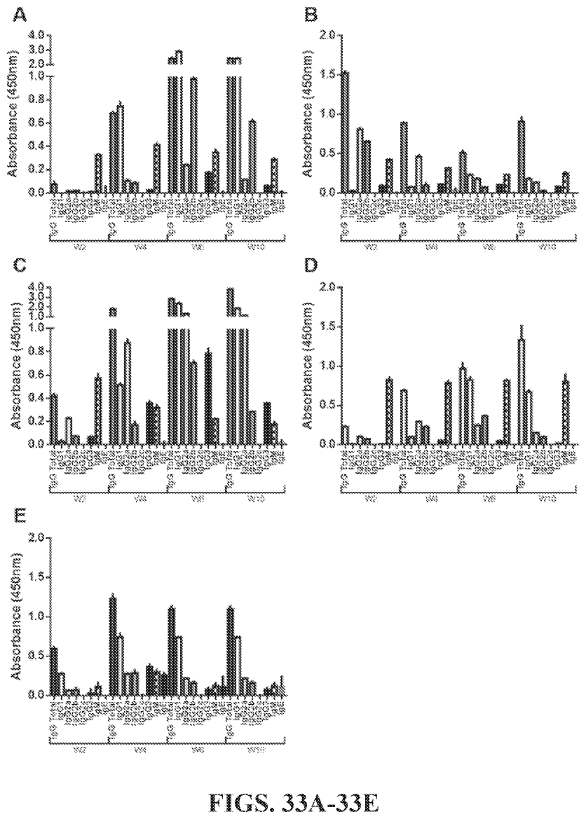

The fate of CPMV-Cy5 particles was traced in more detail by analyzing sections of cervical, axillary and inguinal lymph nodes on days 0, 7, 14, and 28 using antibodies against the markers CD4 (T cells), CD45R (B cells) and CD11c (dendritic cells) for co-localization (FIG. 7). On day 0, Applicant observed the presence of B cell-rich zones (CD45R, red panel) surrounded by T cells (CD4, blue panel) and a random distribution of dendritic cells (CD11c, green panel) within the T cell-rich zone in all lymph nodes. No background was observed from the Cy5 channel (CPMV-Cy5, gray panel). On day 7, Applicant detected the presence of CPMV-Cy5 in the cervical lymph nodes, including the formation of germination centers (FIG. 32, merged panel). A weak signal was also detected in the axillary lymph node but the presence of germination centers was not clear. On day 14, CPMV-Cy5 was present in all lymph nodes and germination centers were clearly present in axillary lymph nodes. The distribution of CPMV-Cy5 predominantly within the T cell-rich zone was more evident in the cervical and inguinal lymph nodes, but the CPMV-Cy5 signal in all lymph nodes was similar. Finally, on day 28, the CPMV-Cy5 signal was not detected in the axillary lymph nodes but a residual signal remained in the cervical lymph nodes and a slightly stronger signal in the inguinal lymph nodes. These observations suggest that CPMV-Cy5 drains initially to the lymph nodes closest to the implant zone, in this case the cervical lymph nodes (FIG. 32, day 7), followed by a broader distribution to all lymph nodes by day 14. Ultimately, the last VNPs from the implant drain to the closest (cervical) lymph nodes and therefore show a stronger signal than the farthest (inguinal) lymph nodes. This corresponds to the degradation profile of the implant, with ˜50% of the CPMV-Cy5 released by day 14 and almost all released by day 28 (FIG. 31D).

Discussion

Beta-coronaviruses share a small number of conserved B-cell and T-cell epitopes on the N and S proteins that suggest it may be possible to develop pan-beta-coronavirus vaccines that protect not only against known species such as SARS-CoV-2 but also new variants and even new species that could, if not tackled preemptively lead to future pandemics.[32-33] The S protein is considered the most suitable vaccine target for coronaviruses because it is responsible for interactions with host-cell receptors and cell fusion, and thus elicits neutralizing antibodies.[17] Applicant therefore selected five B-cell epitopes,[24] two within or adjacent to the RBD on the S1 subunit and three within the heptad repeats that define the stalk region of S2, which promotes membrane fusion (FIG. 26). Importantly, all but one of the epitopes showed 100% sequence identity between SARS-CoV and SARS-CoV-2 and were previously found to be immunogenic or reactive to sera from convalescent SARS-CoV patients.[37-39] Each epitope was (separately) conjugated to CPMV particles to produce soluble vaccines,[40] and Applicant also prepared a pentavalent implant by mixing equimolar amounts of all five candidates with PLGA and PEG to form a slow-release formulation.[51-53]

The display of peptides on the surface of plant viruses has been widely used as a strategy to develop vaccines, either by direct conjugation to virus particles or by the genetic engineering of coat proteins, but it is important to ensure that the peptides are compatible with virus assembly to avoid particle dissolution or aggregation.[54] DLS, TEM and SDS-PAGE was used to confirm the structural integrity of the particles and also to determine the degree of antigen incorporation (FIG. 27). DLS and TEM confirmed that the particles were monodisperse both before and after conjugation, indicating that conjugation neither destabilized the particles (resulting in them breaking into individual coat protein subunits) nor caused them to aggregate and precipitate from solution. SDS-PAGE analysis showed that each VNP presented 46-52 peptides suggesting comparable bioconjugation of all five peptides using the SM-(PEG)₄ linker and highlighting the flexibility of the CPMV platform in accommodating peptides ranging from 8 to 22 amino acid residues and varying pI of 3.7 to 11.8. (Table 7).

The five vaccines were then injected into mice, and Applicant found that plasma from the immunized animals was able to bind to the corresponding peptides in vitro whereas plasma from animals injected with free peptides was not (FIG. 28). These experiments confirmed that the CPMV vaccines were able to induce significant antibody titers after priming and two boosts. Notably, some candidates elicited high antibody titers that remained consistent over the 2-10 week time frame (CPMV-362 and CPMV-988) whereas others elicited initially lower antibody titers that increased over time (CPMV-317, CPMV-1173, and CPMV-1209). This highlights the potency of CPMV, which serves an antigen display and delivery technology, but also as a potent adjuvant that stimulates innate immune cells by signaling through the Toll-like receptors. Applicant recently demonstrated that CPMV signals through TLR-2, TLR-4 and TLR-7.[55] The temporal variability of antibody titers is likely influenced by the intrinsic properties of peptides (Table 7; hydrophobicity, length, or relative positioning of certain residues) and could be evaluated in future studies. For example, it would be useful to establish the attributes of peptides that elicit high antibody titers after single dose when displayed on CPMV, and whether the administration of single doses of CPMV-362 and CPMV-988 would achieve lasting high antibody titers similar to those obtained after the prime-boost schedule. Likewise, the five vaccines also differed in terms of the immune response based on the type of antibody produced. Interestingly, candidate CPMV-362 showed an immediate Th2-biased response whereas all other candidates showed an initial Th1-biased response that switched to a Th2-biased response after 2 weeks (CPMV-317) or 6 weeks (all others). VNP based vaccine platforms displaying multimeric self-epitopes or heterologous epitopes (target vaccine antigen) on the surface promote cross-linking of B-cell receptors (BCRs) that could prime B cells to induce the production of antibodies even without the help of CD4⁺ T cells.[56-57] CPMV, like other sub-200 nm nanoparticles, can diffuse and drain to lymph nodes without presentation on APCs, reaching zones rich in T and B cells in the lymph node periphery.[56-57] Early IgG2a (Th1-biased) responses may reflect fast and direct priming interactions between the CPMV vaccines and B cells in the lymph node, whereas later IgG1 (Th2-biased) responses after two boosts may favor APC presentation. Accordingly, although CPMV was thought to be a clear Th1 adjuvant for peptide vaccines,[50],[58] every new peptide/epitope must be tested on a case-by-case basis.

An early Th1 response was observed based on antibody isotypes, but the T-cell response did not mirror this response. Applicant expected to see the production of IFN-γ, a signature cytokine for the Th1 profile, after co-culturing the splenocytes from vaccinated mice with the same antigens used for the CPMV vaccines. The peptide alone may not be enough to stimulate the release of IFN-γ from T cells during the stimulation of splenocytes from vaccinated animals in vitro (FIG. 30) or the Th1-biased isotype may reflect a direct priming interaction between the CPMV vaccines and B cells, without the input of T cells to control isotype switching. These observations should be investigated in more detail in the future.

ELISAs confirmed that all five vaccines generated high titers of antibodies that bound to the corresponding peptides and to recombinant SARS-CoV-2 S protein (FIG. 29). However, in vitro neutralization assays against SARS-CoV and SARS-CoV-2 showed that plasma from only three candidates, namely CPMV-988, CPMV-1173 and CPMV-1209, were able to neutralize SARS-CoV. Nevertheless, none of the candidates were able to neutralize SARS-CoV-2 (Table 8). Possible explanations include the inaccessibility of corresponding epitopes on the S protein to the antibodies due to the conformation of the protein or the presence of post-translational modifications such as N-linked glycans, or the inability of antibodies binding these epitopes to block interactions with hACE-2 or cause conformational changes that prevent receptor interactions.[59]

The inability of the CPMV-317 and CPMV-362 candidates to neutralize either virus may reflect the unique properties of the displayed peptides: the small size of peptide 317 (eight residues) and/or the high pI of both peptides (9.5 and 11.8, respectively) may interfere with their ability to fold properly when displayed on the CPMV particle, thus eliciting antibodies that lack neutralizing efficacy. Notably, only CPMV-317 and CPMV-362 elicited an IgG1-predominant response, and these were also the only two candidates that did not neutralize SARS-CoV. Interestingly, although the other epitopes 988, 1173 and 1209 show 100% sequence identity in the SARS-CoV and SARS-CoV-2 S proteins, the antibodies elicited by the CPMV-based vaccines were only able to neutralize SARS-CoV. It is possible that the epitopes fold differently in the two viruses due to differences in the flanking residues that alter the overall conformation of the S protein.[60] However, a more likely explanation is the difference in glycosylation between the two viruses. There are 22 N-linked glycan sites on the SARS-CoV-2 S protein compared to 23 on SARS-CoV, with 18 of the sites common to both viruses, and the glycan shield density is lower in SARS-CoV than SARS-CoV-2 and MERS.[61-62] The epitopes used in CPMV-1173 (KNHTSPDVDLGDISGIN) and CPMV-1209 (EIDRLNEVAKNLNESLIDLQEL) both contain a conserved glycosylation site (underlined), representing positions 1155 and 1176 in the SARS-CoV S protein, and positions 1173 and 1194 in the SARS-CoV-2 S protein. The selective in vitro neutralization of SARS-CoV but not SARS-CoV-2, at least in the case of CPMV-1173 and CPMV-1209, may therefore reflect the different glycan shield density of each virus. However, it is unclear how this phenomenon affects CPMV-998, which does not contain a conserved glycosylation site and warrants further studies.

The COVID-19 pandemic highlights the need for innovation in vaccine design but also the need for more effective vaccine delivery strategies. The roll-out of mass vaccinations was burdened by the requirement of storage at ultra-low temperatures, delivery via injection thus requiring medical staff, and the requirement of a prime-boost vaccination schedule. Plant virus nanotechnologies hold potential to overcome the cold chain, because these materials are stable under various environmental conditions. Toward overcoming the need for repeated injections, Applicant evaluated the efficacy of a sustained release vaccine implant incorporating all five candidate vaccines. The five CPMV-based vaccines were formulated as a pentavalent implant by hot-melt extrusion with PLGA and PEG8000.[44],[51-53] Imaging studies revealed sustained release of CPMV in vivo and trafficking of CPMV to B and T cell rich regions of the draining lymph nodes. While antibody titers against the CPMV carrier were 32-fold lower from implant CPMV vs soluble injected CPMV vaccines (1:6400 vs 1:204800, respectively; FIG. 36). Applicant found that the IgG titers against the target epitopes produced using the CPMV/PLGA implants releasing the pentavalent vaccines were not as high as those induced by the corresponding soluble vaccines (FIG. 30 for implant vs FIG. 28 for soluble vaccines). The formulation process, which involves freezing, lyophilization and heat extrusion, could be the underlying factor and influence the immunogenicity of the particles. DLS and TEM data were consistent with intact CPMV being released from the polymer blends (FIG. 35); however, the lyophilization step resulted in loss of the RNA cargo (FIG. 34), which is consistent with our previous findings.[63] CPMV genomic RNA has been identified as a TLR-7 agonist, and its absence during antigen processing can mitigate the self-adjuvant properties of CPMV vaccines.[55] The difference of the antibody titers of CPMV implants compared to prime-boost formulations requires further analysis and may reflect a combination of RNA loss or other structural changes that influence subsequent interactions with (and activation of) innate immune cells. Future work will explore the addition of cryoprotectants before or during the lyophilization step to maintain the RNA cargo or addition of other TLR agonists as adjuvants. Furthermore, it cannot be ruled out that conformational changes of the epitope or carrier impact vaccine efficacy. In previous work with VLPs from a bacteriophage presenting human papilloma virus or cardiovascular disease-related epitopes, Applicant reported matched efficacy of the implant vs. soluble vaccine.[52-53] Another difference between the soluble prime-boost and slow-release implant is of course the dosing; 2 bolus doses vs. sustained by slower doses delivered by the implant. More research is needed to identify whether changes in antibody titers are based on the carrier or epitope properties post hot-melt extrusion or the delivery process itself.

Applicant screened five B-cell epitopes originally identified in the convalescent sera from recovered SARS patients by displaying them on the surface of CPMV. Three of these epitopes (peptides 988, 1173 and 1209) were found to be suitable for vaccine design. Immunization of mice using soluble formulation in a prime-boost-boost strategy elicited high antibody titers that neutralized SARS-CoV in vitro. The neutralizing vaccines (CPMV-988, CPMV-1173, and CPMV-1209) showed an early Th1-biased antibody profile (2-4 weeks) transitioning to a slightly Th2-biased profile after 6 weeks, just after the second boost. A pentavalent vaccine comprising all five peptides displayed on CPMV was administered as a slow-release implant, antibody titers were generated not as high as elicited by the soluble formulation and maintained the specificity against S protein. Sequence analysis revealed that the three epitopes (-988, -1173, and -1209) were 100% identical in SARS-CoV and SARS-CoV-2, but none of the vaccines were able to neutralize SARS-CoV-2 suggesting differences in the structural context perhaps caused by conformational changes or the presence of N-linked glycans. Plant virus nanotechnologies offer high thermal stability, thus overcoming the need for cold chain storage and distribution. The technology presented here therefore offers a highly versatile vaccination platform that can be pivoted toward other diseases and applications that are not limited to infectious diseases.

Methods

CPMV propagation. VNPs based on CPMV were propagated and purified as previously described.[64] Purified VNPs were stored in 0.1 M potassium phosphate (KP) buffer (pH 7.0) at 4° C. VNP concentrations were determined by UV spectroscopy at 260 nm using the molar extinction coefficient ε_{CPMV}=8.1 ml mg⁻¹ cm⁻¹.

Antigen characterization in silico. Applicant selected five B-cell epitopes[24] from the SARS-CoV-2 S protein (accession no. YP_009724390.1) as follows: 317=KGIYQTSN, 362=ATRFASVYAWNRKRISN, 988=AISSVLNDILSRLDKVE, 1173=KNHTSPDVDLGDISGIN, and 1209=EIDRLNEVAKNLNESLIDLQEL (FIG. 26). Applicant used an online peptide calculator (https://pepcalc.com/) to predict the molecular weights and isoelectric points. Applicant determined the sequence identity compared to the SARS-CoV S protein (accession no. YP_009825051.1) using protein BLAST (https://blast.ncbi.nlm.nih.gov/).

Synthesis and formulation of CPMV vaccines. The five B-cell epitopes appended with an N-terminal cysteine residue and triple glycine (GGG) linker were purchased from GenScript, with the following peptide sequences: 317=C-GGG-FKGIYQTSN, 362=C-GGG-ATRFASVYAWNRKRISN, 988=C-GGG-AISSVLNDILSRLDKVE, 1173=C-GGG-KNHTSPDVDLGDISGIN, and 1209=C-GGG-EIDRLNEVAKNLNESLIDLQEL. Using Applicant's two-step protocol,[65] each peptide epitope was conjugated to the CPMV capsid via the heterobifunctional N-hydroxysuccinimide (NHS)-PEG₄-maleimide linker SM-PEG₄ (Thermo Fisher Scientific) targeting the surface exposed lysine residues. Briefly, wild-type CPMV particles (2 mg/ml in KP buffer) were reacted with a 3000-fold molar excess of the SM-PEG₄ linker at room temperature for 2 h, followed by a 6000-fold molar excess of each peptide overnight. The resulting vaccines CPMV-317, CPMV-362, CPMV-988, CPMV-1173, and CPMV-1209 were purified using Amicon spin columns with a cut-off of 100 kDa (Sigma-Aldrich), resuspended in sterile KP buffer, and stored at 4° C.

Characterization of CPMV vaccines. To verify peptide conjugation, 10 μg of unmodified CPMV and purified CPMV vaccines (CPMV-317, CPMV-362, CPMV-988, CPMV-1173, and CPMV-1209) were compared by sodium dodecylsulfate polyacrylamide gel electrophoresis (SDS-PAGE) under reducing conditions on NuPAGE 4-12% Bis-Tris protein gels (Thermo Fisher Scientific) and stained with GelCode Blue Safe (ThermoFisher Scientific). Gel images were acquired using the ProteinSimple FluorChem® imaging system and lane density analysis with ImageJ 1.44o (http://imagej.nih.gov/ij) was used to determine the number of peptides conjugated per VNP. Particle integrity was confirmed by transmission electron microscopy (TEM) using a Tecnai Spirit G2 Bio TWIN (FEI Technologies) following 2% (w/v) uranyl acetate staining. Particle size was measured by dynamic light scattering (DLS) on a Zetasizer Nano (Malvern Instruments) at 25° C. in plastic disposable cuvettes.

Preparation of CPMV implants by hot-melt extrusion. Poly(lactic-co-glycolic acid) (PLGA) implants were prepared as previously described using our desktop melt-processing system.[45],[51-53] Briefly, PLGA powder with a 50:50 L:G ratio and a molecular weight of 10-15 kDa (Akina) was passed through a 45-mesh sieve (Sigma-Aldrich) for implant formulation. Lyophilized CPMV-317, CPMV-362 CPMV-988, CPMV-1173, and CPMV-1209 were mixed in equal amounts and then combined with PLGA and PEG8000 (Fisher Scientific) in the following ratio: 75% PLGA, 10% VNPs and 15% PEG8000 (w/w %). The components were mixed by vortexing, loaded into the hot melt-processing system, and heated to 70° C. for 90 s. Implants were extruded at a pressure of 10 psi applied to the piston. Implants were dried and stored with desiccants until use. CPMV-Cy5 implants were processed in the same manner.

Immunization. All animal experiments were carried out in compliance with guidelines from the UC San Diego Institutional Animal Care and Use Committee. Eight-week-old male BALB/c mice (Jackson Laboratory) were kept under standard conditions with food and water provided ad libitum. Five mice were assigned per experimental group. For the subcutaneous injection of liquid formulations, each CPMV vaccine candidate was concentrated at 1 mg/ml in phosphate-buffered saline (PBS; 137 mM NaCl, 2.7 mM KCl, 10 mM Na₂HPO₄, 1.8 mM KH₂PO₄, pH 7.4) and 100 μl was injected three times (100 μg/dose) at 2-week intervals (prime+two boosts). For the implant, a single dose containing 300 μg of each vaccine candidate (mimicking the 100 μg per dose subcutaneous injection) was administered using an 18G needle (BD Sciences) behind the neck. Applicant also administered 5 μg per dose of the free peptides (equivalent to peptide present in a 100 μg dose of CPMV-peptide vaccine) to a control group. Retro-orbital blood was collected in lithium/heparin-treated tubes (Thomas Scientific) just before injection or implantation (week 0) and then at weeks 2, 4, 6 and 10. Plasma was collected by centrifugation at 2000×g for 10 min at room temperature and was stored at −80° C.

IgG titers against peptides and S protein. Endpoint total IgG titers against each peptide epitope were determined by enzyme-linked immunosorbent assay (ELISA) in 96-well maleimide-activated plates (Thermo Fisher Scientific). Briefly, the plates were coated with 100 μl per well of each peptide (25 μg/ml in coating buffer: 0.1 M sodium phosphate, 0.15 M sodium chloride, 10 mM EDTA, pH 7.2) overnight at 4° C. After three washes with 200 μl per well of PBS+0.5% Tween-20 (PBST), the plates were blocked for 1 h at room temperature using 200 μl per well of 10 μg/ml 1-cysteine (Sigma-Aldrich). After washing again as above, plasma from immunized animals was prepared as two-fold serial dilutions in coating buffer and added to the plates. After incubation for 1 h at room temperature and another washing step, binding was detected using a horseradish peroxidase (HRP)-labeled goat anti-mouse IgG secondary antibody (Thermo Fisher Scientific) diluted 1:5000 in PBST (100 μl per well) for 1 h at room temperature. After a final washing step, Applicant added 100 μl per well of 1-Step Ultra TMB substrate (Thermo Fisher Scientific) and incubated for 10 min before stopping the reaction with 100 μl per well of 2 M H₂SO₄.

The IgG titer against SARS-CoV-2 S-protein was determined as described above but using 96-well nickel-activated plates (Thermo Fisher Scientific) coated with 200 ng His-tagged S protein per well (GenScript). Plasma samples were diluted five-fold in PBS and the same secondary antibody dilution and substrate was used to develop the assay as described above. The absorbance was read at 450 nm on a Tecan microplate reader. The endpoint antibody titers were defined as the reciprocal serum dilution at which the absorbance exceeded twice the background value (blank wells without plasma samples).

Antibody isotyping. The ELISA method set out above was adapted for antibody isotyping by testing samples from weeks 2, 4, 6 and 10 (diluted 1:1000 in coating buffer) with the following HRP-labeled secondary antibodies (Abcam) and dilutions: goat anti-mouse IgG1 (1:5000), IgG2a (1:1000), IgG2b (1:5000), IgG2c (1:5000), IgG3 (1:5000), IgM (1:5000), and IgE (1:1000). The IgG1/IgG2a ratio was reported for each group and a ratio higher than 1 was considered to indicate a Th2 response.

ELISpot assays. Briefly, 96-well ELISPOT plates (Cellular Technology) were coated with a 1:166 dilution of the anti-mouse interferon gamma (IFN-γ) and interleukin-4 (IL-4) capture antibodies overnight at 4° C. Splenocyte suspensions collected from three mice, 2 or 10 weeks post-immunization with each CPMV vaccine candidate, were added to the plates (5×10⁵ cells per well) following stimulation with 100 μl of medium alone (negative control), free peptide epitopes (20 μg/ml), unmodified CPMV (10 μg/ml), or 50 ng/ml phorbol 12-myristate 13-acetate (PMA) and 1 μg/ml ionomycin (Sigma-Aldrich) (positive control) at 37° C. and 5% CO₂ for 24 h. The plates were washed with PBST and then incubated with a 1:1000 dilution of FITC-labeled anti-mouse IFN-γ and a 1:666 dilution of biotin-labeled anti-mouse IL-4 antibodies at room temperature for 2 h. The plates were then washed with PBST and incubated at room temperature for 1 h with streptavidin-alkaline phosphatase (AP) and anti-FITC-HRP secondary antibodies (diluted 1:1000). Plates were washed with PBST and distilled water, then incubated with AP substrate for 15 min at room temperature, washed with distilled water and incubated with HRP substrate for 10 min at room temperature. Plates were then rinsed with water and air-dried at room temperature overnight. Colored spots were quantified using an S6 ENTRY Analyzer (Immunospot). The splenocytes from each animal were tested in triplicate for each stimulant.

Neutralization assays. The primary cytopathic effect assay[66] was carried out via the Preclinical Services offered by The National Institute of Allergy and Infectious Diseases (NIAID). SARS-CoV strain Urbani and SARS-CoV-2 strain USA_WA1/2020 were used to test neutralizing plasma. Briefly, confluent or near-confluent monolayers of Vero 76 cells were prepared in 96-well disposable microplates the day before testing. Cells were maintained in MEM (Sigma-Aldrich) supplemented with 5% fetal bovine serum (FBS) and were tested in the same medium with the FBS concentration reduced to 2% and supplemented with 50 μg/ml gentamicin. The pooled plasma samples (week 6 post-immunization) representing each CPMV vaccine candidate and unmodified CPMV as a negative control were prepared as 10-fold serial dilutions. Five microwells were used per dilution: three for infected cultures and two for uninfected toxicity cultures. Controls consisted of six wells that were infected and not treated (virus controls) and six that were untreated and uninfected (cell controls) on every plate. Plasma samples were mixed with the virus (1:1 ratio) and incubated for 1 h at 37° C. The growth medium was then removed from the cells and the plasma/virus mixture was applied (0.1 ml per well). For the virus infection controls, the virus was added typically at ˜60 CCID₅₀ (50% cell culture infectious dose) in 0.1 ml of medium. Medium without virus was added to the toxicity control and cell control wells. Plates were incubated at 37° C. in a 5% CO₂ incubator until a cytopathic effect CPE>80% was observed in the virus control wells. The plates were then stained with 0.011% neutral red for ˜2 h at 37° C. in a 5% CO₂ incubator. The neutral red medium was removed, and the cells rinsed with PBS to remove residual dye. The PBS was completely removed, and the incorporated neutral red was eluted with 50% Sorensen's citrate buffer/50% ethanol for at least 30 min. The dye content in each well, proportional to the number of living cells, was quantified by spectrophotometry at 540 nm. The dye content in each set of wells was converted to a percentage of the dye present in untreated control wells and normalized against the virus control. The 50% effective concentration (EC₅₀, virus inhibition) and 50% cytotoxic concentration (CC₅₀, cell inhibition) were calculated by regression analysis. The CC₅₀/EC₅₀ quotient was used to calculate the selectivity index (SI) and plasma samples with SI≥10 were considered as neutralizing.

Immunofluorescence imaging of lymph nodes. Cy5-labeled CPMV nanoparticles were prepared for imaging studies. Fluorescent CPMV-Cy5 particles were synthesized by conjugating N-hydroxysuccinimide-activated esters of Sulfo-Cy5 (NHS-Sulfo-Cy5, Lumiprobe) to the CPMV capsid via the surface exposed lysine residues. Briefly, a 1500 molar excess of NHS-sulfo-Cy5 was reacted overnight with CPMV in 0.1 M KP buffer (pH 7.4) plus 10% (v/v) DMSO and a protein concentration of 2 mg/ml. Following the reaction, Cy5-conjugated particles were purified from the excess unreacted dye by ultracentrifugation (112000×g, 1 h). The pellet was then resuspended in 0.1 M KP buffer. The CPMV concentration and number of dye molecules per capsid were determined by UV/vis spectrophotometry using the CPMV extinction coefficient (εCPMV)=8.1 mL mg⁻¹ cm⁻¹ at 260 nm and the NHS-sulfo-Cy5 specific molar extinction coefficient (εsulfo-Cy5)=27,1000 L mol⁻¹ cm⁻¹ at 647 nm. CPMV-Cy5 implants were prepared as described above and introduced subcutaneously via an 18G needle by pushing the implant out of the needle using a sterilized stainless-steel wire (0.51 mm diameter). Fluorescence images were acquired on an IVIS 200 imaging system at different time points and were analyzed using Living Image v3.0. To determine the fate of VNPs in the implant, cervical, axillary and inguinal lymph nodes were collected from the mice in 10% formalin-buffered solution (Sigma-Aldrich) on days 0, 7, 14, 21, 28 and 35 after CPMV-Cy5 implant administration. Two mice were euthanized on each day. The lymph nodes were embedded in paraffin and tissue sections were stained for the cell surface markers CD11c (LS Bio, 1:25), CD45R (Abcam, 1:50) and CD4 (Abcam, 1:100). A donkey anti-rabbit-IgG secondary antibody conjugated to AlexaFluor 488 (Invitrogen, 1:500) was used to detect primary antibody binding. The sections were imaged on a Nikon A1R confocal microscope with an ×20, 0.75 numerical aperture dry objective. Portions from two lymph node sections were imaged for each cell marker and the corresponding channels were superimposed by aligning the DAPI signal to form composite images using Nikon Analysis software.

Statistical analysis. Data are presented as mean±SEM. Single comparisons based on an unpaired, two-tailed t-test were carried out using SPSS Statistics software or GraphPad Prism 6. Differences were considered significant at p<0.05. The number of replicates is described for each experiment.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### Embodiments

1. A method for one or more of the following in a subject in need thereof: treating or preventing a cardiovascular disease, treating or preventing an atherosclerosis, treating or preventing a hypercholesterolemia, treating or preventing a lipid dyshomeostasis, preventing a heart attack, preventing a stroke, reducing a statin administration dose or frequency or both, reducing a cholesterol level, reducing an oxidized cholesterol level, reducing a low-density lipoprotein cholesterol (LDL-C) level, reducing a level or an activity of one or more cholesterol checkpoint protein(s), producing an antibody recognizing and binding to one or more cholesterol checkpoint protein(s), triggering, enhancing, or prolonging an immune response to one or more cholesterol checkpoint protein(s), or delivering at least one epitope(s) of one or more cholesterol checkpoint protein(s) to the subject, comprising, or consisting essentially of, or consisting of administering to the subject one or more virus or virus-like particle(s), wherein each virus or virus-like particle comprises at least one epitope of the cholesterol checkpoint protein(s), and optionally comprising two or more epitopes that may be the same or different from each other.

2. The method of Embodiment 1, wherein one or more of the following is achieved: delivering at least one epitope of the cholesterol checkpoint protein(s) to the subject, producing an antibody recognizing and binding to the one or more cholesterol checkpoint protein(s) in the subject, triggering, enhancing, or prolonging an immune response to the one or more cholesterol checkpoint protein(s), reducing the level or the activity of the one or more cholesterol checkpoint protein(s) in the subject, reducing the total cholesterol level in the subject, reducing the oxidized cholesterol level in the subject, reducing the LDL-C level in the subject, reducing a statin administration dose or frequency or both, treating or preventing a cardiovascular disease, treating or preventing an atherosclerosis, treating or preventing a hypercholesterolemia, treating or preventing a lipid dyshomeostasis, preventing a heart attack, or preventing a stroke.

3. The method of Embodiment 1 or 2, wherein the epitope comprises, or consists essentially of, or consists of a fragment of the cholesterol checkpoint protein. Additionally or alternatively, a fragment of the cholesterol checkpoint protein comprises, or consists essentially of, or consists of the epitope.

4. The method of any one of Embodiments 1-3, wherein the cholesterol checkpoint protein(s) is selected from any one or any two or all three of: proprotein convertase subtilisin/kexin-9 (PCSK9), apolipoprotein B (ApoB), or cholesteryl ester transfer protein (CETP).

5. The method of any one of Embodiments 1-4, wherein each of the virus or virus-like particle(s) comprises at least one epitope peptide comprising, or consisting essentially of, or consisting of an amino acid sequence selected from KTTKQSFDLSVKAQYKKNKH (SEQ ID NO: 1, which is a fragment of ApoB), FGFPEHLLVDFLQSLS (SEQ ID NO: 2, which is a fragment of CETP), or NVPEEDGTRFHRQASKC (SEQ ID NO: 3, which is a fragment of PSCK9).

6. The method of any one of Embodiments 1-5, wherein the method comprises, or consists essentially of, or further consists of administering to the subject a plurality of virus or virus-like particles, each of the virus or virus-like particles comprising at least one epitope(s), or one or more peptide(s) each of which comprises, or consists essentially of, or further consists of at least one of the epitope(s), or one or more fragment(s) of the cholesterol checkpoint protein(s) each of which comprises, or consists essentially of, or further consists of at least one of the epitopes, the epitopes being the same or different from each other.

7. The method of any one of Embodiments 1-6, wherein the method comprises, or consists essentially of, or further consists of administering to the subject three virus or virus-like particles, wherein a first virus or virus-like particle comprises an epitope peptide comprising, or consisting essentially of, or consisting of an amino acid sequence of KTTKQSFDLSVKAQYKKNKH (SEQ ID NO: 1), a second virus or virus-like particle comprises an epitope peptide comprising, or consisting essentially of, or consisting of an amino acid sequence of FGFPEHLLVDFLQSLS (SEQ ID NO: 2), and a third virus or virus-like particle comprises an epitope peptide comprising, or consisting essentially of, or consisting of an amino acid sequence of NVPEEDGTRFHRQASKC (SEQ ID NO: 3).

8. The method of any one of Embodiments 1-6, wherein the method comprises, or consists essentially of, or further consists of administering to the subject two virus or virus-like particles, wherein a first virus or virus-like particle comprises an epitope peptide comprising, or consisting essentially of, or consisting of an amino acid sequence selected from one of KTTKQSFDLSVKAQYKKNKH (SEQ ID NO: 1), FGFPEHLLVDFLQSLS (SEQ ID NO: 2), or NVPEEDGTRFHRQASKC (SEQ ID NO: 3), and a second virus or virus-like particle comprises the rest two amino acid sequencers in one or two epitope peptide(s).

9. The method of any one of Embodiments 1-5, wherein the method comprises, or consists essentially of, or further consists of administering to the subject one virus or virus-like particle which comprises at least two epitope(s), or one or more peptide(s) each of which comprises one or more of the at least two epitope(s), or one or more fragment(s) of the cholesterol checkpoint protein(s) each of which comprises one or more of the at least one epitope(s), wherein the epitopes may be the same are different from each other.

10. The method of any one of Embodiments 1-5 and 9, wherein the method comprises, or consists essentially of, or further consists of administering to the subject one virus or the virus-like particle which comprises the following three amino acid sequences KTTKQSFDLSVKAQYKKNKH (SEQ ID NO: 1), FGFPEHLLVDFLQSLS (SEQ ID NO: 2), and NVPEEDGTRFHRQASKC (SEQ ID NO: 3) in one or two or three epitope peptide(s).

11. The method of any one of Embodiments 1-10, wherein the epitope(s), a peptide comprising, or consisting essentially of, or consisting of at least one of the epitope(s), or a protein fragment comprising, or consisting essentially of, or consisting of at least one of the epitope(s) is present on the outer surface of the virus or the virus-like particle.

12. The method of any one of Embodiments 1-10, wherein the epitope(s), a peptide comprising, or consisting essentially of, or consisting of at least one of the epitope(s), or a protein fragment comprising, or consisting essentially of, or consisting of at least one of the epitope(s) is conjugated directly or indirectly to the virus or the virus-like particle, or a coat protein of the virus or the virus-like particle.

13. The method of any one of Embodiments 1-12, wherein the epitope(s), a peptide comprising, or consisting essentially of, or consisting of at least one of the epitope(s), or a protein fragment comprising, or consisting essentially of, or consisting of at least one of the epitope(s) is conjugated indirectly by a method comprising, or consisting essentially of, or yet further consisting of a linker to the virus or virus-like particle, or a coat protein of the virus or the virus-like particle.

14. The method of Embodiment 13, wherein the linker comprises, or consists essentially of, or further consists of an amino acid sequence of GSG, GPSL, or GGSGGGSG, or wherein the linker is an SM(PEG)₈ bifunctional linker comprising, or consisting essentially of, or consisting of an NHS group and a maleimide group, or wherein the linker comprises, or consists essentially of, or further consists of an N-terminal cysteine residue conjugated to triple glycine (GGG) and an N-hydroxysuccinimide-PEG4-maleimide linker SM-PEG4.

15. The method of any one of Embodiments 1-14, wherein the C-terminus or the N-terminus of the epitope(s), a peptide comprising, or consisting essentially of, or consisting of at least one of the epitope(s), or a protein fragment comprising, or consisting essentially of, or consisting of at least one of the epitope(s) is conjugated directly or indirectly to the N-terminus or the C-terminus of a coat protein of the virus or the virus-like particle.

16. The method of any one of Embodiments 1-15, wherein the virus or the virus-like particle is a bacteriophage virus or virus like particle, or a plant virus or virus like particle.

17. The method of any one of Embodiments 1-16, wherein the virus or the virus-like particle is a bacteriophage Qβ virus or virus-like particle.

18. The method of any one of Embodiments 1-16, wherein the virus or the virus-like particle is a plant picornavirus virus or virus-like particle, or a filamentous plant virus or virus-like particle.

19. The method of Embodiment 17 or 18, wherein the plant virus or the virus-like particle is of the or Alphafexiviridae family.

20. The method of Embodiment 16 or 18, wherein the plant virus or virus-like particle is a cowpea mosaic virus-like particle or a potato virus X virus-like particle.

21. The method of Embodiment 16 or 18, wherein the plant virus or the virus-like particle is a rod-shaped virus or virus-like particle.

22. The method of Embodiment 21, wherein the rod-shaped virus or the virus-like particle is a tobacco mosaic virus or virus-like particle.

23. The method of any one of Embodiment 1-22, wherein the level is in a biological sample of the subject that is optionally selected from: plasma, peripheral blood, or serum.

24. The method of any one of Embodiments 1-23, wherein the method does not comprises repeating the administering step.

25. The method of any one of Embodiments 1-24, wherein the virus or virus-like particle(s) is administrated to the subject in a slow-release implant.

26. The method of any one of Embodiments 1-25, wherein the virus or virus-like particle(s) is encapsulated within a degradable polymer matrix.

27. The method of Embodiment 26, wherein the degradable polymer matrix comprises, or consists essentially of, or further consists of a melt processable degradable polymer material that is biocompatible and, upon degradation, produces substantially non-toxic products, wherein the melt processable degradable polymer material is a melt processable biodegradable polymer, and wherein the degradable polymer material has a melt temperature below the degradation temperature of the virus or virus-like particle(s).

28. The method of Embodiment 27, wherein the degradable polymer material comprises, or consists essentially of, or further consists of poly(lactic-co-glycolic acid) (PLGA) or a copolymer thereof.

29. The method of any one of Embodiments 25-28, wherein the slow-release implant comprises, or consists essentially of, or consists of one or more of: about 50% to about 99% PLGA, about 1% to about 50% virus or virus-like particle(s), or PEG8000, or wherein the slow-release implant comprises, or consists essentially of, or further consists of one or more of: about 80% PLGA, about 10% virus or virus-like particle(s), or about 10% PEG8000 optionally by weight, or wherein the slow-release implant comprises, or consists essentially of, or further consists of one or more of: about 75% PLGA, about 10% VNPs, or about 15% PEG8000, optionally wherein the % is indicated a weight percentage.

30. The method of any one of Embodiments 25-29, wherein the slow-release implant is administered by a microneedle patch or by injection.

31. The method of any one of Embodiments 1-23 and 25-30, comprising repeating the administering step for about once, about twice, about three times, about four times, about five times, or more, and wherein two subsequent administrations are about 1 day to about 1 year apart, or about 1 week apart, or about 2 weeks apart, or about 3 weeks apart, or about 4 weeks apart, or about 1 month apart, or about 2 months apart, or about 3 months apart, or about 6 months apart.

32. The method of Embodiment 31, wherein the administering step is not repeated for more than 6 times, or more than 7 times, or more than 8 times, or more than 9 times, or more than 10 times, or more than 15 times, or more than 20 times.

33. The method of any one of Embodiments 1-32, whereby the epitope(s) does not activate a T cell or a cytotoxic T cell in the subject.

34. The method of any one of Embodiments 1-33, wherein the method does not comprise administering to the subject an additional adjuvant.

35. The method of any one of Embodiments 1-34, wherein the virus or virus-like particle(s) comprises at least one epitope from each of PCSK9, ApoB, and CETP, and whereby achieving one or more of the following effects in synergy: reducing the total cholesterol level in the subject, reducing the oxidized cholesterol level in the subject, reducing the LDL-C level in the subject, reducing a statin administration dose or frequency or both, treating or preventing a cardiovascular disease, treating or preventing an atherosclerosis, treating or preventing a hypercholesterolemia, treating or preventing a lipid dyshomeostasis, preventing a heart attack, or preventing a stroke.

36. A method for one or more of the following in a subject in need thereof: treating or preventing an infectious disease or another disease caused by a pathogen, producing an antibody recognizing and binding to one or more pathogen(s) causing the disease, triggering, enhancing, or prolonging an immune response to one or more pathogen(s) causing the disease, or delivering at least one epitope(s) of the pathogen(s) causing the disease to the subject comprising, or consisting essentially of, or consisting of administering to the subject one or more virus or virus-like particle(s), each of which comprises at least one epitope of the pathogen and optionally comprising two or more epitopes that may be the same or different from each other.

37. The method of Embodiment 36, wherein the pathogen is Human papillomavirus (HPV).

38. The method of Embodiment 37, wherein the disease is an HPV infection, or a cancer, or both.

39. The method of Embodiment 38, wherein the cancer is selected from: a cervical cancer, an oropharyngeal cancer, an anal cancer, a penile cancer, a vaginal cancer, or a vulva cancer.

40. The method of any one of Embodiments 36-39, wherein the epitope(s) is of an HPV capsid protein.

41. The method of any one of Embodiments 37-40, wherein the HPV is selected from one or more of HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, or HPV82.

42. The method of Embodiment 40 or 41, wherein the capsid protein comprises, or consists essentially of, or further consists of an HPV capsid protein L1 or an HPV capsid protein L2, or both.

43. The method of any one of Embodiments 37-42, wherein the epitope(s) is of a HPV16 capsid protein L2.

44. The method of any one of Embodiments 37-43, wherein the epitope(s) is present in a peptide comprising, or consisting essentially of, or consisting of an amino acid sequence of QLYKTCKQAGTCPPD (SEQ ID NO: 4, which is amino acid 17 to amino acid 31 of HPV16 L2 protein), or a fragment of an HPV capsid protein aligned with SEQ ID NO: 4.

45. The method of Embodiment 36, wherein the pathogen is a coronavirus.

46. The method of Embodiment 45, wherein the coronavirus is a severe acute respiratory syndrome (SARS) associated coronavirus (SARS-CoV).

47. The method of Embodiment 46, wherein the SARS-CoV comprises, or consists essentially of, or further consists of SARS-CoV-1 (also referred to herein as SARS), or SARS-CoV-2, or both SARS-CoV-1 and SARS-CoV-2.

48. The method of any one of Embodiments 36 and 45-47, wherein the epitope(s) is of a spike protein (S protein) of a coronavirus.

49. The method of any one of Embodiments 45-48, wherein the epitope(s) is present in one or more peptide(s) comprising, or consisting essentially of, or consisting of at least one amino acid sequence(s) selected from KGIYQTSN (SEQ ID NO: 5, amino acid (aa) 310 to aa 317 of SARS-CoV-2 S protein), AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), EIDRLNEVAKNLNESLIDLQEL (SEQ ID NO: 8, amino acid (aa) 1182 to aa 1209 of SARS-CoV-2 S protein), ATRFASVYAWNRKRISN (SEQ ID NO: 9, amino acid (aa) 346 to aa 362 of SARS-CoV-2 S protein), YNSASFSTFKCYGVSPTK (SEQ ID NO: 10, aa 369 to aa 386 of SARS-CoV-2 S protein), LPDPSKPSKRSFIED (SEQ ID NO: 11, aa 806 to aa 820 of SARS-CoV-2 S protein), FRKSN (SEQ ID NO: 12, aa 456 to aa 460 of SARS-CoV-2 S protein), PSKPSKRSFIEDLLFNKV (SEQ ID NO: 13, aa 809 to aa 826 of SARS-CoV-2 S protein), TESNKKFLPFQQFGRDIA (SEQ ID NO: 14, aa 553 to aa 570 of SARS-CoV-2 S protein), TESNKKFLPFQQ (SEQ ID NO: 15, aa 553 to aa 564 of SARS-CoV-2 S protein), HADQLTPTWRVY (SEQ ID NO: 16, aa 625 to aa 636 of SARS-CoV-2 S protein), FKEELDKYFKNH (SEQ ID NO: 17, aa 1148 to aa 1159 of SARS-CoV-2 S protein), FASTEKSNIIRGWIF (SEQ ID NO: 18, aa 92 to aa 106 of SARS-CoV-2 S protein), PFLGVYYHKNNKSWM (SEQ ID NO: 19, aa 135 to aa 153 of SARS-CoV-2 S protein), EVRQIAPGQTGKIAD (SEQ ID NO: 20, aa 406 to aa 420 of SARS-CoV-2 S protein), NNLDSKVGGNYNYLYR (SEQ ID NO: 22, aa 439 to aa 454 of SARS-CoV-2 S protein), LFRKSNLKPFERDIS (SEQ ID NO: 22, aa 455 to aa 469 of SARS-CoV-2 S protein), or a fragment of a coronavirus S protein aligned with each thereof, the epitopes being the same or different from each other.

50. The method of any one of Embodiments 45-49, wherein the epitope(s) is present in one or more peptide(s) comprising, or consisting essentially of, or consisting of at least one amino acid sequence(s) selected from AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), EIDRLNEVAKNLNESLIDLQEL (SEQ ID NO: 8, amino acid (aa) 1182 to aa 1209 of SARS-CoV-2 S protein), YNSASFSTFKCYGVSPTK (aa 369 to aa 386 of SARS-CoV-2 S protein), PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), PFLGVYYHKNNKSWM (aa 135 to aa 153 of SARS-CoV-2 S protein), or a fragment of a coronavirus S protein aligned with each thereof, or wherein the epitope(s) is present in one or more peptide(s) comprising, or consisting essentially of, or consisting of at least one amino acid sequence(s) selected from AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), or EIDRLNEVAKNLNESLIDLQEL (SEQ ID NO: 8, amino acid (aa) 1182 to aa 1209 of SARS-CoV-2 S protein), or a fragment of a coronavirus S protein aligned with each thereof, or wherein the epitope(s) is present in one or more peptide(s) comprising, or consisting essentially of, or consisting of at least one amino acid sequence(s) selected from YNSASFSTFKCYGVSPTK (aa 369 to aa 386 of SARS-CoV-2 S protein), PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), PFLGVYYHKNNKSWM (aa 135 to aa 153 of SARS-CoV-2 S protein), or a fragment of a coronavirus S protein aligned with each thereof, or wherein the epitope(s) is present in one or more peptide(s) comprising, or consisting essentially of, or consisting of at least one amino acid sequence(s) selected from PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), or a fragment of a coronavirus S protein aligned with each thereof.

51. The method of any one of Embodiments 36-50, comprising, or consisting essentially of, or consisting of administering to the subject one or more virus or virus-like particles, each of which comprises at least one epitope(s), or one or more peptide(s) each of which comprises, or consists essentially of, or further consists of at least one of the epitope(s), or one or more protein fragment(s) each of which comprises, or consists essentially of, or further consists of at least one of the epitope(s).

52. The method of any one of Embodiments 36-51, comprising, or consisting essentially of, or consisting of administering to the subject one or more virus or virus-like particles, each of which comprises as least one peptide as disclosed.

53. The method of any one of Embodiments 36-52, comprising, or consisting essentially of, or consisting of administering to the subject two or more virus or virus-like particles, each of which comprises at least one epitope(s), or one or more peptide(s) each of which comprises, or consists essentially of, or further consists of at least one of the epitope(s), or one or more protein fragment(s) each of which comprises, or consists essentially of, or further consists of at least one of the epitope(s).

54. The method of any one of Embodiments 36-53, comprising, or consisting essentially of, or consisting of administering to the subject two or more virus or virus-like particles, each of which comprises at least one peptide as disclosed.

55. The method of any one of Embodiments 36-52, comprising, or consisting essentially of, or consisting of administering to the subject one virus or virus-like particle which comprises as least one epitope or peptide as disclosed.

56. The method of any one of Embodiments 36-52 and 55, comprising, or consisting essentially of, or consisting of administering to the subject one virus or virus-like particle comprising at least two epitope(s), or one or more peptide(s) each of which comprises, or consists essentially of, or further consists of at least one of the epitope(s), or one or more protein fragment(s) each of which comprises, or consists essentially of, or further consists of at least one of the epitope(s).

57. The method of any one of Embodiments 36-52 and 55-56, comprising, or consisting essentially of, or consisting of administering to the subject one virus or virus-like particle comprising at least two peptides as disclosed.

58. The method of any one of Embodiments 36-57, wherein the epitope(s), a peptide comprising thereof, or a protein fragment comprising thereof is present on the outer surface of the virus or virus-like particle.

59. The method of any one of Embodiments 36-58, wherein the epitope(s), a peptide comprising, or consisting essentially of, or consisting of at least one of the epitope(s), or a protein fragment comprising, or consisting essentially of, or consisting of at least one of the epitope(s) is conjugated directly or indirectly to the virus or virus-like particle, or a coat protein of the virus or virus-like particle.

60. The method of any one of Embodiments 36-59, wherein the epitope(s), a peptide comprising, or consisting essentially of, or consisting of at least one of the epitope(s), or a protein fragment comprising, or consisting essentially of, or consisting of at least one of the epitope(s) is conjugated indirectly comprising, or consisting essentially of, or yet further consisting of a linker to the virus or virus-like particle or a coat protein of the virus or virus-like particle.

61. The method of Embodiment 60, wherein the linker comprises, or consists essentially of, or further consists of an amino acid sequence of GSG, GPSL, or GGSGGGSG, or wherein the linker is an SM(PEG)₈ bifunctional linker comprising, or consisting essentially of, or consisting of an NHS group and a maleimide group, or wherein the linker comprises, or consists essentially of, or further consists of an N-terminal cysteine residue conjugated to triple glycine (GGG) and an N-hydroxysuccinimide-PEG4-maleimide linker SM-PEG4.

62. The method of any one of Embodiments 36-61, wherein the C-terminus or the N-terminus of the epitope(s), a peptide comprising, or consisting essentially of, or consisting of at least one of the epitope(s), or a protein fragment comprising, or consisting essentially of, or consisting of at least one of the epitope(s) is conjugated directly or indirectly to the N-terminus or the C-terminus of a coat protein of the virus or virus-like particle.

63. The method of any one of Embodiments 36-62, wherein the virus or virus-like particle is a bacteriophage virus or virus like particle, or a plant virus or virus like particle.

64. The method of any one of Embodiments 36-63, wherein the virus or virus-like particle is a bacteriophage Qβ virus or virus like particle.

65. The method of any one of Embodiments 36-64, wherein the virus or virus-like particle is a plant picornavirus virus or virus like particle, or a filamentous plant virus or virus-like particle.

66. The method of Embodiment 63 or 65, wherein the plant virus or virus-like particle is of the or Alphafexiviridae family.

67. The method of Embodiment 63 or 65, wherein the plant virus or virus-like particle is a cowpea mosaic virus-like particle or a potato virus X virus-like particle.

68. The method of Embodiment 63 or 65, wherein the plant virus particle or virus-like particle is a rod-shaped virus or virus-like particle.

69. The method of Embodiment 68, wherein the rod-shaped virus or virus-like particle is a tobacco mosaic virus or virus-like particle.

70. The method of any one of Embodiments 36-69, wherein the method does not comprises repeating the administering step.

71. The method of any one of Embodiments 36-70, wherein the virus or virus-like particle(s) is administrated to the subject in a slow-release implant.

72. The method of any one of Embodiments 36-71, wherein the virus or virus-like particle(s) is encapsulated within a degradable polymer matrix.

73. The method of Embodiment 72, wherein the degradable polymer matrix comprises, or consists essentially of, or further consists of a melt processable degradable polymer material that is biocompatible and, upon degradation, produces substantially non-toxic products, wherein the melt processable degradable polymer material is a melt processable biodegradable polymer, and wherein the degradable polymer material has a melt temperature below the degradation temperature of the virus or virus-like particle(s).

74. The method of Embodiment 73, wherein the degradable polymer material comprises, or consists essentially of, or further consists of poly(lactic-co-glycolic acid) (PLGA) or a copolymer thereof.

75. The method of any one of Embodiments 71-74, wherein the slow-release implant comprises, or consists essentially of, or further consists of one or more of: about 50% to about 99% PLGA, about 1% to about 50% virus or virus-like particle(s), or PEG8000, or wherein the slow-release implant comprises, or consists essentially of, or further consists of one or more of: about 80% PLGA, about 10% virus or virus-like particle(s), or about 10% PEG8000, or wherein the slow-release implant comprises, or consists essentially of, or further consists of one or more of: about 75% PLGA, about 10% VNPs, or about 15% PEG8000, optionally wherein the % indicates a weight percentage.

76. The method of any one of Embodiments 71-75, wherein the slow-release implant is loaded into a microneedle patch.

77. The method of any one of Embodiments 36-69 and 71-76, comprising repeating the administering step for about once, about twice, about three times, about four times, about five times, or more, and wherein two subsequent administrations are about 1 day to about 1 year apart, or about 1 week apart, or about 2 weeks apart, or about 3 weeks apart, or about 4 weeks apart, or about 1 month apart, or about 2 months apart, or about 3 months apart, or about 6 months apart.

78. The method of Embodiment 77, wherein the administering step is not repeated for more than 6 times, or more than 7 times, or more than 8 times, or more than 9 times, or more than 10 times, or more than 15 times, or more than 20 times.

79. The method of any one of Embodiments 36-78, whereby the epitope(s) does not activate a T cell or a cytotoxic T cell in the subject.

80. The method of any one of Embodiments 36-79, wherein the method does not comprise administering to the subject an additional adjuvant.

81. The method of any one of Embodiments 36-80, wherein more than one epitopes are delivered by the virus or virus-like particle(s), whereby showing a synergistic effect.

82. A kit for use in a method of any one of Embodiment 1-81, comprising, or consisting essentially of, or consisting of an optional instruction for use and at least one of: the one or more virus or virus-like particle(s) optionally comprising the at least one epitope(s), one or more of the epitope(s), one or more peptide(s) or protein fragment(s) each of which comprises, or consists essentially of, or further consists of at least one of the epitope(s), or one or more of the peptides as disclosed herein.

83. A composition comprising, or consisting essentially of, or consisting of an optional carrier and one or more virus or virus-like particle(s), each of which comprises at least one epitope of a pathogen causing a disease or each of which comprises at least one epitope of one or more cholesterol checkpoint protein(s) and optionally comprising two or more epitopes that may be the same or different from each other.

84. The composition of Embodiment 83, wherein the carrier is a pharmaceutically acceptable carrier).

85. The composition of Embodiment 83 or 84, wherein the cholesterol checkpoint protein(s) is selected from any one or any two or all three of: proprotein convertase subtilisin/kexin-9 (PCSK9), apolipoprotein B (ApoB), or cholesteryl ester transfer protein (CETP)

86. The composition of any one of Embodiments 83-85, wherein each of the virus or virus-like particle(s) comprises at least one peptide comprising, or consisting essentially of, or consisting of an amino acid sequence selected from KTTKQSFDLSVKAQYKKNKH (SEQ ID NO: 1), FGFPEHLLVDFLQSLS (SEQ ID NO: 2), or NVPEEDGTRFHRQASKC (SEQ ID NO: 3).

87. The composition of Embodiment 83 or 84, wherein the pathogen is a Human papillomavirus (HPV).

88. The composition of any one of Embodiments 83-84 and 87, wherein the epitope(s) is of an HPV capsid protein.

89. The composition of Embodiment 87 or 88, wherein the HPV is selected from HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, or HPV82.

90. The composition of Embodiment 88 or 89, wherein the capsid protein comprises, or consists essentially of, or further consists of an HPV capsid protein L1, or an HPV capsid protein L2, or both.

91. The composition of any one of Embodiments 83-84 and 87-90, wherein the epitope(s) is present in a peptide comprising, or consisting essentially of, or consisting of an amino acid sequence of QLYKTCKQAGTCPPD (SEQ ID NO: 4, which is amino acid 17 to amino acid 31 of HPV16 L2 protein), or a fragment of an HPV capsid protein aligned with SEQ ID NO: 4.

92. The composition of Embodiment 83 or 84, wherein the pathogen is a coronavirus.

93. The composition of any one of Embodiments 83-84 and 92, wherein the epitope(s) is of a coronavirus spike protein (S protein).

94. The composition of any one of Embodiments 83-84 and 92-93, wherein the epitope(s) is present in one or more peptide(s) comprising, or consisting essentially of, or consisting of at least one amino acid sequence(s) selected from KGIYQTSN (SEQ ID NO: 5, amino acid (aa) 310 to aa 317 of SARS-CoV-2 S protein), AISSVLNDILSRLDKVE (SEQ ID NO: 6, aa 972 to aa 988 of SARS-CoV-2 S protein), KNHTSPDVDLGDISGIN (SEQ ID NO: 7, aa 1157 to aa 1173 of SARS-CoV-2 S protein), EIDRLNEVAKNLNESLIDLQEL (SEQ ID NO: 8, aa 1182 to aa 1209 of SARS-CoV-2 S protein), ATRFASVYAWNRKRISN (SEQ ID NO: 9, aa 346 to aa 362 of SARS-CoV-2 S protein), YNSASFSTFKCYGVSPTK (SEQ ID NO: 10, aa 369 to aa 386 of SARS-CoV-2 S protein), LPDPSKPSKRSFIED (SEQ ID NO: 11, aa 806 to aa 820 of SARS-CoV-2 S protein), FRKSN (SEQ ID NO: 12, aa 456 to aa 460 of SARS-CoV-2 S protein), PSKPSKRSFIEDLLFNKV (SEQ ID NO: 13, aa 809 to aa 826 of SARS-CoV-2 S protein), TESNKKFLPFQQFGRDIA (SEQ ID NO: 14, aa 553 to aa 570 of SARS-CoV-2 S protein), TESNKKFLPFQQ (SEQ ID NO: 15, aa 553 to aa 564 of SARS-CoV-2 S protein), HADQLTPTWRVY (SEQ ID NO: 16, aa 625 to aa 636 of SARS-CoV-2 S protein), FKEELDKYFKNH (SEQ ID NO: 17, aa 1148 to aa 1159 of SARS-CoV-2 S protein), FASTEKSNIIRGWIF (SEQ ID NO: 18, aa 92 to aa 106 of SARS-CoV-2 S protein), PFLGVYYHKNNKSWM (SEQ ID NO: 19, aa 135 to aa 153 of SARS-CoV-2 S protein), EVRQIAPGQTGKIAD (SEQ ID NO: 20, aa 406 to aa 420 of SARS-CoV-2 S protein), NNLDSKVGGNYNYLYR (SEQ ID NO: 21, aa 439 to aa 454 of SARS-CoV-2 S protein), LFRKSNLKPFERDIS (SEQ ID NO: 22, aa 455 to aa 469 of SARS-CoV-2 S protein), or a fragment of a coronavirus S protein aligned with each thereof, wherein the epitopes that may be the same or different from each other.

95. The composition of any one of Embodiments 83-84 and 92-94, wherein the epitope(s) is present in one or more peptide(s) comprising, or consisting essentially of, or consisting of at least one amino acid sequence(s) selected from AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), EIDRLNEVAKNLNESLIDLQEL (SEQ ID NO: 8, amino acid (aa) 1182 to aa 1209 of SARS-CoV-2 S protein), YNSASFSTFKCYGVSPTK (aa 369 to aa 386 of SARS-CoV-2 S protein), PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), PFLGVYYHKNNKSWM (aa 135 to aa 153 of SARS-CoV-2 S protein), or a fragment of a coronavirus S protein aligned with each thereof.

96. The composition of any one of Embodiments 83-95, comprising, or consisting essentially of, or consisting of one or more virus or virus-like particle(s), wherein each of the virus or virus-like particle comprises at least one peptide as disclosed.

97. The composition of Embodiment 96, wherein each of the at least one peptide comprises, or consists essentially of, or further consists of an amino acid sequence selected from one or more of KTTKQSFDLSVKAQYKKNKH (SEQ ID NO: 1), FGFPEHLLVDFLQSLS (SEQ ID NO: 2), or NVPEEDGTRFHRQASKC (SEQ ID NO: 3).

98. The composition of Embodiment 97, wherein the at least one peptide comprises, or consists essentially of, or further consists of an amino acid sequence of QLYKTCKQAGTCPPD (SEQ ID NO: 4) or a fragment of an HPV capsid protein aligned with SEQ ID NO: 4.

99. The composition of Embodiment 97, wherein each of the at least one peptide comprises, or consists essentially of, or further consists of an amino acid sequence selected from one or more of AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), EIDRLNEVAKNLNESLIDLQEL (SEQ ID NO: 8, amino acid (aa) 1182 to aa 1209 of SARS-CoV-2 S protein), YNSASFSTFKCYGVSPTK (aa 369 to aa 386 of SARS-CoV-2 S protein), PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), PFLGVYYHKNNKSWM (aa 135 to aa 153 of SARS-CoV-2 S protein), or a fragment of a coronavirus S protein aligned with each thereof.

100. The composition of any one of Embodiments 83-99, comprising, or consisting essentially of, or consisting of two or more virus or virus-like particles, each of which comprises at least one epitope(s), or one or more peptide(s) each of which comprises, or consists essentially of, or further consists of at least one of the epitope(s), or one or more protein fragment(s) each of which comprises, or consists essentially of, or further consists of at least one of the epitope(s).

101. The composition of any one of Embodiments 83-100, comprising, or consisting essentially of, or consisting of two or more virus or virus-like particles, each of which comprises at least one peptide as disclosed herein.

102. The composition of any one of Embodiments 83-99, comprising, or consisting essentially of, or consisting of one virus or virus-like particle comprising at least one epitope(s), or one or more peptide(s) each of which comprises, or consists essentially of, or further consists of at least one of the epitope(s), or one or more protein fragment(s) each of which comprises, or consists essentially of, or further consists of at least one of the epitope(s).

103. The composition of any one of Embodiments 83-99 and 102, comprising, or consisting essentially of, or consisting of one virus or virus-like particle comprising at least one peptide as disclosed herein.

104. The composition of any one of Embodiments 83-103, wherein the epitope(s), a peptide comprising thereof, or a protein fragment comprising thereof is present on the outer surface of the virus or virus-like particle.

105. The composition of any one of Embodiments 83-104, wherein the epitope(s), a peptide comprising, or consisting essentially of, or consisting of at least one of the epitope(s), or a protein fragment comprising, or consisting essentially of, or consisting of at least one of the epitope(s) is conjugated directly or indirectly to the virus or virus-like particle, or a coat protein of the virus or virus-like particle.

106. The composition of any one of Embodiments 83-105, wherein the epitope(s), a peptide comprising, or consisting essentially of, or consisting of at least one of the epitope(s), or a protein fragment comprising, or consisting essentially of, or consisting of at least one of the epitope(s) is conjugated indirectly by a method comprising, or consisting essentially of, or yet further consisting of a linker to the virus or virus-like particle, or a coat protein of the virus or virus-like particle.

107. The composition of Embodiment 106, wherein the linker comprises, or consists essentially of, or further consists of an amino acid sequence of GSG, GPSL, or GGSGGGSG, or wherein the linker is an SM(PEG)₈ bifunctional linker comprising, or consisting essentially of, or consisting of an NHS group and a maleimide group, or wherein the linker comprises, or consists essentially of, or further consists of an N-terminal cysteine residue conjugated to triple glycine (GGG) and an N-hydroxysuccinimide-PEG4-maleimide linker SM-PEG4.

108. The composition of any one of Embodiments 83-107, wherein the C-terminus or the N-terminus of the epitope(s), a peptide comprising, or consisting essentially of, or consisting of at least one of the epitope(s), or a protein fragment comprising, or consisting essentially of, or consisting of at least one of the epitope(s) is conjugated directly or indirectly to the N-terminus or the C-terminus of a coat protein of the virus or virus-like particle.

109. The composition of any one of Embodiments 83-108, wherein the virus or virus-like particle is a bacteriophage virus or virus-like particle, or a plant virus or virus like particle.

110. The composition of any one of Embodiments 83-109, wherein the virus or virus-like particle is a bacteriophage Qβ virus or virus-like particle.

111. The composition of any one of Embodiments 83-109, wherein the virus or virus-like particle is a plant picornavirus virus or virus-like particle, or a filamentous plant virus or virus-like particle.

112. The composition of Embodiment 109 or 111, wherein the plant virus or virus-like particle is of the or Alphafexiviridae family.

113. The composition of Embodiment 109 or 111, wherein the plant virus or virus-like particle is a cowpea mosaic virus-like particle or a potato virus X virus-like particle.

114. The composition of Embodiment 109 or 111, wherein the plant virus particle or virus-like particle is a rod-shaped virus or virus-like particle.

115. The composition of Embodiment 114, wherein the rod-shaped virus or virus-like particle is a tobacco mosaic virus or virus-like particle.

116. The composition of any one of Embodiments 83-115, wherein the virus or virus-like particle(s) is in a slow-release implant.

117. The composition of any one of Embodiments 83-116, wherein the virus or virus-like particle(s) is encapsulated within a degradable polymer matrix.

118. The composition of Embodiment 117, wherein the degradable polymer matrix comprises, or consists essentially of, or further consists of a melt processable degradable polymer material that is biocompatible and, upon degradation, produces substantially non-toxic products, wherein the melt processable degradable polymer material is a melt processable biodegradable polymer, and wherein the degradable polymer material has a melt temperature below the degradation temperature of the virus or virus-like particle(s).

119. The composition of Embodiment 118, wherein the degradable polymer material comprises, or consists essentially of, or further consists of poly(lactic-co-glycolic acid) (PLGA) or a copolymer thereof.

120. The composition of any one of Embodiments 116-119, wherein the slow-release implant comprises, or consists essentially of, or further consists of one or more of: about 50% to about 99% PLGA, about 1% to about 50% virus or virus-like particle(s), or PEG8000, or wherein the slow-release implant comprises, or consists essentially of, or further consists of one or more of: about 80% PLGA, about 10% virus or virus-like particle(s), or about 10% PEG8000 optionally by weight, or wherein the slow-release implant comprises, or consists essentially of, or further consists of one or more of: about 75% PLGA, about 10% VNPs, or about 15% PEG8000 optionally by weight.

121. The composition of any one of Embodiments 116-120, wherein the slow-release implant is loaded into a microneedle patch suitable for self-administration.

122. The composition of any one of Embodiments 83-121, wherein the composition does not comprise an additional adjuvant.

Other aspects are set forth within the following claims.

### REFERENCES FOR EXPERIMENT NO. 1

1. World Health Organization (2016) Descriptive note 3 17 May/2017, http://www.who.int/mediacentre/factsheets/fs317/en/ [accessed: Nov. 1, 2020]
2. A. Gisterå, G. K. Hansson, Nature Reviews Nephrology 2017, 13, 368.
3. J. G. Robinson, D. D. Heistad, K. A. A. Fox, Atherosclerosis 2015, 243, 593.
4. M. S. Sabatine, et al., New England Journal of Medicine 2015, 372, 1500.
5. J. G. Robinson, et al., New England Journal of Medicine 2015, 372, 1489.
6. S. E. Nissen, et al., JAMA—Journal of the American Medical Association 2016, 315, 1580.
7. ICER, Institute for Clinical and Economic Review. PCSK9 Inhibitors for Treatment of High Cholesterol: Effectiveness, Value, and Value-Based Price Benchmarks Final Report. Published Feb. 24, 2019.
8. M. D. Shin, et al., Nature Nanotechnology 2020, 15, 646.
9. B. Chackerian, K. M. Frietze, Expert Review of Vaccines 2016, 15, 561.
10. S. Shukla, et al., Biomaterials Science 2020, 8, 5489.
11. M. F. Bachmann, et al., Philosophical Transactions of the Royal Society B: Biological Sciences 2011, 366, 2815.
12. G. N. Fredrikson, et al., Arteriosclerosis, Thrombosis, and Vascular Biology 2003, 23, 879.
13. R. Klingenberg, et al., Arteriosclerosis, Thrombosis, and Vascular Biology 2010, 30, 946.
14. K. Kobiyama, et al., K. Ley, European Journal of Immunology 2018, 48, 1580.
15. G. Qi, et al., Peptides 2011, 32, 790.
16. C. W. Rittershaus, et al., Arteriosclerosis, Thrombosis, and Vascular Biology 2000, 20, 2106.
17. Y. Pan, et al. Y. Zhou, H. Wu, X. Chen, X. Hu, H. Zhang, Z. Zhou, Z. Qiu, Y. Liao, Scientific Reports 2017, 7, 1.
18. C. Landlinger, et al., European Heart Journal 2017, 38, 2499.
19. E. Crossey, et al., Vaccine 2015, 33, 5747.
20. R. Kawakami, et al., PLoS ONE 2018, 13, 1.
21. H. Jing, et al., Vaccine 2011, 29, 4102.
22. A. C. Tissot, et al., European Journal of Immunology 2013, 43, 716.
23. M. H. Davidson, et al., Atherosclerosis 2003, 169, 113.
24. Clinical trial NCT01284582 https://clinicaltrials.gov/ [accessed: 11/01/2020]
25. Clinical trial NCT02508896 https://clinicaltrials.gov/ [accessed: 11/01/2020]
26. P. W. Lee, et al., ACS Nano 2017, 11, 8777.
27. D. M. Wirth, J. K. Pokorski, Polymer 2019, 181, 121802.
28. S. Shao, et al., Vaccines 2021, 9 (1), 66.
29. S. D. Brown, et al., Biochemistry 2009, 48, 47, 11155-11157.
30. F. D. Finkelman, et al., Annual Review of Immunology 1990, 8, 303.
31. Z. Zeng, et al., American Journal of Translational Research 2018, 10, 1817.
32. H. Tanigawa, et al., Circulation 2007, 116, 1267.
33. V. S. Sabbisetti, et al., Journal of the American Society of Nephrology 2014, 25, 2177 LP.
34. M. R. McGill, EXCLI Journal 2016, 15, 817.
35. H. Kollmus, et al., Mammalian Genome 2020, 31, 30.

### REFERENCES FOR EXPERIMENT NO. 2

1. Centers for Disease Control and Prevention. Genital HPV Infection—CDC Fact Sheet; Centers for Disease Control and Prevention: Atlanta, GA, USA, 2014.
2. Zhai, L.; Tumban, E. Gardasil-9: A global survey of projected efficacy. Antivir. Res. 2016, 130, 101-109.
3. Petrosky, E. et al., L. E. Use of 9-valent human papillomavirus (HPV) vaccine: Updated HPV vaccination recommendations of the advisory committee on immunization practices. Mmwr. Morb. Mortal. Wkly. Rep. 2015, 64, 300.
4. Centers for Disease Control and Prevention. FDA licensure of bivalent human papillomavirus vaccine (HPV2, Cervarix) for use in females and updated HPV vaccination recommendations from the Advisory Committee on Immunization Practices (ACIP). Mmwr. Morb. Mortal. Wkly. Rep. 2010, 59, 626.
5. Centers for Disease Control and Prevention. HPV Vaccine Information for Clinicians—Fact Sheet; Centers for Disease Control and Prevention: Atlanta, GA, USA, 2012.
6. Kirby, T. FDA approves new upgraded Gardasil 9. Lancet Oncol. 2015, 16, e56.
7. Walker, T. Y. et al., S. National, regional, state, and selected local area vaccination coverage among adolescents aged 13-17 years—United States, 2018. Morb. Mortal. Wkly. Rep. 2019, 68, 718.
8. Stokley, S., et al., L. Human papillomavirus vaccination coverage among adolescents, 2007-2013, and postlicensure vaccine safety monitoring, 2006-2014—United States. Mmwr. Morb. Mortal. Wkly. Rep. 2014, 63, 620.
9. Wigle, J. et al., Global Delivery of Human Papillomavirus Vaccines Human papillomavirus HPV Vaccine Immunization Global Progress; Pediatr Clin North Am. 2016, 63(1), 81-95.
10. Reagan-Steiner, S. et al., National, regional, state, and selected local area vaccination coverage among adolescents aged 13-17 years-United States, 2014. Mmwr. Morb. Mortal. Wkly. Rep. 2015, 64, 784.
11. Trus, B. et al., J. Localization of the HPV-16 minor capsid protein L2 by difference imaging. Microsc. Microanal. 2005, 11, 642-643.
12. Buck, C. B. et al., Arrangement of L2 within the papillomavirus capsid. J. Virol. 2008, 82, 5190-5197.
13. Chen, X. S. et al., Structure of small virus-like particles assembled from the L1 protein of human papillomavirus 16. Mol. Cell 2000, 5, 557-567.
14. European Medicines Agency. Gardasil 9 Public Assessment Report; European Medicines Agency: Amsterdam, The Netherlands, 2015.
15. Tyler, M. et al., Immunization with a consensus epitope from human papillomavirus L2 induces antibodies that are broadly neutralizing. Vaccine 2014, 32, 4267-4274.
16. Tumban, E. et al., A pan-HPV vaccine based on bacteriophage PP7 VLPs displaying broadly cross-neutralizing epitopes from the HPV minor capsid protein, L2. PLoS ONE 2011, 6, e23310.
17. Zhai, L. et al., A novel candidate HPV vaccine: MS2 phage VLP displaying a tandem HPV L2 peptide offers similar protection in mice to Gardasil-9. Antivir. Res. 2017, 147, 116-123.
18. Shao, S. et al., Functionalization of cobalt porphyrin-phospholipid bilayers with his-tagged ligands and antigens. Nat. Chem. 2015, 7, 438-446.
19. Shukla, S. et al., Plant viral nanoparticles-based HER2 vaccine: Immune response influenced by differential transport, localization and cellular interactions of particulate carriers. Biomaterials 2017, 121, 15-27.
20. Xia, Y. et al., Exploiting the pliability and lateral mobility of Pickering emulsion for enhanced vaccination. Nat. Mater. 2018, 17, 187-194.
21. Yue, H.; Ma, G. Polymeric micro/nanoparticles: Particle design and potential vaccine delivery applications. Vaccine 2015, 33, 5927-5936.
22. Demento, S. L. et al., Role of sustained antigen release from nanoparticle vaccines in shaping the T cell memory phenotype. Biomaterials 2012, 33, 4957-4964.
23. Desai, K. G. H.; Schwendeman, S. P. Active self-healing encapsulation of vaccine antigens in PLGA microspheres. J Control. Release Off. J. Control. Release Soc. 2013, 165, 62-74.
24. Irvine, D. J.; Aung, A.; Silva, M. Controlling timing and location in vaccines. Adv. Drug Deliv. Rev. 2020, 158, 91-115.
25. Lee, P. W. et al., Biodegradable viral nanoparticle/polymer implants prepared via melt-processing. ACS Nano 2017, 11, 8777-8789.
26. Mohsen, M. O. et al., Delivering adjuvants and antigens in separate nanoparticles eliminates the need of physical linkage for effective vaccination. J. Control. Release 2017, 251, 92-100.
27. Bao, Q. et al., M. Phage-based vaccines. Adv. Drug Deliv. Rev. 2019, 145, 40-56.
28. Isarov, S. A. et al., “Graft-to” Protein/Polymer Conjugates Using Polynorbornene Block Copolymers. Biomacromolecules 2016, 17, 641-648.
29. Wirth, D. M. et al., Design and fabrication of a low-cost pilot-scale melt-processing system. Polymer 2019, 181, 121802.
30. Day, P. M. et al., A human papillomavirus (HPV) in vitro neutralization assay that recapitulates the in vitro process of infection provides a sensitive measure of HPV L2 infection-inhibiting antibodies. Clin. Vaccine Immunol. 2012, 19, 1075-1082.
31. Ahlborg, N. et al., Immunogenicity of chimeric multiple antigen peptides based on Plasmodium falciparum antigens: Impact of epitope orientation. Vaccine 1998, 16, 38-44.
32. Monso, M. et al., Influence of conjugation chemistry and B epitope orientation on the immune response of branched s. Bioconjugate Chem. 2013, 24, 578-585.

### REFERENCES FOR EXPERIMENT NO. 3

1. Wang, C.; Horby, P. W.; Hayden, F. G.; Gao, G. F. A Novel Coronavirus Outbreak of Global Health Concern. The Lancet 2020, 395 (10223), 470-473. https://doi.org/10.1016/S0140-6736(20)30185-9.
2. Corey, B. L.; Mascola, J. R.; Fauci, A. S.; Collins, F. S. A Strategic Approach to COVID-19 Vaccine R&D. Science 2020, 368 (6494), 948-950. https://doi.org/10.1126/science.abc5312.
3. https://ourworldindata.org/covid-vaccinations (Last accessed on 04/01/2021)
4. Shukla, S.; Myers, J. T.; Woods, S. E.; Gong, X.; Czapar, A. E.; Commandeur, U.; Huang, A. Y.; Levine, A. D.; Steinmetz, N. F. Plant Viral Nanoparticles-Based HER2 Vaccine: Immune Response Influenced by Differential Transport, Localization and Cellular Interactions of Particulate Carriers. Biomaterials 2017, 121, 15-27. https://doi.org/10.1016/j.biomaterials.2016.12.030.
5. Mohsen, M. O.; Augusto, G.; Bachmann, M. F. The 3Ds in Virus-like Particle Based-Vaccines: “Design, Delivery and Dynamics.” Immunological Reviews 2020, 296 (1), 155-168. https://doi.org/10.1111/imr.12863.
6. Bachmann, M. F.; Jennings, G. T. Vaccine Delivery: A Matter of Size, Geometry, Kinetics and Molecular Patterns. Nature Reviews Immunology 2010, 10 (11), 787-796. https://doi.org/10.1038/nri2868.
7. Lin, T.; Chen, Z.; Usha, R.; Stauffacher, C. V.; Dai, J. B.; Schmidt, T.; Johnson, J. E. The Refined Crystal Structure of Cowpea Mosaic Virus at 2.8 AResolution. Virology 1999, 265 (1), 20-34. https://doi.org/10.1006/viro.1999.0038.
8. Rybicki, E. P. Plant-Based Vaccines against Viruses. Virology Journal 2014, 11 (1), 1-20. https://doi.org/10.1186/s12985-014-0205-0.
9. Lizotte, P. H.; Wen, A. M.; Sheen, M. R.; Fields, J.; Rojanasopondist, P.; Steinmetz, N. F.; Fiering, S. In Situ Vaccination with Cowpea Mosaic Virus Nanoparticles Suppresses Metastatic Cancer. Nature Nanotechnology 2016, 11 (3), 295-303. https://doi.org/10.1038/nnano.2015.292.
10. Shukla, S.; Jandzinski, M.; Wang, C.; Gong, X.; Bonk, K. W.; Keri, R. A.; Steinmetz, N. F. A Viral Nanoparticle Cancer Vaccine Delays Tumor Progression and Prolongs Survival in a HER2+Tumor Mouse Model. Advanced Therapeutics 2019, 2 (4), 1800139. https://doi.org/10.1002/adtp.201800139.
11. Chariou, P. L.; Ortega-Rivera, O. A.; Steinmetz, N. F. Nanocarriers for the Delivery of Medical, Veterinary, and Agricultural Active Ingredients. ACS Nano 2020, 14 (3), 2678-2701. https://doi.org/10.1021/acsnano.0c00173.
12. Ortega-Rivera, O. A.; Pokorski, J. K.; Steinmetz, N. F. A Single-Dose, Implant-Based, Trivalent Virus-like Particle Vaccine against “Cholesterol Checkpoint” Proteins. Advanced Therapeutics 2021, U.S. Pat. Nos. 2,100,014, 2,100,014. https://doi.org/10.1002/adtp.202100014.
13. 1 Shao, S.; Ortega-Rivera, O. A.; Ray, S.; Pokorski, J. K.; Steinmetz, N. F. A Scalable Manufacturing Approach to Single Dose Vaccination against HPV. Vaccines 2021, 9 (1), 1-15. https://doi.org/10.3390/vaccines9010066.
14. Boone, C. E.; Wang, C.; Lopez-Ramirez, M. A.; Beiss, V.; Shukla, S.; Chariou, P. L.; Kupor, D.; Rueda, R.; Wang, J.; Steinmetz, N. F. Active Microneedle Administration of Plant Virus Nanoparticles for Cancer in Situ Vaccination Improves Immunotherapeutic Efficacy. ACS Applied Nano Materials 2020, 3 (8), 8037-8051. https://doi.org/10.1021/acsanm.0c01506.
15. Shin, M. D.; Shukla, S.; Chung, Y. H.; Beiss, V.; Chan, S. K.; Ortega-Rivera, O. A.; Wirth, D. M.; Chen, A.; Sack, M.; Pokorski, J. K.; Steinmetz, N. F. COVID-19 Vaccine Development and a Potential Nanomaterial Path Forward. Nature Nanotechnology 2020, 15 (8), 646-655. https://doi.org/10.1038/s41565-020-0737-y.
16. Chung, Y. H.; Beiss, V.; Fiering, S. N.; Steinmetz, N. F. Covid-19 Vaccine Frontrunners and Their Nanotechnology Design. ACS Nano 2020, 14 (10), 12522-12537. https://doi.org/10.1021/acsnano.0c07197.
17. Wu, Y.; Wang, F.; Shen, C.; Peng, W.; Li, D.; Zhao, C.; Li, Z.; Li, S.; Bi, Y.; Yang, Y.; Gong, Y.; Xiao, H.; Fan, Z.; Tan, S.; Wu, G.; Tan, W.; Lu, X.; Fan, C.; Wang, Q.; Liu, Y.; Zhang, C.; Qi, J.; Gao, G. F.; Gao, F.; Liu, L. A Non-Competing Pair of Human Neutralizing Antibodies Block COVID-19 Virus Binding to Its Receptor ACE2. medRxiv 2020, 1278 (June), 1274-1278. https://doi.org/10.1101/2020.05.01.20077743.
18. Liu, Z.; VanBlargan, L. A.; Bloyet, L. M.; Rothlauf, P. W.; Chen, R. E.; Stumpf, S.; Zhao, H.; Errico, J. M.; Theel, E. S.; Liebeskind, M. J.; Alford, B.; Buchser, W. J.; Ellebedy, A. H.; Fremont, D. H.; Diamond, M. S.; Whelan, S. P. J. Identification of SARS-CoV-2 Spike Mutations That Attenuate Monoclonal and Serum Antibody Neutralization. Cell Host and Microbe 2021, 29 (3), 477-488.e4. https://doi.org/10.1016/j.chom.2021.01.014.
19. Wang, Q.; Zhang, L.; Kuwahara, K.; Li, L.; Liu, Z.; Li, T.; Zhu, H.; Liu, J.; Xu, Y.; Xie, J.; Morioka, H.; Sakaguchi, N.; Qin, C.; Liu, G. Immunodominant SARS Coronavirus Epitopes in Humans Elicited Both Enhancing and Neutralizing Effects on Infection in Non-Human Primates. ACS Infectious Diseases 2016, 2 (5), 361-376. https://doi.org/10.1021/acsinfecdis.6b00006.
20. Lee, W. S.; Wheatley, A. K.; Kent, S. J.; DeKosky, B. J. Antibody-Dependent Enhancement and SARS-CoV-2 Vaccines and Therapies. Nature Microbiology 2020, 5 (10), 1185-1191. https://doi.org/10.1038/s41564-020-00789-5.
21. Hotez, P. J.; Corry, D. B.; Bottazzi, M. E. COVID-19 Vaccine Design: The Janus Face of Immune Enhancement. Nature Reviews Immunology 2020, 20 (6), 347-348. https://doi.org/10.1038/s41577-020-0323-4.
22. Wen, A. M.; Lee, K. L.; Yildiz, I.; Bruckman, M. A.; Shukla, S.; Steinmetz, N. F. Viral Nanoparticles for in Vivo Tumor Imaging. Journal of visualized experiments: JoVE 2012, No. 69. https://doi.org/10.3791/4352.
23. Huo, J.; Zhao, Y.; Ren, J.; Zhou, D.; Duyvesteyn, H. M. E.; Ginn, H. M.; Carrique, L.; Malinauskas, T.; Ruza, R. R.; Shah, P. N. M.; Tan, T. K.; Rijal, P.; Coombes, N.; Bewley, K. R.; Tree, J. A.; Radecke, J.; Paterson, N. G.; Supasa, P.; Mongkolsapaya, J.; Screaton, G. R.; Carroll, M.; Townsend, A.; Fry, E. E.; Owens, R. J.; Stuart, D. I. Neutralization of SARS-CoV-2 by Destruction of the Prefusion Spike. Cell Host and Microbe 2020, 28 (3), 445-454.e6. https://doi.org/10.1016/j.chom.2020.06.010.
24. Wang, H.; Wu, X.; Zhang, X.; Hou, X.; Liang, T.; Wang, D.; Teng, F.; Dai, J.; Duan, H.; Guo, S.; Li, Y.; Yu, X. SARS-CoV-2 Proteome Microarray for Mapping COVID-19 Antibody Interactions at Amino Acid Resolution. ACS Central Science 2020, 6 (12), 2238-2249. https://doi.org/10.1021/acscentsci.0c00742.
25. Poh, C. M.; Carissimo, G.; Wang, B.; Amrun, S. N.; Lee, C. Y. P.; Chee, R. S. L.; Fong, S. W.; Yeo, N. K. W.; Lee, W. H.; Torres-Ruesta, A.; Leo, Y. S.; Chen, M. I C.; Tan, S. Y.; Chai, L. Y. A.; Kalimuddin, S.; Kheng, S. S. G.; Thien, S. Y.; Young, B. E.; Lye, D. C.; Hanson, B. J.; Wang, C. I.; Renia, L.; Ng, L. F. P. Two Linear Epitopes on the SARS-CoV-2 Spike Protein That Elicit Neutralising Antibodies in COVID-19 Patients. Nature Communications 2020, 11 (1). https://doi.org/10.1038/s41467-020-16638-2.
26. Li, Y.; Lai, D. yun; Zhang, H. nan; Jiang, H. wei; Tian, X.; Ma, M. liang; Qi, H.; Meng, Q. feng; Guo, S. juan; Wu, Y.; Wang, W.; Yang, X.; Shi, D. wei; Dai, J. biao; Ying, T.; Zhou, J.; Tao, S. ce. Linear Epitopes of SARS-CoV-2 Spike Protein Elicit Neutralizing Antibodies in COVID-19 Patients. Cellular and Molecular Immunology 2020, 17 (10), 1095-1097. https://doi.org/10.1038/s41423-020-00523-5.
27. Lu, S.; Xie, X. xiu; Zhao, L.; Wang, B.; Zhu, J.; Yang, T. rui; Yang, G. wen; Ji, M.; Lv, C. ping; Xue, J.; Dai, E. hei; Fu, X. ming; Liu, D. qun; Zhang, L.; Hou, S. jie; Yu, X. lin; Wang, Y. ling; Gao, H. xia; Shi, X. han; Ke, C. wen; Ke, B. xia; Jiang, C. guo; Liu, R. tian. The Immunodominant and Neutralization Linear Epitopes for SARS-CoV-2. Cell Reports 2021, 34 (4), 108666. https://doi.org/10.1016/j.celrep.2020.108666.
28. Lee, P. W.; Shukla, S.; Wallat, J. D.; Danda, C.; Steinmetz, N. F.; Maia, J.; Pokorski, J. K. Biodegradable Viral Nanoparticle/Polymer Implants Prepared via Melt-Processing. ACS Nano 2017, 11 (9), 8777-8789. https://doi.org/10.1021/acsnano.7b02786.
29. Wirth, D. M.; Pokorski, J. K. Design and Fabrication of a Low-Cost Pilot-Scale Melt-Processing System. Polymer 2019, 181 (July), 121802. https://doi.org/10.1016/j.polymer.2019.121802.
30. Reed, L. J.; Muench, H. A Simple Method of Estimating Fifty Percent Endpoints. Am J Hyg 1938, 27, 493-98.
31. Porta, C.; Spall, V. E.; Loveland, J.; Johnson, J. E.; Barker, P. J.; Lomonossoff, G. P. Development of Cowpea Mosaic Virus as a High-Yielding System for the Presentation of Foreign Peptides. Virology 1994, 202 (2), 949-955. https://doi.org/10.1006/viro.1994.1417.
32. Usha, R.; Rohll, J. B.; Spall, V. E.; Shanks, M.; Maule, A. J.; Johnson, J. E.; Lomonossofft, G. P. Expression of an Animal Virus Antigenic Site on the Surface of a Plant Virus Particle. Virology. 1993, pp 366-374. https://doi.org/10.1006/viro.1993.1598.
33. Raja, K. S.; Wang, Q.; Gonzalez, M. J.; Manchester, M.; Johnson, J. E.; Finn, M. G. Hybrid Virus-Polymer Materials. 1. Synthesis and Properties of PEG-Decorated Cowpea Mosaic Virus. Biomacromolecules 2003, 4 (3), 472-476. https://doi.org/10.1021/bm025740+.
34. Corbett, K. S.; Edwards, D. K.; Leist, S. R.; Abiona, O. M.; Boyoglu-Barnum, S.; Gillespie, R. A.; Himansu, S.; Schafer, A.; Ziwawo, C. T.; DiPiazza, A. T.; Dinnon, K. H.; Elbashir, S. M.; Shaw, C. A.; Woods, A.; Fritch, E. J.; Martinez, D. R.; Bock, K. W.; Minai, M.; Nagata, B. M.; Hutchinson, G. B.; Wu, K.; Henry, C.; Bahl, K.; Garcia-Dominguez, D.; Ma, L. Z.; Renzi, I.; Kong, W. P.; Schmidt, S. D.; Wang, L.; Zhang, Y.; Phung, E.; Chang, L. A.; Loomis, R. J.; Altaras, N. E.; Narayanan, E.; Metkar, M.; Presnyak, V.; Liu, C.; Louder, M. K.; Shi, W.; Leung, K.; Yang, E. S.; West, A.; Gully, K. L.; Stevens, L. J.; Wang, N.; Wrapp, D.; Doria-Rose, N. A.; Stewart-Jones, G.; Bennett, H.; Alvarado, G. S.; Nason, M. C.; Ruckwardt, T. J.; McLellan, J. S.; Denison, M. R.; Chappell, J. D.; Moore, I. N.; Morabito, K. M.; Mascola, J. R.; Baric, R. S.; Carfi, A.; Graham, B. S. SARS-CoV-2 MRNA Vaccine Design Enabled by Prototype Pathogen Preparedness. Nature 2020, 586 (7830), 567-571. https://doi.org/10.1038/s41586-020-2622-0.
35. Ambuhl P. M.; Tissot A. C.; Fulurija A.; Maurer P.; Nussberger J.; Sabat R.; Nief V.; Schellekens C.; Sladko K.; Roubicek K.; Pfister T.; Rettenbacher M.; Volk H. D.; Wagner F.; Müller P.; Jennings G. T.; Bachmann M. F. A vaccine for hypertension based on virus-like particles: preclinical efficacy and phase I safety and immunogenicity. J Hypertens. 2007 January; 25(1):63-72. https://doi.org/10.1097/HJH.0b013e32800ff5d6.
36. Pan, Y.; Zhou, Y.; Wu, H.; Chen, X.; Hu, X.; Zhang, H.; Zhou, Z.; Qiu, Z.; Liao, Y. A Therapeutic Peptide Vaccine Against PCSK9. Scientific Reports 2017, 7 (1), 1-10. https://doi.org/10.1038/s41598-017-13069-w.
37. Khan, F.; Porter, M.; Schwenk, R.; DeBot, M.; Saudan, P.; Dutta, S. Head-to-Head Comparison of Soluble vs. Qβ VLP Circumsporozoite Protein Vaccines Reveals Selective Enhancement of NANP Repeat Responses. PLoS ONE 2015, 10 (11), 1-18. https://doi.org/10.1371/journal.pone.0142035.
38. Cai, H.; Shukla, S.; Wang, C.; Masarapu, H.; Steinmetz, N. F. Heterologous Prime-Boost Enhances the Antitumor Immune Response Elicited by Plant-Virus-Based Cancer Vaccine. Journal of the American Chemical Society 2019, 141 (16), 6509-6518. https://doi.org/10.1021/jacs.9b01523.
39. Schmitz, N.; Beerli, R. R.; Bauer, M.; Jegerlehner, A.; Dietmeier, K.; Maudrich, M.; Pumpens, P.; Saudan, P.; Bachmann, M. F. Universal Vaccine against Influenza Virus: Linking TLR Signaling to Anti-Viral Protection. European Journal of Immunology 2012, 42 (4), 863-869. https://doi.org/10.1002/eji.201041225.
40. Lopez-Ramirez, M. A.; Soto, F.; Wang, C.; Rueda, R.; Shukla, S.; Silva-Lopez, C.; Kupor, D.; McBride, D. A.; Pokorski, J. K.; Nourhani, A.; Steinmetz, N. F.; Shah, N. J.; Wang, J. Built-In Active Microneedle Patch with Enhanced Autonomous Drug Delivery. Adv. Mater. 2020, 32, 1905740. https://doi.org/10.1002/adma.201905740
41. Schnyder, J. L.; De Pijper, C. A.; Garcia Garrido, H. M.; Daams, J. G.; Goorhuis, A.; Stijnis, C.; Schaumburg, F.; Grobusch, M. P. Fractional Dose of Intradermal Compared to Intramuscular and Subcutaneous Vaccination—A Systematic Review and Meta-Analysis. Travel Medicine and Infectious Disease 2020, 37 (August), 101868. https://doi.org/10.1016/j.tmaid.2020.101868.
42. Combadiere, B.; Liard, C. Transcutaneous and Intradermal Vaccination. Human vaccines 2011, 7 (8), 811-827. https://doi.org/10.4161/hv.7.8.16274.
43. Kabashima, K.; Honda, T.; Ginhoux, F.; Egawa, G. The Immunological Anatomy of the Skin. Nature Reviews Immunology 2019, 19 (1), 19-30. https://doi.org/10.1038/s41577-018-0084-5.
44. Finkelman F. D.; Holmes J.; Katona I M.; Urban J. F Jr.; Beckmann M. P.; Park L. S.; Schooley K. A.; Coffman R. L.; Mosmann T. R.; Paul W. E. Lymphokine control of in vivo immunoglobulin isotype selection. Annu Rev Immunol. 1990, 8, 303-33. https://doi.org/10.1146/annurev.iy.08.040190.001511.
45. Mercatelli, D.; Giorgi, F. M. Geographic and Genomic Distribution of SARS-CoV-2 Mutations. Frontiers in Microbiology, 2020, 11 (July), 1-13. https://doi.org/10.3389/fmicb.2020.01800.
46. https://covariants.org/
47. https://www.gisaid.org/
48. Coutard, B.; Valle, C.; de Lamballerie, X.; Canard, B.; Seidah, N. G.; Decroly, E. The Spike Glycoprotein of the New Coronavirus 2019-NCoV Contains a Furin-like Cleavage Site Absent in CoV of the Same Clade. Antiviral Research 2020, 176 (February), 104742. https://doi.org/i0.1016/j. antiviral.2020.104742.
49. Guruprasad, L. Human SARS CoV-2 Spike Protein Mutations. Proteins: Structure, Function and Bioinformatics 2021, 89 (5), 569-576. https://doi.org/i0.1002/prot.26042.
50. Walls, A. C.; Park, Y. J.; Tortorici, M. A.; Wall, A.; McGuire, A. T.; Veesler, D. Structure, Function, and Antigenicity of the SARS-CoV-2 Spike Glycoprotein. Cell 2020, 181 (2), 281-292.e6. https://doi.org/10.1016/j.cell.2020.02.058.
51. Laffeber, C.; de Koning, K.; Kanaar, R.; Lebbink, J. H. G. Experimental Evidence for Enhanced Receptor Binding by Rapidly Spreading SARS-CoV-2 Variants. bioRxiv, 2021, 2021.02.22.432357. https://doi.org/10.1101/2021.02.22.432357.
52. 52. Huang, Q.; Ji, K.; Tian, S.; Wang, F.; Huang, B.; Tong, Z.; Tan, S.; Hao, J.; Wang, Q.; Tan, W.; Gao, G. F.; Yan, J. A Single-Dose MRNA Vaccine Provides a Long-Term Protection for HACE2 Transgenic Mice from SARS-CoV-2. Nature Communications 2021, 12 (1), 1-10. https://doi.org/10.1038/s41467-021-21037-2.

### REFERENCES FOR EXPERIMENT NO. 4

1. Almeida J D, Berry D M, Cunningham C H, Hamre D, Hofstad M S, Mallucci L, McIntosh K, Tyrrell D A (1968) Virology: Coronaviruses. Nature 220:650.
2. Wertheim J O, Chu D K, Peiris J S, Kosakovsky Pond S L, Poon L L (2013). A case for the ancient origin of coronaviruses. J Virol 87:7039-7045.
3. Jevsnik M, Ursic T, Zigon N, Lusa L, Krivec U, Petrovec M (2012) Coronavirus infections in hospitalized pediatric patients with acute respiratory tract disease. BMC Infect Dis 12:365.
4. Chan-Yeung M, Xu R H (2003) SARS: epidemiology. Respirology 8 Suppl 1:S9-14.
5. Zumla A, Hui D S, Perlman S (2015) Middle East respiratory syndrome. Lancet 386:995-1007.
6. Wang C, Horby P W, Hayden F G, Gao G F (2020) A novel coronavirus outbreak of global health concern. Lancet 395:470-473.
7. Johns Hopkins University of Medicine (2021) COVID-19 Dashboard. Online resource (https://coronavirus.jhu.edu/map.html). Accessed 22/6/2021.
8. London School of Hygiene & Tropical Medicine (2021) COVID-19 vaccine tracker. Online resource (https://vac-Ishtm.shinyapps.io/ncov_vaccine_landscape/). Accessed 22/6/2021.
9. Capell T, Twyman R M, Armario-Najera V, Ma J K-C, Schillberg S, Christou P (2020) Potential applications of plant biotechnology against SARS-CoV-2. Trends Plant Sci 25:635-643.
10. Lu G, Wang Q, Gao G F (2015) Bat-to-human: spike features determining ‘host jump’ of coronaviruses SARS-CoV, MERS-CoV, and beyond. Trends Microbiol 23:468-478.
11. Li F, Li W, Farzan M, Harrison S C (2005) Structure of SARS coronavirus spike receptor-binding domain complexed with receptor. Science 309:1864-1868.
12. Lu G, Hu Y, Wang Q, Qi J, Gao F, Li Y, Zhang Y, Zhang W, Yuan Y, Bao J (2013) Molecular basis of binding between novel human coronavirus MERS-CoV and its receptor CD26. Nature 500:227-231.
13. Yan R, Zhang Y, Li Y, Xia L, Guo Y, Zhou Q (2020) Structural basis for the recognition of SARS-CoV-2 by full-length human ACE2. Science 367:1444-1448.
14. Li W, Moore M J, Vasilieva N, Sui J, Wong S K, Berne M A, Somasundaran M, Sullivan J L, Luzuriaga K, Greenough T C (2003) Angiotensin-converting enzyme 2 is a functional receptor for the SARS coronavirus. Nature 426:450-454.
15. Hoffmann M, Kleine-Weber H, Schroeder S, Kruger N, Herrler T, Erichsen S, Schiergens T S, Herrler G, Wu N H, Nitsche A (2020) SARS-CoV-2 cell entry depends on ACE2 and TMPRSS2 and is blocked by a clinically proven protease inhibitor. Cell 181:271-280.
16. Raj V S, Mou H, Smits S L, Dekkers D H, Müller M A, Dijkman R, Muth D, Demmers J A, Zaki A, Fouchier R A (2013) Dipeptidyl peptidase 4 is a functional receptor for the emerging human coronavirus-EMC. Nature 495:251-254.
17. Jiang S, Hillyer C, Du L (2020) Neutralizing antibodies against SARS-CoV-2 and other human coronaviruses. Trends Immunol 41:355-359.
18. Su S, Du L, Jiang S (2021) Learning from the past: development of safe and effective COVID-19 vaccines. Nat Rev Microbiol 19:211-219.
19. Yang J, Wang W, Chen Z, Lu S, Yang F, Bi Z, Bao L, Mo F, Li X, Huang Y (2020) A vaccine targeting the RBD of the S protein of SARS-CoV-2 induces protective immunity. Nature 586:572-577.
20. Wang N, Shang J, Jiang S, Du L (2020) Subunit vaccines against emerging pathogenic human coronaviruses. Front Microbiol 11:298.
21. Dai L, Zheng T, Xu K, Han Y, Xu L, Huang E, An Y, Cheng Y, Li S, Liu M, Yang M, Li Y, Cheng H, Yuan Y, Zhang W, Ke C, Wong G, Qi J, Qin C, Yan J, Gao G F (2020) A universal design of betacoronavirus vaccines against COVID-19, MERS, and SARS. Cell 182:722-733.
22. Ward B J, Gobeil P, Seguin A, Atkins J, Boulay I, Charbonneau P Y, Couture M, D'Aoust M A, Dhaliwall J, Finkle C, Hager K, Mahmood A, Makarkov A, Cheng M P, Pillet S, Schimke P, St-Martin S, Trepanier S, Landry N (2021) Phase 1 randomized trial of a plant-derived virus-like particle vaccine for COVID-19. Nat Med 27:1071-1078.
23. Hu B, Guo H, Zhou P, Shi Z L (2020) Characteristics of SARS-CoV-2 and COVID-19. Nat Rev Microbiol 19:141-154.
24. Ahmed S F, Quadeer A A, McKay M R (2020) Preliminary identification of potential vaccine targets for the COVID-19 coronavirus (SARS-CoV-2) based on SARS-CoV immunological studies. Viruses 12:254.
25. Shin M D, Shukla S, Chung Y H, Beiss V, Chan S K, Ortega-Rivera O A, Wirth D M, Chen A, Sack M, Pokorski J K, Steinmetz N F (2020) COVID-19 vaccine development and a potential nanomaterial path forward. Nat Nanotechnol 15:646-655.
26. Zhu Y, Yu D, Han Y, Yan H, Chong H, Ren L, Wang J, Li T, He Y (2020) Cross-reactive neutralization of SARS-CoV-2 by serum antibodies from recovered SARS patients and immunized animals. Sci Adv 6:eabc9999.
27. Zang J, Gu C, Zhou B, Zhang C, Yang Y, Xu S, Zhang X, Zhou Y, Bai L, Wu Y (2020) Immunization with the receptor-binding domain of SARS-CoV-2 elicits antibodies cross-neutralizing SARS-CoV-2 and SARS-CoV without antibody-dependent enhancement. Cell Discov 6:61.
28. Tai W, Zhang X, Drelich A, Shi J, Hsu J C, Luchsinger L, Hillyer C D, Tseng C K, Jiang S, Du L (2020) A novel receptor-binding domain (RBD)-based mRNA vaccine against SARS-CoV-2. Cell Res 30:932-935.
29. Tai W, Zhang X, He Y, Jiang S, Du L (2020) Identification of SARS-CoV RBD-targeting monoclonal antibodies with cross-reactive or neutralizing activity against SARS-CoV-2. Antiviral Res 179:104820.
30. Pinto D, Park Y J, Beltramello M, Walls A C, Tortorici M A, Bianchi S, Jaconi S, Culap K, Zatta F, De Marco A (2020) Cross-neutralization of SARS-CoV-2 by a human monoclonal SARS-CoV antibody. Nature 583:290-295.
31. Wec A Z, Wrapp D, Herbert A S, Maurer D P, Haslwanter D, Sakharkar M, Jangra R K, Dieterle M E, Lilov A, Huang D (2020) Broad neutralization of SARS-related viruses by human monoclonal antibodies. Science 369:731-736.
32. Cao M, Su X, Jiang S (2021) Broad-spectrum anti-coronavirus vaccines and therapeutics to combat the current COVID-19 pandemic and future coronavirus disease outbreaks. Stem Cell Rep 16:398-411.
33. Cohen J (2021) Vaccines that can protect against many coronaviruses could prevent another pandemic. Science online article (https://doi.org/10.1126/science.abi9939).
34. Lucchese G (2020) Epitopes for a 2019-nCoV vaccine. Cell Mol Immunol 17:539-540.
35. Grifoni A, Sidney J, Zhang Y, Scheuermann R H, Peters B, Sette A (2020) A sequence homology and bioinformatic approach can predict candidate targets for immune responses to SARS-CoV-2. Cell Host Microbe 27:671-680.
36. Baruah V, Bose S (2020) Immunoinformatics-aided identification of T cell and B cell epitopes in the surface glycoprotein of 2019-nCoV. J Med Virol 92:495-500.
37. Guo J P, Petric M, Campbell W, McGeer P L (2004) SARS corona virus peptides recognized by antibodies in the sera of convalescent cases. Virology 324:251-256.
38. He Y, Zhou Y, Wu H, Luo B, Chen J, Li W, Jiang S (2004) Identification of immunodominant sites on the spike protein of severe acute respiratory syndrome (SARS) coronavirus: implication for developing SARS diagnostics and vaccines. J Immunol 173:4050-4057.
39. Wang Q, Zhang L, Kuwahara K, Li L, Liu Z, Li T, Zhu H, Liu J, Xu Y, Xie J, Morioka H, Sakaguchi N, Qin C, Liu G (2016) Immunodominant SARS coronavirus epitopes in humans elicited both enhancing and neutralizing effects on infection in non-human primates. ACS Infect Dis 2:361-376.
40. Steinmetz N F, Lin T, Lomonossoff G P, Johnson J E (2009) Structure-based engineering of an icosahedral virus for nanomedicine and nanotechnology. Curr Top Microbiol Immunol 327:23-58.
41. ClinicalTrials.gov (2021) Study of a recombinant coronavirus-like particle COVID-19 vaccine in adults. Online resource (https://clinicaltrials.gov/ct2/show/NCT04636697). Accessed 22/6/2021.
42. Lee P W, Pokorski J K (2018) Poly(lactic-co-glycolic acid) devices: production and applications for sustained protein delivery. WIRES Nanomed Nanobiotechnol 10:e1516.
43. Lee P W, Maia J, Pokorski J K (2017) Milling solid proteins to enhance activity after melt-encapsulation. Int J Pharmaceut 533:254-265.
44. Lee P W, Shukla S, Wallat J D, Danda K C, Maia J, Steinmetz N F, Pokorski J K (2017) Virus-like particle laden biodegradable implants prepared via melt processing. ACS Nano 11:8777-8789.
45. Lee P W, Isarov S A, Wallat J D, Molugu S K, Shukla S, Sun J E P, Zhang J, Zheng Y, Lucius Dougherty M, Konkolewicz D, Stewart P L, Steinmetz N F, Hore M J A, Pokorski J K (2015) Polymer structure and conformation alter immune recognition of virus-like particle-polymer conjugates. J Am Chem Soc 139:3312-3315.
46. Lee P W, Towslee J H, Maia J, Pokorski J K (2015) PEGylation to improve protein stability during melt processing. Macromol Biosci 15:1332-1337.
47. Coutard B, Valle C, de Lamballerie X, Canard B, Seidah N G, Decroly E (2020) The spike glycoprotein of the new coronavirus 2019-NCoV contains a furin-like cleavage site absent in CoV of the same clade. Antiviral Res 176:104742.
48. Guruprasad L (2021) Human SARS CoV-2 spike protein mutations. Prot Struct Funct Bioinfor Mat 89:569-576.
49. Chatterji A, Ochoa W F, Paine M, Ratna B R, Johnson J E, Lin T (2004) New addresses on an addressable virus nanoblock; uniquely reactive Lys residues on cowpea mosaic virus. Chemistry & Biology 11 (6), 855-63.
50. Cai H, Shukla S, Wang C, Masarapu H, Steinmetz N F (2019) Heterologous prime-boost enhances the antitumor immune response elicited by plant-virus-based cancer vaccine. J Am Chem Soc 141:6509-6518.
51. Wirth D M, Pokorski J K (2019) Design and fabrication of a low-cost pilot-scale melt-processing system. Polymer 181:121802.
52. Shao S, Ortega-Rivera O A, Ray S, Pokorski J K, Steinmetz N F (2021) A scalable manufacturing approach to single dose vaccination against HPV. Vaccines 9:66.
53. Ortega-Rivera O A, Pokorski J K, Steinmetz N F (2021) A single-dose, implant-based, trivalent virus-like particle vaccine against “cholesterol checkpoint” proteins. Adv Therapeut (online first, https://doi.org/10.1002/adtp.202100014).
54. Le D H T, Hu H, Commandeur U, Steinmetz N F (2017) Chemical addressability of potato virus X for its applications in bio/nanotechnology. J Struct Biol 200:360-368.
55. Mao C, Beiss V, Fields J, Steinmetz N F, Fiering S (2021) Cowpea mosaic virus stimulates antitumor immunity through recognition by multiple MYD88-dependent toll-like receptors. Biomaterials 275:120914.
56. Mohsen M O, Augusto G, Bachmann M F (2020) The 3Ds in virus-like particle based-vaccines: “design, delivery and dynamics.” Immunol Rev 296:155-168.
57. Bachmann M F, Jennings G T (2010) Vaccine delivery: a matter of size, geometry, kinetics and molecular patterns. Nat Rev Immunol 10:787-796.
58. Shukla S, Myers J T, Woods S E, Gong X, Czapar A E, Commandeur U, Huang A Y, Levine A D, Steinmetz N F (2017) Plant viral nanoparticles-based HER2 vaccine: immune response influenced by differential transport, localization and cellular interactions of particulate carriers. Biomaterials 121:15-27.
59. Huo J, Zhao Y, Ren J, Zhou D, Duyvesteyn H M E, Ginn H M, Carrique L, Malinauskas T, Ruza R R, Shah P N M, Tan T K, Rijal P, Coombes N, Bewley K R, Tree J A, Radecke J, Paterson N G, Supasa P, Mongkolsapaya J, Screaton G R, Carroll M, Townsend A, Fry E E, Owens R J, Stuart D I (2020) Neutralization of SARS-CoV-2 by Destruction of the Prefusion Spike. Cell Host and Microbe 28 (3), 445-454.e6.
60. Jallow S, Leligdowicz A, Kramer H B, Onyango C, Cotten M, Wright C, Whittle H C, Mcmichael A, Dong T, Kessler B M, Rowland-Jones S L (2015) The Presence of Prolines in the Flanking Region of an Immunodominant HIV-2 Gag Epitope Influences the Quality and Quantity of the Epitope Generated. European Journal of Immunology 45 (8), 2232-2242.
61. Watanabe Y, Berndsen Z T, Raghwani J, Seabright G E, Allen J D, Pybus O G, McLellan J S, Wilson I A, Bowden T A, Ward A B, Crispin M (2020) Vulnerabilities in coronavirus glycan shields despite extensive glycosylation. Nat Commun 11:2688.
62. Watanabe Y, Allen J D, Wrapp D, McLellan J S, Crispin M (2020) Site-specific glycan analysis of the SARS-CoV-2 spike. Science 369:330-333.
63. Zheng Y, Lee P W, Wang C, Thomas L D, Stewart P L, Steinmetz N F, Pokorski J K (2019) Freeze-drying to produce efficacious CPMV virus-like particles. Nano Lett 19:2099-2105.
64. Wen A M, Lee K L, Yildiz I, Bruckman M A, Shukla S, Steinmetz N F (2012) Viral nanoparticles for in vivo tumor imaging. J Vis Exp (69):e4352.
65. Shukla S, Jandzinski M, Wang C, Gong X, Bonk K W, Keri R A, Steinmetz N F (2019) A viral nanoparticle cancer vaccine delays tumor progression and prolongs survival in a HER2+ tumor mouse model. Adv Therapeut 2:1800139.
66. Reed L J, Muench H (1938) A simple method of estimating fifty percent endpoints. Am J Hyg 27:493-498.

1. A method for or one or more of the following in a subject in need thereof:
(a) preventing or treating an COVID infection in a subject in need thereof, comprising administering to the subject an effective amount of one or more virus or virus-like particle(s), wherein each virus or virus-like particle comprises at least one a polypeptide selected from: VLP KGIYQTSN (SEQ ID NO: 5, amino acid (aa) 310 to aa 317 of SARS-CoV-2 S protein), AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), EIDRLNEVAKNLNESLIDLQEL (SEQ ID NO: 8, amino acid (aa) 1182 to aa 1209 of SARS-CoV-2 S protein), ATRFASVYAWNRKRISN (SEQ ID NO: 9, amino acid (aa) 346 to aa 362 of SARS-CoV-2 S protein), YNSASFSTFKCYGVSPTK (SEQ ID NO: 10, aa 369 to aa 386 of SARS-CoV-2 S protein), LPDPSKPSKRSFIED (SEQ ID NO: 11, aa 806 to aa 820 of SARS-CoV-2 S protein), FRKSN (SEQ ID NO: 12, aa 456 to aa 460 of SARS-CoV-2 S protein), PSKPSKRSFIEDLLFNKV (SEQ ID NO: 13, aa 809 to aa 826 of SARS-CoV-2 S protein), TESNKKFLPFQQFGRDIA (SEQ ID NO: 14, aa 553 to aa 570 of SARS-CoV-2 S protein), TESNKKFLPFQQ (SEQ ID NO: 15, aa 553 to aa 564 of SARS-CoV-2 S protein), HADQLTPTWRVY (SEQ ID NO: 16, aa 625 to aa 636 of SARS-CoV-2 S protein), FKEELDKYFKNH (SEQ ID NO: 17, aa 1148 to aa 1159 of SARS-CoV-2 S protein), FASTEKSNIIRGWIF (SEQ ID NO: 18, aa 92 to aa 106 of SARS-CoV-2 S protein), PFLGVYYHKNNKSWM (SEQ ID NO: 19, aa 135 to aa 153 of SARS-CoV-2 S protein), EVRQIAPGQTGKIAD (SEQ ID NO: 20, aa 406 to aa 420 of SARS-CoV-2 S protein), NNLDSKVGGNYNYLYR (SEQ ID NO: 22, aa 439 to aa 454 of SARS-CoV-2 S protein), LFRKSNLKPFERDIS (SEQ ID NO: 22, aa 455 to aa 469 of SARS-CoV-2 S protein); or
(b) treating or preventing a cardiovascular disease, treating or preventing an atherosclerosis, treating or preventing a hypercholesterolemia, treating or preventing a lipid dyshomeostasis, preventing a heart attack, preventing a stroke, reducing a statin administration dose or frequency or both, reducing a cholesterol level, reducing an oxidized cholesterol level, reducing a low-density lipoprotein cholesterol (LDL-C) level, reducing a level or an activity of one or more cholesterol checkpoint protein(s), producing an antibody recognizing and binding to one or more cholesterol checkpoint protein(s), triggering, enhancing, or prolonging an immune response to one or more cholesterol checkpoint protein(s), or delivering at least one epitope(s) of one or more cholesterol checkpoint protein(s) to the subject, comprising administering to the subject one or more virus or virus-like particle(s), wherein each virus or virus-like particle comprises at least one epitope of the cholesterol checkpoint protein(s), and optionally comprising two or more epitopes that may be the same or different from each other.

2. The method of claim 1, wherein treatment comprises one or more of delivering at least one epitope of the cholesterol checkpoint protein(s) to the subject, producing an antibody recognizing and binding to the one or more cholesterol checkpoint protein(s) in the subject, triggering, enhancing, or prolonging an immune response to the one or more cholesterol checkpoint protein(s), reducing the level or the activity of the one or more cholesterol checkpoint protein(s) in the subject, reducing the total cholesterol level in the subject, reducing the oxidized cholesterol level in the subject, reducing the LDL-C level in the subject, reducing a statin administration dose or frequency or both, treating or preventing a cardiovascular disease, treating or preventing an atherosclerosis, treating or preventing a hypercholesterolemia, treating or preventing a lipid dyshomeostasis, preventing a heart attack, or preventing a stroke.

3. The method of claim 1, wherein the epitope comprises a fragment of the cholesterol checkpoint protein.

4. The method of claim 1, wherein the cholesterol checkpoint protein(s) is selected from any one or any two or all three of: proprotein convertase subtilisin/kexin-9 (PCSK9), apolipoprotein B (ApoB), or cholesteryl ester transfer protein (CETP).

5. The method of claim 1, wherein each of the virus or virus-like particle(s) comprises at least one epitope peptide comprising an amino acid sequence selected from (a) a fragment of ApoB (KTTKQSFDLSVKAQYKKNKH (SEQ ID NO: 1)), (b) a fragment of CETP (FGFPEHLLVDFLQSLS (SEQ ID NO: 2)), or (c) which is a fragment of PSCK9 (NVPEEDGTRFHRQASKC (SEQ ID NO: 3)), or an equivalent of each thereof.

6. The method of claim 1, wherein each of the least one epitope of the cholesterol checkpoint protein(s) is conjugated to the c-terminus of a coat protein (CP) of the virus-like particle, optionally through a peptide linker.

7. The method of claim 1, wherein the method comprises administering to the subject a plurality of virus or virus-like particles, each of the virus or virus-like particles comprising at least one epitope(s), or one or more peptide(s) each of which comprises at least one of the epitope(s), or one or more fragment(s) of the cholesterol checkpoint protein(s) each of which comprises at least one of the epitopes, the epitopes being the same or different from each other; or
wherein the method comprises administering to the subject three virus or virus-like particles, wherein a first virus or virus-like particle comprises an epitope peptide comprising an amino acid sequence of KTTKQSFDLSVKAQYKKNKH (SEQ ID NO: 1), a second virus or virus-like particle comprises an epitope peptide comprising an amino acid sequence of FGFPEHLLVDFLQSLS (SEQ ID NO: 2), and a third virus or virus-like particle comprises an epitope peptide comprising an amino acid sequence of NVPEEDGTRFHRQASKC (SEQ ID NO: 3), or an equivalent of each thereof; or wherein the method comprises administering to the subject two virus or virus-like particles, wherein a first virus or virus-like particle comprises an epitope peptide comprising an amino acid sequence selected from one of KTTKQSFDLSVKAQYKKNKH (SEQ ID NO: 1), FGFPEHLLVDFLQSLS (SEQ ID NO: 2), or NVPEEDGTRFHRQASKC (SEQ ID NO: 3) or an equivalent of each thereof, and a second virus or virus-like particle comprises the rest two amino acid sequencers in one or two epitope peptide(s); or
wherein the method comprises administering to the subject two virus or virus-like particles, wherein a first virus or virus-like particle comprises an epitope peptide comprising an amino acid sequence selected from one of KTTKQSFDLSVKAQYKKNKH (SEQ ID NO: 1), FGFPEHLLVDFLQSLS (SEQ ID NO: 2), or NVPEEDGTRFHRQASKC (SEQ ID NO: 3) or an equivalent of each thereof, and a second virus or virus-like particle comprises the rest two amino acid sequencers in one or two epitope peptide(s); or
wherein the method comprises administering to the subject one virus or virus-like particle which comprises at least two epitope(s), or one or more peptide(s) each of which comprises one or more of the at least two epitope(s), or one or more fragment(s) of the cholesterol checkpoint protein(s) each of which comprises one or more of the at least one epitope(s), wherein the epitopes may be the same are different from each other; or
wherein the method comprises administering to the subject one virus or the virus-like particle which comprises the following three amino acid sequences KTTKQSFDLSVKAQYKKNKH (SEQ ID NO: 1), FGFPEHLLVDFLQSLS (SEQ ID NO: 2), and NVPEEDGTRFHRQASKC (SEQ ID NO: 3) or an equivalent of each thereof, in one or two or three epitope peptide(s) or
wherein the epitope(s), a peptide comprising at least one of the epitope(s), or a protein fragment comprising at least one of the epitope(s) is present on the outer surface of the virus or the virus-like particle; or
wherein the epitope(s), a peptide comprising at least one of the epitope(s), or a protein fragment comprising at least one of the epitope(s) is present on the outer surface of the virus or the virus-like particle; or
wherein the epitope(s), a peptide comprising at least one of the epitope(s), or a protein fragment comprising at least one of the epitope(s) is conjugated directly or indirectly to the virus or the virus-like particle, or a coat protein of the virus or the virus-like particle; or
wherein the epitope(s), a peptide comprising at least one of the epitope(s), or a protein fragment comprising at least one of the epitope(s) is conjugated indirectly comprising, or consisting essentially of, or yet further comprising of a linker to the virus or virus-like particle, or a coat protein of the virus or the virus-like particle.

8.-14. (canceled)

15. The method of claim 1, wherein the linker comprises an amino acid sequence of (GSG)n, (GPSL)n, or (GGSGGGSG)n, wherein n is an integer from 1 to 15, or wherein the linker is an SM(PEG)8 bifunctional linker comprising an NHS group and a maleimide group, or wherein the linker comprises an N-terminal cysteine residue conjugated to triple glycine (GGG) and an N-hydroxysuccinimide-PEG4-maleimide linker SM-PEG4.

16. (canceled)

17. The method of claim 1, wherein the virus or the virus-like particle is a bacteriophage virus or virus like particle, or a plant virus or virus like particle; or
wherein the virus or the virus-like particle is a bacteriophage Q virus or virus-like particle; or
wherein the virus or the virus-like particle is a plant picornavirus virus or virus-like particle, or a filamentous plant virus or virus-like particle.

18.-36. (canceled)

37. A method for one or more of the following in a subject in need thereof: treating or preventing an infectious disease or another disease caused by a pathogen, producing an antibody recognizing and binding to one or more pathogen(s) causing the disease, triggering, enhancing, or prolonging an immune response to one or more pathogen(s) causing the disease, or delivering at least one epitope(s) of the pathogen(s) causing the disease to the subject comprising administering to the subject one or more virus or virus-like particle(s), each of which comprises at least one epitope of the pathogen and optionally comprising two or more epitopes that may be the same or different from each other.

38.-82. (canceled)

83. A kit for use in a method of claim 1, comprising an optional instruction for use and at least one of: the one or more virus or virus-like particle(s) optionally comprising the at least one epitope(s), one or more of the epitope(s), one or more peptide(s) or protein fragment(s) each of which comprises at least one of the epitope(s), or one or more of the peptides as disclosed herein.

84. A composition comprising an optional carrier and one or more virus or virus-like particle(s), each of which comprises at least one epitope of a pathogen causing a disease or each of which comprises at least one epitope of one or more cholesterol checkpoint protein(s) and optionally comprising two or more epitopes that may be the same or different from each other.

85.-94. (canceled)

95. The composition of claim 84, wherein the epitope(s) is present in one or more peptide(s) comprising at least one amino acid sequence(s) selected from KGIYQTSN (SEQ ID NO: 5, amino acid (aa) 310 to aa 317 of SARS-CoV-2 S protein), AISSVLNDILSRLDKVE (SEQ ID NO: 6, aa 972 to aa 988 of SARS-CoV-2 S protein), KNHTSPDVDLGDISGIN (SEQ ID NO: 7, aa 1157 to aa 1173 of SARS-CoV-2 S protein), EIDRLNEVAKNLNESLIDLQEL (SEQ ID NO: 8, aa 1182 to aa 1209 of SARS-CoV-2 S protein), ATRFASVYAWNRKRISN (SEQ ID NO: 9, aa 346 to aa 362 of SARS-CoV-2 S protein), YNSASFSTFKCYGVSPTK (SEQ ID NO: 10, aa 369 to aa 386 of SARS-CoV-2 S protein), LPDPSKPSKRSFIED (SEQ ID NO: 11, aa 806 to aa 820 of SARS-CoV-2 S protein), FRKSN (SEQ ID NO: 12, aa 456 to aa 460 of SARS-CoV-2 S protein), PSKPSKRSFIEDLLFNKV (SEQ ID NO: 13, aa 809 to aa 826 of SARS-CoV-2 S protein), TESNKKFLPFQQFGRDIA (SEQ ID NO: 14, aa 553 to aa 570 of SARS-CoV-2 S protein), TESNKKFLPFQQ (SEQ ID NO: 15, aa 553 to aa 564 of SARS-CoV-2 S protein), HADQLTPTWRVY (SEQ ID NO: 16, aa 625 to aa 636 of SARS-CoV-2 S protein), FKEELDKYFKNH (SEQ ID NO: 17, aa 1148 to aa 1159 of SARS-CoV-2 S protein), FASTEKSNIIRGWIF (SEQ ID NO: 18, aa 92 to aa 106 of SARS-CoV-2 S protein), PFLGVYYHKNNKSWM (SEQ ID NO: 19, aa 135 to aa 153 of SARS-CoV-2 S protein), EVRQIAPGQTGKIAD (SEQ ID NO: 20, aa 406 to aa 420 of SARS-CoV-2 S protein), NNLDSKVGGNYNYLYR (SEQ ID NO: 21, aa 439 to aa 454 of SARS-CoV-2 S protein), LFRKSNLKPFERDIS (SEQ ID NO: 22, aa 455 to aa 469 of SARS-CoV-2 S protein), or a fragment of a coronavirus S protein aligned with each thereof, wherein the epitopes that may be the same or different from each other; or
wherein the epitope(s) is present in one or more peptide(s) comprising at least one amino acid sequence(s) selected from AISSVLNDILSRLDKVE (SEQ ID NO: 6, amino acid (aa) 972 to aa 988 of SARS-CoV-2 S protein), KNHTSPDVDLGDISGIN (SEQ ID NO: 7, amino acid (aa) 1157 to aa 1173 of SARS-CoV-2 S protein), EIDRLNEVAKNLNESLIDLQEL (SEQ ID NO: 8, amino acid (aa) 1182 to aa 1209 of SARS-CoV-2 S protein), YNSASFSTFKCYGVSPTK (aa 369 to aa 386 of SARS-CoV-2 S protein), PSKPSKRSFIEDLLFNKV (aa 809 to aa 826 of SARS-CoV-2 S protein), TESNKKFLPFQQFGRDIA (aa 553 to aa 570 of SARS-CoV-2 S protein), HADQLTPTWRVY (aa 625 to aa 636 of SARS-CoV-2 S protein), PFLGVYYHKNNKSWM (aa 135 to aa 153 of SARS-CoV-2 S protein), or a fragment of a coronavirus S protein aligned with each thereof.

96.-117. (canceled)

118. The composition of claim 84, wherein the virus or virus-like particle(s) is encapsulated within a degradable polymer matrix: optionally wherein the degradable polymer matrix comprises a melt processable degradable polymer material that is biocompatible and, upon degradation, produces substantially non-toxic products, wherein the melt processable degradable polymer material is a melt processable biodegradable polymer, and wherein the degradable polymer material has a melt temperature below the degradation temperature of the virus or virus-like particle(s).

119.-124. (canceled)


SEQUENCE LISTING (53 sequences)

SEQ ID NO: 1 - protein, 20 aa, Artificial Sequence
  Description of Artificial Sequence Synthetic peptide
KTTKQSFDLSVKAQYKKNKH

SEQ ID NO: 2 - protein, 16 aa, Artificial Sequence
  Description of Artificial Sequence Synthetic peptide
FGFPEHLLVDFLQSLS

SEQ ID NO: 3 - protein, 17 aa, Artificial Sequence
  Description of Artificial Sequence Synthetic peptide
NVPEEDGTRFHRQASKC

SEQ ID NO: 4 - protein, 15 aa, Alphapapillomavirus 9
QLYKTCKQAGTCPPD

SEQ ID NO: 5 - protein, 8 aa, Severe acute respiratory syndrome coronavirus 2
KGIYQTSN

SEQ ID NO: 6 - protein, 17 aa, Severe acute respiratory syndrome coronavirus 2
AISSVLNDILSRLDKVE

SEQ ID NO: 7 - protein, 17 aa, Severe acute respiratory syndrome coronavirus 2
KNHTSPDVDLGDISGIN

SEQ ID NO: 8 - protein, 22 aa, Severe acute respiratory syndrome coronavirus 2
EIDRLNEVAKNLNESLIDLQEL

SEQ ID NO: 9 - protein, 17 aa, Severe acute respiratory syndrome coronavirus 2
ATRFASVYAWNRKRISN

SEQ ID NO: 10 - protein, 18 aa, Severe acute respiratory syndrome coronavirus 2
YNSASFSTFKCYGVSPTK

SEQ ID NO: 11 - protein, 15 aa, Severe acute respiratory syndrome coronavirus 2
LPDPSKPSKRSFIED

SEQ ID NO: 12 - protein, 5 aa, Severe acute respiratory syndrome coronavirus 2
FRKSN

SEQ ID NO: 13 - protein, 18 aa, Severe acute respiratory syndrome coronavirus 2
PSKPSKRSFIEDLLFNKV

SEQ ID NO: 14 - protein, 18 aa, Severe acute respiratory syndrome coronavirus 2
TESNKKFLPFQQFGRDIA

SEQ ID NO: 15 - protein, 12 aa, Severe acute respiratory syndrome coronavirus 2
TESNKKFLPFQQ

SEQ ID NO: 16 - protein, 12 aa, Severe acute respiratory syndrome coronavirus 2
HADQLTPTWRVY

SEQ ID NO: 17 - protein, 12 aa, Severe acute respiratory syndrome coronavirus 2
FKEELDKYFKNH

SEQ ID NO: 18 - protein, 15 aa, Severe acute respiratory syndrome coronavirus 2
FASTEKSNIIRGWIF

SEQ ID NO: 19 - protein, 15 aa, Severe acute respiratory syndrome coronavirus 2
PFLGVYYHKNNKSWM

SEQ ID NO: 20 - protein, 15 aa, Severe acute respiratory syndrome coronavirus 2
EVRQIAPGQTGKIAD

SEQ ID NO: 21 - protein, 16 aa, Severe acute respiratory syndrome coronavirus 2
NNLDSKVGGNYNYLYR

SEQ ID NO: 22 - protein, 16 aa, Severe acute respiratory syndrome coronavirus 2
NNLDSKVGGNYNYLYR

SEQ ID NO: 23 - protein, 15 aa, Severe acute respiratory syndrome coronavirus 2
LFRKSNLKPFERDIS

SEQ ID NO: 24 - protein, 133 aa, Escherichia virus Qbeta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 25 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 26 - protein, 15 aa, Artificial Sequence
  Description of Artificial Sequence Synthetic peptide
  SITE (1)..(15): This sequence may encompass 1-5 “Gly Ser Gly” repeating units
GSGGSGGSGGSGGSG

SEQ ID NO: 27 - protein, 20 aa, Artificial Sequence
  Description of Artificial Sequence Synthetic peptide
  SITE (1)..(20): This sequence may encompass 1-5 “Gly Gly Ser Gly” repeating units
GGSGGGSGGGSGGGSGGGSG

SEQ ID NO: 28 - protein, 20 aa, Artificial Sequence
  Description of Artificial Sequence Synthetic peptide
  SITE (1)..(20): This sequence may encompass 1-5 “Gly Pro Ser Leu” repeating units
GPSLGPSLGPSLGPSLGPSL

SEQ ID NO: 29 - protein, 40 aa, Artificial Sequence
  Description of Artificial Sequence Synthetic polypeptide
  SITE (1)..(40): This sequence may encompass 1-5 “Gly Gly Ser Gly Gly Gly Ser Gly” repeating units
GGSGGGSGGGSGGGSGGGSGGGSGGGSGGGSGGGSGGGSG

SEQ ID NO: 30 - protein, 4 aa, Artificial Sequence
  Description of Artificial Sequence Synthetic peptide
GPSL

SEQ ID NO: 31 - protein, 8 aa, Artificial Sequence
  Description of Artificial Sequence Synthetic peptide
GGSGGGSG

SEQ ID NO: 32 - protein, 13 aa, Artificial Sequence
  Description of Artificial Sequence Synthetic peptide
CGGGFKGIYQTSN

SEQ ID NO: 33 - protein, 21 aa, Artificial Sequence
  Description of Artificial Sequence Synthetic peptide
CGGGATRFASVYAWNRKRISN

SEQ ID NO: 34 - protein, 21 aa, Artificial Sequence
  Description of Artificial Sequence Synthetic peptide
CGGGAISSVLNDILSRLDKVE

SEQ ID NO: 35 - protein, 21 aa, Artificial Sequence
  Description of Artificial Sequence Synthetic peptide
CGGGKNHTSPDVDLGDISGIN

SEQ ID NO: 36 - protein, 26 aa, Artificial Sequence
  Description of Artificial Sequence Synthetic peptide
CGGGEIDRLNEVAKNLNESLIDLQEL

SEQ ID NO: 37 - protein, 45 aa, Artificial Sequence
  Description of Artificial Sequence Synthetic polypeptide
  SITE (1)..(45): This sequence may encompass 1-15 “Gly Ser Gly” repeating units
GSGGSGGSGGSGGSGGSGGSGGSGGSGGSGGSGGSGGSGGSGGSG

SEQ ID NO: 38 - protein, 60 aa, Artificial Sequence
  Description of Artificial Sequence Synthetic polypeptide
  SITE (1)..(60): This sequence may encompass 1-15 “Gly Pro Ser Leu” repeating units
GPSLGPSLGPSLGPSLGPSLGPSLGPSLGPSLGPSLGPSLGPSLGPSLGPSLGPSLGPSL

SEQ ID NO: 39 - protein, 120 aa, Artificial Sequence
  Description of Artificial Sequence Synthetic polypeptide
  SITE (1)..(120): This sequence may encompass 1-15 “Gly Gly Ser Gly Gly Gly Ser Gly” repeating units
GGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 40 - protein, 24 aa, Artificial Sequence
  Description of Artificial Sequence Synthetic peptide
QLYKTCKQAGTCPPDGGSGGGSGC

SEQ ID NO: 41 - protein, 4 aa, Artificial Sequence
  Description of Artificial Sequence Synthetic peptide
CGGG

SEQ ID NO: 42 - protein, 4 aa, Artificial Sequence
  Description of Artificial Sequence Synthetic peptide
GSGG

SEQ ID NO: 43 - protein, 4 aa, Artificial Sequence
  Description of Artificial Sequence Synthetic peptide
GGSG

SEQ ID NO: 44 - protein, 31 aa, Severe acute respiratory syndrome coronavirus 2
GTGVLTESNKKFLPFQQFGRDIDDTTDAVRD

SEQ ID NO: 45 - protein, 31 aa, Severe acute respiratory syndrome coronavirus 2
GTGVLTESNKKFLPFQQFGRDIADTTDAVRD

SEQ ID NO: 46 - protein, 31 aa, Severe acute respiratory syndrome-related coronavirus
GTGVLTPSSKRFQPFQQFGRDVSDFTDSVRD

SEQ ID NO: 47 - protein, 25 aa, Severe acute respiratory syndrome coronavirus 2
EVPVAIHADQLTPTWRVYSTGSNVF

SEQ ID NO: 48 - protein, 25 aa, Severe acute respiratory syndrome-related coronavirus
DVSTAIHADQLTPAWRIYSTGNNVF

SEQ ID NO: 49 - protein, 33 aa, Severe acute respiratory syndrome coronavirus 2
NFSQILPDPSKPSKRSFIEDLLFNKVTLADAGF

SEQ ID NO: 50 - protein, 33 aa, Severe acute respiratory syndrome-related coronavirus
NFSQILPDPLKPTKRSFIEDLLFNKVTLADAGF

SEQ ID NO: 51 - protein, 24 aa, Artificial Sequence
  Description of Artificial Sequence Synthetic peptide
CGGSGGGSGQLYKTCKQAGTCPPD

SEQ ID NO: 52 - protein, 20 aa, Artificial Sequence
  Description of Artificial Sequence Synthetic peptide
QLYKTCKQAGTCPPDGPSLC

SEQ ID NO: 53 - protein, 20 aa, Artificial Sequence
  Description of Artificial Sequence Synthetic peptide
CGPSLQLYKTCKQAGTCPPD